(19) United States
(12) Patent Application Publication
(10) Pub. No.: US 2017/0119896 A1
(43) Pub. Date: May 4, 2017

(54) TERMINALLY MODIFIED POLYMERS AND CONJUGATES THEREOF

(71) Applicant: Mersana Therapeutics, Inc., Cambridge, MA (US)

(72) Inventors: Aleksandr V. Yurkovetskiy, Littleton, MA (US); Venu R. Gurijala, Lancaster, MA (US); Joshua D. Thomas, Natick, MA (US); Timothy B. Lowinger, Carlisle, MA (US)

(21) Appl. No.: 15/260,084

(22) Filed: Sep. 8, 2016

Related U.S. Application Data
(63) Continuation of application No. 13/934,931, filed on Jul. 3, 2013, now abandoned.
(60) Provisional application No. 61/668,179, filed on Jul. 5, 2012.
(60) Provisional application No. 61/794,304, filed on Mar. 15, 2013.

(51) Int. Cl. A61K 31/00; C07K 16/32; C08G 2/30; A61K 38/07; A61K 38/22

(52) U.S. Cl. CPC A61K 47/48192; A61K 47/48692; A61K 47/48415; A61K 38/07; A61K 47/48561; A61K 47/48669; A61K 47/48569; C07K 16/32; A61K 38/2278; C08G 2/30; C07K 2317/24; C08G 2230/00

(57) ABSTRACT

A terminally modified polymer is provided herein. At least one terminus of the polymer is —O—(CH₂)₂-L^{M} or —O—CH₂—CH(OH)—CH₂—CR¹═CR²R³. L^{M}, R¹, R², and R³ are defined herein Also disclosed are terminal conjugates comprising the polymer and a pharmaceutically useful modifier, as well as compositions comprising the conjugates, methods of their preparation, and methods of treating various disorders with the conjugates or their compositions.

### RELATED APPLICATIONS

This application is a continuation application of U.S. application Ser. No. 13/934,931, filed Jul. 3, 2013, which claims the benefit of and priority under 35 USC §119(e) to U.S. Provisional Application Nos. 61/668,179, filed Jul. 5, 2012; and 61/794,304, filed Mar. 15, 2013. The contents of each of these applications are hereby incorporated by reference in their entireties.

### BACKGROUND OF THE INVENTION

Traditionally, pharmaceuticals have primarily consisted of small molecules that are dispensed orally (as solid pills and liquids) or as injectables. Over the past three decades, formulations (i.e., compositions that control the route and/or rate of drug delivery and allow delivery of the therapeutic agent at the site where it is needed) have become increasingly common and complex. In addition, due to recent advances in genetic and cell engineering technologies, proteins known to exhibit various pharmacological actions in vivo are capable of production in large amounts for pharmaceutical applications. The availability of such recombinant proteins has engendered advances in protein formulation and chemical modification. Nevertheless, many questions and challenges regarding the development of new treatments as well as the mechanisms with which to administer them remain to be addressed. For example, many drugs exhibit limited or otherwise reduced potencies and therapeutic effects because they are either generally subject to partial degradation before they reach a desired target in the body, or accumulate in tissues other than the target, or both.

One objective in the field of drug delivery systems, therefore, is to deliver medications intact to specifically targeted areas of the body through a system that can stabilize the drug and control the in vivo transfer of the therapeutic agent utilizing either physiological or chemical mechanisms, or both. Over the past decade, materials such as polymeric microspheres, polymer micelles, soluble polymers and hydrogel-type materials have been shown to be effective in enhancing drug targeting specificity, lowering systemic drug toxicity, improving treatment absorption rates, and providing protection for pharmaceuticals against biochemical degradation, and thus have shown great potential for use in biomedical applications, particularly as components of drug delivery devices.

Synthetic polymers commonly used in medical applications and biomedical research include polyethyleneglycol (pharmacokinetics and immune response modifier), polyvinyl alcohol (drug carrier), and poly(hydroxypropylmetacrylamide) (drug carrier). In addition, natural polymers are also used in biomedical applications. For instance, dextran, hydroxyethylstarch, albumin and partially hydrolyzed proteins find use in applications ranging from plasma substitute, to radiopharmaceutical to parenteral nutrition. In general, synthetic polymers may offer greater advantages than natural materials in that they can be tailored to give a wider range of properties and more predictable lot-to-lot uniformity than can materials from natural sources. Synthetic polymers also represent a more reliable source of raw materials, one free from concerns of infection or immunogenicity. Methods of preparing polymeric materials are well known in the art. However, synthetic methods that successfully lead to the preparation of polymeric materials that exhibit adequate biodegradability, biocompatibility, hydrophilicity and minimal toxicity for biomedical use are scarce.

Accordingly, there is a need to design and modify engineer low-toxicity, biodegradable, biocompatible, hydrophilic polymers and conjugates thereof comprising pharmaceutically useful modifiers. Such polymer conjugates would find use in several applications, including components for biomedical preparations, pharmaceutical formulations, medical devices, implants, and the packaging/delivery of therapeutic, diagnostic and prophylactic agents.

### SUMMARY OF THE INVENTION

The present invention relates to a terminally modified polymer that is biodegradable, biocompatible and is capable of covalently conjugating with a pharmaceutically useful modifier (“M”) in a controllable manner. In particular, the terminally modified polymer is modified only at one of its terminus with a functional group that is capable of covalently conjugating with only one M, e.g., a protein-based recognition molecule (PBRM) or a therapeutic agent having a molecular weight ≦5 kDa (“D”).

In one aspect, the invention encompasses a terminally modified polymer for covalently conjugating with an M, wherein:
the polymer is a polyacetal or polyketal with a molecular weight between about 0.5 and about 300 kDa (e.g., 1 kDa to about 150 kDa or about 2 kDa to about 75 kDa),
at least one terminus of the polymer is —O—(CH₂)₂-L^{M} or —O—CH₂—CH(OH)—CH₂—CR¹═CR²R³, and
L^{M} is a linker capable of covalently conjugating with M and comprises a nitrogen-containing moiety selected from the group consisting of —NR¹, —NR¹C(═X¹)—, —NR¹C(═X¹)Y—, —NR¹NR²—, —NR¹NR²C(═X)—, —NR¹NR²C(═X¹)Y—, —NR¹SO₂—, and —NR¹SO₂NR²—, with the NR¹ moiety attached directly or indirectly to the polymer in the order as written, in which X¹ is O, S, or NR³ and Y is O, S, or NR⁴, and each of R¹, R², R³, and R⁴ independently is H or an aliphatic, heteroaliphatic, carbocyclic, or heterocyclic moiety.

The terminally modified polymer can include one or more of the following features when applicable.

The polymer does not contain —O—(CH₂)₂-L^{M} or —O—CH₂—CH(OH)—CH₂—CR¹═CR²R³ along the backbone of the polymer.

The polymer contains only one —O—(CH₂)₂-L^{M} or —O—CH₂—CH(OH)—CH₂—CR¹═CR²R³.

At least one terminus of the polymer is —O—(CH₂)₂-L^{M}.

L^{M} further includes

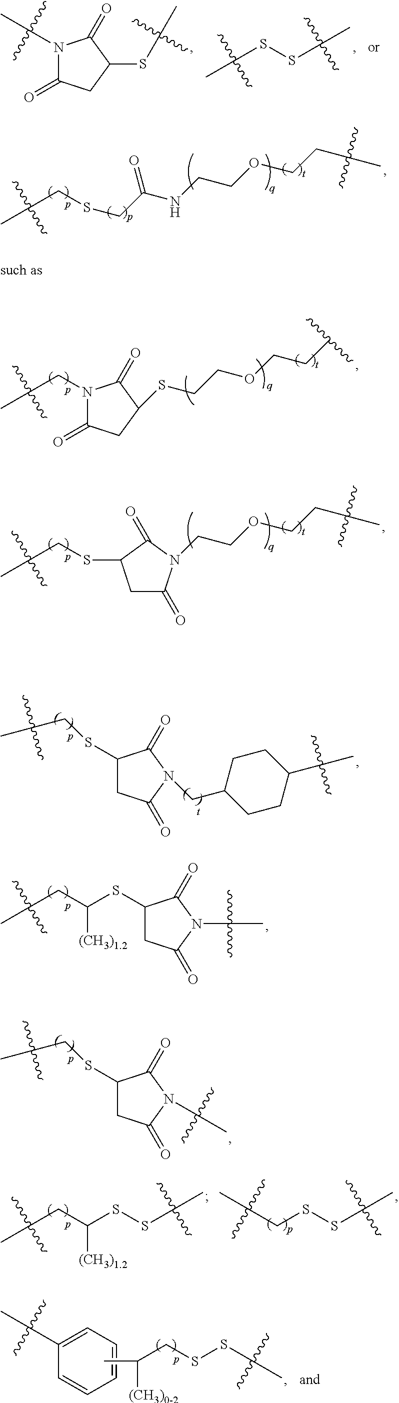

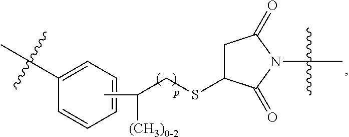

in which q is an integer from 0 to 12 and each of p and t independently is an integer from 0 to 3.

At least one terminus of the polymer is —O—CH₂—CH(OH)—CH₂—CR¹═CR²R³.

Each of R¹, R², and R³ is H.

The polymer is a polyacetal, such as poly(1-hydroxymethylethylene hydroxymethyl-formal), i.e., PHF.

For conjugating a PBRM, the terminally modified polymer of the invention comprises a polyacetal, e.g., a PHF having a molecular weight (i.e., MW of the unmodified PHF) ranging from about 0.5 kDa to about 150 kDa (e.g., about 2 kDa to about 110 kDa; or about 2 kDa to about 75 kDa.

For conjugating a PBRM having a molecular weight of 40 kDa or greater (e.g., 80 kDa or greater), terminally modified polymer of the invention comprises a polyacetal, e.g., a PHF having a molecular weight (i.e., MW of the unmodified PHF) ranging from about 2 kDa to about 25 kDa (e.g., about 4-15 kDa or about 4-10 kDa).

For conjugating a PBRM having a molecular weight of 200 kDa or less (e.g., 80 kDa or less), terminally modified polymer of the invention comprises a polyacetal, e.g., a PHF having a molecular weight (i.e., MW of the unmodified PHF) ranging from about 20 kDa to about 75 kDa (e.g., about 25-55 kDa).

The terminally modified polymer is of the following structure:

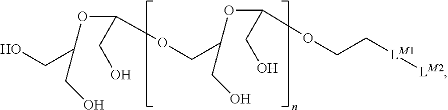

wherein
n is an integer between 1 and about 1100,
L^{M1} is —NR¹, —NR¹C(═X¹)—, —NR¹C(═X¹)Y—, —NR¹NR²—, —NR¹NR²C(═X¹)—, —NR¹NR²C(═X¹)Y—, —NR¹SO₂—, or —NR¹SO₂NR²—, with the NR¹ moiety attached to the polymer in the order as written, and
L^{M2} is —(CH₂)ₘ—W, with (CH₂)ₘ connected to L^{M1}, in which m is an integer between 0 and 20, and W, when not conjugated with M, is a functional group suitable for coupling (e.g., covalently conjugating) with M or W is an aliphatic, heteroaliphatic, carbocyclic, or heterocyclic moiety, wherein the aliphatic, heteroaliphatic, carbocyclic, or heterocycloalkyl moiety comprises a functional group suitable for coupling with M.

In one embodiment, each of R¹, R², R³ and R⁴ independently is H, or unsubstituted or substituted C₁₋₆ alkyl (e.g., C₁₋₆ alkyl substituted with amino, maleimide, carboxylic acid, ester, or other substituents disclosed herein).

The terminally modified polymer can further contain a pharmaceutically useful modifier (“M”) covalently attached to the polymer along the backbone of the polymer. M can be attached to the polymer directly or indirectly, e.g., via a linker. M can be a protein based recognition-molecule (“PBRM”) or a therapeutic agent having a molecular weight ≦5 kDa (“D”). When M is a PBRM, it can be connected to the backbone of the terminally modified polymer via L^{P} and when M is D, it can be connected to the backbone of the terminally modified polymer via L^{D}.

L^{D} can be a linker having the structure:

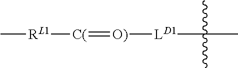

with R^{L1} connected to an oxygen atom of the polymeric carrier and L^{D1} connected to D, and

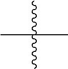

denotes direct or indirect attachment of D to L^{D1}, and L^{D} can contain a biodegradable bond so that when the bond is broken, D is released from the polymeric carrier in an active form for its intended therapeutic effect; L^{D1} can be a carbonyl-containing moiety; L^{P} can be a linker different from L^{D} and having the structure: —R^{L2}—C(═O)-L^{P1} with R^{L2} connected to an oxygen atom of the polymeric carrier and L^{P1} suitable for connecting directly or indirectly to a PBRM; each of R^{L1} and R^{L2} independently can be absent, alkyl, heteroalkyl, cycloalkyl, or heterocycloalkyl; and L^{P1} can be a moiety containing a functional group that is capable of forming a covalent bond with a functional group of a PBRM.

L^{P} can be a linker having the structure:

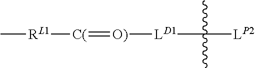

in which L^{P2} is a moiety containing a functional group that is capable of forming a covalent bond with a functional group of a PBRM, and

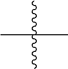

denotes direct or indirect attachment of L^{P2} to L^{D1}.

The functional group of L^{P1} or L^{P2} can be selected from —SR^{p}, —S—S-LG, maleimido, and halo, in which LG is a leaving group and R^{p} is H or a sulfur protecting group.

L^{D1} can include —X—(CH₂)ᵥ—C(═O)— with X directly connected to the carbonyl group of R^{L1}—C(═O), in which X is CH₂, O, or NH, and v is an integer from 1 to 6.

L^{P1} or L^{P2} can contain a biodegradable bond.

Each of R^{L1} and R^{L2} can be absent.

Each PBRM independently can be a protein, a peptide, a peptide mimetic, an antibody, or an antibody fragment.

Each occurrence of D independently can be selected from vinca alkaloids, auristatins, tubulysins, duocarmycins, PI3 kinases, MEK inhibitors, KSP inhibitors, and derivatives thereof.

The invention also features a polymer conjugate (i.e., a terminal conjugate) comprising a terminally modified polymer described above and a pharmaceutically useful modifier (“M”) covalently conjugated with L^{M} or —O—CH₂—CH(OH)—CH₂—CR¹═CR²R³ of the terminally modified polymer.

The polymer conjugate (i.e., a terminal conjugate) of the invention can include one or more of the following features when applicable.

The terminal conjugate is of formula (I):

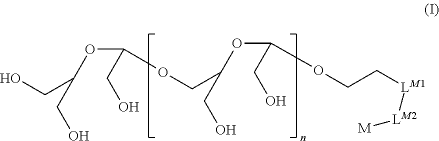

wherein
n is an integer between 1 and about 1100,
L^{M1} is —NR¹, —NR¹C(═X¹)—, —NR¹C(═X¹)Y—, —NR¹NR²—, —NR¹NR²C(═X¹)—, —NR¹NR²C(═X¹)Y—, —NR¹SO₂—, or —NR¹SO₂NR²—, with the NR¹ moiety attached to the polymer in the order as written, and
L^{M2} is —(CH₂)—ₘ—W—, with (CH₂)ₘ connected to L^{M1}, in which m is an integer between 0 and 20, and W, prior to conjugating with M, is a functional group suitable for coupling with M or W is an aliphatic, heteroaliphatic, carbocyclic, or heterocyclic moiety, wherein the aliphatic, heteroaliphatic, carbocyclic, or heterocycloalkyl moiety comprises a functional group suitable for coupling with M.

The polymer conjugate contains only one -L^{M}-M.

M is selected from the group consisting of proteins, antibodies, antibody fragments, peptides, drugs, hormones, cytokines, enzymes, enzyme substrates, receptor ligands, lipids, nucleotides, nucleosides, metal complexes, antibiotics, antigens, immunomodulators, and antiviral compounds.

M has a molecular weight ≦200 kDa, e.g., M is a PBRM.

M has a molecular weight ≦10 kDa.

W, prior to conjugating with M, is selected from:

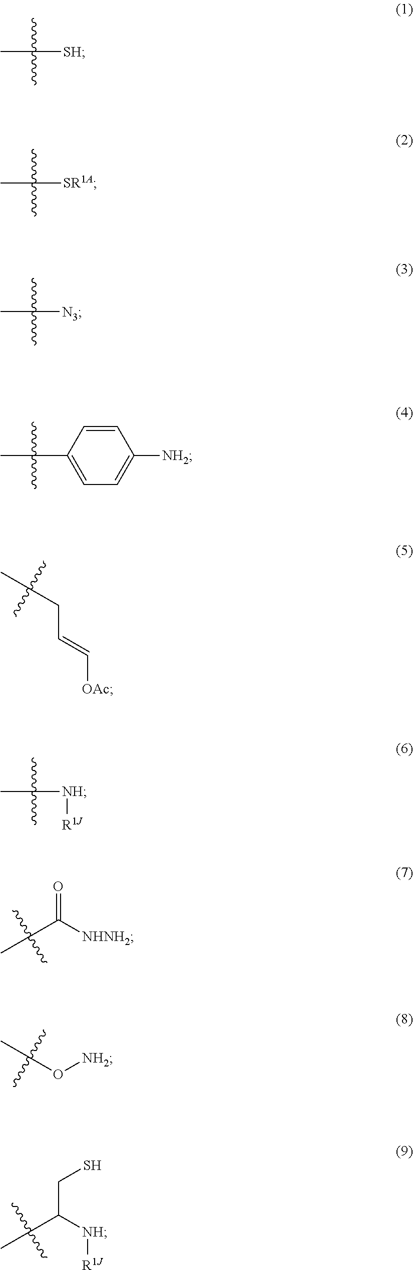

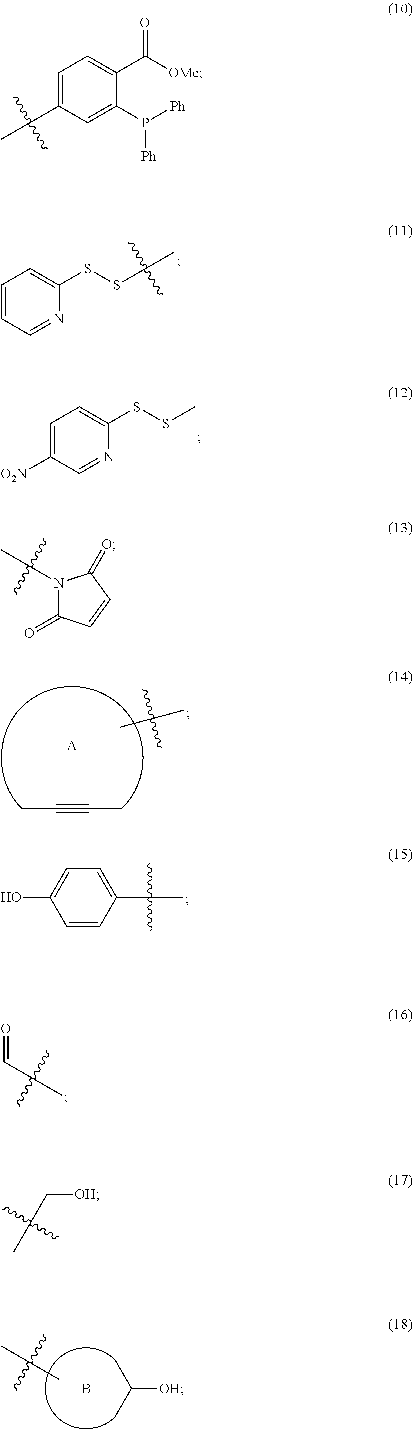

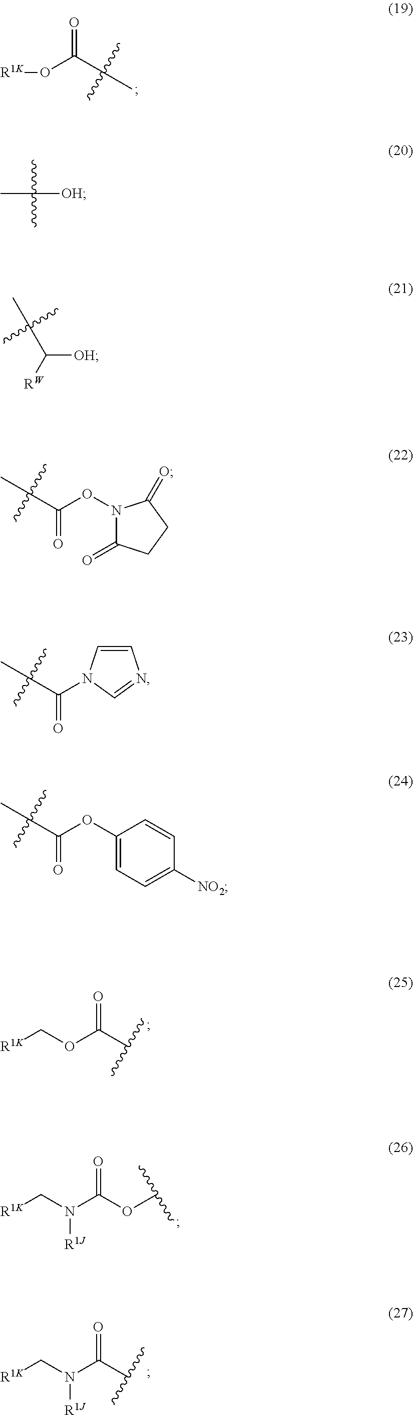

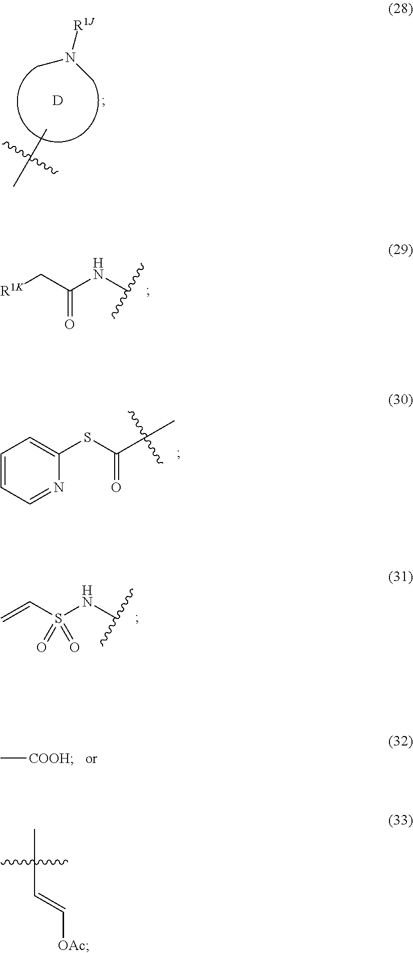

in which R^{1A} is a sulfur protecting group, each of ring A and B, independently, is cycloalkyl or heterocycloalkyl, R^{W} is an aliphatic, heteroaliphatic, carbocyclic or heterocycloalkyl moiety; ring D is heterocycloalkyl; R^{1J} is hydrogen, an aliphatic, heteroaliphatic, carbocyclic, or heterocycloalkyl moiety; and R^{1K} is a leaving group (e.g., halide or RC(O)O— in which R is hydrogen, an aliphatic, heteroaliphatic, carbocyclic, or heterocycloalkyl moiety).

Each R^{1A} independently is

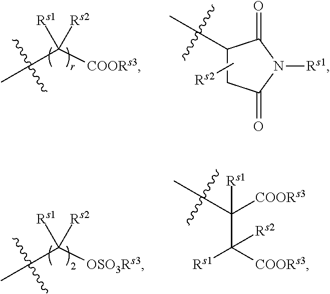

in which r is 1 or 2 and each of R^{s1}, R^{s2}, and R^{s3} is hydrogen, an aliphatic, heteroaliphatic, carbocyclic, or heterocycloalkyl moiety.

Ring A can be C₃₋₈ cycloalkyl or 5-19 membered heterocycloalkyl.

Ring A can be

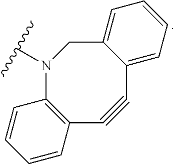 [SMILES: *N1Cc2ccccc2C#Cc2ccccc21]

Ring B can be C₃₋₈ cycloalkyl or 3-12 membered heterocycloalkyl.

Ring D can be piperazinyl or piperidinyl.

Each of R^{s1}, R^{s2}, and R^{s3} can be hydrogen or C₁₋₆ alkyl.

In another aspect, the invention provides compositions comprising the terminal conjugates, methods for their preparation, and methods of use thereof in the treatment of various disorders, including, but not limited to cancer.

In yet another aspect, the invention provides a method of synthesizing the terminally modified polymer described herein. The method includes providing a polyacetal or polyketal that has a terminal aldehyde group; reductively aminating the terminal aldehyde group to form a terminal amino group; and modifying the terminal amino group so as to obtain the terminally modified polymer of interest.

Also, the invention features a method of synthesizing a terminally modified polymer, at least one terminus of which is —O—CH₂—CH(OH)—CH₂—CR¹═CR²R³. The method includes providing a polyacetal or polyketal that has a terminal aldehyde group; and reacting the terminal aldehyde group with Z—CH₂—CR¹═CR²R³ to obtain the terminally modified polymer of interest. Z is halo.

Further, the invention features a method of synthesizing a terminally modified polymer described herein. The method includes providing a polyacetal or polyketal, wherein at least one terminus of the polyacetal or polyketal is —O—(CH₂)₂—NH₂; and reacting the —O—(CH₂)₂—NH₂ with

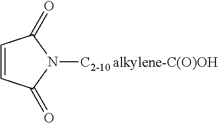

to obtain the terminally modified polymer.

In yet another aspect, the invention relates to a method of diagnosing a disorder in a subject suspected of having the disorder. The method comprises administering an effective amount of the conjugate described herein to the subject suspected of having the disorder or performing an assay to detect a target antigen/receptor in a sample from the subject so as to determine whether the subject expresses target antigen or receptor.

Unless otherwise defined, all technical and scientific terms used herein have the same meaning as commonly understood by one of ordinary skill in the art to which this invention belongs. In the specification, the singular forms also include the plural unless the context clearly dictates otherwise. Although methods and materials similar or equivalent to those described herein can be used in the practice or testing of the present invention, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting.

Advantages of the terminal modified polymer and terminal conjugate of the protein (or drug) per polymer chain include, enhanced plasma half life, reduced antigenicity and immunogenicity, increased solubility, increased stability and decreased proteolytic degradation of the protein (or drug) when compared with the non-conjugated counterparts. The factors which effect the foregoing properties include, but are not limited to, the nature of the protein (or drug), the chemistries (i.e. particular linkers) used to attach the polymer to the protein (or drug) and the location of the polymer-modified sites on the protein (or drug).

Another advantage of the terminal modified polymer and terminal conjugate of the invention is a 1:1 ratio of protein (or drug) per polymer chain. The advantages of this 1:1 ratio include control the loading of the protein (or drug) to optimize efficacy and to ensure dose to dose consistency by ensuring that the number of conjugated polymer molecules per protein is the same and that each polymer molecule is specifically covalently conjugated to the same amino acid residue in each protein molecule. The specific conjugation also avoids a wide distribution of conjugation products and a mixture thereof. Accordingly, purification of a conjugate obtained from the terminally modified polymer is easier and more cost effective. Further, the specific conjugation afforded by the terminally modified polymer also reduces the risk of a reduction or even a total loss of bioactivity of the protein (or drug). See, e.g., US 2011/0269974. Other advantages of the terminal conjugate of the invention include reduced modification of the polymeric carrier to maintain the biocompatibility and/or pharmacokinetics of the carrier.

Other features and advantages of the invention will be apparent from the following detailed description and claims.

### DETAILED DESCRIPTION OF CERTAIN EMBODIMENTS OF THE INVENTION

The present invention is based at least in part on an unexpected discovery of new methods of conjugating a peptide, protein, antibody, or drug to a terminally modified polyacetal. The new methods greatly enhance the yield and purity of the terminal conjugates. The terminally modified polyacetal conjugated with an M results in preservation of the activity of M.

Accordingly, the present disclosure provides the new methods and novel terminally modified polymers for covalently conjugating with an M. The present invention also provides novel terminally modified polymer-M conjugates (i.e., terminal conjugates), synthetic methods for making the conjugates, pharmaceutical compositions containing them and various uses of the conjugates.

### Definition/Terminology

Certain compounds of the present invention, and definitions of specific functional groups are also described in more detail herein. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference. Furthermore, it will be appreciated by one of ordinary skill in the art that the synthetic methods, as described herein, utilize a variety of protecting groups.

The use of the articles “a”, “an”, and “the” in both the following description and claims are to be construed to cover both the singular and the plural, unless otherwise indicated herein or clearly contradicted by context. The terms “comprising”, “having”, “including”, and “containing” are to be construed as open terms (i.e., meaning “including but not limited to”) unless otherwise noted. Additionally whenever “comprising” or another open-ended term is used in an embodiment, it is to be understood that the same embodiment can be more narrowly claimed using the intermediate term “consisting essentially of” or the closed term “consisting of.”

Recitation of ranges of values are merely intended to serve as a shorthand method of referring individually to each separate value falling within the range, unless otherwise indicated herein, and each separate value is incorporated into the specification as if it were individually recited herein. A range used herein, unless otherwise specified, includes the two limits of the range. For example, the expressions “x being an integer between 1 and 6” and “x being an integer of 1 to 6” both mean “x being 1, 2, 3, 4, 5, or 6”.

“Protecting group”: as used herein, the term protecting group means that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. For example, in certain embodiments, certain exemplary oxygen protecting groups may be utilized. These oxygen protecting groups include, but are not limited to methyl ethers, substituted methyl ethers (e.g., MOM (methoxymethyl ether), MTM (methylthiomethyl ether), BOM (benzyloxymethyl ether), and PMBM (p-methoxybenzyloxymethyl ether)), substituted ethyl ethers, substituted benzyl ethers, silyl ethers (e.g., TMS (trimethylsilyl ether), TES (triethylsilylether), TIPS (triisopropylsilyl ether), TBDMS (t-butyldimethylsilyl ether), tribenzyl silyl ether, and TBDPS (t-butyldiphenyl silyl ether), esters (e.g., formate, acetate, benzoate (Bz), trifluoroacetate, and dichloroacetate), carbonates, cyclic acetals and ketals. In certain other exemplary embodiments, nitrogen protecting groups are utilized. Nitrogen protecting groups, as well as protection and deprotection methods are known in the art. Nitrogen protecting groups include, but are not limited to, carbamates (including methyl, ethyl and substituted ethyl carbamates (e.g., Troc), amides, cyclic imide derivatives, N-Alkyl and N-Aryl amines, imine derivatives, and enamine derivatives. In yet other embodiments, certain exemplary sulphur protecting groups may be utilized. The sulfur protecting groups include, but are not limited to those oxygen protecting group describe above as well as aliphatic carboxylic acid (e.g., acrylic acid), maleimide, vinyl sulfonyl, and optionally substituted maleic acid. Certain other exemplary protecting groups are detailed herein, however, it will be appreciated that the present invention is not intended to be limited to these protecting groups; rather, a variety of additional equivalent protecting groups can be readily identified using the above criteria and utilized in the present invention. Additionally, a variety of protecting groups are described in “Protective Groups in Organic Synthesis” Third Ed. Greene, T. W. and Wuts, P. G., Eds., John Wiley & Sons, New York: 1999, the entire contents of which are hereby incorporated by reference.

“Leaving group” refers to a molecular fragment that departs with a pair of electrons in heterolytic bond cleavage. Leaving groups can be anions or neutral molecules. Leaving groups include, but are not limited to halides such as Cl⁻, Br⁻, and I⁻, sulfonate esters, such as para-toluenesulfonate (“tosylate”, TsO⁻), and RC(O)O⁻ in which R is hydrogen, an aliphatic, heteroaliphatic, carbocyclic, or heterocycloalkyl moiety.

All methods described herein can be performed in any suitable order unless otherwise indicated herein or otherwise clearly contradicted by context. The use of any and all examples, or exemplary language (e.g., “such as”) provided herein, is intended merely to better illustrate the invention and is not to be construed as a limitation on the scope of the claims unless explicitly otherwise claimed. No language in the specification is to be construed as indicating that any non-claimed element is essential to what is claimed.

“Antibody” refers to an immunoglobulin molecule of the class IgG including but not limited to IgG subclasses (IgG1, 2, 3 and 4) and class IgM which is able to specifically bind to a specific epitope on an antigen. Antibodies can be intact immunoglobulins derived from natural sources or from recombinant sources and can be immunoreactive portions of intact immunoglobulins. Antibodies may exist in a variety of forms including, for example, polyclonal antibodies, monoclonal antibodies, camelized single domain antibodies, intracellular antibodies (“intrabodies”), recombinant antibodies, anti-idiotypic antibodies, domain antibodies, linear antibody, multispecific antibody, antibody fragments, such as, Fv, Fab, Fab′, Fab′-SH, F(ab′)₂, single chain variable fragment antibodies (scFv), Fc, pFc′, scFvFc, disulfide Fv (dsfv), bispecific antibodies (bc-scFv) such as BiTE antibodies; camelid antibodies, resurfaced antibodies, humanized antibodies, fully human antibodies, single-domain antibody (sdAb, also known as NANOBODY®), chimeric antibodies, chimeric antibodies comprising at least one human constant region, dual-affinity antibodies such as, dual-affinity retargeting proteins (DART™), divalent (or bivalent) single-chain variable fragments (di-scFvs, bi-scFvs) including but not limited to minibodies, diabodies, triabodies or tribodies, tetrabodies, and the like, and multivalent antibodies. “Antibody fragment” refers to at least a portion of the variable region of the immunoglobulin molecule that binds to its target, i.e., the antigen-binding region. As used herein, the term “antibody” refers to both the full-length antibody and antibody fragments unless otherwise specified.

“Protein based recognition-molecule” or “PBRM” refers to a molecule that recognizes and binds to a cell surface marker or receptor such as, a transmembrane protein, surface immobilized protein, or proteoglycan. Examples of PBRMs include but are not limited to, antibodies (e.g., Trastuzumab, Cetuximab, Rituximab, Bevacizumab, Epratuzumab, Veltuzumab, Labetuzumab) or peptides (LHRH receptor targeting peptides, EC-1 peptides, AOD-like peptides), lipocalins, such as, for example, anticalins, proteins such as, for example, interferons, lymphokines, growth factors, colony stimulating factors, and the like, peptides or peptide mimics, and the like. The protein based recognition molecule, in addition to targeting the terminal conjugate to a specific cell, tissue or location, may also have certain therapeutic effect such as antiproliferative (cytostatic and/or cytotoxic) activity against a target cell or pathway. The protein based recognition molecule comprises or may be engineered to comprise at least one chemically reactive group such as, —COOH, primary amine, secondary amine —NHR, —SH, or a chemically reactive amino acid moiety or side chains such as, for example, tyrosine, histidine, cysteine, or lysine.

“Biocompatible” as used herein is intended to describe compounds that exert minimal destructive or host response effects while in contact with body fluids or living cells or tissues. Thus a biocompatible group, as used herein, refers to an aliphatic, cycloalkyl, heteroaliphatic, heterocycloalkyl, aryl, or heteroaryl moiety, which falls within the definition of the term biocompatible, as defined above and herein. The term “Biocompatibility” as used herein, is also taken to mean that the compounds exhibit minimal interactions with recognition proteins, e.g., naturally occurring antibodies, cell proteins, cells and other components of biological systems, unless such interactions are specifically desirable. Thus, substances and functional groups specifically intended to cause the above minimal interactions, e.g., drugs and prodrugs, are considered to be biocompatible. Preferably (with exception of compounds intended to be cytotoxic, such as, e.g., antineoplastic agents), compounds are “biocompatible” if their addition to normal cells in vitro, at concentrations similar to the intended systemic in vivo concentrations, results in less than or equal to 1% cell death during the time equivalent to the half-life of the compound in vivo (e.g., the period of time required for 50% of the compound administered in vivo to be eliminated/cleared), and their administration in vivo induces minimal and medically acceptable inflammation, foreign body reaction, immunotoxicity, chemical toxicity and/or other such adverse effects. In the above sentence, the term “normal cells” refers to cells that are not intended to be destroyed or otherwise significantly affected by the compound being tested.

“Biodegradable”: As used herein, “biodegradable” polymers are polymers that are susceptible to biological processing in vivo. As used herein, “biodegradable” compounds or moieties are those that, when taken up by cells, can be broken down by the lysosomal or other chemical machinery or by hydrolysis into components that the cells can either reuse or dispose of without significant toxic effect on the cells. The term “biocleavable” as used herein has the same meaning of “biodegradable”. The degradation fragments preferably induce little or no organ or cell overload or pathological processes caused by such overload or other adverse effects in vivo. Examples of biodegradation processes include enzymatic and non-enzymatic hydrolysis, oxidation and reduction. Suitable conditions for non-enzymatic hydrolysis of the biodegradable terminal conjugates (or their components, e.g., the biodegradable polymeric carrier and the linkers between the carrier and the antibody or the drug molecule) described herein, for example, include exposure of the biodegradable conjugates to water at a temperature and a pH of lysosomal intracellular compartment. Biodegradation of some terminal conjugates (or their components, e.g., the biodegradable polymeric carrier and the linkers between the carrier and the antibody or the drug molecule), can also be enhanced extracellularly, e.g. in low pH regions of the animal body, e.g. an inflamed area, in the close vicinity of activated macrophages or other cells releasing degradation facilitating factors. In certain preferred embodiments, the effective size of the polymer carrier at pH-7.5 does not detectably change over 1 to 7 days, and remains within 50% of the original polymer size for at least several weeks. At pH-5, on the other hand, the polymer carrier preferably detectably degrades over 1 to 5 days, and is completely transformed into low molecular weight fragments within a two-week to several-month time frame. Polymer integrity in such tests can be measured, for example, by size exclusion HPLC. Although faster degradation may be in some cases preferable, in general it may be more desirable that the polymer degrades in cells with the rate that does not exceed the rate of metabolization or excretion of polymer fragments by the cells. In preferred embodiments, the polymers and polymer biodegradation byproducts are biocompatible.

“Bioavailability”: The term “bioavailability” refers to the systemic availability (i.e., blood/plasma levels) of a given amount of drug or compound administered to a subject. Bioavailability is an absolute term that indicates measurement of both the time (rate) and total amount (extent) of drug or compound that reaches the general circulation from an administered dosage form.

“Hydrophilic”: The term “hydrophilic” as it relates to substituents on the polymer monomeric units does not essentially differ from the common meaning of this term in the art, and denotes chemical moieties which contain ionizable, polar, or polarizable atoms, or which otherwise may be solvated by water molecules. Thus a hydrophilic group, as used herein, refers to an aliphatic, cycloalkyl, heteroaliphatic, heterocycloalkyl, aryl or heteroaryl moiety, which falls within the definition of the term hydrophilic, as defined above. Examples of particular hydrophilic organic moieties which are suitable include, without limitation, aliphatic or heteroaliphatic groups comprising a chain of atoms in a range of between about one and twelve atoms, hydroxyl, hydroxyalkyl, amine, carboxyl, amide, carboxylic ester, thioester, aldehyde, nitryl, isonitryl, nitroso, hydroxylamine, mercaptoalkyl, heterocycle, carbamates, carboxylic acids and their salts, sulfonic acids and their salts, sulfonic acid esters, phosphoric acids and their salts, phosphate esters, polyglycol ethers, polyamines, polycarboxylates, polyesters and polythioesters. In preferred embodiments of the present invention, at least one of the polymer monomeric units include a carboxyl group (COOH), an aldehyde group (CHO), a methylol (CH₂OH) or a glycol (for example, CHOH—CH₂OH or CH—(CH₂OH)₂).

The term “hydrophilic” as it relates to the polymers of the invention generally does not differ from usage of this term in the art, and denotes polymers comprising hydrophilic functional groups as defined above. In a preferred embodiment, hydrophilic polymer is a water-soluble polymer. Hydrophilicity of the polymer can be directly measured through determination of hydration energy, or determined through investigation between two liquid phases, or by chromatography on solid phases with known hydrophobicity, such as, for example, C4 or C18.

“Polymeric Carrier”: The term polymeric carrier, as used herein, refers to a polymer or a modified polymer, which is suitable for covalently attaching to or can be covalently attached to one or more modifiers such as drug molecules or PBRMs with a designated linker.

“Terminus” or “termini” of a polymer or a polymeric carrier as used herein refers to one of the two ends of the backbone of the polymer or polymeric carrier when the polymer or polymeric carrier is linear or refers to one of the three or more ends of the backbone of the polymer or polymeric carrier when the polymer or polymeric carrier is branched. In other words, the term “terminus” of a polymer does not include any appending groups distributed along the polymer backbone such as the —CH₂OH group along the backbone of PHF. The term “terminally modified polymer” thus refers to a polymer whose terminus has been modified.

The term “terminal conjugate” as used herein refers to a polymer-modifier conjugate, in which the modifier is connected to one of the termini of the polymer. The terminal conjugate optionally can further contain one or more modifiers along the backbone of the polymer.

“Physiological conditions”: The phrase “physiological conditions”, as used herein, relates to the range of chemical (e.g., pH, ionic strength) and biochemical (e.g., enzyme concentrations) conditions likely to be encountered in the extracellular fluids of living tissues. For most normal tissues, the physiological pH ranges from about 7.0 to 7.4. Circulating blood plasma and normal interstitial liquid represent typical examples of normal physiological conditions.

“Polysaccharide”, “carbohydrate” or “oligosaccharide”: The terms “polysaccharide”, “carbohydrate”, or “oligosaccharide” are known in the art and refer, generally, to substances having chemical formula (CH₂O)ₙ, where generally n>2, and their derivatives. Carbohydrates are polyhydroxyaldehydes or polyhydroxyketones, or change to such substances on simple chemical transformations, such as hydrolysis, oxidation or reduction. Typically, carbohydrates are present in the form of cyclic acetals or ketals (such as, glucose or fructose). These cyclic units (monosaccharides) may be connected to each other to form molecules with few (oligosaccharides) or several (polysaccharides) monosaccharide units. Often, carbohydrates with well defined number, types and positioning of monosaccharide units are called oligosaccharides, whereas carbohydrates consisting of mixtures of molecules of variable numbers and/or positioning of monosaccharide units are called polysaccharides. The terms “polysaccharide”, “carbohydrate”, and “oligosaccharide”, are used herein interchangeably. A polysaccharide may include natural sugars (e.g., glucose, fructose, galactose, mannose, arabinose, ribose, and xylose) and/or derivatives of naturally occurring sugars (e.g., 2′-fluororibose, 2′-deoxyribose, and hexose).

“Pharmaceutically useful group or entity”: As used herein, this term refers to a compound or fragment thereof, or an organic moiety which, when associated with the polyal conjugates of the present invention, can exert some biological or diagnostic function or activity when administered to a subject, or enhance the therapeutic, diagnostic or preventive properties of the polyal conjugates in biomedical applications, or improve safety, alter biodegradation or excretion, or is detectable. Examples of suitable pharmaceutically useful groups or entities include hydrophilicity/hydrophobicity modifiers, pharmacokinetic modifiers, biologically active modifiers, detectable modifiers.

“Modifier” as used herein refers to an organic, inorganic or bioorganic moiety that is covalently incorporated into a carrier. Modifiers can be small molecules or macromolecules, and can belong to any chemical or pharmaceutical class, e.g., nucleotides, chemotherapeutic agents, antibacterial agents, antiviral agents, immunomodulators, hormones or analogs thereof, enzymes, inhibitors, alkaloids and therapeutic radionuclides. In certain embodiments, chemotherapeutic agents include, but are not limited to, topoisomerase I and II inhibitors, alkylating agents, anthracyclines, doxorubicin, cisplastin, carboplatin, vincristine, mitromycine, taxol, camptothecin, antisense oligonucleotides, ribozymes, and dactinomycines. In certain embodiments, modifiers according to the invention include, but are not limited to, biomolecules, small molecules, therapeutic agents, pharmaceutically useful groups or entities, a protein-based recognition molecules (PBRM), macromolecules, diagnostic labels, chelating agents, hydrophilic moieties, dispersants, charge modifying agents, viscosity modifying agents, surfactants, coagulation agents and flocculants, to name a few.

A modifier can have one or more pharmaceutical functions, e.g., biological activity and pharmacokinetics modification. Pharmacokinetics modifiers can include, for example, antibodies, antigens, receptor ligands, hydrophilic, hydrophobic or charged groups. Biologically active modifiers include, for example, therapeutic drugs and prodrugs, antigens, immunomodulators. Detectable modifiers include diagnostic labels, such as radioactive, fluorescent, paramagnetic, superparamagnetic, ferromagnetic, X-ray modulating, X-ray-opaque, ultrasound-reflective, and other substances detectable by one of available clinical or laboratory methods, e.g., scintigraphy, NMR spectroscopy, MRI, X-ray tomography, sonotomography, photoimaging, radioimmunoassay. Viral and non-viral gene vectors are considered to be modifiers.

“Macromolecule” as used herein refers to molecules, whether naturally-occurring or artificially created (e.g., via chemical synthesis) that have a relatively high molecular weight, generally above 1500 g/mole Preferred macromolecules are biologically active in that they exert a biological function in animals, preferably mammals, more preferably humans. Examples of macromolecules include proteins, enzymes, growth factors, cytokines, peptides, polypeptides, polylysine, proteins, lipids, polyelectrolytes, immunoglobulins, DNA, RNA, ribozymes, plasmids, and lectins. For the purpose of this invention, supramolecular constructs such as viruses and protein associates (e.g., dimers) are considered to be macromolecules. When associated with the polyal conjugates of the invention, a macromolecule may be chemically modified prior to being associated with said biodegradable biocompatible polyal conjugate.

“Small molecule”: As used herein, the term “small molecule” refers to molecules, whether naturally-occurring or artificially created (e.g., via chemical synthesis) that have a relatively low molecular weight. Preferred small molecules are biologically active in that they produce a local or systemic effect in animals, preferably mammals, more preferably humans. In certain preferred embodiments, the small molecule is a drug and the small molecule is referred to as “drug molecule” or “drug” or “therapeutic agent”. In certain embodiments, the drug molecule has MW less than or equal to about 5 kDa. In other embodiments, the drug molecule has MW less than or equal to about 1.5 kDa. In embodiments, the drug molecule is selected from vinca alkaloids, auristatins, tubulysins, duocarmycins, kinase inhibitors, MEK inhibitors, KSP inhibitors, and derivatives thereof. Preferably, though not necessarily, the drug is one that has already been deemed safe and effective for use by an appropriate governmental agency or body, e.g., the FDA. For example, drugs for human use listed by the FDA under 21 C.F.R. §§330.5, 331 through 361, and 440 through 460; drugs for veterinary use listed by the FDA under 21 C.F.R. §§500 through 589, incorporated herein by reference, are all considered suitable for use with the present hydrophilic polymers.

Classes of drug molecules that can be used in the practice of the present invention include, but are not limited to, anti-cancer substances, radionuclides, vitamins, anti-AIDS substances, antibiotics, immunosuppressants, anti-viral substances, enzyme inhibitors, neurotoxins, opioids, hypnotics, anti-histamines, lubricants, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic substances, anti-emetics, imaging agents. Many large molecules are also drugs.

A more complete, although not exhaustive, listing of classes and specific drugs suitable for use in the present invention may be found in “Pharmaceutical Substances: Syntheses, Patents, Applications” by Axel Kleemann and Jurgen Engel, Thieme Medical Publishing, 1999 and the “Merck Index: An Encyclopedia of Chemicals, Drugs, and Biologicals”, Edited by Susan Budavari et al., CRC Press, 1996, both of which are incorporated herein by reference. In preferred embodiments, the drug used in this invention is a therapeutic agent that has antiproliferative (cytostatic and/or cytotoxic) activity against a target cell or pathway. The drug may have a chemically reactive group such as, for example, —COOH, primary amine, secondary amine —NHR, —OH, —SH, —C(O)H, —C(O)R, —C(O)NHR^{2b}, C(S)OH, —S(O)₂OR^{2b}, —P(O)₂OR^{2b}, —CN, —NC or —ONO, in which R is an aliphatic, heteroaliphatic, carbocyclic or heterocycloalkyl moiety and R^{2b} is a hydrogen, an aliphatic, heteroaliphatic, carbocyclic, or heterocyclic moiety.

“Drug derivative” or “modified drug” or the like as used herein, refers to a compound that comprises the drug molecule intended to be delivered by the conjugate of the invention and a functional group capable of attaching the drug molecule to the polymeric carrier.

“Active form” as used herein refers to a form of a compound that exhibits intended pharmaceutical efficacy in vivo or in vitro. In particular, when a drug molecule intended to be delivered by the conjugate of the invention is released from the conjugate, the active form can be the drug itself or its derivatives, which exhibit the intended therapeutic properties. The release of the drug from the conjugate can be achieved by cleavage of a biodegradable bond of the linker which attaches the drug to the polymeric carrier. The active drug derivatives accordingly can comprise a portion of the linker.

“Diagnostic label”: As used herein, the term diagnostic label refers to an atom, group of atoms, moiety or functional group, a nanocrystal, or other discrete element of a composition of matter, that can be detected in vivo or ex vivo using analytical methods known in the art. When associated with a conjugate of the present invention, such diagnostic labels permit the monitoring of the conjugate in vivo. Alternatively or additionally, constructs and compositions that include diagnostic labels can be used to monitor biological functions or structures. Examples of diagnostic labels include, without limitation, labels that can be used in medical diagnostic procedures, such as, radioactive isotopes (radionuclides) for gamma scintigraphy and Positron Emission Tomography (PET), contrast agents for Magnetic Resonance Imaging (MRI) (for example paramagnetic atoms and superparamagnetic nanocrystals), contrast agents for computed tomography and other X-ray-based imaging methods, agents for ultrasound-based diagnostic methods (sonography), agents for neutron activation (e.g., boron, gadolinium), fluorophores for various optical procedures, and, in general moieties which can emit, reflect, absorb, scatter or otherwise affect electromagnetic fields or waves (e.g. gamma-rays, X-rays, radiowaves, microwaves, light), particles (e.g. alpha particles, electrons, positrons, neutrons, protons) or other forms of radiation, e.g. ultrasound.

“Aliphatic”: In general, the term aliphatic, as used herein, includes both saturated and unsaturated, straight chain (i.e., unbranched) or branched aliphatic hydrocarbons, which are optionally substituted with one or more functional groups. As will be appreciated by one of ordinary skill in the art, “aliphatic” is intended herein to include, but is not limited to, alkyl, alkenyl, alkynyl moieties. Thus, as used herein, the term “alkyl” includes straight and branched alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups. In certain embodiments, as used herein, “lower alkyl” is used to indicate those alkyl groups (substituted, unsubstituted, branched or unbranched) having about 1-6 carbon atoms.

“Alkenyl”: the term alkenyl denotes a monovalent group derived from a hydrocarbon moiety having at least one carbon-carbon double bond by the removal of a single hydrogen atom. “Substituted alkenyl” groups are substituted with one or more functional groups. Substituents include, but are not limited to, any of the substituents mentioned below, i.e., the substituents recited below resulting in the formation of a stable compound. Alkenyl groups include, for example, ethenyl, propenyl, butenyl, 1-methyl-2-buten-1-yl, and the like.

“Alkynyl”: the term alkynyl as used herein refers to a monovalent group derived from a hydrocarbon having at least one carbon-carbon triple bond by the removal of a single hydrogen atom. “Substituted alkenyl” groups are substituted with one or more functional groups. Substituents include, but are not limited to, any of the substituents mentioned below, i.e., the substituents recited below resulting in the formation of a stable compound. Representative alkynyl groups include ethynyl, 2-propynyl (propargyl), 1-propynyl, and the like.

In certain embodiments, the alkyl, alkenyl and alkynyl groups employed in the invention contain about 1-20 aliphatic carbon atoms. In certain other embodiments, the alkyl, alkenyl, and alkynyl groups employed in the invention contain about 1-10 aliphatic carbon atoms. In yet other embodiments, the alkyl, alkenyl, and alkynyl groups employed in the invention contain about 1-8 aliphatic carbon atoms. In still other embodiments, the alkyl, alkenyl, and alkynyl groups employed in the invention contain about 1-6 aliphatic carbon atoms. In yet other embodiments, the alkyl, alkenyl, and alkynyl groups employed in the invention contain about 1-4 carbon atoms. Illustrative aliphatic groups thus include, but are not limited to, for example, methyl, ethyl, n-propyl, isopropyl, allyl, n-butyl, sec-butyl, isobutyl, tert-butyl, n-pentyl, sec-pentyl, isopentyl, tert-pentyl, n-hexyl, sec-hexyl, moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl and the like.

“Alkylene” as used herein, the term alkylene by itself or part of another term refers to a saturated, branched or straight chain having two monovalent radical centers derived by the removal of two hydrogen atoms from the same or two different carbon atoms of a parent alkane. Alkylene radicals include, but are not limited to, methylene, 1,2, ethylene, 1,3-propyl, and the like. Suitable alkylenes include, but are not limited to methylene, ethylene, propylene, butylene, pentylene, hexylene, heptylene, ocytylene, nonylene, decalene, and the like. The term “cycloalkylene” similarly refers to bivalent cycloalkyl. Cycloalkylene radicals include, but are not limited to, 1,1-cyclopentylene, 1,2-cyclopentylene, 1,1-cyclobutylene, 1,3-cyclobutylene, etc.

“Heteroaliphatic”: as used herein, the term heteroaliphatic refers to aliphatic moieties in which one or more carbon atoms in the main chain have been substituted with a heteroatom. Thus, a heteroaliphatic group refers to an aliphatic chain which contains one or more oxygen, sulfur, nitrogen, phosphorus or silicon atoms, e.g., in place of carbon atoms. Heteroaliphatic moieties may be branched or linear unbranched. In certain embodiments, heteroaliphatic moieties are substituted (“substituted heteroaliphatic”) by independent replacement of one or more of the hydrogen atoms thereon with one or more moieties including, but not limited to aliphatic; heteroaliphatic; cycloalkyl; heterocycloalkyl; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —NO₂; —CN; —CF₃; —CH₂CF₃; —CHCl₂; —CH₂OH; —CH₂CH₂OH; —CH₂NH₂; —CH₂SO₂CH₃; - or -GR^{G1} wherein G is —O—, —S—, —NR^{G2-}, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O—, —C(═NR^{G2})NR^{G3}—, —OC(═NR^{G2})—, —NR^{G2}C(═NR^{G3})—, —NR^{G2}SO₂—, —NR^{G2}SO₂NR^{G3}—, or —SO₂NR^{G2}—, wherein each occurrence of R^{G1}, R^{G2} and R^{G3} independently includes, but is not limited to, hydrogen, halogen, or an optionally substituted aliphatic, heteroaliphatic, cycloalkyl, heterocycloalkyl, aryl, heteroaryl, alkylaryl, or alkylheteroaryl moiety. Additional examples of generally applicable substituents are illustrated by the specific embodiments shown in the Examples that are described herein.

“Cycloalkyl”: as used herein, the term cycloalkyl refers to a saturated or unsaturated nonaromatic hydrocarbon mono- or multi-ring system having 3 to 30 carbon atoms (e.g., C₃-C₁₀). Suitable cycloalkyls include, but are not limited to cyclopropyl, cyclobutyl, cyclopentyl, cyclohexyl, cycloheptyl, cyclooctyl, cyclopentenyl, cyclohexenyl, cycloheptenyl, cycloheptynyl, adamantyl, and the like.

“Heterocycloalkyl” as used herein refers to a saturated or unsaturated nonaromatic 3-8 membered monocyclic, 8-12 membered bicyclic, or 11-19 membered tricyclic ring system having one or more heteroatoms (such as O, N, S, or Se), unless specified otherwise. In certain embodiments, the term “heterocycloalkyl” refers to a non-aromatic 5-, 6-, 7- or 8-membered ring or a polycyclic group, including, but not limited to a bi- or tri-cyclic group comprising fused six-membered rings having between one and three heteroatoms independently selected from oxygen, sulfur and nitrogen, wherein (i) each 5-membered ring has 0 to 2 double bonds and each 6-membered ring has 0 to 2 double bonds, (ii) the nitrogen and sulfur heteroatoms may optionally be oxidized, (iii) the nitrogen heteroatom may optionally be quaternized, and (iv) any of the above heterocycloalkyl; rings may be fused to an aryl or heteroaryl ring. Examples of heterocycloalkyl groups include, but are not limited to, piperidinyl, piperazinyl, pyrrolidinyl, dioxanyl, tetrahydrofuranyl, tetrahydrothienyl, isoindolinyl, indolinyl, imidazolidinyl, pyrazolidinyl, oxazolidinyl, isoxazolidinyl, triazolidinyl, tetrahyrofuranyl, oxiranyl, azetidinyl, oxetanyl, thietanyl, 1,2,3,6-tetrahydropyridinyl, tetrahydro-2H-pyranyl, 3,6-dihydro-2H-pyranyl, morpholinyl, and the like.

“Aryl”: as used herein, refers to groups with aromaticity, including “conjugated,” or multicyclic systems with at least one aromatic ring and do not contain any heteroatom in the ring structure. Examples include phenyl, benzyl, 1,2,3,4-tetrahydronaphthalenyl, etc.

“Heteroaryl”: as used herein, refers to aryl groups, as defined above, except having from one to four heteroatoms in the ring structure, and may also be referred to as “aryl heterocycles” or “heteroaromatics.” As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidized (i.e., N→O and S(O)ₚ, where p=1 or 2). It is to be noted that total number of S and O atoms in the aromatic heterocycle is not more than 1. Examples of heteroaryl include pyridyl, pyrazinyl, pyrimidinyl, pyrrolyl, pyrazolyl, imidazolyl, thiazolyl, isothiazolyl, tetrazolyl, oxazolyl, isooxazolyl, thiadiazolyl, oxadiazolyl, thiophenyl, furanyl, quinolinyl, isoquinolinyl, tetrazolyl, pyridazinyl, quinazolinyl, dihydroquinazolyl, and tetrahydroquinazolyl and the like.

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, naphthrydine, indole, benzofuran, purine, benzofuran, deazapurine, indolizine.

In the case of multicyclic aromatic rings, only one of the rings needs to be aromatic (e.g., 2,3-dihydroindole), although all of the rings may be aromatic (e.g., quinoline). The second ring can also be fused or bridged.

“Carbocycle” or “carbocyclic moiety” as used herein, is intended to include any stable monocyclic, bicyclic or tricyclic ring having the specified number of carbons, any of which may be saturated, unsaturated, or aromatic. Carbocycle includes cycloalkyl and aryl. For example, a C₃-C₁₄ carbocycle is intended to include a monocyclic, bicyclic or tricyclic ring having 3, 4, 5, 6, 7, 8, 9, 10, 11, 12, 13 or 14 carbon atoms. Examples of carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, cyclooctyl, cyclooctenyl, cyclooctadienyl, fluorenyl, phenyl, naphthyl, indanyl, adamantyl and tetrahydronaphthyl. Bridged rings are also included in the definition of carbocycle, including, for example, [3.3.0]bicyclooctane, [4.3.0]bicyclononane, [4.4.0]bicyclodecane and [2.2.2]bicyclooctane. A bridged ring occurs when one or more carbon atoms link two non-adjacent carbon atoms. In one embodiment, bridge rings are one or two carbon atoms. It is noted that a bridge always converts a monocyclic ring into a tricyclic ring. When a ring is bridged, the substituents recited for the ring may also be present on the bridge. Fused (e.g., naphthyl, tetrahydronaphthyl) and spiro rings are also included.

“Heterocycle” or “heterocyclic moiety” as used herein, includes any ring structure (saturated, unsaturated, or aromatic) which contains at least one ring heteroatom (e.g., N, O or S). Heterocycle includes heterocycloalkyl and heteroaryl. Examples of heterocycles include, but are not limited to, morpholine, pyrrolidine, tetrahydrothiophene, piperidine, piperazine and tetrahydrofuran.

Examples of heterocyclic groups include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, 1,2,4-oxadiazol5(4H)-one, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl and xanthenyl. Multiple-ring heterocycle can include fused, bridged or spiro rings.

The cycloalkyl, heterocycloalkyl, aryl, or heteroaryl ring (or the carbocyclic or heterocyclic group) can be substituted at one or more ring positions (e.g., the ring-forming carbon or heteroatom such as N) with such substituents as described above, for example, aliphatic; heteroaliphatic; cycloalkyl; heterocycloalkyl; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —NO₂; —CN; —CF₃; —CH₂CF₃; —CHCl₂; —CH₂OH; —CH₂CH₂OH; —CH₂NH₂; —CH₂SO₂CH₃; - or -GR^{G1} wherein G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O—, —C(═NR^{G2})NR^{G3}—, —OC(═NR^{G2})—, —NR^{G2}C(═NR^{G3})—, —NR^{G2}SO₂—, —NR^{G2}SO₂NR^{G3}—, or —SO₂NR^{G2}—, wherein each occurrence of R^{G1}, R^{G2} and R^{G3} independently includes, but is not limited to, hydrogen, halogen, or an optionally substituted aliphatic, heteroaliphatic, cycloalkyl, heterocycloalkyl; aryl, heteroaryl, alkylaryl, or alkylheteroaryl moiety. Aryl and heteroaryl groups can also be fused or bridged with cycloalkyl or heterocyclic rings, which are not aromatic so as to form a multicyclic system (e.g., tetralin, methylenedioxyphenyl).

“Alkoxy” (or “alkyloxy”): as used herein, the term alkoxy (or alkyloxy) refers to an alkyl group, as previously defined, attached to the parent molecular moiety through an oxygen atom (“alkoxy”). In certain embodiments, the alkyl group contains about 1-20 aliphatic carbon atoms. In certain other embodiments, the alkyl group contains about 1-10 aliphatic carbon atoms. In yet other embodiments, the alkyl group contains about 1-8 aliphatic carbon atoms. In still other embodiments, the alkyl group contains about 1-6 aliphatic carbon atoms. In yet other embodiments, the alkyl group contains about 1-4 aliphatic carbon atoms. Examples of alkoxy groups, include but are not limited to, methoxy, ethoxy, propoxy, isopropoxy, n-butoxy, tert-butoxy, neopentoxy and n-hexoxy.

“Aryloxy”: as used herein, the term aryloxy refers to an aryl group, as defined herein, attached to the parent molecular moiety through an oxygen atom. Examples of aryloxy groups include but are not limited to phenoxy and napthyloxy.

“Heteroaryloxy”: as used herein, the term heteroaryloxy refers to a heteroaryl group, as defined herein, attached to the parent molecular moiety through an oxygen atom. Examples of heteroaryloxy groups include but are not limited to, quinolyloxy and isoquinolizinyloxy.

“Amine”: the term amine refers to a group having the structure —N(R)₂ wherein each occurrence of R is independently hydrogen, or an aliphatic or heteroaliphatic moiety, or the R groups, taken together, may form a heterocyclic moiety. In certain instances, an amine group can be charged (protonized) or quarternized, e.g., —HN⁺(R)₂ or —N⁺(R)₃

“Alkylamino”: as used herein, the term alkylamino refers to a group having the structure —NHR′ wherein R′ is alkyl, as defined herein. The term “aminoalkyl” refers to a group having the structure NH₂R′—, wherein R′ is alkyl, as defined herein. In certain embodiments, the alkyl group contains about 1-20 aliphatic carbon atoms. In certain other embodiments, the alkyl group contains about 1-10 aliphatic carbon atoms. In yet other embodiments, the alkyl, alkenyl, and alkynyl groups employed in the invention contain about 1-8 aliphatic carbon atoms. In still other embodiments, the alkyl group contains about 1-6 aliphatic carbon atoms. In yet other embodiments, the alkyl group contains about 1-4 aliphatic carbon atoms. Examples of alkylamino include, but are not limited to, methylamino, ethylamino, iso-propylamino and the like.

“Alkylthio” (or “thioalkyl”) means an alkyl group as defined herein with the indicated number of carbon atoms attached through a sulfur atom. C₁₋₆ alkylthio, is intended to include C₁, C₂, C₃, C₄, C₅, and C₆ alkylthio groups. C₁₋₈ alkylthio, is intended to include C₁, C₂, C₃, C₄, C₅, C₆, C₇, and C₈ alkylthio groups. The thioalkyl groups can be substituted with groups such as alkyl, alkenyl, alkynyl, halogen, hydroxyl, alkylcarbonyloxy, arylcarbonyloxy, alkoxycarbonyloxy, aryloxycarbonyloxy, carboxylate, carboxyacid, alkylcarbonyl, arylcarbonyl, alkoxycarbonyl, aminocarbonyl, alkylaminocarbonyl, dialkylaminocarbonyl, alkylthiocarbonyl, alkoxyl, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl alkylaryl, or an aryl or heteroaryl moieties.

“Thiocarbonyl” or “thiocarboxy” includes compounds and moieties which contain a carbon connected with a double bond to a sulfur atom.

“Thioether” includes moieties which contain a sulfur atom bonded to two carbon atoms or heteroatoms. Examples of thioethers include, but are not limited to alkthioalkyls, alkthioalkenyls and alkthioalkynyls. The term “alkthioalkyls” include moieties with an alkyl, alkenyl or alkynyl group bonded to a sulfur atom which is bonded to an alkyl group. Similarly, the term “alkthioalkenyls” refers to moieties wherein an alkyl, alkenyl or alkynyl group is bonded to a sulfur atom which is covalently bonded to an alkenyl group; and alkthioalkynyls” refers to moieties wherein an alkyl, alkenyl or alkynyl group is bonded to a sulfur atom which is covalently bonded to an alkynyl group.

“Arylthio” (or “thioaryl”) means an aryl group as defined herein with the indicated number of carbon atoms attached through a sulfur atom.

“Carboxylic acid” as used herein refers to a compound comprising a group of formula —CO₂H.

“Dicarboxylic acid” refers to a compound comprising two groups of formula —CO₂H.

“Halo, halide and halogen”: The terms halo, halide and halogen as used herein refer to an atom selected from fluorine, chlorine, bromine, and iodine.

“Methylol”: The term methylol as used herein refers to an alcohol group of the structure —CH₂OH.

“Hydroxyalkyl”: As used herein, the term hydroxyalkyl refers to an alkyl group, as defined above, bearing at least one OH group.

“Mercaptoalkyl”: The term mercaptoalkyl as used therein refers to an alkyl group, as defined above, bearing at least one SH group

“Acyl” includes moieties that contain the acyl radical (—C(O)—) or a carbonyl group. “Substituted acyl” includes acyl groups where one or more of the hydrogen atoms are replaced by, for example, alkyl groups, alkynyl groups, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aryl or heteroaryl moiety.

“Hydrocarbon”: The term hydrocarbon, as used herein, refers to any chemical group comprising hydrogen and carbon. The hydrocarbon may be substituted or unsubstituted. The hydrocarbon may be unsaturated, saturated, branched, unbranched, cyclic, polycyclic, or heterocyclic. Illustrative hydrocarbons include, for example, methyl, ethyl, n-propyl, iso-propyl, cyclopropyl, allyl, vinyl, n-butyl, tert-butyl, ethynyl, cyclohexyl, methoxy, diethylamino, heterocycloalkyl, aryl, heteroaryl, thioalkyl, and the like. As would be known to one skilled in this art, all valencies must be satisfied in making any substitutions.

“Alkylaryl” as used herein refers to an aryl group substituted with one or more alkyl groups (e.g., methylphenyl).

“Alkylarylamino” as used herein refers to —N R^{G4}R^{G5}, wherein R^{G4} is alkyl, as defined herein, and R^{G5} is an aryl, as defined herein, or at least one of R^{G4} and R^{G5} is an alkylaryl as defined herein.

“Substituted”: The terms substituted, whether preceded by the term “optionally” or not, and substituent, as used herein, refers to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Heteroatoms such as nitrogen may have hydrogen substituents and/or any permissible substituents of organic compounds described herein which satisfy the valencies of the heteroatoms. Examples of substituents include, but are not limited to aliphatic; heteroaliphatic; cycloalkyl; heterocycloalkyl; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —NO₂; —CN; —CF₃; —CH₂CF₃; —CHCl₂; —CH₂OH; —CH₂CH₂OH; —CH₂NH₂; —CH₂SO₂CH₃; - or -GR^{G1} wherein G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O, —C(═NR^{G2})NR^{G3}—, —OC(═NR^{G2})—, —NR^{G2}C(═NR^{G3})—, —NR^{G2}SO₂—, —NR^{G2}SO₂NR^{G3}—, or —SO₂NR^{G2}—, wherein each occurrence of R^{G1}, R^{G2} and R^{G3} independently includes, but is not limited to, hydrogen, halogen, or an optionally substituted aliphatic, heteroaliphatic, cycloalkyl, heterocycloalkyl, aryl, heteroaryl, alkylaryl, or alkylheteroaryl moiety. Additional examples of generally applicable substituents are illustrated by the specific embodiments shown in the Examples that are described herein.

The following are more general terms used throughout the present application:

“Animal”: The term animal, as used herein, refers to humans as well as non-human animals, at any stage of development, including, for example, mammals, birds, reptiles, amphibians, fish, worms and single cells. Cell cultures and live tissue samples are considered to be pluralities of animals. Preferably, the non-human animal is a mammal (e.g., a rodent, a mouse, a rat, a rabbit, a monkey, a dog, a cat, a primate, or a pig). An animal may be a transgenic animal or a human clone. The term “subject” encompasses animals.

“Efficient amount”: In general, as it refers to an active agent or drug delivery device, the term “efficient amount” refers to the amount necessary to elicit the desired biological response. As will be appreciated by those of ordinary skill in this art, the efficient amount of an agent or device may vary depending on such factors as the desired biological endpoint, the agent to be delivered, the composition of the encapsulating matrix, the target tissue, etc. For example, the efficient amount of microparticles containing an antigen to be delivered to immunize an individual is the amount that results in an immune response sufficient to prevent infection with an organism having the administered antigen.

“Natural amino acid” as used herein refers to any one of the common, naturally occurring L-amino acids found in naturally occurring proteins: glycine (Gly), alanine (Ala), valine (Val), leucine (Leu), isoleucine (Ile), lysine (Lys), arginine (Arg), histidine (His), proline (Pro), serine (Ser), threonine (Thr), phenylalanine (Phe), tyrosine (Tyr), tryptophan (Trp), aspartic acid (Asp), glutamic acid (Glu), asparagine (Asn), glutamine (Gln), cysteine (Cys) and methionine (Met).

“Unnatural amino acid” as used herein refers to any amino acid which is not a natural amino acid. This includes, for example, amino acids that comprise α-, β-, ω-, D-, L-amino acyl residues. More generally, the unnatural amino acid comprises a residue of the general formula

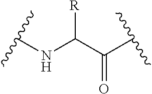

wherein the side chain R is other than the amino acid side chains occurring in nature. Exemplary unnatural amino acids, include, but are not limited to, sarcosine (N-methylglycine), citrulline (cit), homocitrulline, β-ureidoalanine, thiocitrulline, hydroxyproline, allothreonine, pipecolic acid (homoproline), α-aminoisobutyric acid, tert-butylglycine, tert-butylalanine, allo-isoleucine, norleucine, α-methylleucine, cyclohexylglycine, β-cyclohexylalanine, β-cyclopentylalanine, α-methylproline, phenylglycine, α-methylphenylalanine and homophenylalanine.

“Amino acyl”: More generally, the term amino acyl, as used herein, encompasses natural amino acid and unnatural amino acids.

“Polyamide”: refers to homo- or hetero-polymers of natural amino acid and unnatural amino acids. Illustrative homo-polymers include, but are not limited to, poly-lysine, poly-arginine, poly-γ-glutaric acid, and the like. Illustrative hetero-polymers include, but are not limited to, polymers comprising peptides fragments selected from peptidases, lysozymes, metalloproteinases, and the like.

“PHF” refers to poly(1-hydroxymethylethylene hydroxymethyl-formal).

As used herein, the terms “polymer unit”, “monomeric unit”, “monomer”, “monomer unit”, “unit” all refer to a repeatable structural unit in a polymer.

The present invention is intended to include all isotopes of atoms occurring in the present compounds. Isotopes include those atoms having the same atomic number but different mass numbers. By way of general example and without limitation, isotopes of hydrogen include tritium and deuterium. Isotopes of carbon include C-13 and C-14.

The present invention is intended to include all isomers of the compound, which refers to and includes, optical isomers, and tautomeric isomers, where optical isomers include enantiomers and diastereomers, chiral isomers and non-chiral isomers, and the optical isomers include isolated optical isomers as well as mixtures of optical isomers including racemic and non-racemic mixtures; where an isomer may be in isolated form or in a mixture with one or more other isomers.

### Polymers or Polymeric Carriers

In certain exemplary embodiments, the conjugates of the invention find use in biomedical applications, such as drug delivery and tissue engineering, and the carrier is biocompatible and biodegradable. In certain embodiments, the carrier is a soluble polymer, nanoparticle, gel, liposome, micelle, suture, implant, etc. In certain embodiment, the term “soluble polymer” encompasses biodegradable biocompatible polymer such as a polyal (e.g., hydrophilic polyacetal or polyketal). In certain other embodiments, the carrier is a fully synthetic, semi-synthetic or naturally-occurring polymer. In certain other embodiments, the carrier is hydrophilic.

In certain exemplary embodiments, the carriers used in the present invention are biodegradable biocompatible polyals comprising at least one hydrolysable bond in each monomer unit positioned within the main chain. This ensures that the degradation process (via hydrolysis/cleavage of the monomer units) will result in fragmentation of the polymer conjugate to the monomeric components (i.e., degradation), and confers to the polymer conjugates of the invention their biodegradable properties. The properties (e.g., solubility, bioadhesivity and hydrophilicity) of biodegradable biocompatible polymer conjugates can be modified by subsequent substitution of additional hydrophilic or hydrophobic groups. Examples of biodegradable biocompatible polymers suitable for practicing the invention can be found inter alia in U.S. Pat. Nos. 5,811,510; 5,863,990; 5,958,398; 7,838,619, 7,790,150 and 8,030,459; each of the above listed patent documents is incorporated herein by reference in its entirety. Guidance on the significance, preparation, and applications of this type of polymers may be found in the above-cited documents. In certain embodiments, it is anticipated that the present invention will be particularly useful in combination with the above-referenced patent documents, as well as U.S. Pat. Nos. 5,582,172 and 6,822,086, each of the above listed patent documents is incorporated herein by reference in its entirety.

The conjugates of this invention are hydrophilic, hydrolysable and comprise drug molecules (e.g., vinca alkaloids, non-natural camptothecin compounds, auristatins, tubulysins, duocarmycins, PI3 kinases, MEK inhibitors, KSP inhibitors, and derivatives thereof) and/or antibodies (e.g., Trastuzumab, Cetuximab, Rituximab, Bevacizumab, Epratuzumab, Veltuzumab, Labetuzumab, Lintuzumab) or peptides (LHRH receptor targeting peptides, EC-1 peptide) or proteins (e.g., insulin, transferrin, interferon) covalently attached to the polymer carrier via linkages that contain one or more biodegradable bonds. Thus, in certain exemplary embodiments, carriers suitable for practicing the present invention are polyals having at least one acetal/ketal oxygen atom in each monomer unit positioned within the main chain. As discussed above, this ensures that the degradation process (via hydrolysis/cleavage of the polymer acetal/ketal groups) will result in fragmentation of the polyal conjugate to low molecular weight components (i.e., degradation).

In certain embodiments, biodegradable biocompatible polymer carriers, used for preparation of polymer conjugates of the invention, are naturally occurring polysaccharides, glycopolysaccharides, and synthetic polymers of polyglycoside, polyacetal, polyamide, polyether, and polyester origin and products of their oxidation, fictionalization, modification, cross-linking, and conjugation.

In certain other embodiments, the carrier is a hydrophilic biodegradable polymer selected from the group consisting of carbohydrates, glycopolysaccharides, glycolipids, glycoconjugates, polyacetals, polyketals, and derivatives thereof.

In certain exemplary embodiments, the carrier is a naturally occurring linear and/or branched biodegradable biocompatible homopolysaccharide selected from the group consisting of cellulose, amylose, dextran, levan, fucoidan, carraginan, inulin, pectin, amylopectin, glycogen and lixenan.

In certain other exemplary embodiments, the carrier is a naturally occurring linear and branched biodegradable biocompatible heteropolysaccharide selected from the group consisting of agarose, hyluronan, chondroitinsulfate, dermatansulfate, keratansulfate, alginic acid and heparin.

In yet other exemplary embodiments, the polymeric carrier comprises a copolymer of a polyacetal/polyketal and a hydrophilic polymer selected from the group consisting of polyacrylates, polyvinyl polymers, polyesters, polyorthoesters, polyamides, polypeptides, and derivatives thereof.

In yet another embodiment, the polymeric carrier is dextrin that is produced by the hydrolysis of a starch obtained from various natural products such as, for example, wheat, rice, maize and tapioca. Depending on the structure of the starch starting material each dextrin comprises a unique distribution of α-1,4 linkages and α-1,6 linkages. Since the rate of biodegradability of α-1,6 linkages is typically less than that for α-1,4 linkages, preferably the percentage of α-1,6 linkages is less than 10% and more preferably less than 5%. In one embodiment the molecular weight of the dextrin is in the range of about 1 kDa to about 200 kDa, more preferably from about 2 kDa to about 75 kDa (e.g., 2-55 kDa).

In certain embodiments, the carrier comprises polysaccharides activated by selective oxidation of cyclic vicinal diols of 1,2-, 1,4-, 1,6-, and 2,6-pyranosides, and 1,2-, 1,5-, 1,6-furanosides, or by oxidation of lateral 6-hydroxy and 5,6-diol containing polysaccharides prior to conjugation with drug molecules or PBRMs.

In still other embodiments, the polymeric carrier comprises a biodegradable biocompatible polyacetal wherein at least a subset of the polyacetal repeat structural units have the following chemical structure:

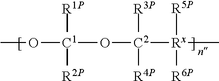

wherein for each occurrence of the n bracketed structure, one of R^{1P} and R^{2P} is hydrogen, and the other is a biocompatible group and includes a carbon atom covalently attached to C¹; R^{x} is a carbon atom covalently attached to C²; n″ is an integer; each occurrence of R^{3P}, R^{4P}, R⁵ and R^{6P} is a biocompatible group and is independently hydrogen or an organic moiety; and for each occurrence of the bracketed structure n, at least one of R^{1P}, R^{2P}, R^{3P}, R^{4P}, R^{5P} and R^{6P} comprises a functional group suitable for coupling. In certain embodiments, the functional group is a hydroxyl moiety.

In one embodiment, the polymeric carrier comprises activated hydrophilic biodegradable biocompatible polymers comprising from 0.1% to 100% polyacetal moieties whose backbone is represented by the following chemical structure:

(—CH₂—CHR₇—O—CHR₈—O—)ₒ

wherein:
R₇ and R₈ are independently hydrogen, hydroxyl, hydroxy alkyl (e.g., —CH₂OH, —CH(OH)—CH₂OH), —CHO, —CH(OH)—CHO or -carbonyl; and
o is an integer from 20 to 2000.

In yet other embodiments, the polymeric carrier comprises a biodegradable biocompatible polyketal wherein at least a subset of the polyketal repeatable structural units have the following chemical structure:

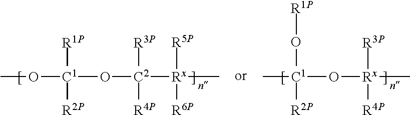

wherein each occurrence of R^{1P} and R^{2P} is a biocompatible group and R^{x}, R^{3P}, R^{4P}, R^{5P}, R^{6P} and are as defined herein

In certain embodiments, the ketal units are monomers of Formula (IIa) or (IIb):

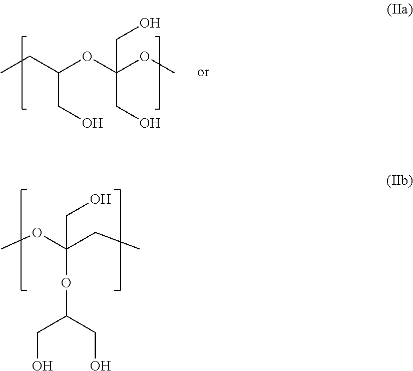

Biodegradable, biocompatible polyketal polymers and their methods of making have been described in U.S. Pat. Nos. 5,811,510, 7,790,150 and 7,838,619, which are hereby incorporated by reference in their entirety.

In one embodiment, the polymeric carrier can be obtained from partially oxidized dextran (β1→6)-D-glucose) followed by reduction. In this embodiment, the polymer comprises a random mixture of the unmodified dextran (A), partially oxidized dextran acetal units (B) and exhaustively dextran acetal units (C) of the following structures:

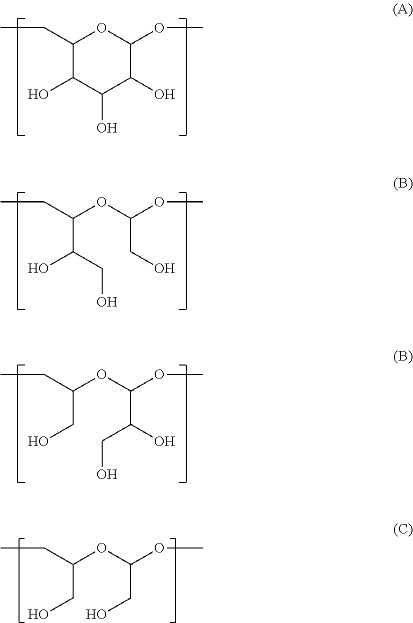

In another embodiment, the polymeric carrier comprises unmodified acetal units, i.e., polyacetal segments. In some embodiments, the polyacetals can be derived from exhaustively oxidized dextran followed by reduction. These polymers have been described in U.S. Pat. No. 5,811,510, which is hereby incorporated by reference for its description of polyacetals at column 2, line 65 to column 8, line 55 and their synthesis at column 10, line 45 to column 11, line 14. In one embodiment, the unmodified polyacetal polymer is a poly(hydroxymethylethylene hydroxymethyl formal) polymer (PHF).

In addition to poly(hydroxymethylethylene hydroxymethyl formal) polymers, the backbone of the polymeric carrier can also comprise co-polymers of poly(hydroxymethylethylene hydroxymethyl formal) blocks and other acetal or non-acetal monomers or polymers. For example, polyethylene glycol polymers are useful as a stealth agent in the polymer backbone because they can decrease interactions between polymer side chains of the appended functional groups. Such groups can also be useful in limiting interactions such as between serum factors and the modified polymer. Other stealth agent monomers for inclusion in the polymer backbone include, for example, ethyleneimine, methacrylic acid, acrylamide, glutamic acid, and combinations thereof.

The acetal or ketal units are present in the modified polymer in an amount effective to promote biocompatibility. The unmodified acetal or ketal unit can be described as a “stealth agent” that provides biocompatibility and solubility to the modified polymers. In addition, conjugation to a polyacetal or polyketal polymer can modify the susceptibility to metabolism and degradation of the moieties attached to it, and influence biodistribution, clearance and degradation.

The unmodified acetal units are monomers of Formula (III):

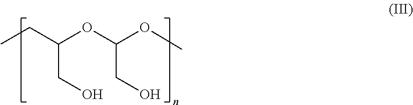

The molar fraction, n, of unmodified polyacetal units is the molar fraction available to promote biocompatibility, solubility and increase half-life, based on the total number of polymer units in the modified polymer. The molar fraction n may be the minimal fraction of unmodified monomer acetal units needed to provide biocompatibility, solubility, stability, or a particular half-life, or can be some larger fraction. The most desirable degree of cytotoxicity is substantially none, i.e., the modified polymer is substantially inert to the subject. However, as is understood by those of ordinary skill in the art, some degree of cytotoxicity can be tolerated depending on the severity of disease or symptom being treated, the efficacy of the treatment, the type and degree of immune response, and like considerations.

In embodiments, at least one terminus of the terminally modified polymer of the invention is —O—(CH₂)₂-L^{M} or —O—CH₂—CH(OH)—CH₂—CR¹═CR²R³, and L^{M} is a linker capable of covalently conjugating with M and comprises a nitrogen-containing moiety selected from the group consisting of —NR¹, —NR¹C(═X¹)—, —NR¹C(═X¹)Y—, —NRNR², —NR¹NR²C(═X¹)—, —NR¹NR²C(═X¹)Y—, —NR¹SO₂—, and —NR¹SO₂NR²—, with the NR¹ moiety attached directly or indirectly to the polymer in the order as written, in which X¹ is O, S, or NR³ and Y is O, S, or NR⁴, and each of R¹, R², R³, and R⁴ independently is H or an aliphatic, heteroaliphatic, carbocyclic, or heterocyclic moiety.

For example, the terminally modified polymer does not contain —O—(CH₂)₂-L^{M} or —O—CH₂—CH(OH)—CH₂—CR¹═CR²R³ along the backbone of the polymer.

For example, the terminally modified polymer contains only one —O—(CH₂)₂-L^{M} or —O—CH₂—CH(OH)—CH₂—CR¹═CR²R³.

For example, at least one terminus of the polymer is —O—(CH₂)₂-L^{M}.

For example, L^{M} further includes

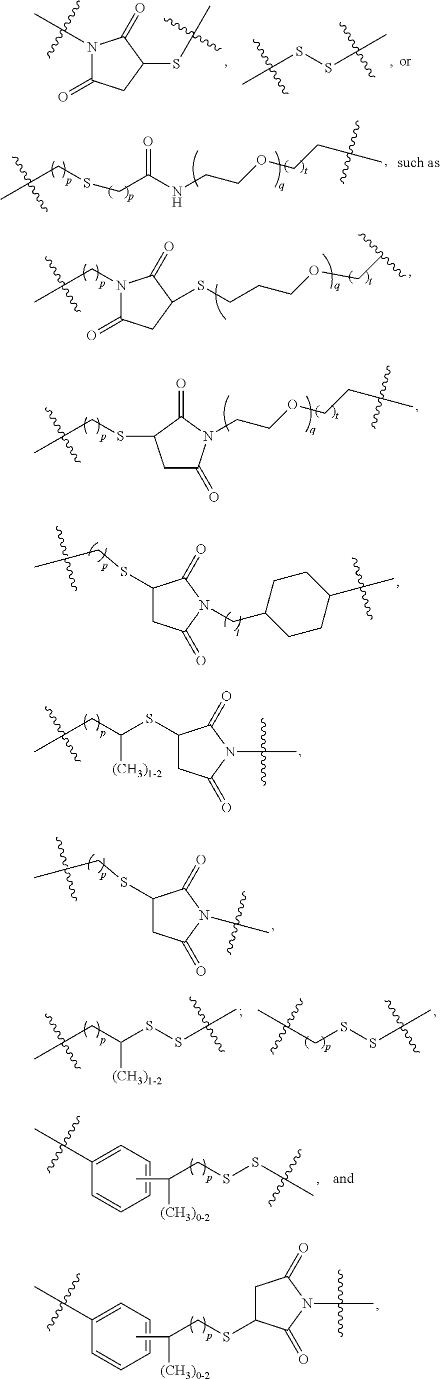

in which q is an integer from 0 to 12 and each of p and t independently is an integer from 0 to 3.

For example, at least one terminus of the polymer is —O—CH₂—CH(OH)—CH₂—CR¹═CR²R³.

For example, each of R¹, R², and R³ is H.

For example, the terminally modified polymer is of the following structure:

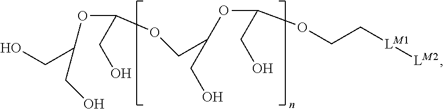

wherein
n is an integer between 1 and about 1100,
L^{M1} is —NR¹, —NR¹C(═X¹)—, —NR¹C(═X¹)Y—, —NR¹NR²—, —NR¹NR²C(═X¹)—, —NR¹NR²C(═X¹)Y—, —NR¹SO₂—, or —NR¹SO₂NR²—, with the NR¹ moiety attached to the polymer in the order as written, and
L^{M2} is —(CH₂)ₘ—W, with (CH₂)ₘ connected to L^{M1}, in which m is an integer between 0 and 20, and W, when not conjugated with M, is a functional group suitable for coupling with M or W is an aliphatic, heteroaliphatic, carbocyclic, or heterocyclic moiety, wherein the aliphatic, heteroaliphatic, carbocyclic, or heterocycloalkyl moiety comprises a functional group suitable for coupling with M.

For example, each of R¹, R², R³ and R⁴ independently is H, or unsubstituted or substituted C₁₋₆ alkyl (e.g., C₁₋₆ alkyl substituted with amino, maleimide, carboxylic acid, ester, or other substituents disclosed herein).

For example, the terminally modified polymer has a backbone of PHF and is modified at the terminal position by selective oxidation of glycolic vicinal diols followed by reductive amination to introduce a primary amine functionality at the terminal position of PHF.

For example, the ratio between M (e.g., a PBRM or drug) and PHF to which M is connected to is 1:1. In other words, M has only one termally modified PHF attached to it.

For example, the ratio between M (e.g., a PBRM) and PHF to which M is connected to is between 1:2 and 1:20 (e.g., between 1:2 and 1:10, between 1:2 and 1:8, between 1:2 and 1:6, or between 1:2 and 1:4). In other words, M has two or more (e.g., about 2-20, 2-10, 2-8, 2-6, or about 2-4) termally modified PHFs attached to it.

In embodiments, the number of terminally modified polymers attached to a PBRM depends on the number of free thiol groups (i.e. cysteine) on PBRM.

For example, the terminally modified PHF is selected from Compounds 1-8 below:

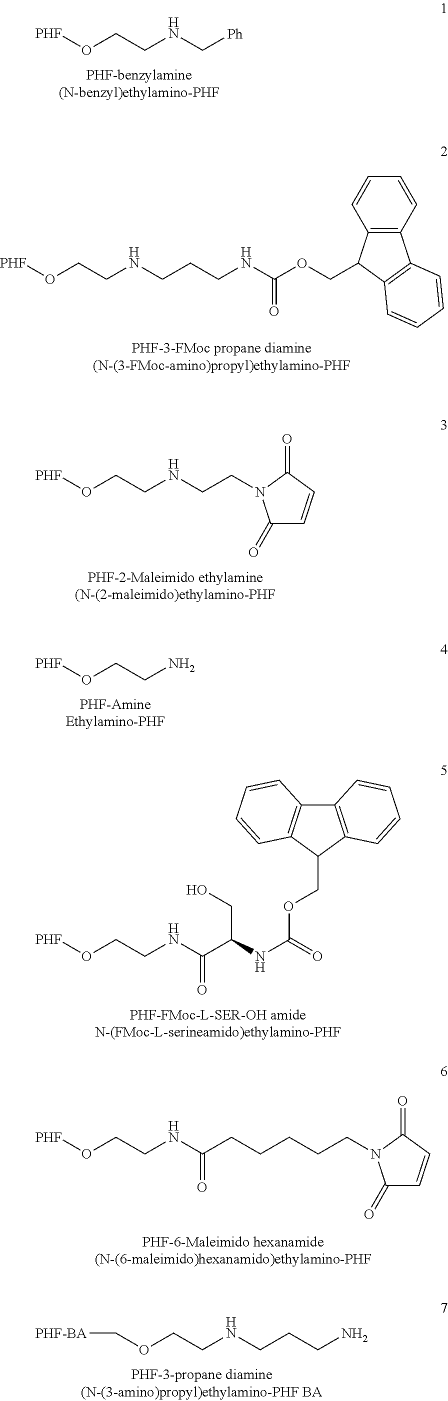

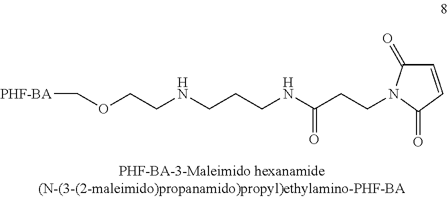

The terminally modified polymer may further contain a pharmaceutically useful modifier (“M”) covalently attached to the polymer along the backbone of the polymer. M can be attached to the polymer directly or indirectly, e.g., via a linker. M can be a protein based recognition-molecule (“PBRM”) or a therapeutic agent having a molecular weight ≦5 kDa (“D”). When M is a PBRM, it can be connected to the backbone of the terminally modified polymer via L^{P} and when M is D, it can be connected to the backbone of the terminally modified polymer via L^{D}.

In one embodiment, the backbone of the terminally modified polymer of this invention comprises units of Formula (IV):

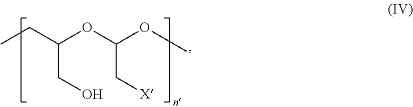

wherein X′ indicates the substituent for the hydroxyl group of the polymer backbone. As shown in Formula (IV) and the other formulae described herein, each polyacetal unit has a single hydroxyl group attached to the glycerol moiety of the unit and an X′ group (or another substituent such as -L^{D}-D or -L^{P}-PBRM) attached to the glycolaldehyde moiety of the unit. This is for convenience only and it should be construed that the polymer having units of Formula (IV) and other formulae described herein can contain a random distribution of units having a X′ group (or another substituent such as -L^{D}-D or -L^{P}-PBRM) attached to the glycolaldehyde moiety of the units and those having a single X′ group (or another substituent such as -L^{D}-D or -L^{P}-PBRM) attached to the glycerol moiety of the units as well as units having two X′ groups (or other substituents such as -L^{D}-D or -L^{P}-PBRM) with one attached to the glycolaldehyde moiety and the other attached to the glycerol moiety of the units.

For example, L^{D} is a linker having the structure:

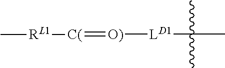

with R^{L1} connected to an oxygen atom of the polymeric carrier and L^{D1} connected to D, and

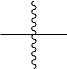

denotes direct or indirect attachment of D to L^{D1}, and L^{D} contains a biodegradable bond so that when the bond is broken, D is released from the polymeric carrier in an active form for its intended therapeutic effect; L^{D1} is a carbonyl-containing moiety; L^{P} is a linker different from L^{D} and having the structure: —R^{L2}—C(═O)-L^{P1} with R^{L2} connected to an oxygen atom of the polymeric carrier and L^{P1} suitable for connecting directly or indirectly to a PBRM; each of R^{L1} and R^{L2} independently is absent, alkyl, heteroalkyl, cycloalkyl, or heterocycloalkyl; and L^{P1} is a moiety containing a functional group that is capable of forming a covalent bond with a functional group of a PBRM.

For example, L^{P} is a linker having the structure:

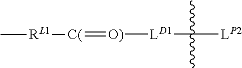

in which L^{P2} is a moiety containing a functional group that is capable of forming a covalent bond with a functional group of a PBRM, and

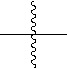

denotes direct or indirect attachment of L^{P2} to L^{D1}.

For example, the functional group of L^{P1} or L^{P2} is selected from —SR^{p}, —S—S-LG, maleimido, and halo, in which LG is a leaving group and R^{p} is H or a sulfur protecting group.

For example, L^{D}1 comprises —X—(CH₂)ᵥ—C(═O)— with X directly connected to the carbonyl group of R^{L1}—C(═O), in which X is CH₂, O, or NH, and v is an integer from 1 to 6.

For example, L^{P1} or L^{P2} contains a biodegradable bond.

For example, each of R^{L1} and R^{L2} is absent.

For example, each PBRM independently can be a protein, a peptide, a peptide mimetic, an antibody, or an antibody fragment.

For example, each occurrence of D independently can be selected from vinca alkaloids, auristatins, tubulysins, duocarmycins, PI3 kinases, MEK inhibitors, KSP inhibitors, and derivatives thereof.

In one embodiment, biodegradable biocompatible polyals suitable for practicing the present invention have a molecular weight of between about 1 kDa and about 150 kDa. In a preferred embodiment of the present invention, the biodegradable biocompatible polyals have a molecular weight of between about 2 kDa and about 75 kDa. For example, the biodegradable biocompatible polyals have a molecular weight (i.e., MW of the unmodified polyal) of between about 2 kDa and about 20 kDa (e.g. about 4-15 kDa or about 4-10 kDa) for conjugation with PBRM with a MW>40 kDa, or the biodegradable biocompatible polyals have a molecular weight (i.e., MW of the unmodified polyal) of between about 20 kDa and about 75 kDa (e.g. about 25-55 kDa or about 25-50 kDa) for conjugation with PBRM with a MW<200 kDa.

In one embodiment, the biodegradable biocompatible polyals suitable for practicing the present invention are first modified at one terminus of the polymer with a linker L^{M} that is capable of covalently conjugating with M, before conjugating with a drug or a PBRM along the backbone of the polymer. For example, the terminally modified polyals may contain subunits of linkers L^{D} or L^{P}, such as —C(═O)—X—(CH₂)ᵥ—C(═O)— with X being CH₂, O, or NH, and v being an integer from 1 to 6, along the backbone of the polyals. Table A below provides some examples of the terminally modified polyals suitable for conjugating with a drug or PBRM or derivatives thereof along the backbone of the polymer. Unless otherwise specified, reference numbers in Tables A through C below correspond to the Example numbers described herein; the term “ND” means not determined; and X is CH₂, O, or NH.

TABLE A

| Ref # | Terminally Modified Polymer Scaffold |
|---|---|
| | 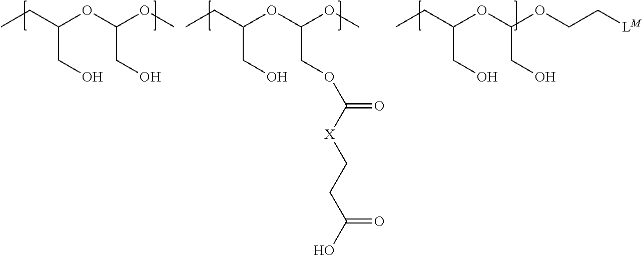 |
| | 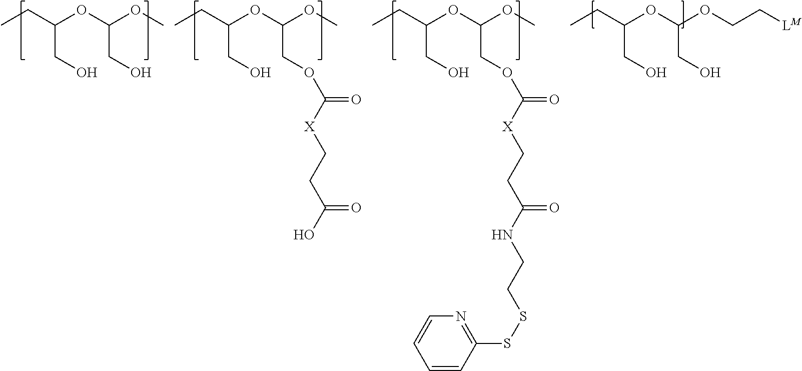 |
| | 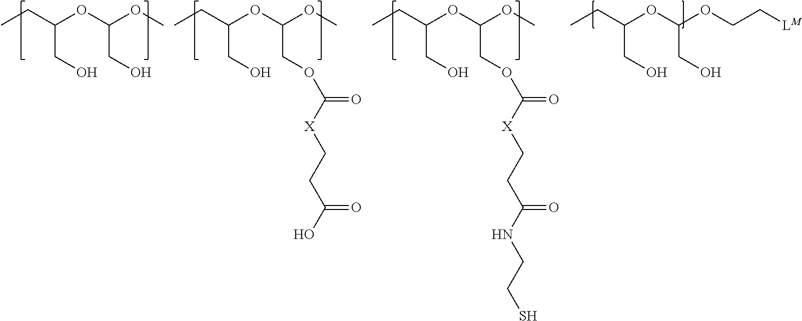 |
| | 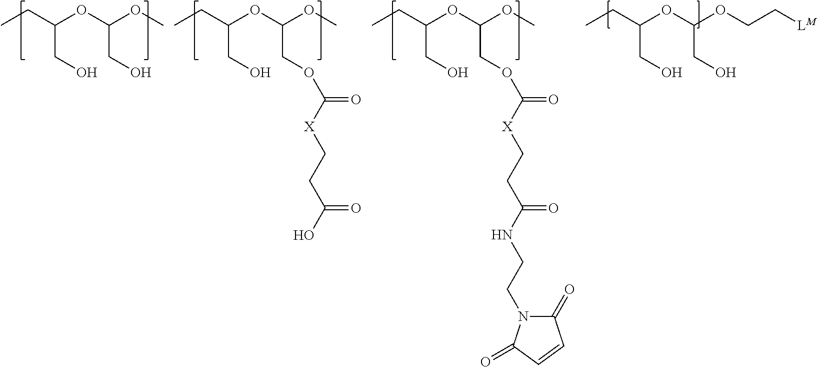 |
| | 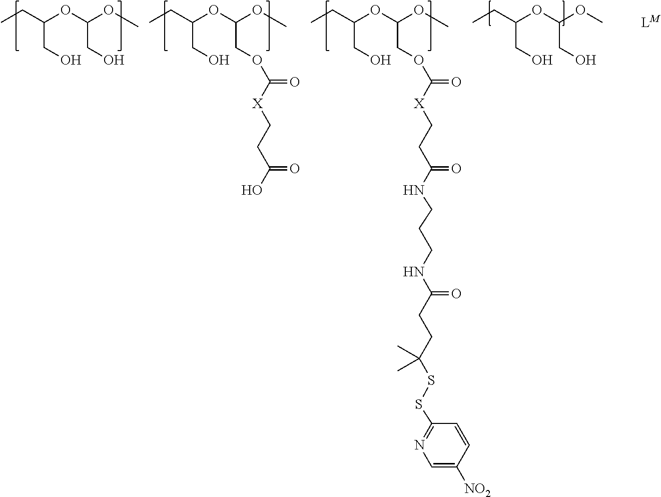 |
| | 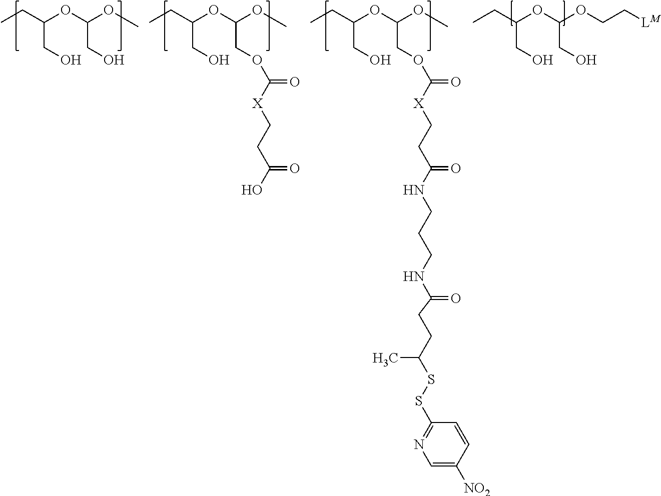 |
| | 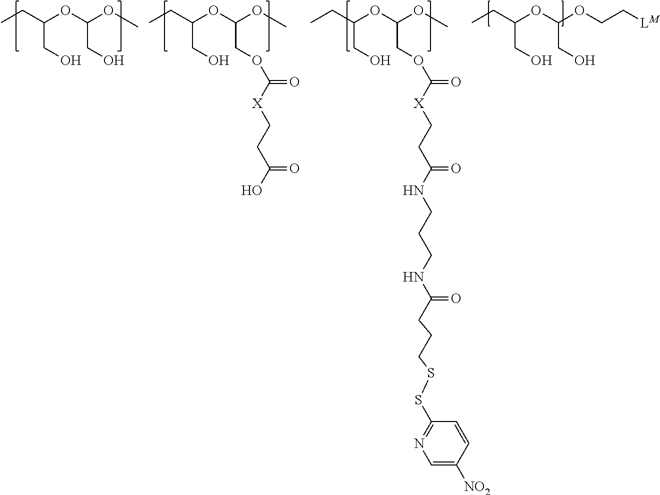 |
| | 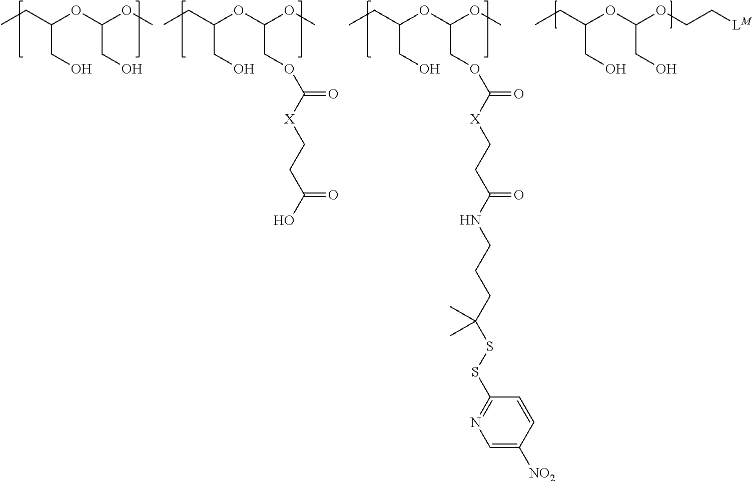 |
| | 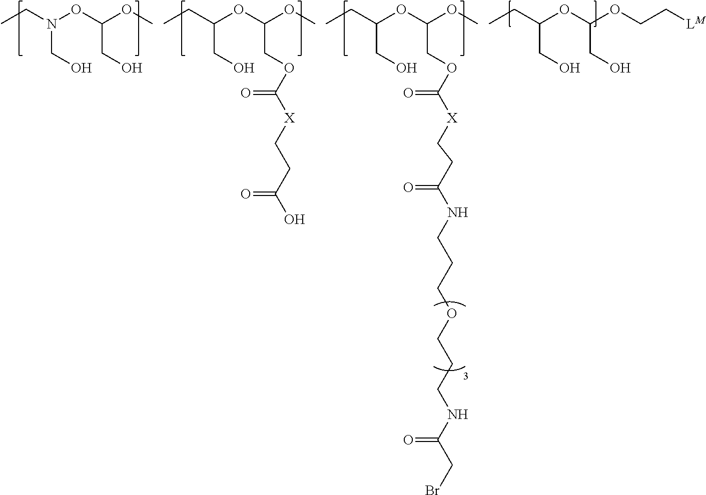 |
| | 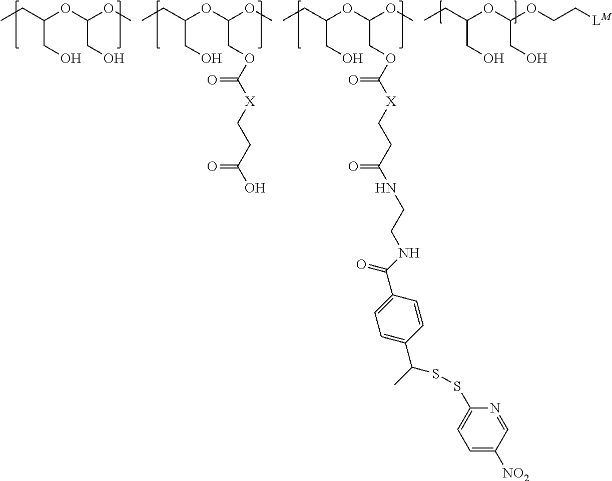 |
| | 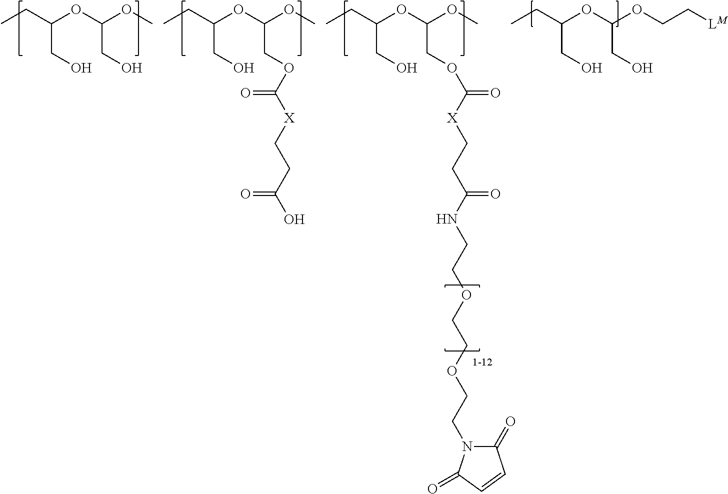 |
| | 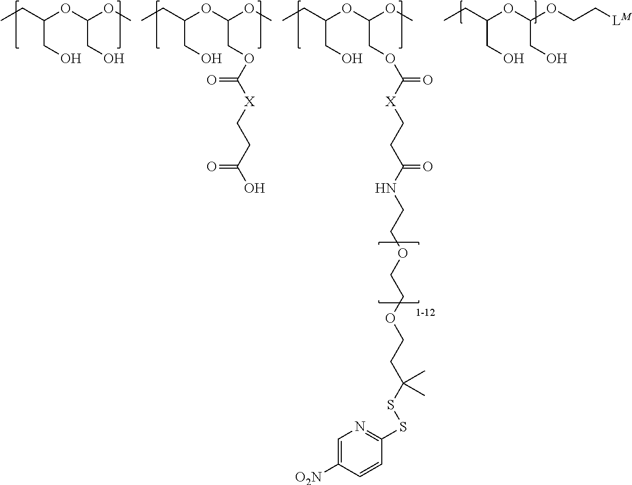 |
| | 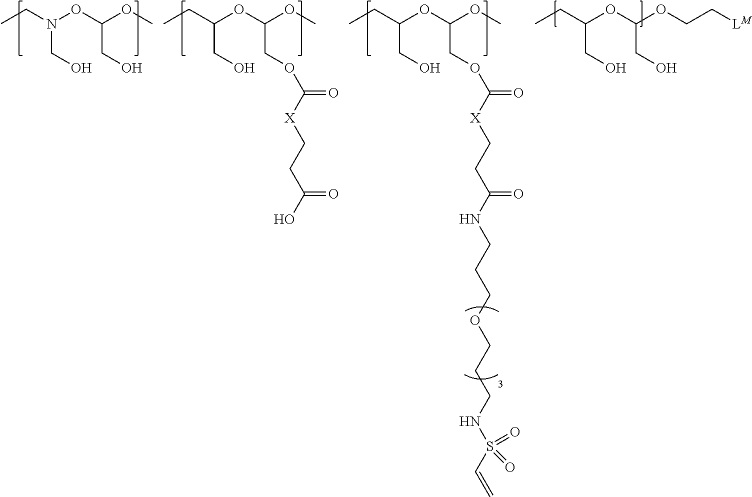 |
| | 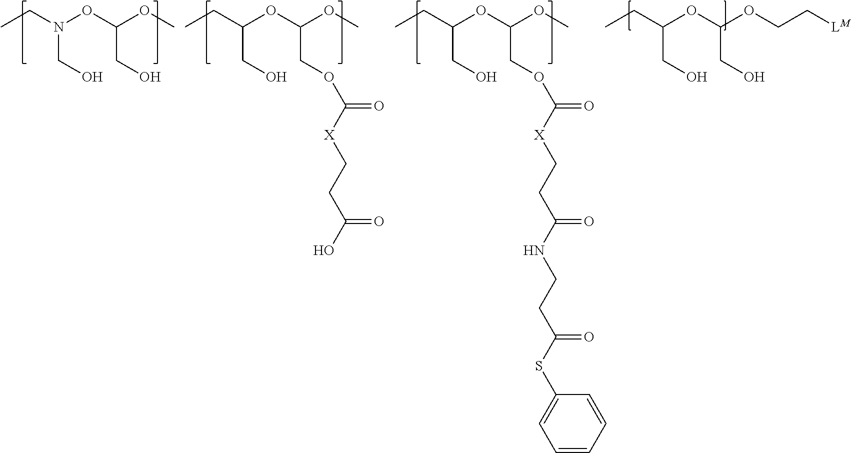 |
| Ex 2 Ex 10 R = H Ex 6 | 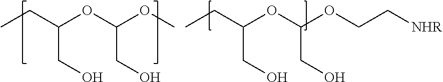 |
| Ex 3 | 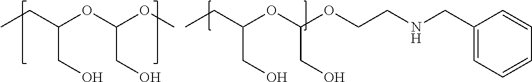 |
| Ex 5 | 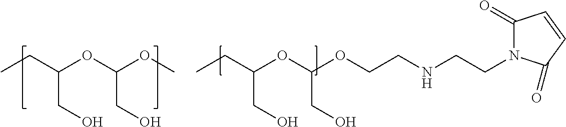 |
| Ex 11 | 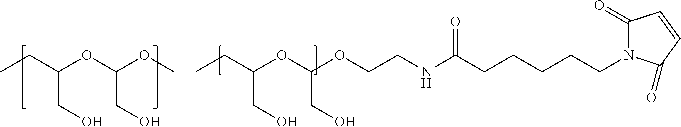 |
| X = NH Ex 13 | 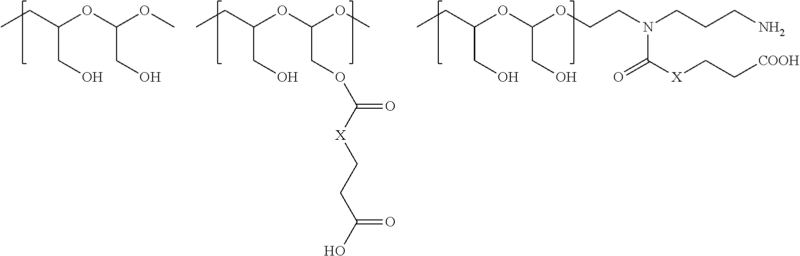 |
| X = NH Ex 14 | 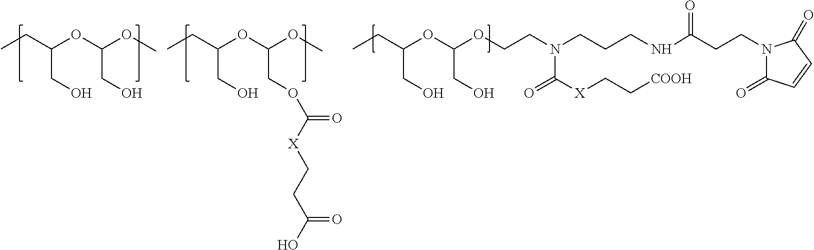 |
| X = CH₂ Ex 16 | 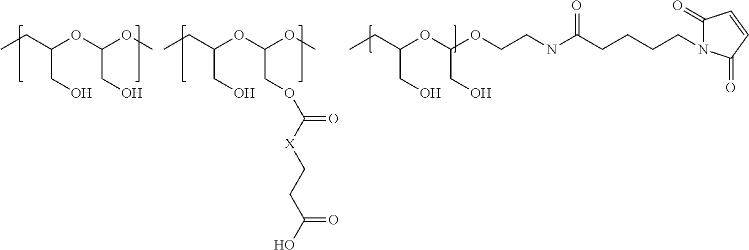 |

### Modifiers (“M”)

In certain embodiments, modifiers according to the invention include, but are not limited to, biomolecules, small molecules, therapeutic agents, pharmaceutically useful groups or entities, microparticles, a protein-based recognition molecules (PBRM), macromolecules, diagnostic labels, chelating agents, hydrophilic moieties, dispersants, charge modifying agents, viscosity modifying agents, surfactants, coagulation agents and flocculants.

### Therapeutic Agents

In certain embodiments, the therapeutic agent is a small molecule having a molecular weight preferably ≦about 5 kDa, more preferably ≦about 4 kDa, more preferably ≦about 3 kDa, most preferably ≦about 1.5 kDa or ≦about 1 kDa.

In certain embodiments, the therapeutic agent has an IC₅₀ of about less than 1 nM.

In another embodiment, the therapeutic agent has an IC₅₀ of about greater than 1 nM, for example, the therapeutic agent has an IC₅₀ of about 1 to 50 nM.

Some therapeutic agents having an IC₅₀ of greater than about 1 nM (e.g., “less potent drugs”) are unsuitable for conjugation with a PBRM using art-recognized conjugation techniques. Without wishing to be bound by theory, such therapeutic agents have a potency that is insufficient for use in targeted PBRM-drug conjugates using conventional techniques as sufficient copies of the drug (i.e., more than 8) cannot be conjugated using art-recognized techniques without resulting in diminished pharmacokinetic and physiochemical properties of the conjugate. However sufficiently high loadings of these less potent drugs can be achieved using the conjugation strategies described herein thereby resulting in high loadings of the therapeutic agent while maintaining the desirable pharmacokinetic and physiochemical properties. Thus, the invention also relates to a PBRM-drug conjugate which includes a PBRM, PHF and at least eight therapeutic agent moieties, wherein the therapeutic agent has an IC₅₀ of greater than about 1 nM.

In certain embodiments, the therapeutic agent is attached to the terminus of the polymer. In certain embodiments, therapeutic agent is attached to the backbone of the polymer directly or indirectly. In certain embodiments, about 0.1 to about 25% monomers comprise a therapeutic agent, more preferably about 0.5 to about 20%, more preferably about 1 to about 15%, and even more preferably about 2 to about 10%.

The small molecule therapeutic agents used in this invention (e.g., antiproliferative (cytotoxic and cytostatic) agents capable of being linked to a polymer carrier) include cytotoxic compounds (e.g., broad spectrum), angiogenesis inhibitors, cell cycle progression inhibitors, PI3K/m-TOR/AKT pathway inhibitors, MAPK signaling pathway inhibitors, kinase inhibitors, protein chaperones inhibitors, HDAC inhibitors, PARP inhibitors, Wnt/Hedgehog signaling pathway inhibitors and RNA polymerase inhibitors.

Broad spectrum cytotoxins include, but are not limited to, DNA-binding or alkylating drugs, microtubule stabilizing and destabilizing agents, platinum compounds, and topoisomerase I inhibitors.

Exemplary DNA-binding or alkylating drugs include, CC-1065 and its analogs, anthracyclines (doxorubicin, epirubicin, idarubicin, daunorubicin) and its analogs, alkylating agents, such as calicheamicins, dactinomycines, mitomycines, pyrrolobenzodiazepines, and the like.

Exemplary CC-1065 analogs include duocarmycin SA, duocarmycin Cl, duocarmycin C2, duocarmycin B2, DU-86, KW-2189, bizelesin, seco-adozelesin, and those described in U.S. Pat. Nos. 5,475,092; 5,595,499; 5,846,545; 6,534,660; 6,586,618; 6,756,397 and 7,049,316. Doxorubicin and its analogs include those described in U.S. Pat. No. 6,630,579. Calicheamicins include those described in U.S. Pat. Nos. 5,714,586 and 5,739,116. Duocarmycins include those described in U.S. Pat. Nos. 5,070,092; 5,101,038; 5,187,186; 6,548,530; 6,660,742; and 7,553,816 B2; and Li et al., Tet Letts., 50:2932-2935 (2009). Pyrrolobenzodiazepines include those described in Denny, Exp. Opin. Ther. Patents., 10(4):459-474 (2000).

Exemplary microtubule stabilizing and destabilizing agents include taxane compounds, such as paclitaxel, docetaxel; maytansinoids, auristatins and analogs thereof, tubulysin A and B derivatives, vinca alkaloid derivatives, epothilones and cryptophycins.

Exemplary maytansinoids or maytansinoid analogs include maytansinol and maytansinol analogs, maytansine or DM-1 and DM-4 are those described in U.S. Pat. Nos. 5,208,020; 5,416,064; 6,333,410; 6,441,163; 6,716,821; RE39,151 and 7,276,497. In certain embodiments, the cytotoxic agent is a maytansinoid, another group of anti-tubulin agents (ImmunoGen, Inc.; see also Chari et al., 1992, Cancer Res. 52:127-131), maytansinoids or maytansinoid analogs. Examples of suitable maytansinoids include maytansinol and maytansinol analogs. Suitable maytansinoids are disclosed in U.S. Pat. Nos. 4,424,219; 4,256,746; 4,294,757; 4,307,016; 4,313,946; 4,315,929; 4,331,598; 4,361,650; 4,362,663; 4,364,866; 4,450,254; 4,322,348; 4,371,533; 6,333,410; 5,475,092; 5,585,499; and 5,846,545.

Exemplary auristatins include auristatin E (also known as a derivative of dolastatin-10), auristatin EB (AEB), auristatin EFP (AEFP), monomethyl auristatin E (MMAE), monomethyl auristatin F (MMAF), auristatin F and dolastatin. Suitable auristatins are also described in U.S. Publication Nos. 2003/0083263, 2011/0020343, and 2011/0070248; PCT Application Publication Nos. WO 09/117531, WO 2005/081711, WO 04/010957; WO 02/088172 and WO01/24763, and U.S. Pat. Nos. 7,498,298; 6,884,869; 6,323,315; 6,239,104; 6,124,431; 6,034,065; 5,780,588; 5,767,237; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, the disclosures of which are incorporated herein by reference in their entirety.

Exemplary tubulysin compounds include compounds described in U.S. Pat. Nos. 7,816,377; 7,776,814; 7,754,885; U.S. Publication Nos. 2011/0021568; 2010/004784; 2010/0048490; 2010/00240701; 2008/0176958; and PCT Application Nos. WO 98/13375; WO 2004/005269; WO 2008/138561; WO 2009/002993; WO 2009/055562; WO 2009/012958; WO 2009/026177; WO 2009/134279; WO 2010/033733; WO 2010/034724; WO 2011/017249; WO 2011/057805; the disclosures of which are incorporated by reference herein in their entirety.

Exemplary vinca alkaloids include vincristine, vinblastine, vindesine, and navelbine (vinorelbine). Suitable Vinca alkaloids that can be used in the present invention are also disclosed in U.S. Publication Nos. 2002/0103136 and 2010/0305149, and in U.S. Pat. No. 7,303,749 B1, the disclosures of which are incorporated herein by reference in their entirety.

Exemplary epothilone compounds include epothilone A, B, C, D, E and F, and derivatives thereof. Suitable epothilone compounds and derivatives thereof are described, for example, in U.S. Pat. Nos. 6,956,036; 6,989,450; 6,121,029; 6,117,659; 6,096,757; 6,043,372; 5,969,145; and 5,886,026; and WO 97/19086; WO 98/08849; WO 98/22461; WO 98/25929; WO 98/38192; WO 99/01124; WO 99/02514; WO 99/03848; WO 99/07692; WO 99/27890; and WO 99/28324; the disclosures of which are incorporated herein by reference in their entirety.

Exemplary cryptophycin compounds are described in U.S. Pat. Nos. 6,680,311 and 6,747,021.

Exemplary platinum compounds include cisplatin (PLATINOL®), carboplatin (PARAPLATIN®), oxaliplatin (ELOXATINE®), iproplatin, ormaplatin, and tetraplatin.

Exemplary topoisomerase I inhibitors include camptothecin, camptothecin, derivatives, camptothecin analogs and non-natural camptothecins, such as, for example, CPT-11 (irinotecan), SN-38, topotecan, 9-aminocamptothecin, rubitecan, gimatecan, karenitecin, silatecan, lurtotecan, exatecan, diflomotecan, belotecan, lurtotecan and S39625. Other camptothecin compounds that can be used in the present invention include those described in, for example, J. Med. Chem., 29:2358-2363 (1986); J. Med. Chem., 23:554 (1980); J. Med. Chem., 30:1774 (1987).

Angiogenesis inhibitors include, but are not limited, MetAP2 inhibitors, VEGF inhibitors, PIGF inhibitors, VGFR inhibitors, PDGFR inhibitors. Exemplary VGFR and PDGFR inhibitors include sorafenib (Nexavar), sunitinib (Sutent) and vatalanib. Exemplary MetAP2 inhibitors include fumagillol derivatives, meaning any compound that includes the fumagillin core structure, including fumagillamine, that inhibits the ability of MetAP-2 to remove NH₂-terminal methionines from proteins as described in Rodeschini et al., J. Org. Chem., 69, 357-373, 2004 and Liu, et al., Science 282, 1324-1327, 1998. Non limiting examples of“fumagillol derivatives” are disclosed in J. Org. Chem., 69, 357, 2004; J. Org. Chem., 70, 6870, 2005; European Patent Application 0 354 787; J. Med. Chem., 49, 5645, 2006; Bioorg. Med. Chem., 11, 5051, 2003; Bioorg. Med. Chem., 14, 91, 2004; Tet. Lett. 40, 4797, 1999; WO99/61432; U.S. Pat. Nos. 6,603,812; 5,789,405; 5,767,293; 6,566,541; and 6,207,704.

Exemplary cell cycle progression inhibitors include CDK inhibitors such as, for example, BMS-387032 and PD0332991; Rho-kinase inhibitors such as, for example GSK429286; checkpoint kinase inhibitors such as, for example, AZD7762; aurora kinase inhibitors such as, for example, AZD1152, MLN8054 and MLN8237; PLK inhibitors such as, for example, BI 2536, BI6727 (Volasertib), GSK461364, ON-01910 (Estybon); and KSP inhibitors such as, for example, SB 743921, SB 715992 (ispinesib), MK-0731, AZD8477, AZ3146 and ARRY-520.

Exemplary PI3K/m-TOR/AKT signaling pathway inhibitors include phosphoinositide 3-kinase (PI3K) inhibitors, GSK-3 inhibitors, ATM inhibitors, DNA-PK inhibitors and PDK-1 inhibitors.

Exemplary PI3 kinases are disclosed in U.S. Pat. No. 6,608,053, and include BEZ235, BGT226, BKM120, CAL101, CAL263, demethoxyviridin, GDC-0941, GSK615, IC87114, LY294002, Palomid 529, perifosine, PF-04691502, PX-866, SAR245408, SAR245409, SF1126, Wortmannin, XL147 and XL765.

Exemplary AKT inhibitors include, but are not limited to AT7867.

Exemplary MAPK signaling pathway inhibitors include MEK, Ras, JNK, B-Raf and p38 MAPK inhibitors.

Exemplary MEK inhibitors are disclosed in U.S. Pat. No. 7,517,994 and include GDC-0973, GSK1120212, MSC1936369B, AS703026, RO5126766 and RO4987655, PD0325901, AZD6244, AZD 8330 and GDC-0973.

Exemplary B-raf inhibitors include CDC-0879, PLX-4032, and SB590885.

Exemplary B p38 MAPK inhibitors include BIRB 796, LY2228820 and SB 202190

Receptor tyrosine kinases (RTK) are cell surface receptors which are often associated with signaling pathways stimulating uncontrolled proliferation of cancer cells and neoangiogenesis. Many RTKs, which over express or have mutations leading to constitutive activation of the receptor, have been identified, including, but not limited to, VEGFR, EGFR, FGFR, PDGFR, EphR and RET receptor family receptors. Exemplary specific RTK targets include ErbB2, FLT-3, c-Kit, and c-Met.

Exemplary inhibitors of ErbB2 receptor (EGFR family) include but not limited to AEE788 (NVP-AEE 788), BIBW2992, (Afatinib), Lapatinib, Erlotinib (Tarceva), and Gefitinib (Iressa).

Exemplary RTK inhibitors targeting more than one signaling pathway (multitargeted kinase inhibitors) include AP24534 (Ponatinib) that targets FGFR, FLT-3, VEGFR-PDGFR and Bcr-Abl receptors; ABT-869 (Linifanib) that targets FLT-3 and VEGFR-PDGFR receptors; AZD2171 that targets VEGFR-PDGFR, Flt-1 and VEGF receptors; CHR-258 (Dovitinib) that targets VEGFR-PDGFR, FGFR, Flt-3, and c-Kit receptors; Sunitinib (Sutent) that targets VEGFR, PDGFR, KIT, FLT-3 and CSF-IR; Sorafenib (Nexavar) and Vatalanib that target VEGFR, PDGFR as well as intracellular serine/threonine kinases in the Raf/Mek/Erk pathway.

Exemplary protein chaperon inhibitors include HSP90 inhibitors. Exemplary HSP90 inhibitors include 17AAG derivatives, BIIB021, BIIB028, SNX-5422, NVP-AUY-922 and KW-2478.

Exemplary HDAC inhibitors include Belinostat (PXD101), CUDC-101, Droxinostat, ITF2357 (Givinostat, Gavinostat), JNJ-26481585, LAQ824 (NVP-LAQ824, Dacinostat), LBH-589 (Panobinostat), MC1568, MGCD0103 (Mocetinostat), MS-275 (Entinostat), PCI-24781, Pyroxamide (NSC 696085), SB939, Trichostatin A and Vorinostat (SAHA).

Exemplary PARP inhibitors include iniparib (BSI 201), olaparib (AZD-2281), ABT-888 (Veliparib), AG014699, CEP 9722, MK 4827, KU-0059436 (AZD2281), LT-673, 3-aminobenzamide, A-966492, and AZD2461.

Exemplary Wnt/Hedgehog signaling pathway inhibitors include vismodegib (R^{G3616}/GDC-0449), cyclopamine (11-deoxojervine) (Hedgehog pathway inhibitors) and XAV-939 (Wnt pathway inhibitor)

Exemplary RNA polymerase inhibitors include amatoxins. Exemplary amatoxins include α-amanitins, β-amanitins, γ-amanitins, ε-amanitins, amanullin, amanullic acid, amaninamide, amanin, and proamanullin.

In one embodiment the drug of the invention is a non-natural camptothecin compound, vinca alkaloid, kinase inhibitor (e.g., PI3 kinase inhibitor (GDC-0941 and PI-103)), MEK inhibitor, KSP inhibitor, RNA polymerase inhibitor, PARP inhibitor, docetaxel, paclitaxel, doxorubicin, duocarmycin, tubulysin, auristatin or a platinum compound. In specific embodiments, the drug is a derivative of SN-38, vindesine, vinblastine, PI-103, AZD 8330, auristatin E, auristatin F, a duocarmycin compound, tubulysin compound, or ARRY-520.

In another embodiment, the drug used in the invention is a combination of two or more drugs, such as, for example, PI3 kinases and MEK inhibitors; broad spectrum cytotoxic compounds and platinum compounds; PARP inhibitors and platinum compounds; broad spectrum cytotoxic compounds and PARP inhibitors.

In one embodiment, the Vinca alkaloid is a compound of Formula (V):

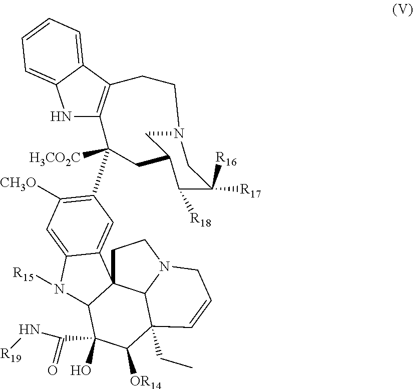

wherein:
R₁₄ is hydrogen, —C(O)—C₁₋₃ alkyl or —C(O)-chloro substituted C₁₋₃ alkyl;
R₁₅ is hydrogen, —CH₃ or —CHO;
when R₁₇ and R₁₈ are taken independently, R₁₈ is hydrogen, and either R₁₆ or R₁₇ is ethyl and the other is hydroxyl;
when R₁₇ and R₁₈ are taken together with the carbon to which they are attached to form an oxiran ring, R₁₆ is ethyl;
R₁₉ is hydrogen, OH, amino group, alkyl amino or —[C(R₂₀R₂₁)]ₐ—R₂₂;
each of R₂₀ and R₂₁ independently is hydrogen, C₁₋₆ alkyl, C₆₋₁₀ aryl, hydroxylated C₆₋₁₀ aryl, polyhydroxylated C₆₋₁₀ aryl, 5 to 12-membered heterocycle, C₃₋₈ cycloalkyl, hydroxylated C₃₋₈ cycloalkyl, polyhydroxylated C₃₋₈ cycloalkyl or a side chain of a natural or unnatural amino acid;
R₂₂ is —OH, —NH₂, —COOH, —R₈₂—C(O)(CH₂)_{c}—C(H)(R₂₃)—N(H)(R₂₃), —R₈₂—C(O)(CH₂)_{d}—(O CH₂—CH₂)_{f}—N(H)(R₂₃) or —R₈₂—(C(O)—CH(X²)—NH)_{d}—R₇₇;
each R₂₃ independently is hydrogen, C₁₋₆ alkyl, C₆₋₁₀ aryl, C₃₋₈ cycloalkyl, —COOH, or —COO—C₁₋₆ alkyl;
X² is a side chain of a natural or unnatural amino acid;
R₇₇ is hydrogen or X² and NR₇₇ form a nitrogen containing heterocyclic moiety;
R₈₂ is —NH or oxygen;
a is an integer from 1 to 6;
c is an integer from 0 to 3;
d is an integer from 1 to 3; and
f is an integer from 1 to 12.

Further examples of Vinca alkaloids are described in US 2010/0305149 and US 2002/0103136.

In one embodiment the Vinca alkaloid of Formula (V) is a compound of Formula (VI):

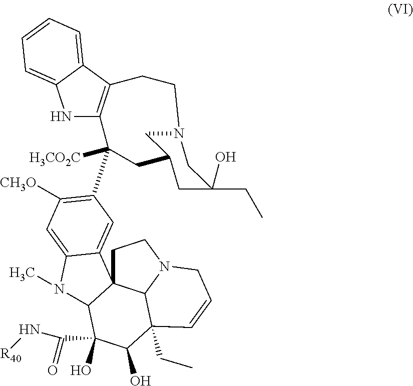

wherein:
R₄₀ is hydrogen, —OH, —NH₂, or any of the following structures:

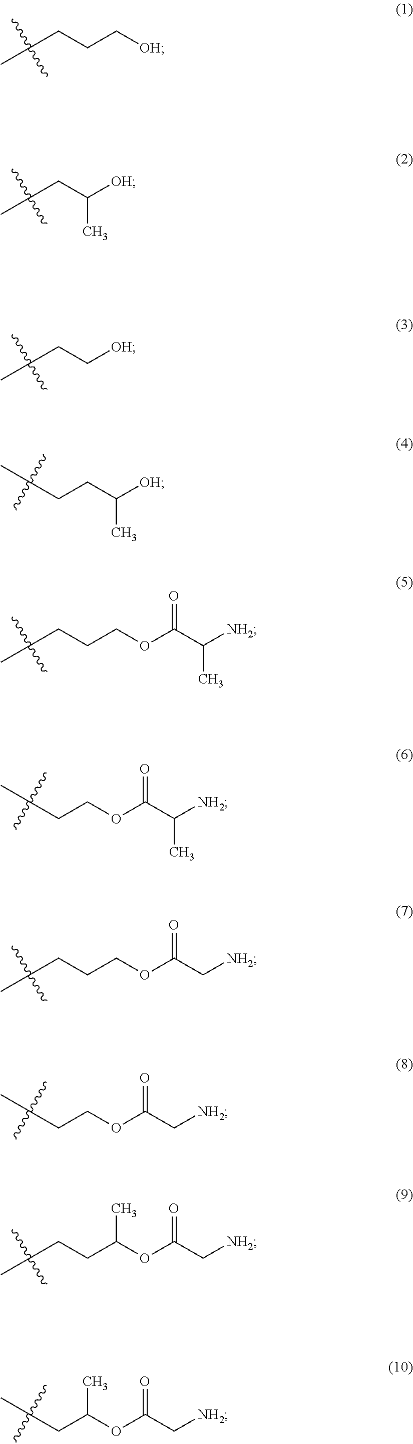

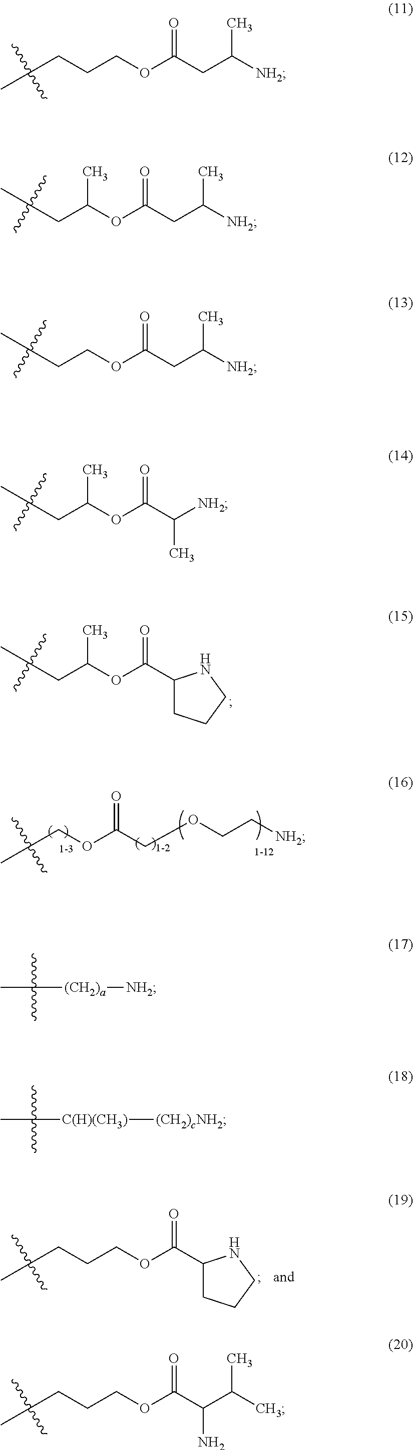

wherein:
a is an integer from 1 to 6; and
c is an integer from 0 to 3.

In one embodiment, R₄₀ is

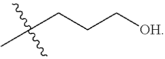 [SMILES: *CCCO]

In another embodiment, non-natural camptothecin is a compound of Formula (VII):

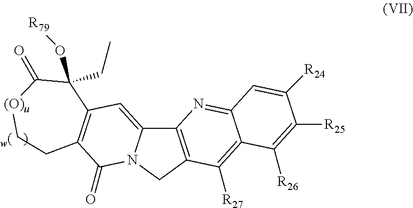

wherein:
R₂₄ is —H, —Cl, —F, —OH or alkyl; or R₂₄ and R₂₅, may be taken together to form an optionally substituted five- or six-membered ring;
R₂₅ is —H, —F, —OH, —CH₃, —CH═N—O-t-Butyl, —CH₂CH₂Si(CH₃)₃, —Si((CH₃)₂)-t-butyl, —O—C(O)—R₂₉;
R₂₉ is —NH₂, —R₂₈—C₁₋₆ alkyl-R₂₂, 5 to 12-membered heterocycloalkyl, R₂₈—C₅₋₁₂ heterocycloalkyl-C₁₋₆ alkyl-R₂₂ or —R₂₈—C₁₋₆ alkyl-C₆₋₁₂ aryl-C₁₋₆ alkyl-R₂₂; or R₂₉ is R₄₇ as defined herein;
R₂₆ is —H, —CH₂—N(CH₃)₂, NH₂, or NO₂;
R₂₇ is ethyl, N-methyl piperidine, cycloalkyl, —CH₂CH₂NHCH(CH₃)₂, or —N-4-methylcyclohexylamine;
R₇₉ is —H or —C(O)—R₂₈—[C(R₂₀R₂₁)]ₐ—R₂₂;
each of R₂₀ and R₂₁ independently is hydrogen, C₁₋₆ alkyl, C₆₋₁₀ aryl, hydroxylated C₆₋₁₀ aryl, polyhydroxylated C₆₋₁₀ aryl, 5 to 12-membered heterocycle, C₃₋₈ cycloalkyl, hydroxylated C₃₋₈ cycloalkyl, polyhydroxylated C₃₋₈ cycloalkyl or a side chain of a natural or unnatural amino acid;
R₂₂ is —OH, —NH₂, —COOH, —R₈₂—C(O)(CH₂)_{c}—C(H)(R₂₃)—N(H)(R₂₃), —R₈₂—C(O)(CH₂)_{d}—(O CH₂—CH₂)_{f}—N(H)(R₂₃), or —R₈₂—(C(O)—CH(X²)—NH)_{d}—R₇₇;
each R₂₃ independently is hydrogen, C₁₋₆ alkyl, C₆₋₁₀ aryl, C₃₋₈ cycloalkyl, —COOH, or —COO—C₁₋₆ alkyl;
X² is a side chain of a natural or unnatural amino acid;
R₇₇ is a hydrogen or X² and NR₇₇ form a nitrogen containing cyclic compound;
R₈₂ is —NH or oxygen;
or R₂₆ and R₂₇ when taken together with the two carbon atoms to which they attach and the third carbon atom connecting the two carbon atoms form an optionally substituted six-membered ring;
R₂₈ is absent, NH or oxygen;
a is an integer from 1 to 6;
c is an integer from 0 to 3;
d is an integer from 1 to 3;
f is an integer from 1 to 12;
u is an integer 0 or 1;
w is an integer 0 or 1; and
with the proviso that the compound of Formula (VII) must contain at least one of R₂₉ and R₇₉.

In one embodiment the non-natural camptothecin compound of Formula (VII) is a compound of Formula (VIII) or Formula (XXV):

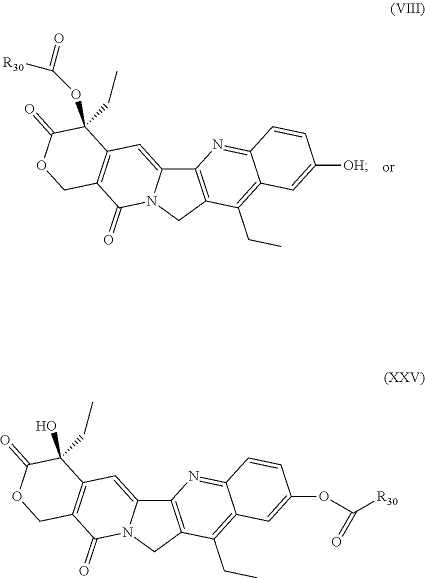

wherein R₃₀ is —NH₂, —R₂₈—C₁₋₆ alkyl-R₂₂, 5 to 12-membered heterocycloalkyl, R₂₈—C₅₋₁₂ heterocycloalkyl-C₁₋₆ alkyl-R₂₂ or —R₂₈—C₁₋₆ alkyl-C₆₋₁₂ aryl-C₁₋₆ alkyl-R₂₂;
R₂₈ is absent, NH or oxygen;
R₂₂ is —OH, —NH₂, —COOH, —R₈₂—C(O)(CH₂)_{c}—C(H)(R₂₃)—N(H)(R₂₃), —R₈₂—C(O)(CH₂)_{d}—(O CH₂—CH₂)_{f}—N(H)(R₂₃) or —R₈₂—(C(O)—CH(X²)—NH)_{d}—R₇₇;
each R₂₃ independently is hydrogen, C₁₋₆ alkyl, C₆₋₁₀ aryl, C₃₋₈ cycloalkyl, —COOH, or —COO—C₁₋₆ alkyl;
X² is a side chain of a natural or unnatural amino acid;
R₇₇ is a hydrogen or X² and NR₇₇ form a nitrogen containing cyclic compound;
R₈₂ is —NH or oxygen;
c is an integer from 0 to 3;
d is an integer from 1 to 3; and
f is an integer from 1 to 12.

In some embodiments R₃₀ is any one of the following structures:

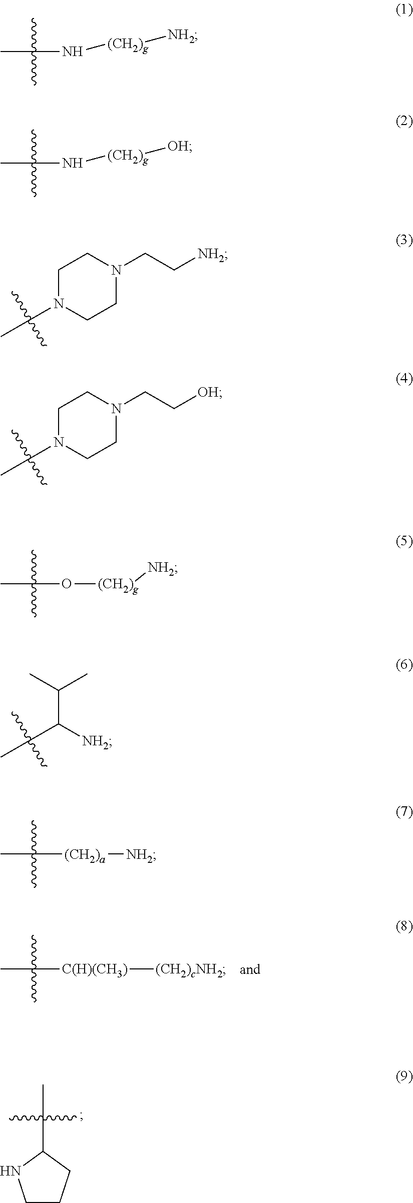

wherein:
a is an integer from 1 to 6;
c is an integer from 0 to 3; and
g is an integer from 2 to 6.

In another embodiment the PI3 kinase is a compound of Formula (IX):

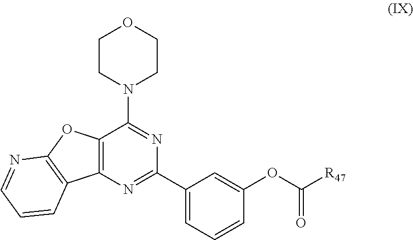

wherein
R₄₇ is an amino group, —R₉—[C(R₂₀R₂₁)]ₐ—R₁₀, —R₉—C₅₋₁₂ heterocycloalkyl-C₁₋₆ alkyl-R₁₀, 5 to 12-membered heterocycloalkyl, or —R₉—C₆₋₁₀ aryl;
each of R₂₀ and R₂₁ independently is hydrogen, C₁₋₆ alkyl, C₆₋₁₀ aryl, hydroxylated C₆₋₁₀ aryl, polyhydroxylated C₆₋₁₀ aryl, 5 to 12-membered heterocycle, C₃-s cycloalkyl, hydroxylated C₃₋₈ cycloalkyl, polyhydroxylated C₃₋₈ cycloalkyl or a side chain of a natural or unnatural amino acid;
R₁₀ is —OH, —NHR₈₃, —N—(R₈₃)R₁₁, —COOH, —R₈₂—C(O)(CH₂)_{c}—C(H)(R₂₃)—N(H)(R₂₃), —R₈₂—C(O)(CH₂)_{d}—(O CH₂—CH₂)_{f}—N(H)(R₂₃), —R₈₂—(C(O)—CH(X²)—NH)_{d}—R₇₇ or —R₈₂—C(O)—[C(R₂₀R₂₁)]ₐ—R₈₂-R₈₃ or

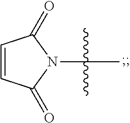 [SMILES: *N1C(=O)C=CC1=O]

each R₂₃ independently is hydrogen, C₁₋₆ alkyl, C₆₋₁₀ aryl, C₃₋₈ cycloalkyl, —COOH, or —COO—C₁₋₆ alkyl;
X² is a side chain of a natural or unnatural amino acid;
R₇₇ is a hydrogen or X² and NR₇₇ form a nitrogen containing cyclic compound;
R₈₂ is —NH or oxygen;
R₉ is absent, N—(R₈₃) or oxygen;
R₈₃ is hydrogen or CH₃;
R₁₁ is:

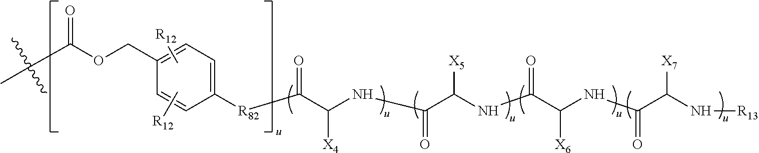

each R₁₂ independently is hydrogen, chloride, —CH₃ or —OCH₃;
R₁₃ is hydrogen or —C(O)—(CH₂)_{d}—(O—CH₂—CH₂)_{f}—NH₂;
R₈₂ is —NH or oxygen;
X₄ is the side chain of lysine, arginine, citrulline, alanine or glycine;
X₅ is the side chain of phenylalanine, valine, leucine, isoleucine or tryptophan;
each of X₆ and X₇ is independently the side chain of glycine, alanine, serine, valine or proline;
a is an integer from 1 to 6;
c is an integer from 0 to 3;
d is an integer from 1 to 3;
f is an integer from 1 to 12; and
each u independently is an integer 0 or 1.
or R₁₁ is —Yᵤ—W′_{q}—R₈₈,
   wherein:
Y is any one of the following structures:

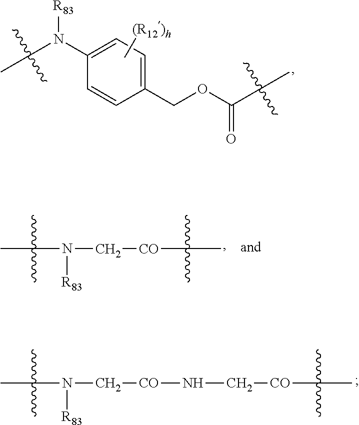

in each of which the terminal NR₈₃ group of Y is proximal to R₈₈;
R₈₃ is hydrogen or CH₃;
each W′ is an amino acid unit;
each R₁₂′ independently is halogen, —C₁₋₈ alkyl, —O—C₁₋₈ alkyl, nitro or cyano;
R₈₈ is hydrogen or —C(O)—(CH₂)_{ff}—(NH—C(O))ₐₐ-Eⱼ-(CH₂)_{bb}—R₈₅
R₈₅ is NH₂, OH or

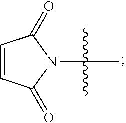 [SMILES: *N1C(=O)C=CC1=O]

E is —CH₂— or —CH₂CH₂O—;
u is an integer 0 or 1;
q is an integer from 0 to 12;
aa is an integer 0 or 1;
bb is an integer 0 or 2;
ff is an integer from 0 to 10;
h is an integer from 0 to 4;
j is an integer from 0 to 12; and
when E is —CH₂—, bb is 0 and j is an integer from 0 to 10; and when E is —CH₂CH₂—O—, bb is 2 and j is an integer from 1 to 12;
or R₁₁ is

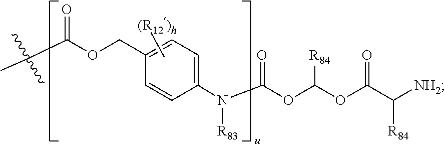

wherein:
R₈₃ is hydrogen or CH₃;
R₈₄ is C₁₋₆ alkyl or C₆₋₁₀ aryl;
each R₁₂′ independently is halogen, —C₁₋₈ alkyl, —O—C₁₋₈ alkyl, nitro or cyano;
h is an integer from 0 to 4; and
u is an integer 0 or 1.

In some embodiments, R₁₁ is:

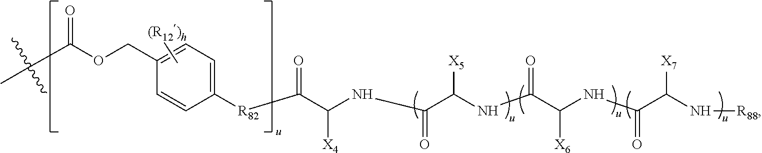

wherein:
each R₁₂′ independently is chloride, —CH₃ or —OCH₃;
R₈₈ is hydrogen or —C(O)—(CH₂)_{ff}—(CH₂—CH₂O)ⱼ—CH₂—CH₂—NH₂;
R₈₂ is —NH or oxygen
X₄ is the side chain of lysine, arginine, citrulline, alanine or glycine;
X₅ is the side chain of phenylalanine, valine, leucine, isoleucine or tryptophan;
each of X₆ and X₇ is independently the side chain of glycine, alanine, serine, valine or proline;
ff is an integer from 1 to 3;
j is an integer from 1 to 12
h is an integer from 0 to 4; and
each u independently is an integer 0 or 1.

In some embodiments

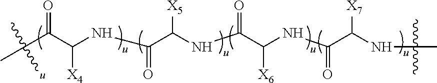

is citrulline-valine; lysine-phenylalanine; citrulline-phenylalanine; citrulline-leucine; citrulline-valine-glycine-glycine; glycine-phenylalanine-glycine-glycine; valine; proline; leucine or isoleucine.

In another embodiment, R₁₁ is any one of the following structures:

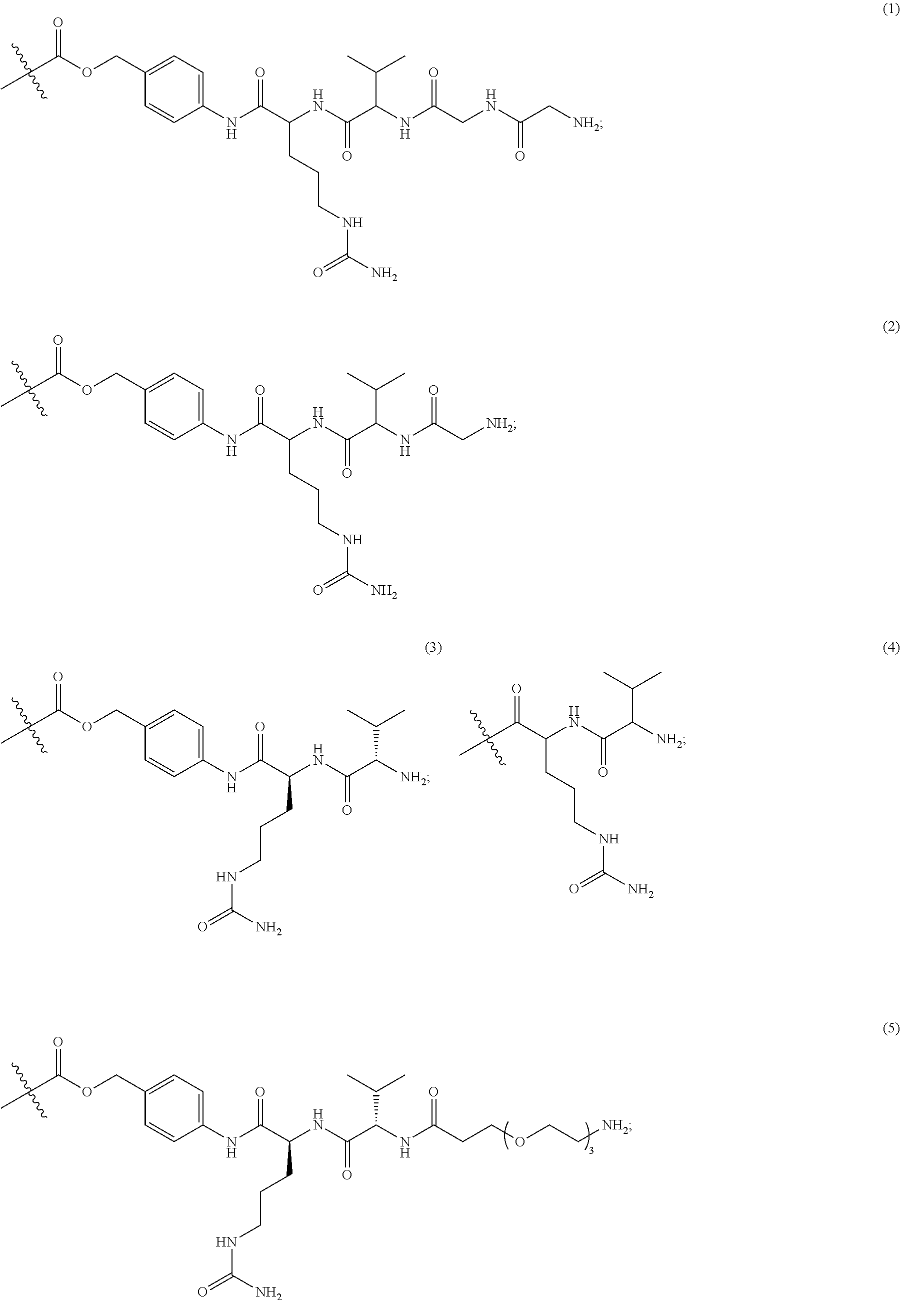

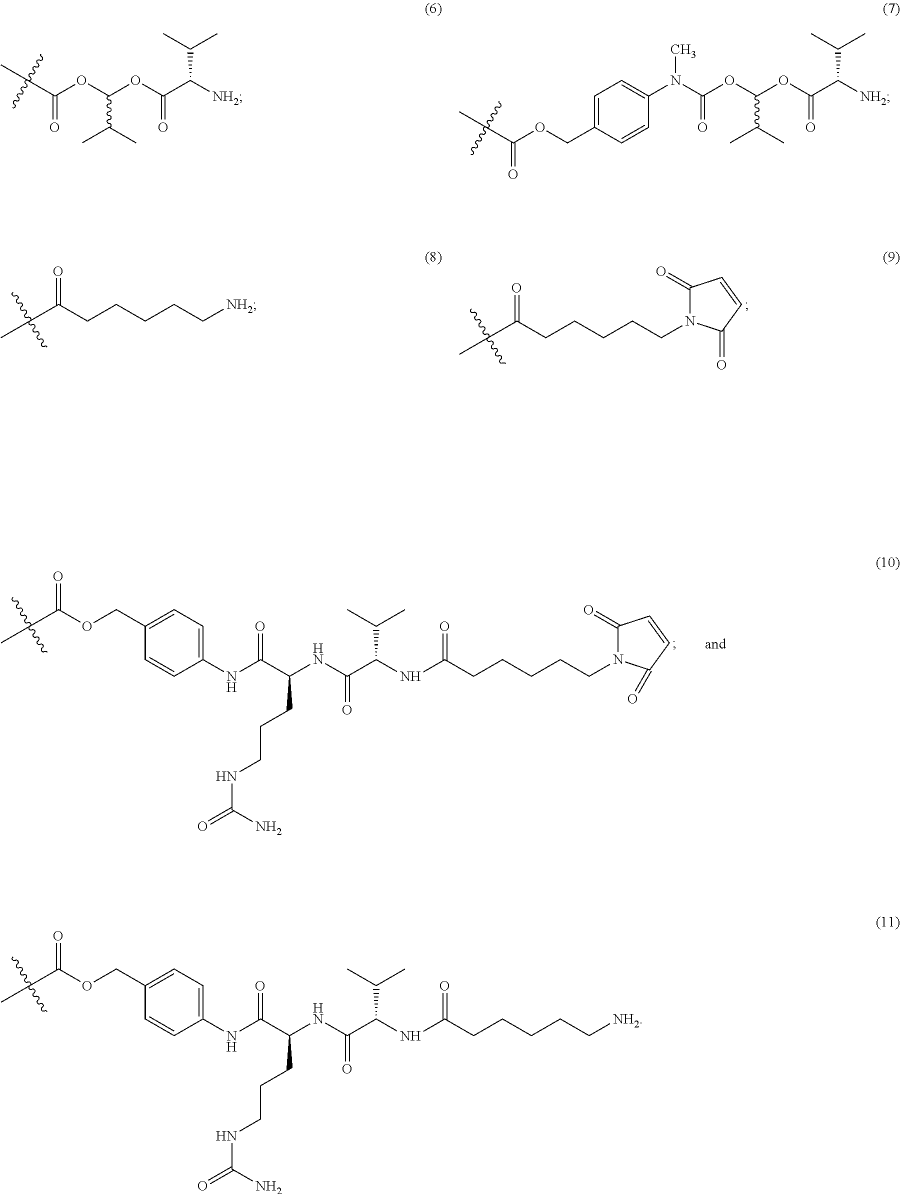 [SMILES: *C(=O)OC(OC(=O)[C@@H](N)C(C)C)C(C)C ; *C(=O)OCc1ccc(N(C)C(=O)OC(OC(=O)[C@@H](N)C(C)C)C(C)C)cc1 ; *C(=O)CCCCCN ; *C(=O)CCCCCN1C(=O)C=CC1=O ; *C(=O)OCc1ccc(NC(=O)[C@H](CCCNC(N)=O)NC(=O)[C@@H](NC(=O)CCCCCN2C(=O)C=CC2=O)C(C)C)cc1 ; *C(=O)OCc1ccc(NC(=O)[C@H](CCCNC(N)=O)NC(=O)[C@@H](NC(=O)CCCCCN)C(C)C)cc1]

In some embodiments R₄₇ is any one of the following structures:

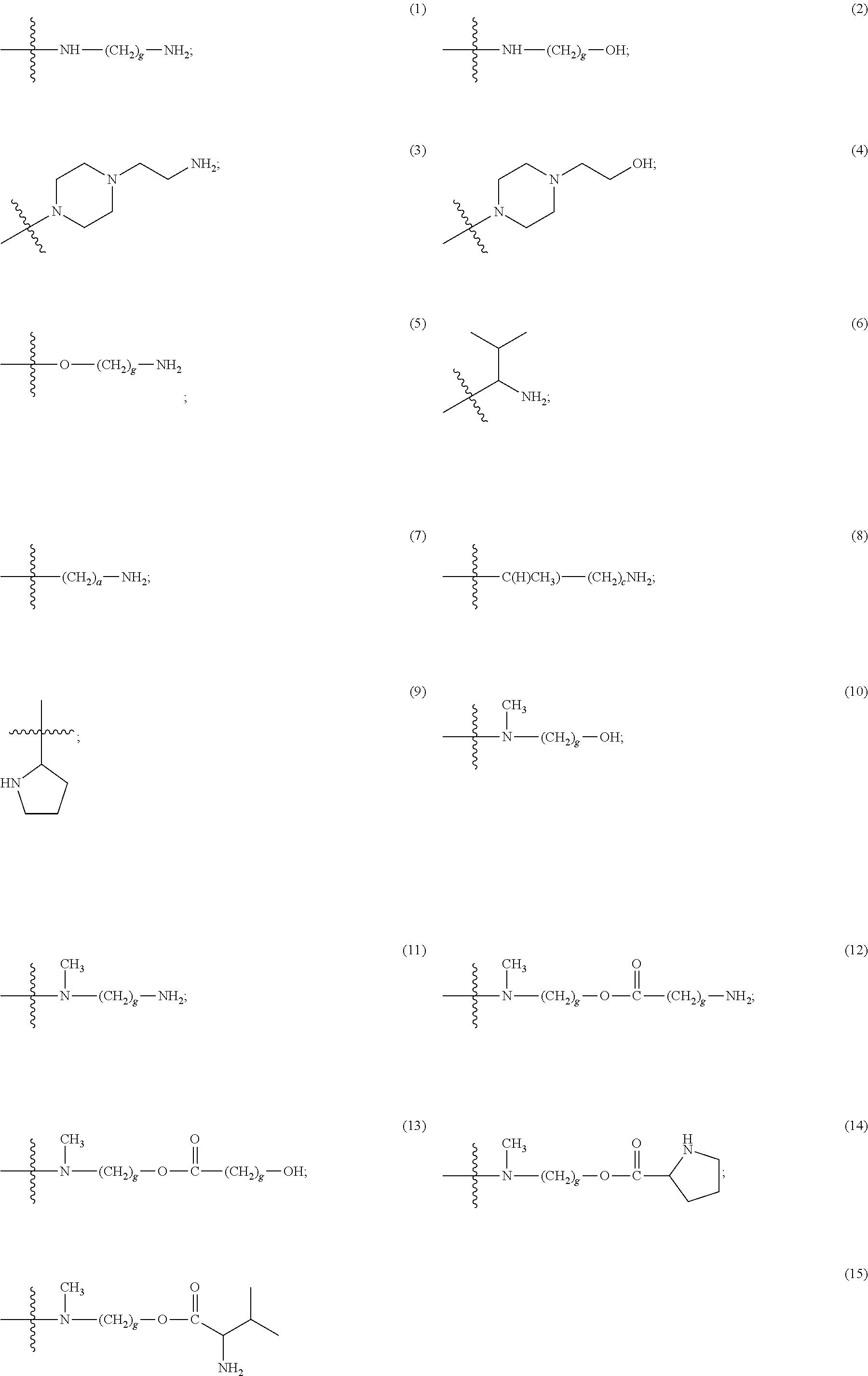

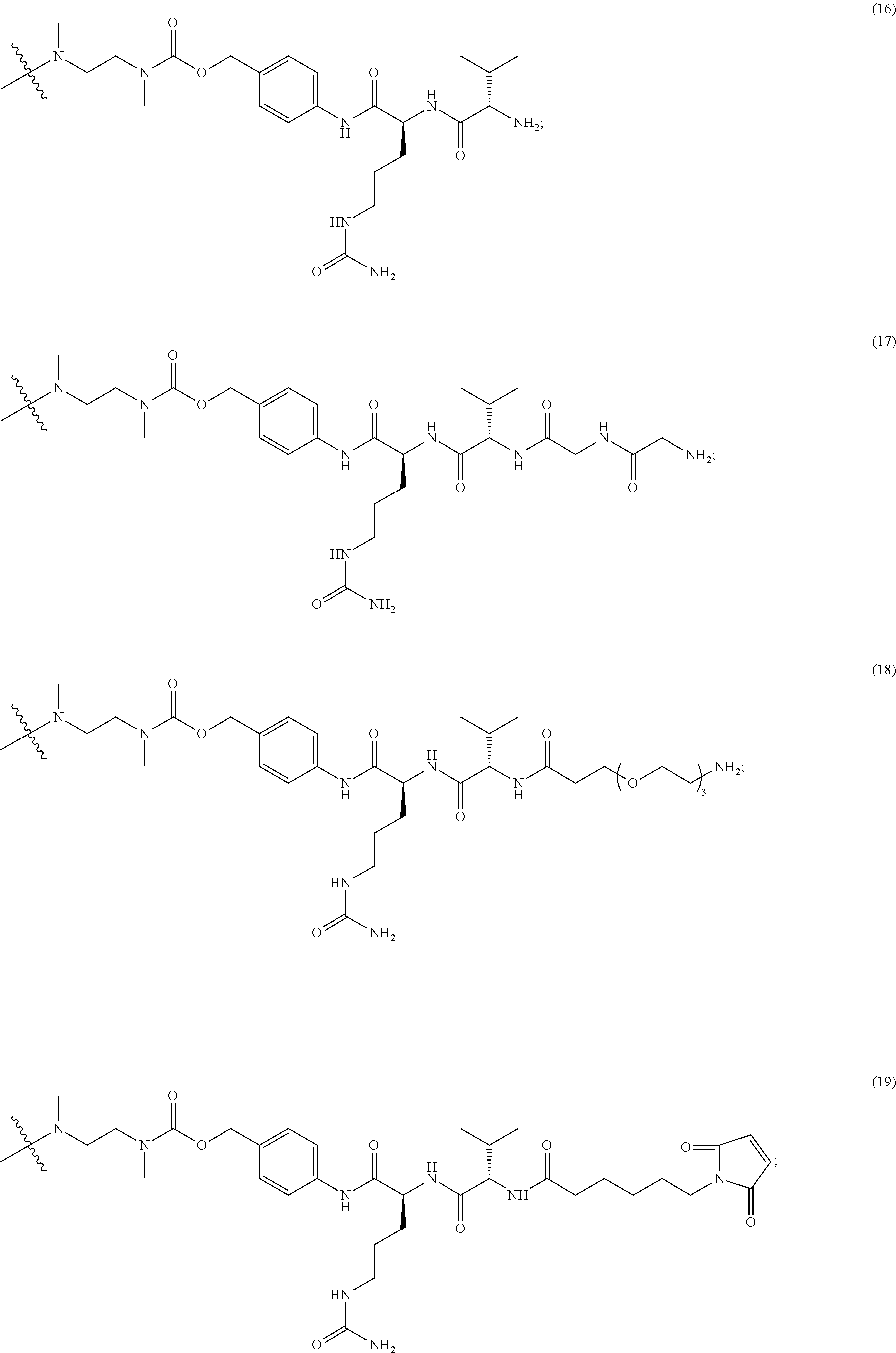

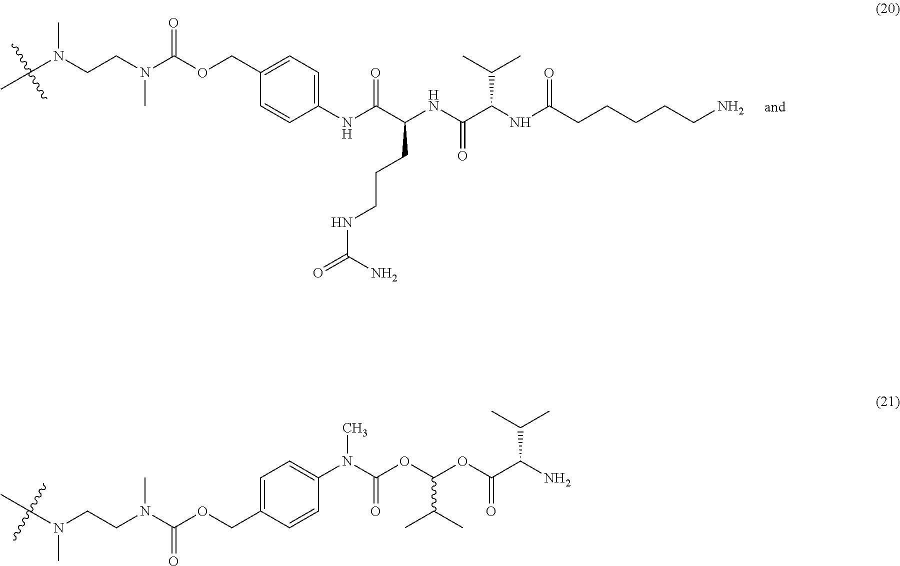

wherein:
a is an integer from 1 to 6;
c is an integer from 0 to 3; and
g is an integer from 2 to 6.

In another embodiment the auristatin is a compound of Formula (X):

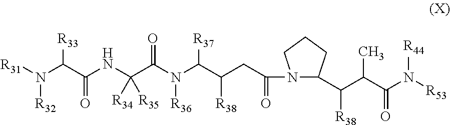

wherein:
each of R₃₁ and R₃₂ independently is hydrogen or C₁₋₈ alkyl and at most one of R₃₁ and R₃₂ is hydrogen;
R₃₃ is hydrogen, C₁₋₈ alkyl, C₃₋₈ carbocycle, C₆₋₁₀ aryl, C₁₋₈ alkyl-C₆₋₁₀ aryl, X¹—(C₃₋₈ carbocycle), C₃₋₈ heterocycle or X¹—(C₃₋₈ heterocycle);
R₃₄ is hydrogen, C₁₋₈ alkyl, C₃₋₈ carbocycle, C₆₋₁₀ aryl, X¹—C₆₋₁₀ aryl, X¹—(C₃₋₈ carbocycle), C₃₋₈ heterocycle or X¹—(C₃₋₈ heterocycle);
R₃₅ is hydrogen or methyl;
or R₃₄ and R₃₅, together with the carbon atom to which they attach form a carbocyclic ring having the formula —(CR₅₅R₄₁)_{b}— wherein each of R₅₅ and R₄₁ independently is hydrogen or C₁₋₈ alkyl and b is an integer from 3 to 7;
R₃₆ is hydrogen or C₁₋₈ alkyl;
R₃₇ is hydrogen, C₁₋₈ alkyl, C₃₋₈ carbocycle, C₆₋₁₀ aryl, —X¹—C₆₋₁₀ aryl, —X¹—(C₃₋₈ carbocycle), C₃₋₈ heterocycle or —X¹—(C₃₋₈ heterocycle);
each R₃₈ independently is hydrogen, OH, C₁₋₈ alkyl, C₃₋₈ carbocycle or O—(C₁₋₈ alkyl);
R₅₃ is:

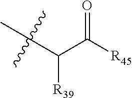

### or R₅₄

R₃₉ is H, C₁₋₈ alkyl, C₆₋₁₀ aryl, —X¹—C₆₋₁₀ aryl, C₃₋₈ carbocycle, C₃₋₈ heterocycle, —X¹—C₃₋₈ heterocycle, —C₁₋₈ alkylene-NH₂, or (CH₂)₂SCH₃
each X¹ independently is C₁₋₁₀ alkylene or C₃₋₁₀ cycloalkylene;
R₄₄ is hydrogen or C₁₋₈ alkyl;
R₄₅ is X³—R₄₂ or NH—R₁₉;
X³ is O or S;
R₁₉ is hydrogen, OH, amino group, alkyl amino or —[C(R₂₀R₂₁)]ₐ—R₂₂;
R₄₂ is an amino group, C₁₋₆ alkyl amino or —[C(R₂₀R₂₁)]ₐ—R₂₂;
each of R₂₀ and R₂₁ independently is hydrogen, C₁₋₆ alkyl, C₆₋₁₀ aryl, hydroxylated C₆₋₁₀ aryl, polyhydroxylated C₆₋₁₀ aryl, 5 to 12-membered heterocycle, C₃₋₈ cycloalkyl, hydroxylated C₃₋₈ cycloalkyl, polyhydroxylated C₃₋₈ cycloalkyl or a side chain of a natural or unnatural amino acid;
R₂₂ is —OH, —NHR₂₃, —COOH, —R₈₂—C(O)(CH₂)_{c}—C(H)(R₂₃)—N(H)(R₂₃), —R₈₂—C(O)(CH₂)_{d}—(O CH₂—CH₂)_{f}—N(H)(R₂₃) or —R₈₂—(C(O)—CH(X²)—NH)_{d}—R₇₇;
each R₂₃ independently is hydrogen, C₁₋₆ alkyl, C₆₋₁₀ aryl, C₃₋₈ cycloalkyl, —COOH, or —COO—C₁₋₆ alkyl;
X² is a side chain of a natural or unnatural amino acid;
R₇₇ is a hydrogen or X² and NR₇₇ form a nitrogen containing cyclic compound;
R₈₂ is —NH or oxygen;
R₅₄ is —C(R₅₆)₂—C(R₅₆)₂—C₆₋₁₀ aryl, —C(R₅₆)₂—C(R₅₆)₂—C₃₋₈ heterocycle or —C(R₅₆)₂—C(R₅₆)₂—C₃₋₈ carbocycle;
R₅₆ is independently selected from H, OH, C₁₋₈ alkyl, C₃₋₈ carbocycle, —O—C₁₋₈ alkyl, —O—C(O)—R₂₉ and —O—R₂₃—O—C₁₋₆ alkyl-NH₂;
R₂₉ is an amino group, 5 to 12-membered heterocycloalkyl, —R₂₈—C₁₋₆ alkyl-R₂₂, R₂₈—C₅₋₁₂ heterocycloalkyl-C₁₋₆ alkyl-R₂₂, —[C(R₂₀R₂₁)]ₐ—R₂₂, or —R₂₈—C₁₋₆ alkyl-C₆₋₁₂ aryl-C₁₋₆ alkyl-R₂₂; or R₂₉ is R₄₇ as defined herein;
R₂₈ is absent, NH or oxygen;
a is an integer from 1 to 6;
c is an integer from 0 to 3;
d is an integer from 1 to 3; and
f is an integer from 1 to 12.

In some embodiments, in the auristatin compound of Formula (X):
R₃₉ is benzyl or

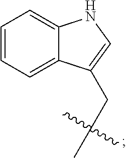 [SMILES: *Cc1c[nH]c2ccccc12]

and
R₄₄ is hydrogen.

In another embodiment the auristatin is a compound of Formula (Xa):

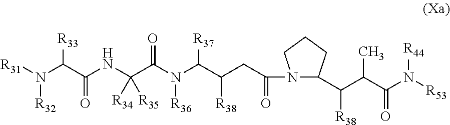

wherein:
R₃₃ through R₃₈, and R₄₄ are as defined herein,
one of R₃₁ and R₃₂ is hydrogen or C₁₋₈ alkyl and the other is:

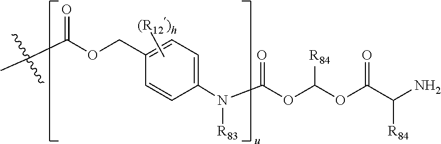

wherein:
R₈₃ is hydrogen or CH₃;
R₈₄ is C₁₋₆ alkyl or C₆₋₁₀ aryl;
each R₁₂′ independently is halogen, —C₁₋₈ alkyl, —O—C₁₋₈ alkyl, nitro or cyano;
h is an integer from 0 to 4; and
u is an integer 0 or 1;
R₅₃ is:

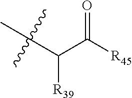

### or R₅₄

R₃₉ is H, C₁₋₈ alkyl, C₆₋₁₀ aryl, —X¹—C₆₋₁₀ aryl, C₃₋₈ carbocycle, C₃₋₈ heterocycle, —X¹—C₃₋₈ heterocycle, —C₁₋₈ alkylene-NH₂, or (CH₂)₂SCH₃,
each X¹ independently is C₁₋₁₀ alkylene or C₃₋₁₀ cycloalkylene;
R₄₅ is X³—R₄₂ or NH—R₁₉;
X³ is O or S;
R₁₉ is hydrogen, OH, amino group, alkyl amino or —[C(R₂₀R₂₁)]ₐ—R₂₂;
R₄₂ is H, an amino group, C₁₋₆ alkyl amino or —[C(R₂₀R₂₁)]ₐ—R₂₂;
each of R₂₀ and R₂₁ independently is hydrogen, C₁₋₆ alkyl, C₆₋₁₀ aryl, hydroxylated C₆₋₁₀ aryl, polyhydroxylated C₆₋₁₀ aryl, 5 to 12-membered heterocycle, C₃₋₈ cycloalkyl, hydroxylated C₃₋₈ cycloalkyl, polyhydroxylated C₃₋₈ cycloalkyl or a side chain of a natural or unnatural amino acid;
R₂₂ is —OH, —NHR₂₃, —COOH, —R₈₂—C(O)(CH₂)_{c}—C(H)(R₂₃)—N(H)(R₂₃), —R₈₂—C(O)(CH₂)_{d}—(O CH₂—CH₂)_{f}—N(H)(R₂₃) or —R₈₂—(C(O)—CH(X²)—NH)_{d}—R₇₇;
each R₂₃ independently is hydrogen, C₁₋₆ alkyl, C₆₋₁₀ aryl, C₃₋₈ cycloalkyl, —COOH, or —COO—C₁₋₆ alkyl;
X² is a side chain of a natural or unnatural amino acid;
R₇₇ is a hydrogen or X² and NR₇₇ form a nitrogen containing cyclic compound;
R₈₂ is —NH or oxygen;
R₅₄ is —C(R₅₆)₂—C(R₅₆)₂—C₆₋₁₀ aryl, —C(R₅₆)₂—C(R₅₆)₂—C₃₋₈ heterocycle or —C(R₅₆)₂—C(R₅₆)₂—C₃₋₈ carbocycle;
R₅₆ is independently selected from H, OH, C₁₋₈ alkyl, C₃₋₈ carbocycle, —O—C₁₋₈ alkyl, —O—C(O)—R₂₉ and —O—R₂₃—O—C₁₋₆ alkyl-NH₂;
R₂₉ is an amino group, 5 to 12-membered heterocycloalkyl, —R₂₈—C₁₋₆ alkyl-R₂₂, R₂₈—C₅₋₁₂ heterocycloalkyl-C₁₋₆ alkyl-R₂₂, —[C(R₂₀R₂₁)]ₐ—R₂₂, or —R₂₈—C₁₋₆ alkyl-C₆₋₁₂ aryl-C₁₋₆ alkyl-R₂₂; or R₂₉ is R₄₇ as defined herein;
R₂₈ is absent, NH or oxygen;
a is an integer from 1 to 6;
c is an integer from 0 to 3;
d is an integer from 1 to 3; and
f is an integer from 1 to 12.

In one embodiment, the auristatin compound of Formula (Xa) is a compound of Formula (XIa) or Formula (XIb):

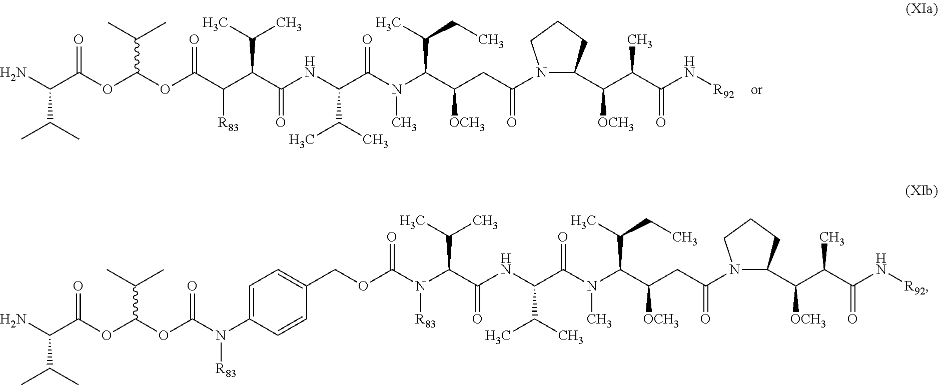

wherein:
R₉₂ is:

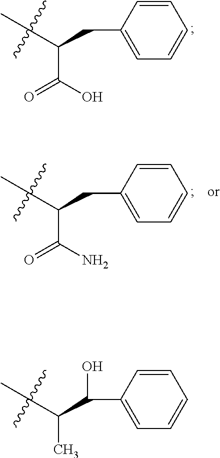 [SMILES: *[C@@H](Cc1ccccc1)C(=O)O ; *[C@@H](Cc1ccccc1)C(N)=O ; *[C@H](C)C(O)c1ccccc1]

R₈₃ is hydrogen or CH₃.

In one embodiment the auristatin of Formula (X) is a compound of Formula (XI), Formula (XII) or Formula (XIII):
wherein the compound of Formula (XI) is:

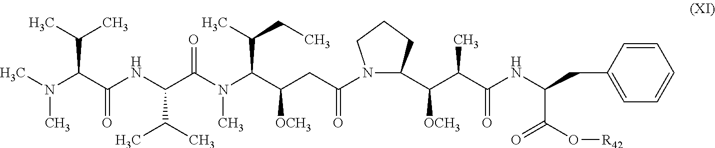

wherein R₄₂ is —CH₃ or any one of the following structures:

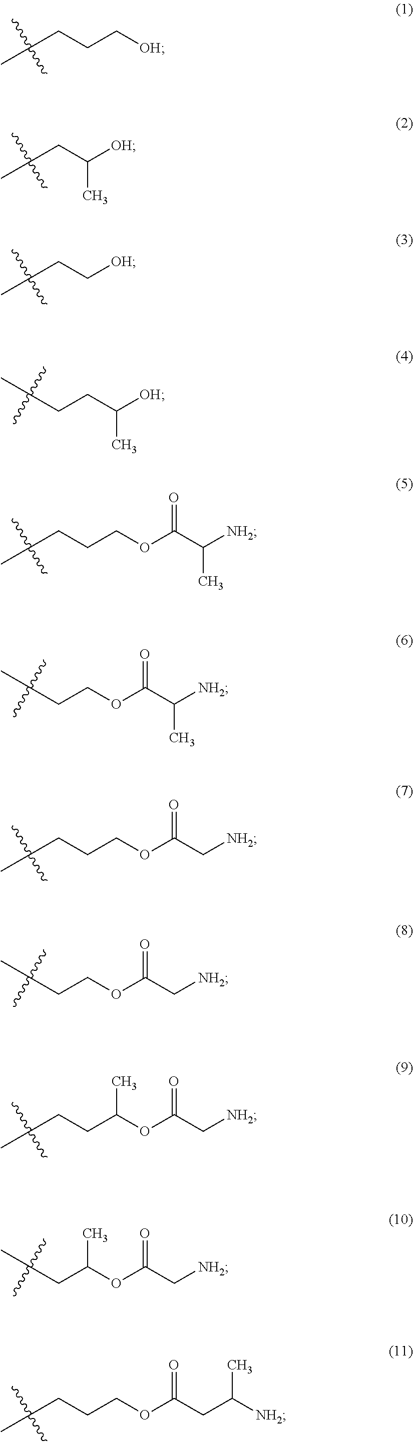

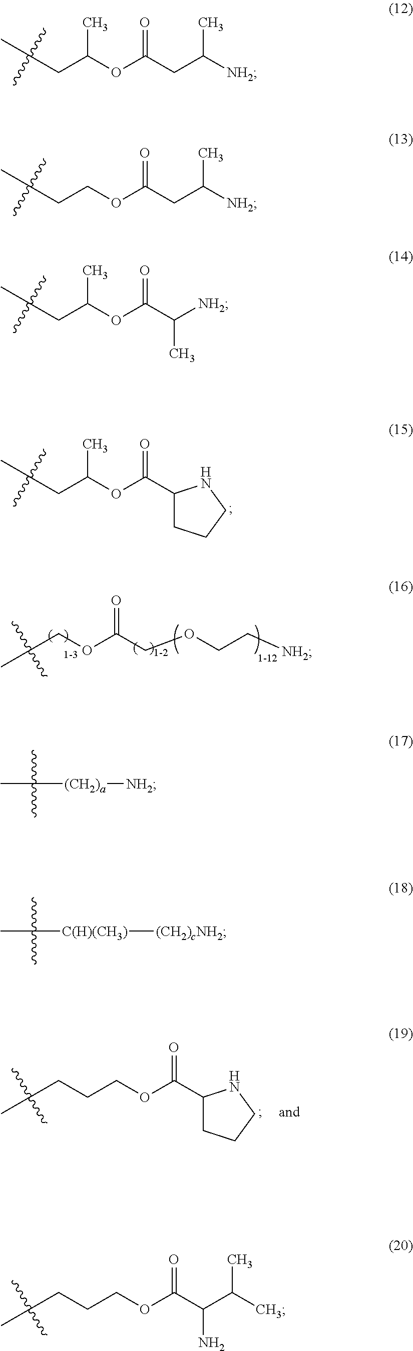

wherein:
a is an integer from 1 to 6; and
c is an integer from 0 to 3;
wherein the compound of Formula (XII) is:

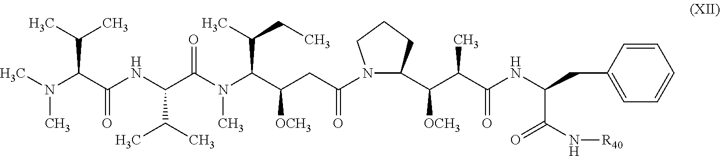

wherein R₄₀ is hydrogen, —OH, —NH₂, or any of the following structures:

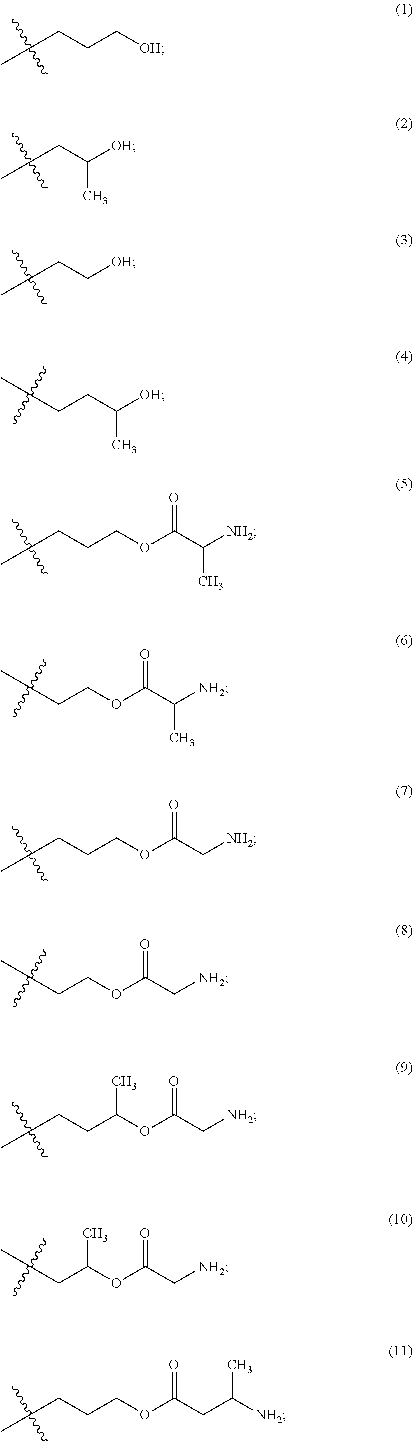

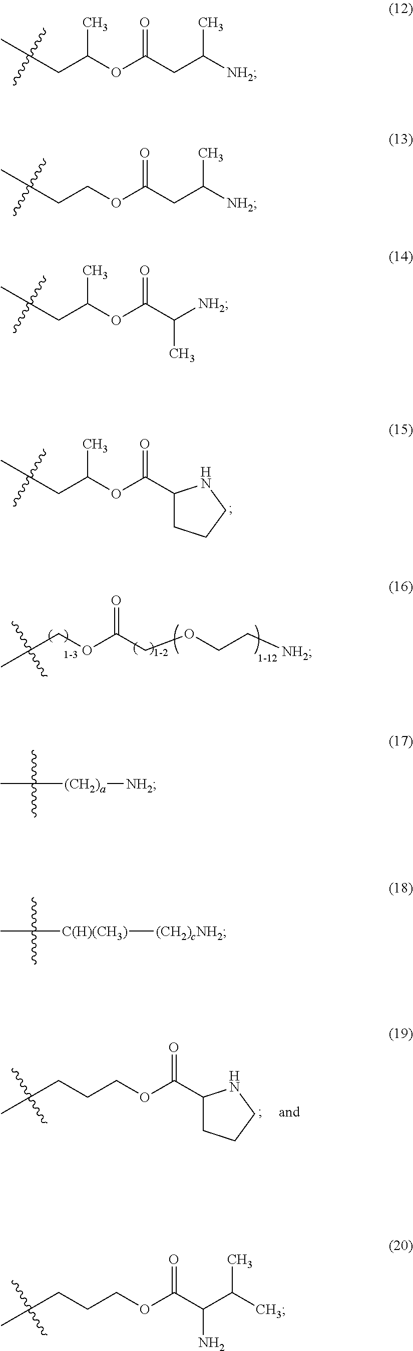

wherein:
a is an integer from 1 to 6; and
c is an integer from 0 to 3.
wherein the compound of Formula (XIII) is:

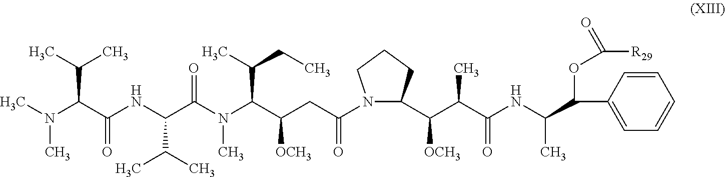

wherein R₂₉ is an amino group, 5 to 12-membered heterocycloalkyl, —R₂₈—C₁₋₆ alkyl-R₂₂, R₂₈—C₅₋₁₂ heterocycloalkyl-C₁₋₆ alkyl-R₂₂, —R₂₈—[C(R₂₀R₂₁)]ₐ—R₂₂, or —R₂₈—C₁₋₆ alkyl-C₆₋₁₂ aryl-C₁₋₆ alkyl-R₂₂; or R₂₉ is R₄₇ as defined herein;
each of R₂₀ and R₂₁ independently is hydrogen, C₁₋₆ alkyl, C₆₋₁₀ aryl, hydroxylated C₆₋₁₀ aryl, polyhydroxylated C₆₋₁₀ aryl, 5 to 12-membered heterocycle, C₃₋₈ cycloalkyl, hydroxylated C₃₋₈ cycloalkyl, polyhydroxylated C₃₋₈ cycloalkyl or a side chain of a natural or unnatural amino acid;
R₂₂ is —OH, —NHR₂₃, —COOH, —R₈₂—C(O)(CH₂)_{c}—C(H)(R₂₃)—N(H)(R₂₃), —R₈₂—C(O)(CH₂)_{d}—(O CH₂—CH₂)_{f}—N(H)(R₂₃) or —R₈₂—(C(O)—CH(X²)—NH)_{d}—R₇₇;
each R₂₃ independently is hydrogen, C₁₋₆ alkyl, C₆₋₁₀ aryl, C₃₋₈ cycloalkyl, —COOH, or —COO—C₁₋₆ alkyl;
X² is a side chain of a natural or unnatural amino acid;
R₇₇ is a hydrogen or X² and NR₇₇ form a nitrogen containing cyclic compound;
R₈₂ is —NH or oxygen;
R₂₈ is absent, NH or oxygen;
a is an integer from 1 to 6;
c is an integer from 0 to 3;
d is an integer from 1 to 3; and
f is an integer from 1 to 12.

In one embodiment, in Formula (XII), R₄₀ is

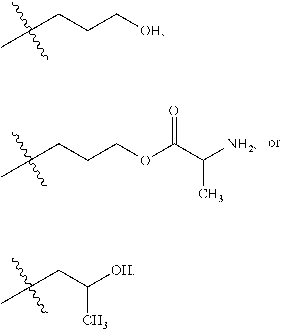

In one embodiment in the compound of Formula (XIII), R₂₉ is —NH₂, 5 membered heterocycloalkyl, —R₂₈—C₁₋₆ alkyl-R₂₂, R₂₈—C₅₋₁₂ heterocycloalkyl-C₁₋₆ alkyl-R₂₂ or —R₂₈—C₁-6 alkyl-C₆₋₁₂ aryl-C₁₋₆ alkyl-R₂₂; or R₂₉ is R₄₇ as defined herein;
R₂₈ is absent, NH or oxygen;
R₂₂ is —OH, —NHR₂₃, —COOH, —R₈₂—C(O)(CH₂)_{c}—C(H)(R₂₃)—N(H)(R₂₃), —R₈₂—C(O)(CH₂)_{d}—(O CH₂—CH₂)_{f}—N(H)(R₂₃) or —R₈₂—(C(O)—CH(X²)—NH)_{d}—R₇₇;
each R₂₃ independently is hydrogen, C₁₋₆ alkyl, C₆₋₁₀ aryl, C₃₋₈ cycloalkyl, —COOH, or —COO—C₁₋₆ alkyl;
X² is a side chain of a natural or unnatural amino acid;
R₇₇ is a hydrogen or X² and NR₇₇ form a nitrogen containing cyclic compound;
R₈₂ is —NH or oxygen;
c is an integer from 0 to 3;
d is an integer from 1 to 3; and
f is an integer from 1 to 12.

In yet another embodiment, R₂₉ is any one of the following structures:

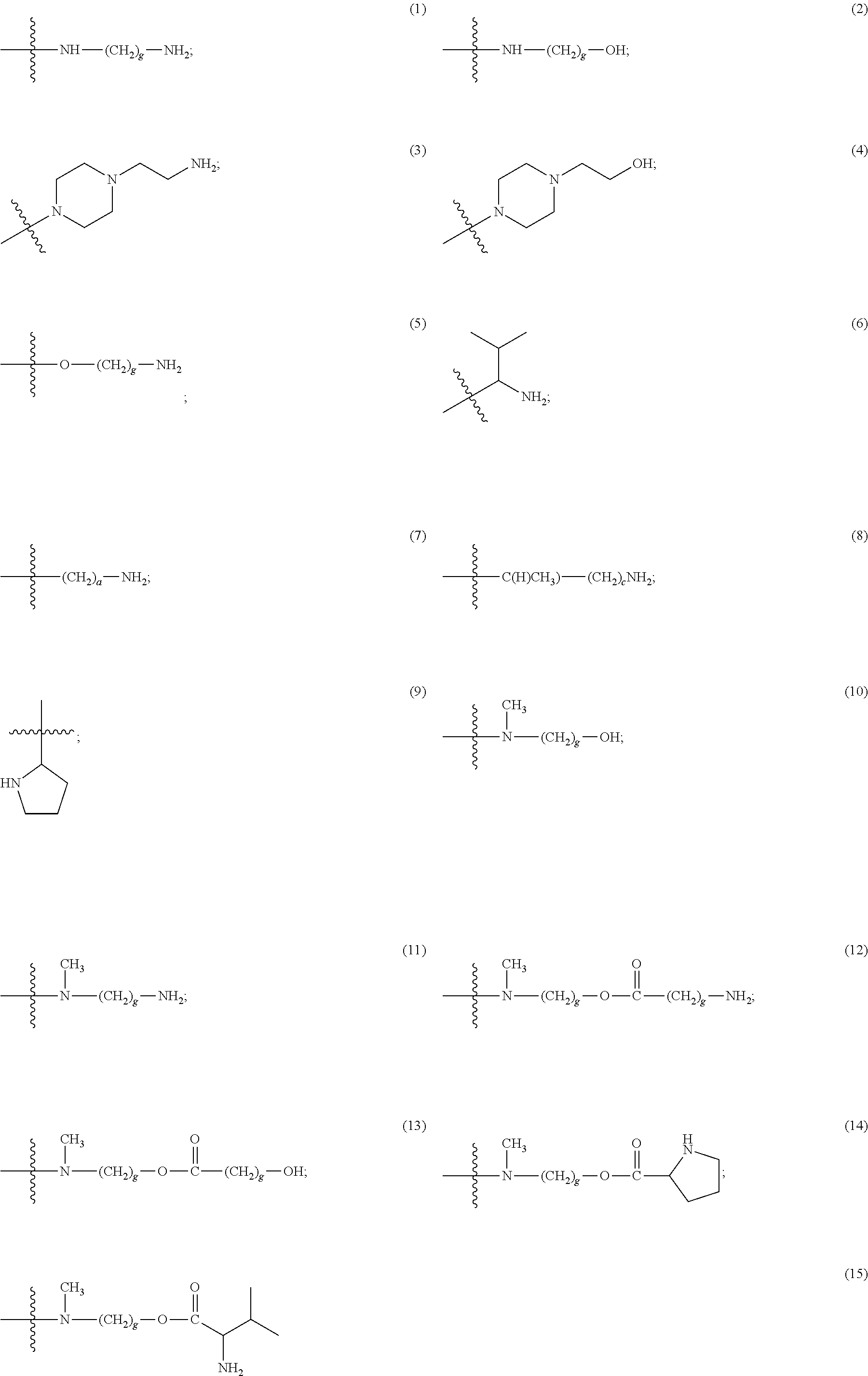

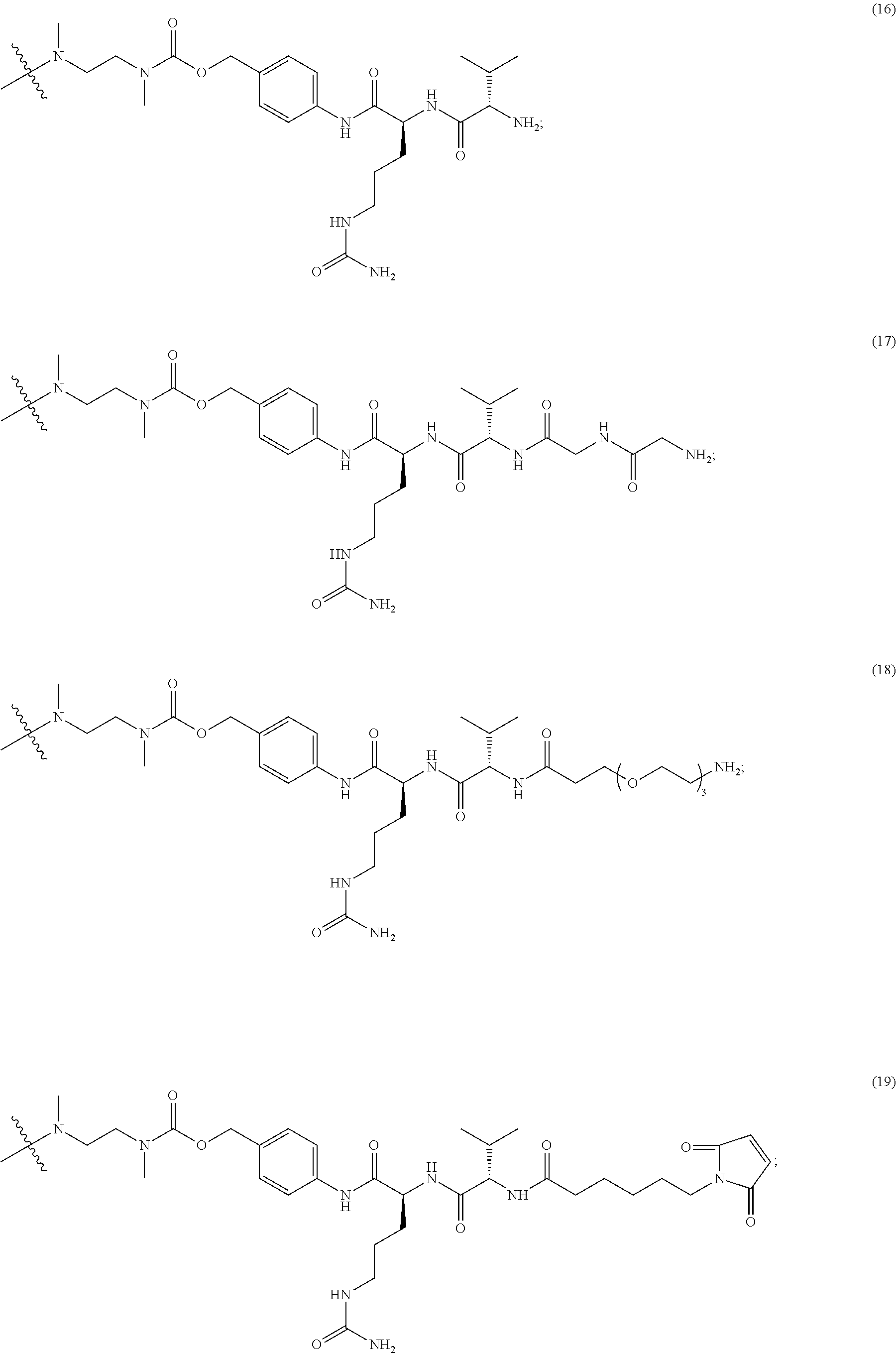

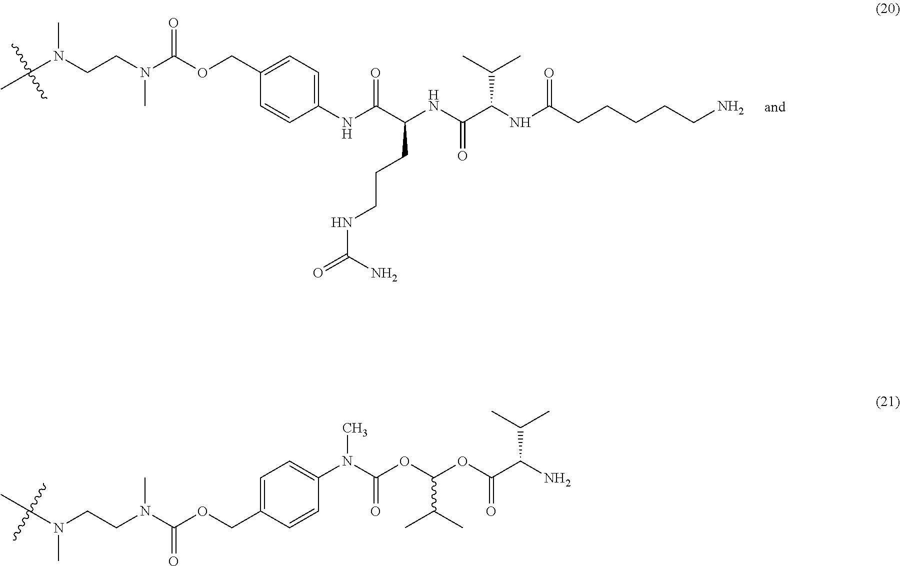

wherein:
a is an integer from 1 to 6;
c is an integer from 0 to 3; and
g is an integer from 2 to 6.

In one embodiment, the MEK inhibitor is a compound of Formula (XIV):

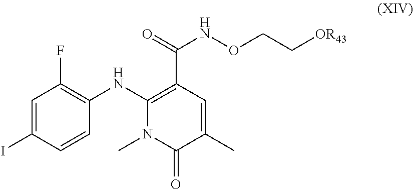

wherein R₄₃ is H or —R₄₆-R₄₇;
each of R₂₀ and R₂₁ independently is hydrogen, C₁₋₆ alkyl, C₆₋₁₀ aryl, hydroxylated C₆₋₁₀ aryl, polyhydroxylated C₆₋₁₀ aryl, 5 to 12-membered heterocycle, C₃₋₈ cycloalkyl, hydroxylated C₃₋₈ cycloalkyl, polyhydroxylated C₃₋₈ cycloalkyl or a side chain of a natural or unnatural amino acid;
R₂₂ is —OH, —NH₂, —COOH, —R₈₂—C(O)(CH₂)_{c}—C(H)(R₂₃)—N(H)(R₂₃), —R₈₂—C(O)(CH₂)_{d}—(O CH₂—CH₂)_{f}—N(H)(R₂₃) or —R₈₂—(C(O)—CH(X²)—NH)_{d}—R₇₇;
each R₂₃ independently is hydrogen, C₁₋₆ alkyl, C₆₋₁₀ aryl, C₃₋₈ cycloalkyl, —COOH, or —COO—C₁₋₆ alkyl;
X² is a side chain of a natural or unnatural amino acid;
R₇₇ is a hydrogen or X² and NR₇₇ form a nitrogen containing cyclic compound;
R₈₂ is —NH or oxygen;
R₄₆ is —C(O)—; —C(O)—O—, —C(O)—NH—, or absent;
R₄₇ is as defined herein;
a is an integer from 1 to 6;
c is an integer from 0 to 3;
d is an integer from 1 to 3; and
f is an integer from 1 to 12.

Further examples of the MEK inhibitor are disclosed in U.S. Pat. No. 7,517,994 B2.

In some embodiments R₄₃ is —C(O)—(CH₂)ₐ—NH₂, or —C(O)—C(H)(CH₃)—(CH₂)_{c}—NH₂; in which a is an integer from 1 to 6; and c is an integer from 0 to 3.

In another embodiment, the duocarmycin compound is a compound of Formula (XV):

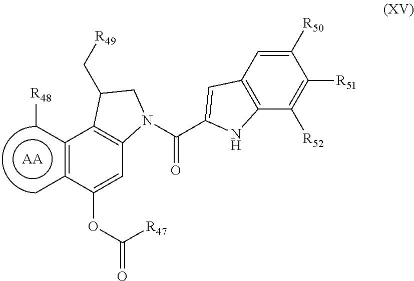

wherein:
R₄₇ is as defined herein;
R₄₈ is hydrogen, —COOC₁₋₆ alkyl, —COOH, —NH₂ or —CH₃;
R₄₉ is Cl, Br or —OH;
R₅₀ is hydrogen, —OCH₃,

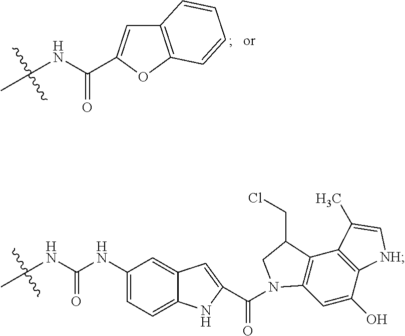 [SMILES: *NC(=O)c1cc2ccccc2o1 ; *NC(=O)Nc1ccc2[nH]c(C(=O)N3CC(CCl)c4c3cc(O)c3[nH]cc(C)c43)cc2c1]

each of R₅₁ and R₅₂ independently is hydrogen or —OCH₃; and
ring AA is either a phenyl or pyrrolyl ring.

Further examples of duocarmycin compounds are disclosed in U.S. Pat. No. 7,553,816.

In one embodiment the duocarmycin compound of Formula (XV) is a compound of Formula (XVI), (XVII), (XVIII) or (XIX):

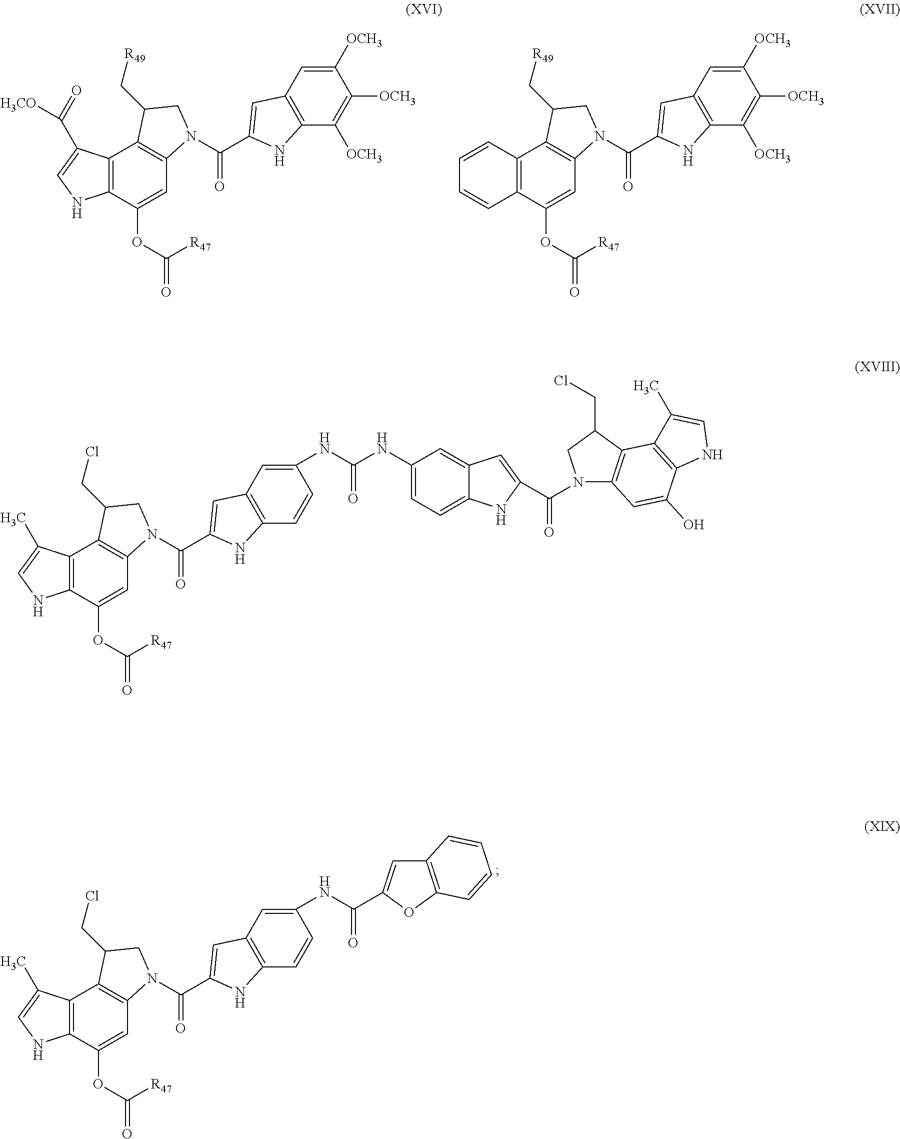

wherein:
R₄₉ is Cl, Br or —OH; and
R₄₇ is as defined herein.

In another embodiment, the duocarmycin compound is a duocarmycin SA compound of Formula (XX): U.S. Pat. No. 5,101,038; or (XXI):

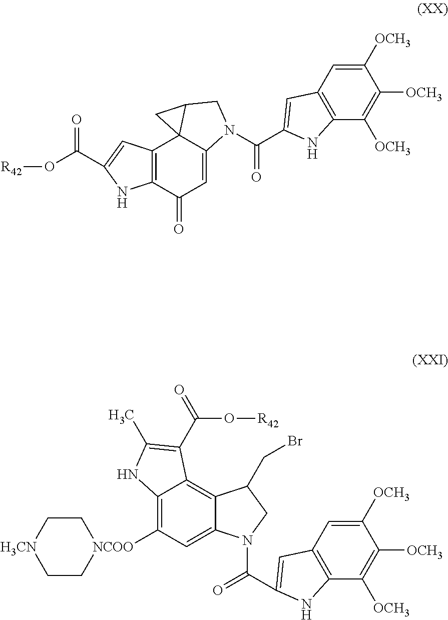

wherein:
R₄₂ is C₁₋₆ alkyl amino or —[C(R₂₀R₂₁)]ₐ—R₂₂;
each of R₂₀ and R₂₁ independently is hydrogen, C₁₋₆ alkyl, C₆₋₁₀ aryl, hydroxylated C₆₋₁₀ aryl, polyhydroxylated C₆₋₁₀ aryl, 5 to 12-membered heterocycle, C₃₋₈ cycloalkyl, hydroxylated C₃₋₈ cycloalkyl, polyhydroxylated C₃₋₈ cycloalkyl or a side chain of a natural or unnatural amino acid;
R₂₂ is —OH, —NH₂, —COOH, —R₈₂—C(O)(CH₂)_{c}—C(H)(R₂₃)—N(H)(R₂₃), —R₈₂—C(O)(CH₂)_{d}—(O CH₂—CH₂)_{f}—N(H)(R₂₃), or —R₈₂—(C(O)—CH(X²)—NH)_{d}—R₇₇;
each R₂₃ independently is hydrogen, C₁₋₆ alkyl, C₆₋₁₀ aryl, C₃₋₈ cycloalkyl, —COOH, or —COO—C₁₋₆ alkyl;
X² is a side chain of a natural or unnatural amino acid;
R₇₇ is a hydrogen or X² and NR₇₇ form a nitrogen containing cyclic compound;
R₈₂ is —NH or oxygen;
a is an integer from 1 to 6;
c is an integer from 0 to 3;
d is an integer from 1 to 3; and
f is an integer from 1 to 12.

In some embodiments, R₄₂ is any one of the following structures:

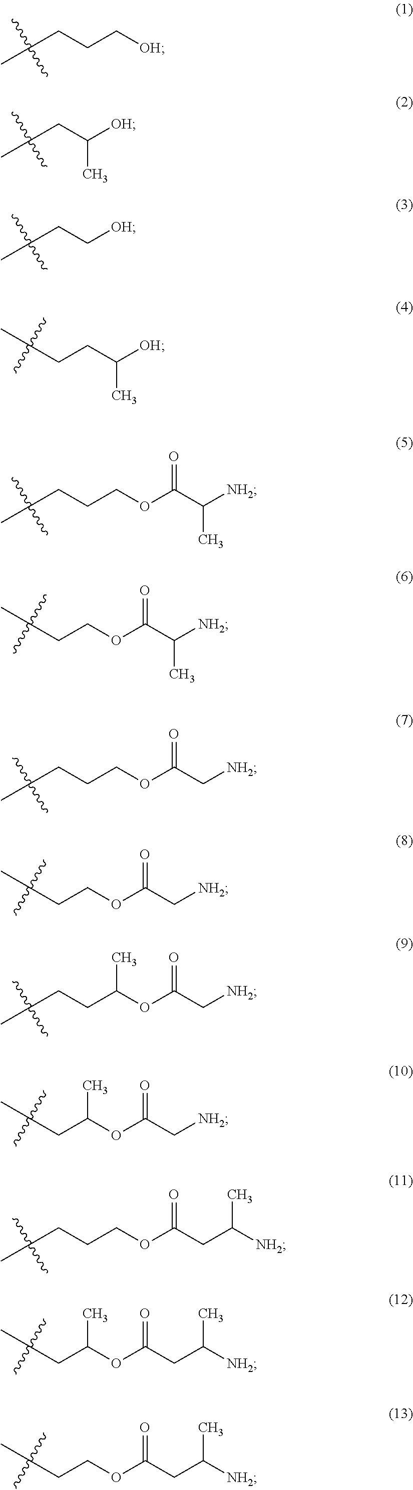

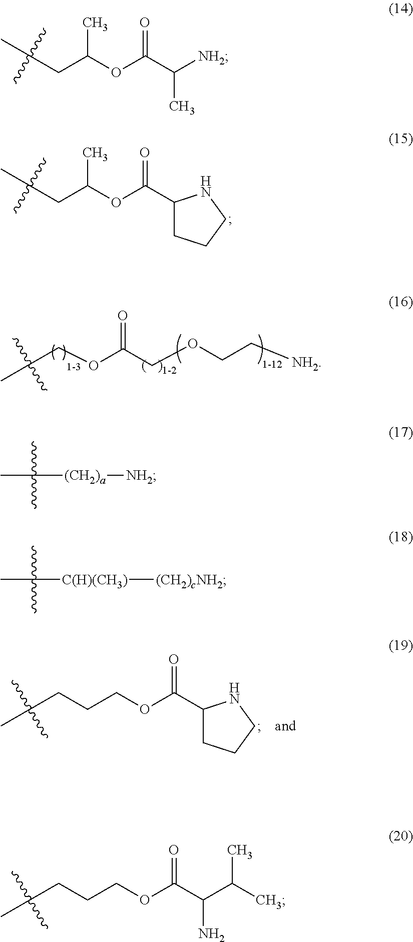

wherein:
a is an integer from 1 to 6; and
c is an integer from 0 to 3.

In another embodiment the tubulysin is a compound of Formula (XXII):

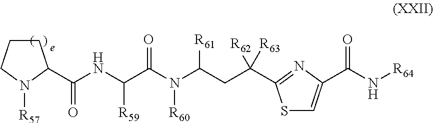

wherein:
R₅₇ is C₁₋₄ alkyl or —C(O)R₅₈;
R₅₈ is C₁₋₆ alkyl, CF₃ or C₆₋₁₀ aryl;
R₅₉ is C₁₋₆ alkyl;
R₆₀ is hydrogen, C₁₋₆ alkyl, C₂₋₇ alkenyl, —CH₂-phenyl, CH₂OR₆₅ or CH₂OCOR₆₆;
R₆₅ is hydrogen, C₁₋₆ alkyl, C₂₋₇ alkenyl, C₆₋₁₀ aryl or C(O)R₆₇;
R₆₇ is C₁₋₆ alkyl, C₂₋₆ alkenyl, C₆₋₁₀ aryl or heteroaryl;
R₆₆ is C₁₋₆ alkyl, —C₆H₅ or —CH₂-phenyl;
R₆₁ is C₁₋₆ alkyl;
R₆₂ is hydrogen, OH, O—C₁₋₄ alkyl or O—C(O)—C₁₋₄ alkyl;
R₆₃ is hydrogen, OH, O—C₁₋₄ alkyl, O—C(O)—C₁₋₄ alkyl, halogen or C₁₋₆ alkyl;
e is an integer between 1 and 3 inclusive;
R₆₄ is.

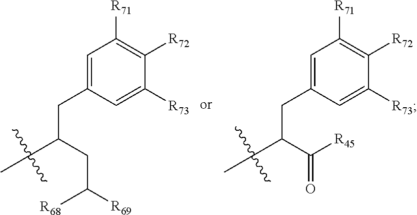

wherein:
R₆₈ is hydrogen or C₁-C₆ alkyl;
R₆₉ is CO₂R₇₀, C(O)—R₇₈, CONHNH₂, OH, NH₂, SH or optionally substituted alkyl, an optionally substituted cycloalkyl, an optionally substituted heteroalkyl or an optionally substituted heterocycloalkyl group;
R₇₀ is an optionally substituted alkyl (i.e. C₁₋₆ alkyl amine), an optionally substituted heteroalkyl or an optionally substituted heterocycloalkyl group;
each of R₇₁ and R₇₃ independently is hydrogen, halo, —NO₂, —CN, —NHR₇₄, C₁₋₆ alkyl, haloalkyl, alkoxy, and haloalkoxy;
R₇₂ is hydrogen, OR₄₃, alkoxy, halogen, —NHR₇₄, —O—C(O)—R₄₇, NO₂, —CN, C₆₋₁₀ aryl, C₁₋₆ alkyl, amino or dialkylamino;
R₇₄ is hydrogen, —CHO, —C(O)—C₁₋₄ alkyl, OH, amino group, alkyl amino or —[C(R₂₀R₂₁)]ₐ—R₂₂;
R₄₃ is H or —R₄₆-R₄₇;
R₄₆ is —C(O)—; —C(O)—O—, —C(O)—NH—, or absent;
R₄₇ is as defined herein;
R₇₈ is X³—R₇₅ or NH—R₁₉;
X³ is O or S;
R₁₉ is hydrogen, OH, amino group, alkyl amino or —[C(R₂₀R₂₁)]ₐ—R₂₂;
R₇₅ is a hydrogen, an amino group, C₁₋₆ alkyl amino or —[C(R₂₀R₂₁)]ₐ—R₂₂;
each of R₂₀ and R₂₁ independently is hydrogen, C₁₋₆ alkyl, C₆₋₁₀ aryl, hydroxylated C₆₋₁₀ aryl, polyhydroxylated C₆₋₁₀ aryl, 5 to 12-membered heterocycle, C₃₋₈ cycloalkyl, hydroxylated C₃₋₈ cycloalkyl, polyhydroxylated C₃₋₈ cycloalkyl or a side chain of a natural or unnatural amino acid;
R₂₂ is —OH, —NH₂, —COOH, —R₈₂—C(O)(CH₂)_{c}—C(H)(R₂₃)—N(H)(R₂₃), —R₈₂—C(O)(CH₂)_{d}—(O CH₂—CH₂)_{f}—N(H)(R₂₃), or —R₈₂—(C(O)—CH(X²)—NH)_{d}—R₇₇;
each R₂₃ independently is hydrogen, C₁₋₆ alkyl, C₆₋₁₀ aryl, C₃₋₈ cycloalkyl, —COOH, or —COO—C₁₋₆ alkyl;
X² is a side chain of a natural or unnatural amino acid;
R₇₇ is a hydrogen or X² and NR₇₇ form a nitrogen containing cyclic compound;
R₈₂ is —NH or oxygen;
R₄₇ is as defined herein;
a is an integer from 1 to 6;
c is an integer from 0 to 3;
d is an integer from 1 to 3;
f is an integer from 1 to 12; and
provided that (i) when R₆₄ is

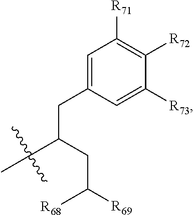

then at least one of R₇₁, R₇₂ and R₇₃ is —NHR₇₄, OR₄₃, or —O—C(O)—R₄₇, or R₆₉ is C(O)R₄₅ in which R₄₅ is X³—R₇₅ or NH—R₁₉; in which each of R₇₄, R₇₅, and R₁₉, independently, is —[C(R₂₀R₂₁)]ₐ—R₂₂, R₄₃ is —R₄₆—R₄₇, R₄₆ is —C(O)—; —C(O)—O—, or —C(O)—NH—, and R₄₇ is an amino group, —R₉—[C(R₂₀R₂₁)]ₐ—R₁₀, —R₉—C₅₋₁₂ heterocycloalkyl-C₁₋₆ alkyl-R₁₀, 5 to 12-membered heterocycloalkyl, or —R₉—C₆₋₁₀ aryl; or
(ii) when R₆₄ is

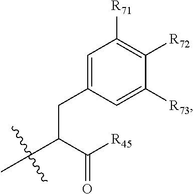

then at least one of R₇₁, R₇₂ and R₇₃ is —NHR₇₄, OR₄₃, or —O—C(O)—R₄₇, or R₄₅ is X³—R₇₅, or NH—R₁₉; in which each of R₇₄, R₇₅, and R₁₉, independently, is —[C(R₂₀R₂₁)]ₐ—R₂₂, R₄₃ is —R₄₆—R₄₇, R₄₆ is —C(O)—; —C(O)—O—, or —C(O)—NH—, and R₄₇ is an amino group, —R₉—[C(R₂₀R₂₁)]ₐ—R₁₀, —R₉—C₅₋₁₂ heterocycloalkyl-C₁₋₆ alkyl-R₁₀, 5 to 12-membered heterocycloalkyl, or —R₉—C₆₋₁₀ aryl.

In some embodiments in the compound of Formula (XXII):
R₅₇ is —CH₃;
R₅₉ is sec-butyl;
R₆₀ is hydrogen, methyl, ethyl, propyl, iso-propyl or iso-butyl;
R₆₁ is iso-propyl,
R₆₂ is hydrogen;
R₆₃ is hydrogen, OH, —O—C₃H₇, O—C(O)—CH₃;
R₆₈ is hydrogen or —CH₃;
R₆₉ is CO₂H, CO₂R₇₀ or C(O)—R₇₈;
R₇₀ is C₁₋₆ alkyl amine;
each of R₇₁ and R₇₃ independently is hydrogen;
R₇₂ is hydrogen, —OR₄₃, OH, F, —CH₃ or —OCH₃;
R₇₈ is OH, —OR₇₅ or —NHR₄₀;
e is the integer 2;
R₄₀ is hydrogen, —OH, —NH₂, or any of the following structures:

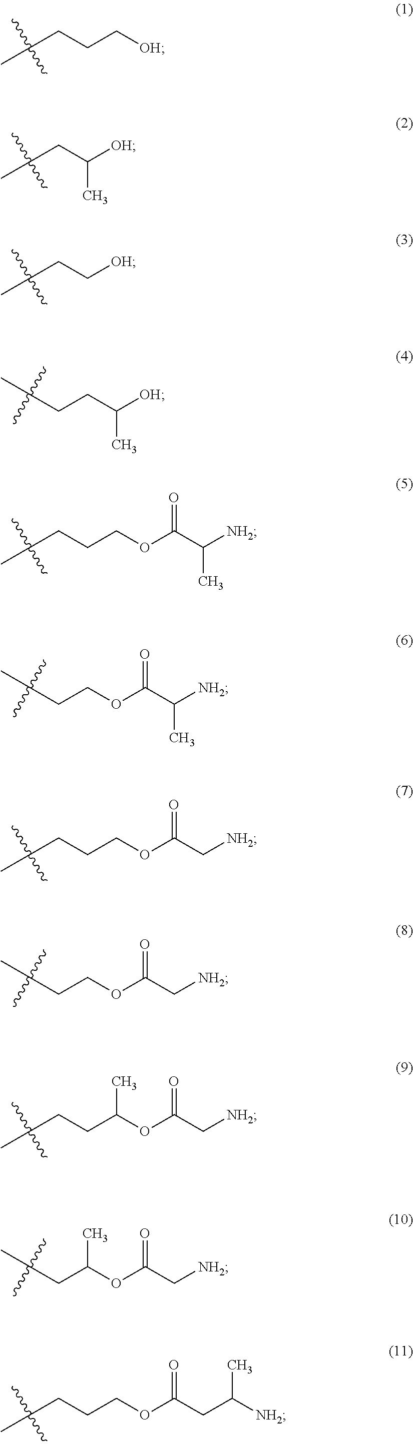

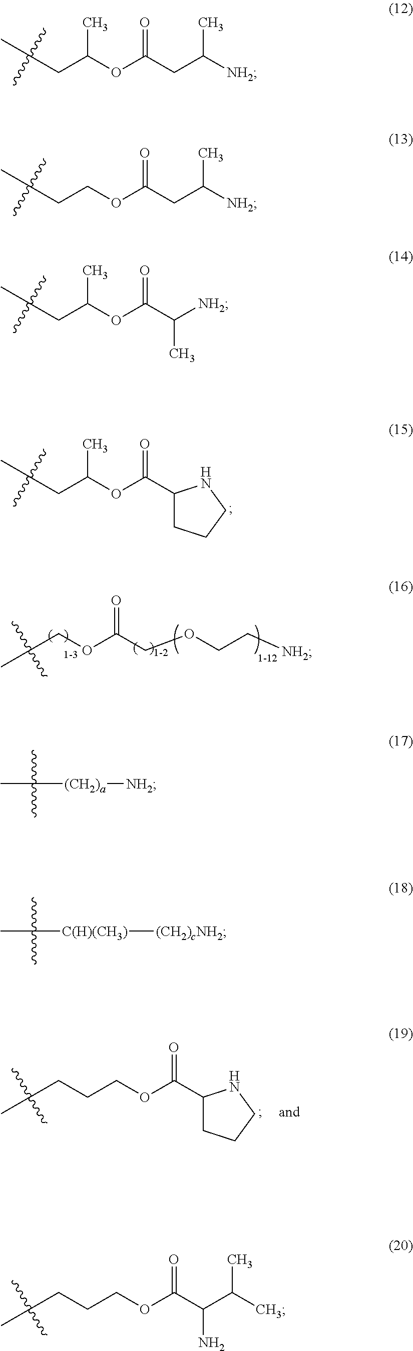

wherein:
a is an integer from 1 to 6;
c is an integer from 0 to 3;
R₇₅ is any one of the following structures:

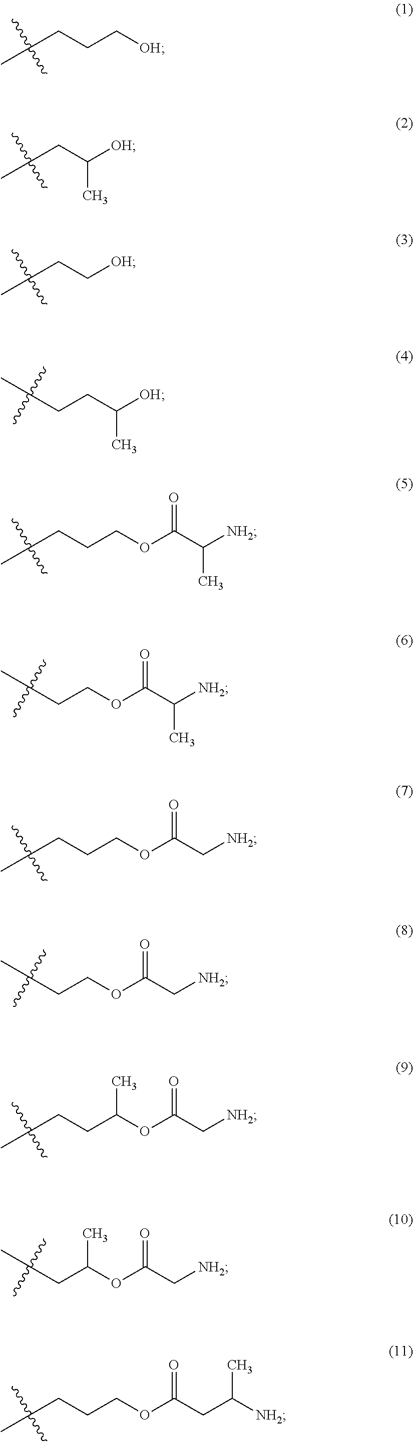

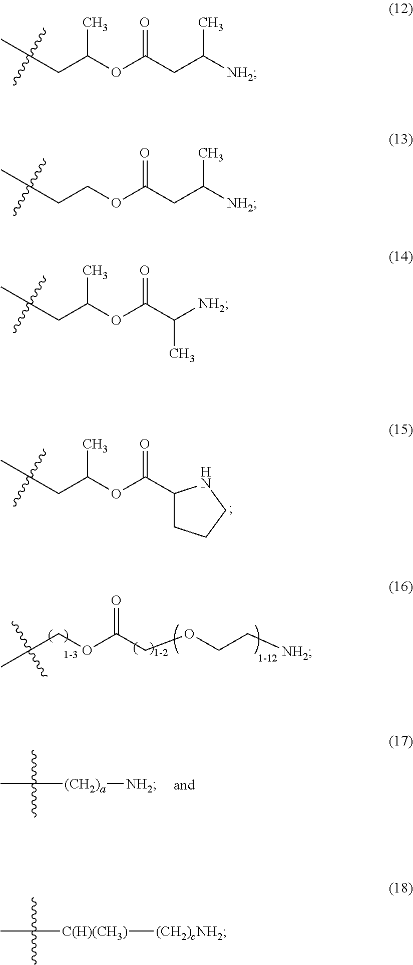

wherein:
a is an integer from 1 to 6; and
c is an integer from 0 to 3;
R₄₃ is hydrogen, —C(O)—(CH₂)ₐ—NH₂, or —C(O)—C(H)(CH₃)—(CH₂)_{c}—NH₂;
   wherein a and c are as defined herein; and
R₄₇ is any one of the following structures:

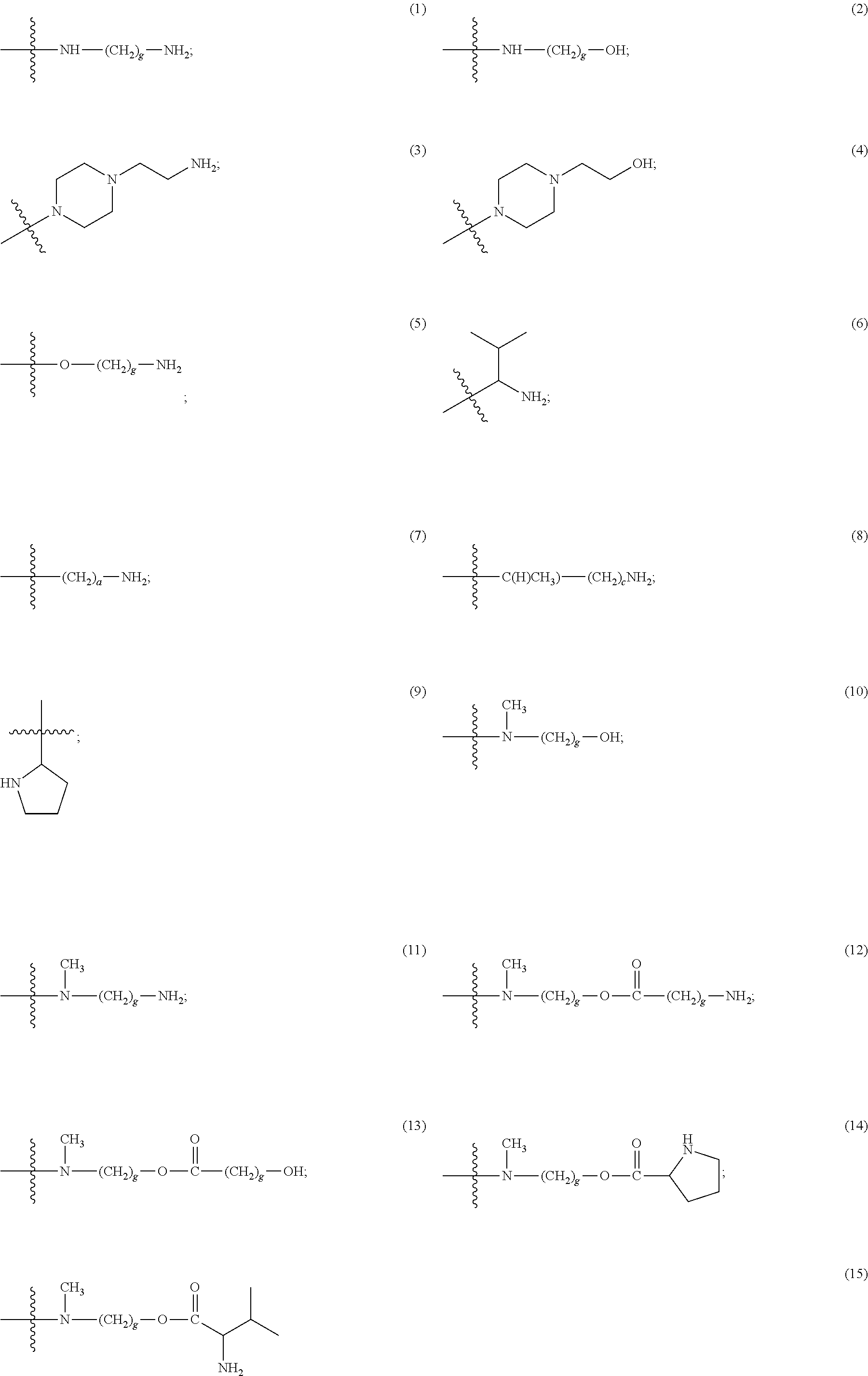

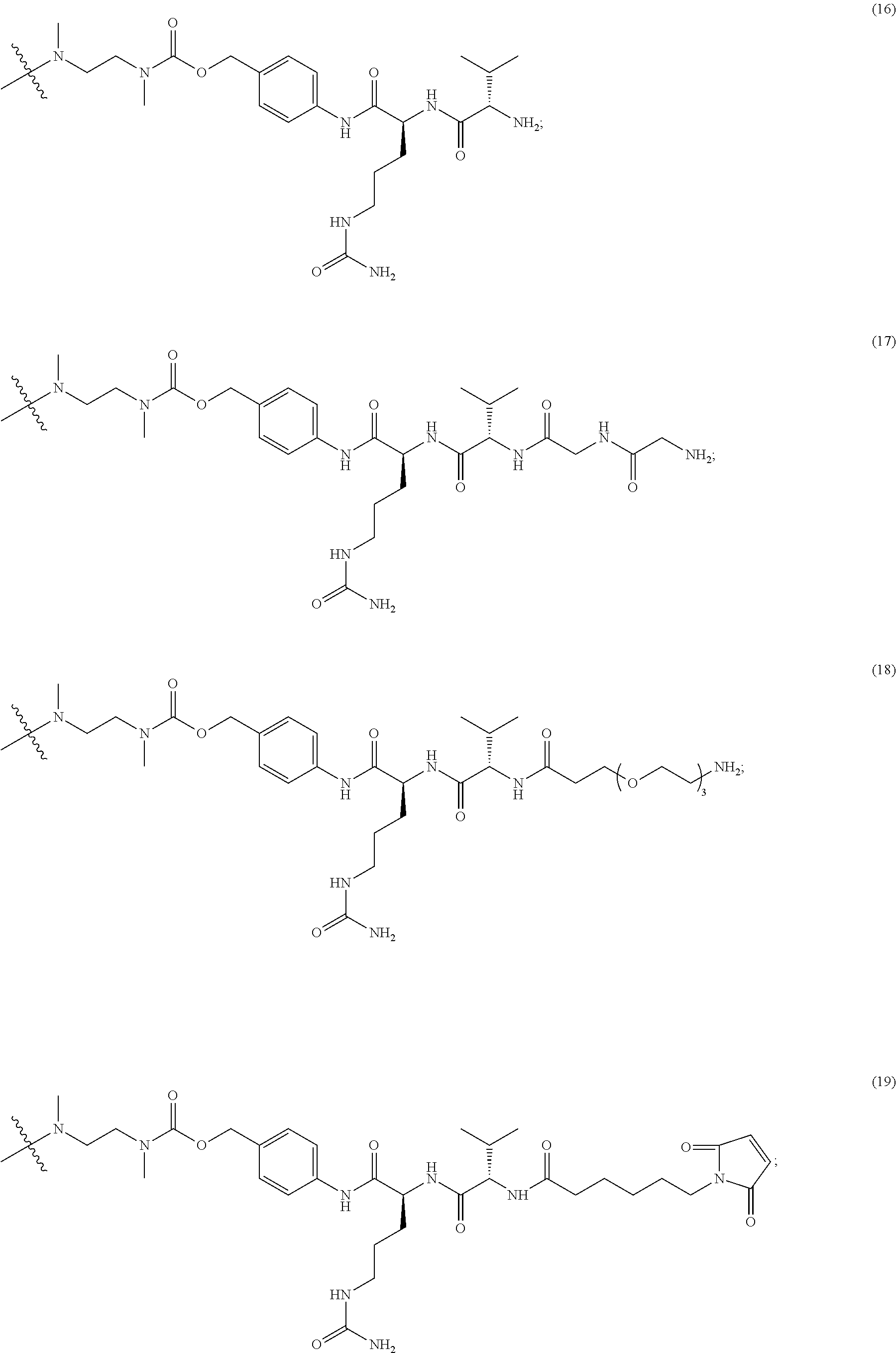

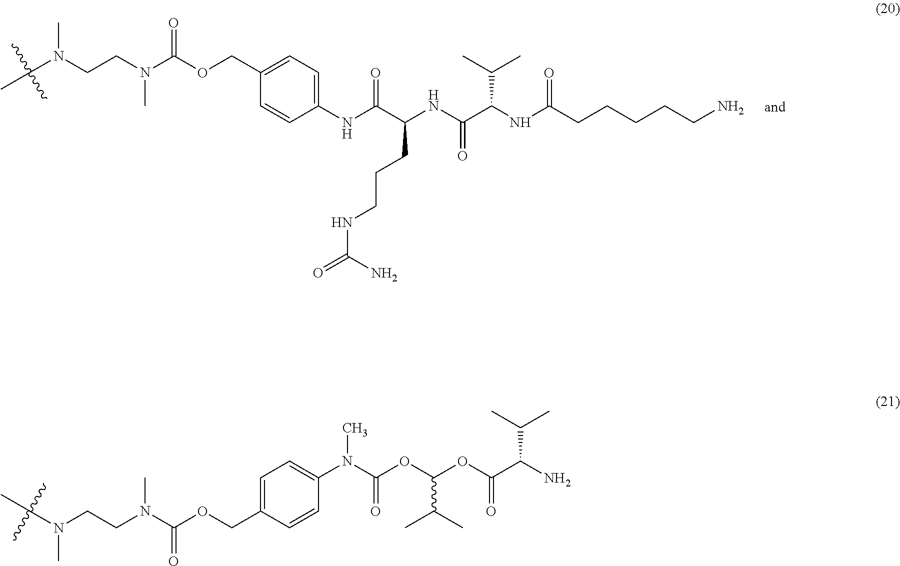

wherein:
a is an integer from 1 to 6;
c is an integer from 0 to 3; and
g is an integer from 2 to 6;
with the proviso that if R₇₂ is —OH, then R₇₅ cannot be hydrogen; if R₆₉ is COOH then R₇₂ must be —OR₄₃ or —O—C(O)—R₄₇.

In some embodiments, the tubulysin of Formula (XXII) is a compound of Formula (XXIII), (XXIIIa), (XXIIIb) or (XXIV):

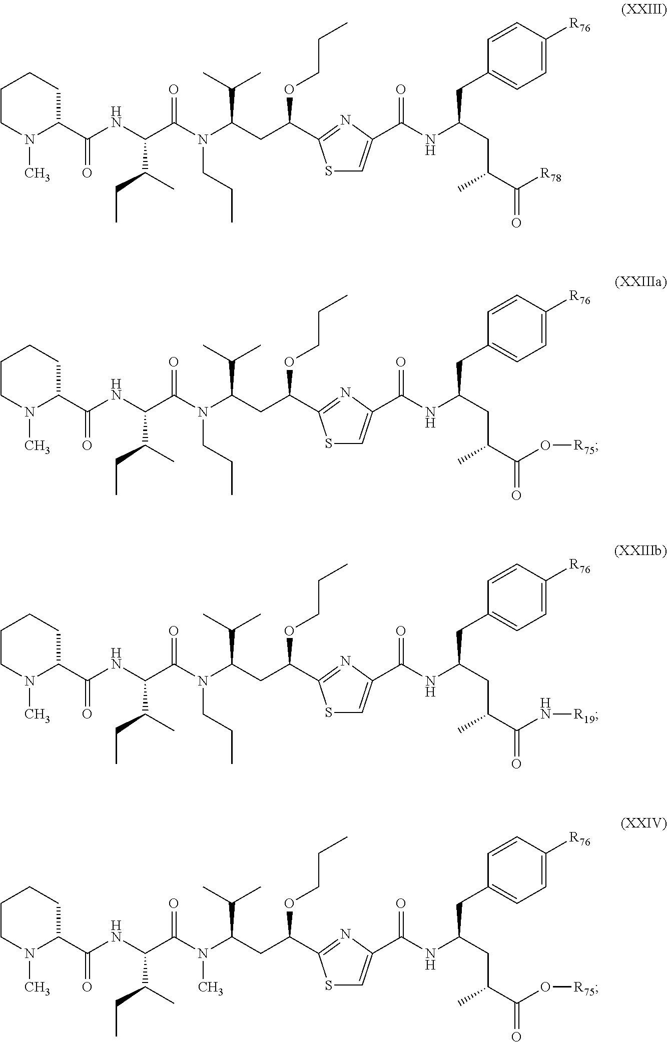

wherein:
R₇₆ is hydrogen, OH, OCH₃, F, —OR₄₃ or —O—C(O)—R₄₇;
wherein R₇₈, R₇₅, R₁₉, R₄₇ and R₄₃ are as defined herein; and
with the proviso that if R₇₆ is —OH, OCH₃ or F, then R₇₅ and R₁₉ cannot be hydrogen.

In one embodiment, R₄₇ is

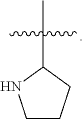 [SMILES: *C1CCCN1]

In another embodiment, R₄₇ is

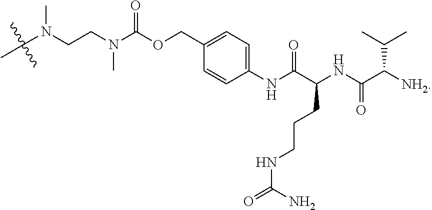 [SMILES: *N(C)CCN(C)C(=O)OCc1ccc(NC(=O)[C@H](CCCNC(N)=O)NC(=O)[C@@H](N)C(C)C)cc1]

In yet another embodiment, R₄₇ is

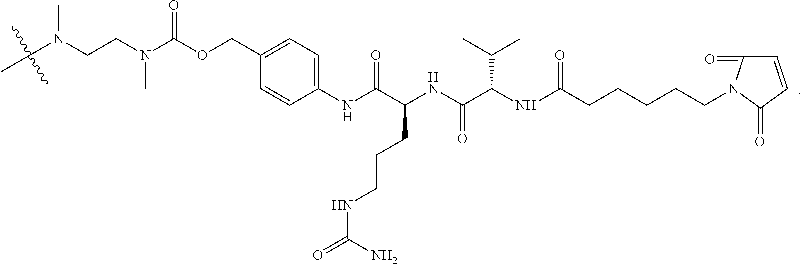 [SMILES: *N(C)CCN(C)C(=O)OCc1ccc(NC(=O)[C@H](CCCNC(N)=O)NC(=O)[C@@H](NC(=O)CCCCCN2C(=O)C=CC2=O)C(C)C)cc1]

In another embodiment, the KSP inhibitor compound is a compound of Formula (XXVI):

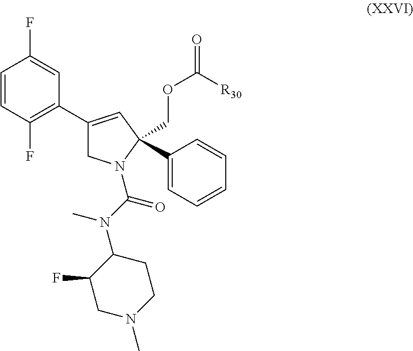

wherein R₃₀ is as defined herein.

In some embodiments R₃₀ is:

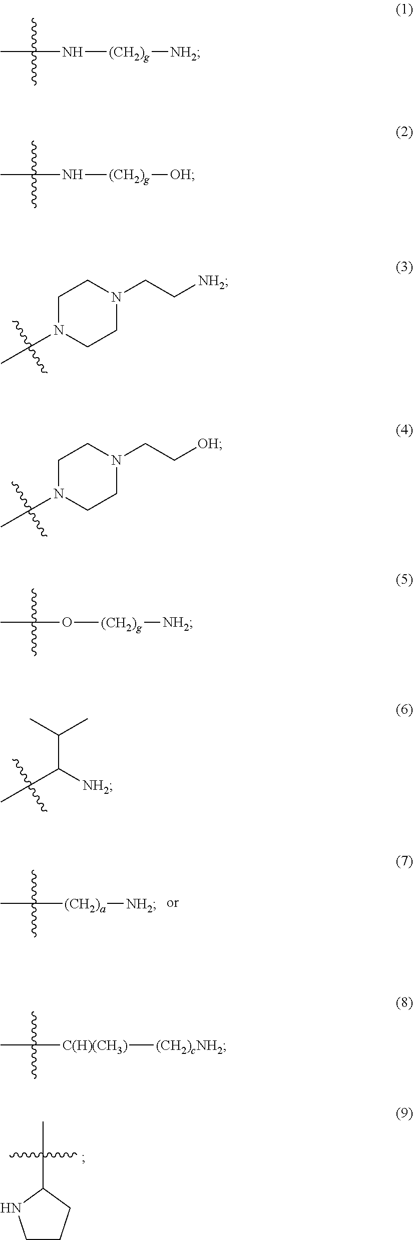

wherein:
a is an integer from 1 to 6;
c is an integer from 0 to 3; and
g is an integer from 2 to 6.

In another embodiment the KSP inhibitor compound is a compound of Formula (XXVII), (XXVIII) or (XXIX):

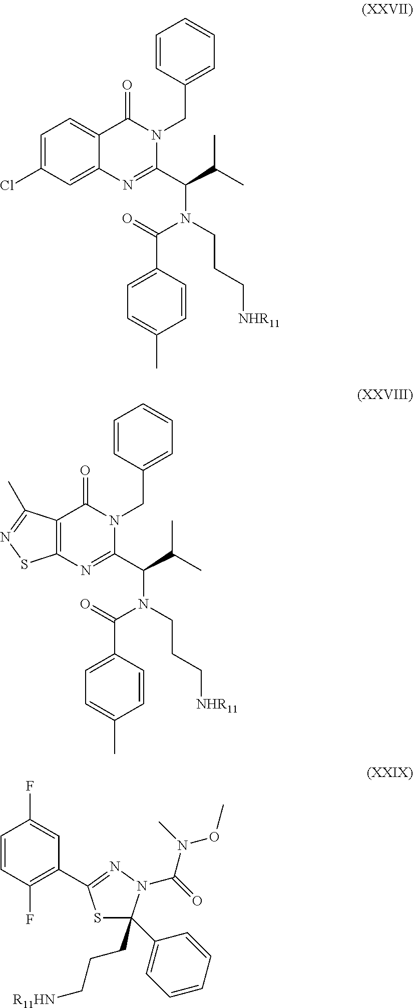

wherein:
R₁₁ is as defined herein.

One skilled in the art of therapeutic agents will readily understand that each of the therapeutic agents described herein can be modified in such a manner that the resulting compound still retains the specificity and/or activity of the original compound. The skilled artisan will also understand that many of these compounds can be used in place of the therapeutic agents described herein. Thus, the therapeutic agents of the present invention include analogues and derivatives of the compounds described herein.

More examples of the therapeutic agents and derivatives thereof suitable for conjugation to the terminally modified polymer of the invention are described in WO 2012/171020 and U.S. Publication No. 2013/0101546, the disclosures of which are incorporated herein by reference in their entirety.

Table B below provides more examples of the therapeutic agents and derivatives thereof suitable for conjugation to form the polymer-drug-protein conjugates or polymer-drug scaffolds of the invention. Spectral data of certain compounds are also provided (ND in the table means “not determined”). These examples may also be the active form of the drug when it is released from the conjugates in vitro or in vivo.

TABLE B
(VI)

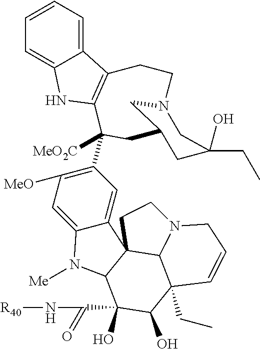

R₄₀

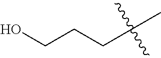 [SMILES: *CCCO]

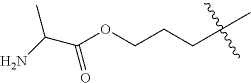 [SMILES: *CCCOC(=O)C(C)N]

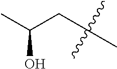 [SMILES: *C[C@H](C)O]

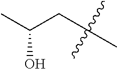 [SMILES: *C[C@@H](C)O]

(IX)

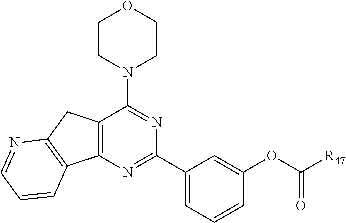

| R₄₇ | m/z |
|---|---|
| 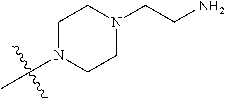 [SMILES: *N1CCN(CCN)CC1] | ND |
| 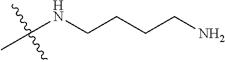 [SMILES: *NCCCCN] | ND |
| 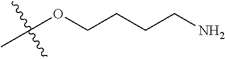 [SMILES: *OCCCCN] | ND |
| 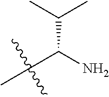 [SMILES: *[C@@H](N)C(C)C] | ND |

(XI)

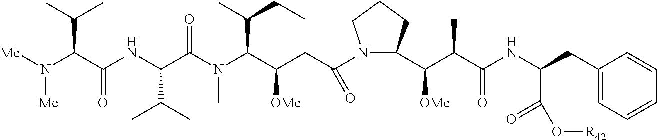

| R₄₂ | m/z |
|---|---|
| H | |
| —CH₃ | 760 |
| 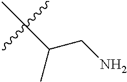 [SMILES: *C(C)CN] | 802.6 |
| 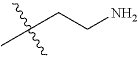 [SMILES: *CCN] | 790 |
| 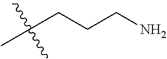 [SMILES: *CCCN] | 804 |

(XII)

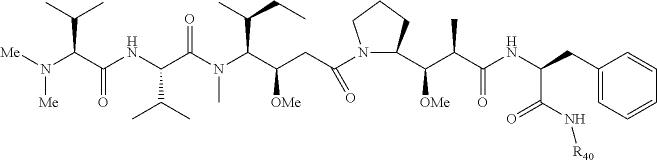

| Ref # | R₄₀ | m/z |
|---|---|---|
| | —H | |
| Ex 17 | 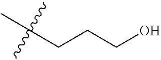 [SMILES: *CCCO] | 803.5 |
| | 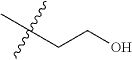 [SMILES: *CCO] | 789.1 |
| Ex 18 | 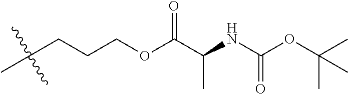 [SMILES: *CCCOC(=O)[C@H](C)NC(=O)OC(C)(C)C] | 974.2 |
| Ex 19 | 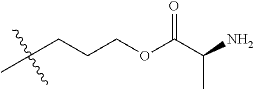 [SMILES: *CCCOC(=O)[C@H](C)N] | 874.5 |
| | 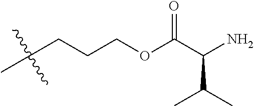 [SMILES: *CCCOC(=O)[C@@H](N)C(C)C] | 902.2 |
| | 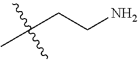 [SMILES: *CCN] | ND |
| | 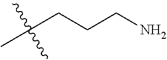 [SMILES: *CCCN] | ND |
| | —OH | 788 |
| | 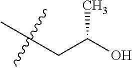 [SMILES: *C[C@H](C)O] | 803.4 |
| | 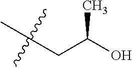 [SMILES: *C[C@@H](C)O] | 803.4 |
| | 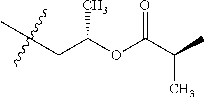 | 874.4 |
| | 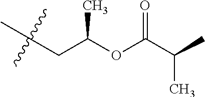 | 874.4 |
| | 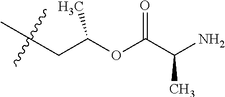 [SMILES: *C[C@H](C)OC(=O)[C@H](C)N] | 874.4 |
| | 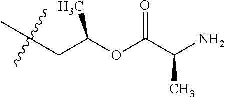 [SMILES: *C[C@@H](C)OC(=O)[C@H](C)N] | 874.4 |
| | 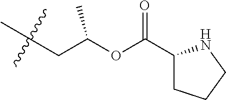 [SMILES: *C[C@H](C)OC(=O)[C@H]1CCCN1] | 900.2 |
| | 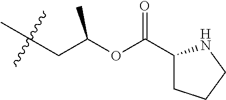 [SMILES: *C[C@@H](C)OC(=O)[C@H]1CCCN1] | 900.2 |
| | 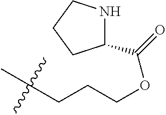 [SMILES: *CCCOC(=O)[C@@H]1CCCN1] | 900.5 |
| | 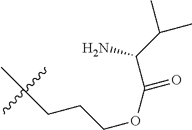 [SMILES: *CCCOC(=O)[C@H](N)C(C)C] | 900.5 |

(XIII)

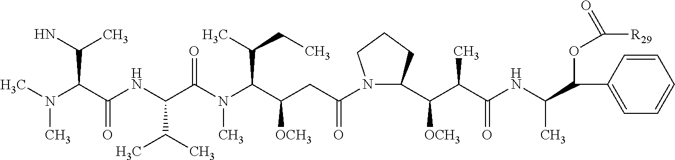

| —C(O)—R₂₉ | m/z |
|---|---|
| 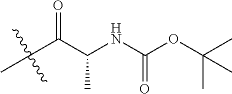 [SMILES: *C(=O)[C@@H](C)NC(=O)OC(C)(C)C] | 903.2 |
| 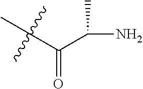 [SMILES: *C(=O)[C@H](C)N] | 803.1 |
| 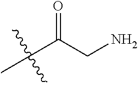 [SMILES: *C(=O)CN] | 790 |
| 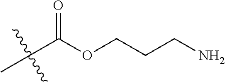 [SMILES: *C(=O)OCCCN] | 832.6 |
| 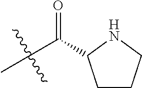 | 829.1 |
| 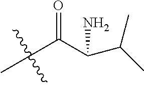 [SMILES: *C(=O)[C@@H](N)C(C)C] | 802 |

(XIV)

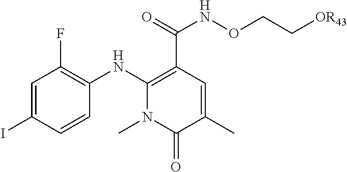

| R₄₃ | m/z |
|---|---|
| 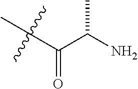 [SMILES: *C(=O)[C@H](C)N] | ND |
| 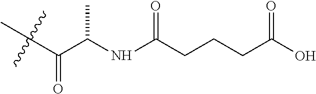 [SMILES: *C(=O)[C@H](C)NC(=O)CCCC(=O)O] | 644.9 |

(XVII)

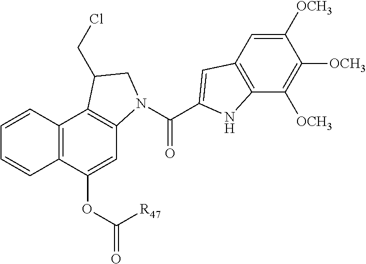

| R₄₇ | m/z |
|---|---|
| 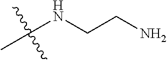 [SMILES: *NCCN] | 553.1 |
| 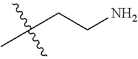 [SMILES: *CCN] | 538.1 |
| 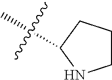 | 564.1 |
| 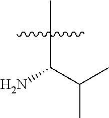 [SMILES: *[C@@H](N)C(C)C] | 566.1 |
| 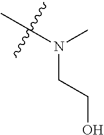 [SMILES: *N(C)CCO] | 568.1 |
| 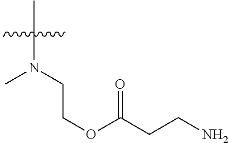 [SMILES: *N(C)CCOC(=O)CCN] | ND |
| 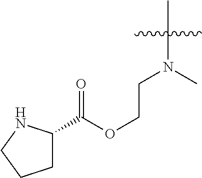 [SMILES: *N(C)CCOC(=O)[C@@H]1CCCN1] | ND |
| 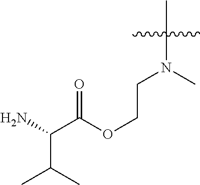 [SMILES: *N(C)CCOC(=O)[C@@H](N)C(C)C] | 667.2 |
| 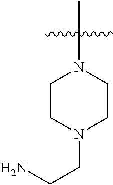 [SMILES: *N1CCN(CCN)CC1] | 622.2 |
| 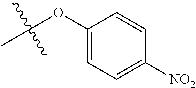 [SMILES: *Oc1ccc([N+](=O)[O-])cc1] | 632.02 |
| 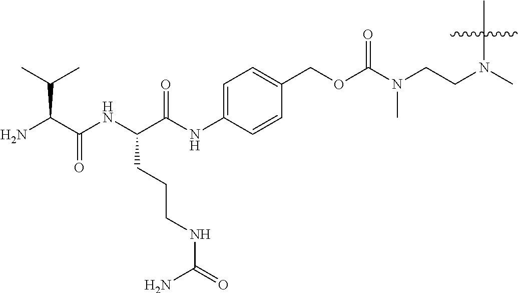 [SMILES: *N(C)CCN(C)C(=O)OCc1ccc(NC(=O)[C@H](CCCNC(N)=O)NC(=O)[C@@H](N)C(C)C)cc1] | 986.2 |
| 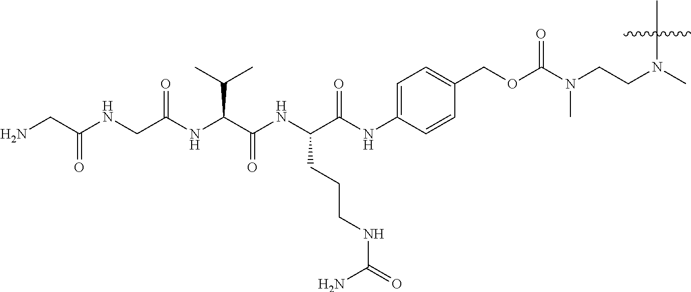 [SMILES: *N(C)CCN(C)C(=O)OCc1ccc(NC(=O)[C@H](CCCNC(N)=O)NC(=O)[C@@H](NC(=O)CNC(=O)CN)C(C)C)cc1] | ND |
| 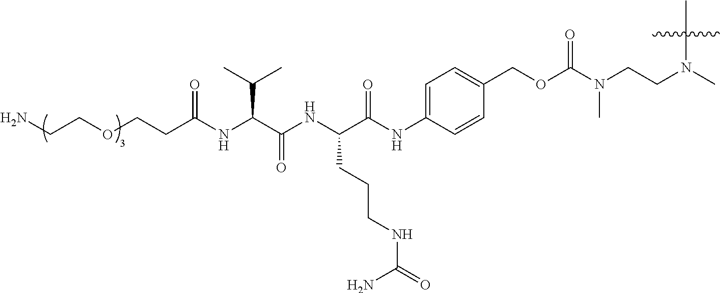 | ND |

(XXIII)

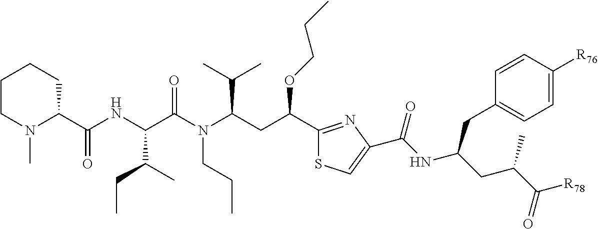

| R₇₆ | —R₇₈ | m/z |
|---|---|---|
| OH | —OH | 772.1 |
| OH | —OCH₃ | 786.4 |
| OH | —NH₂ | 771.4 |
| OH | 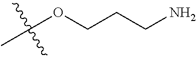 [SMILES: *OCCCN] | 829.4 |
| OH | 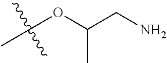 [SMILES: *OC(C)CN] | ND |
| 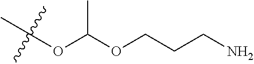 [SMILES: *OC(C)OCCCN] | OH | ND |
| 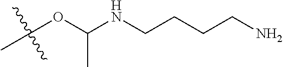 [SMILES: *OC(C)NCCCCN] | —OCH₃ | 900.4 |
| 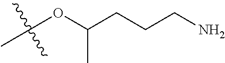 [SMILES: *OC(C)CCCN] | —OH | ND |
| 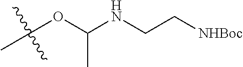 [SMILES: *OC(C)NCCNC(=O)OC(C)(C)C] | —OCH₃ | ND |
| 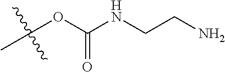 [SMILES: *OC(=O)NCCN] | —OCH₃ | ND |
| 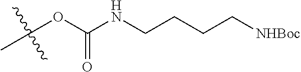 [SMILES: *OC(=O)NCCCCNC(=O)OC(C)(C)C] | —OCH₃ | 1000.5 |
| 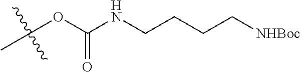 [SMILES: *OC(=O)NCCCCNC(=O)OC(C)(C)C] | —OH | 986.5 |
| 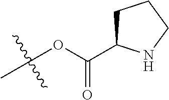 [SMILES: *OC(=O)[C@H]1CCCN1] | —OH | 869.4 |
| 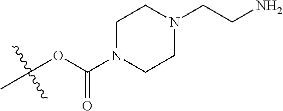 [SMILES: *OC(=O)N1CCN(CCN)CC1] | —OH | 927.3 |
| 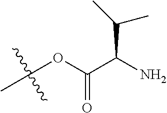 [SMILES: *OC(=O)[C@H](N)C(C)C] | —OH | 871.4 |
| F | 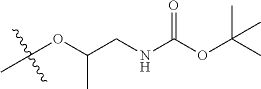 [SMILES: *OC(C)CNC(=O)OC(C)(C)C] | ND |
| F | 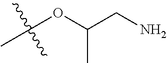 [SMILES: *OC(C)CN] | ND |
| 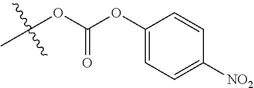 [SMILES: *OC(=O)Oc1ccc([N+](=O)[O-])cc1] | 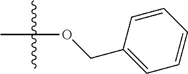 [SMILES: *OCc1ccccc1] | 1027.2 |
| —OH | 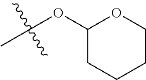 [SMILES: *OC1CCCCO1] | |
| —OH | 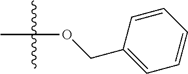 [SMILES: *OCc1ccccc1] | 862.5 |
| 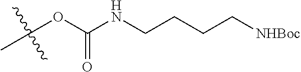 [SMILES: *OC(=O)NCCCCNC(=O)OC(C)(C)C] | 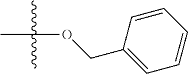 [SMILES: *OCc1ccccc1] | 1076.4 |
| 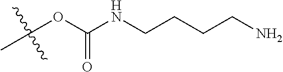 [SMILES: *OC(=O)NCCCCN] | —OH | 886.3 |
| 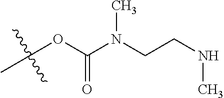 [SMILES: *OC(=O)N(C)CCNC] | —OH | 886.4 |
| 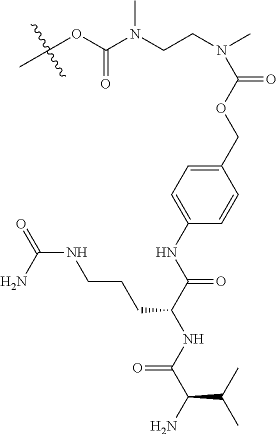 [SMILES: *OC(=O)N(C)CCN(C)C(=O)OCc1ccc(NC(=O)[C@@H](CCCNC(N)=O)NC(=O)[C@H](N)C(C)C)cc1] | —OH | 1291.7 |
| 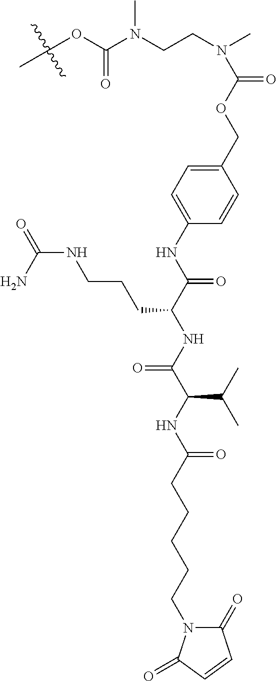 [SMILES: *OC(=O)N(C)CCN(C)C(=O)OCc1ccc(NC(=O)[C@@H](CCCNC(N)=O)NC(=O)[C@H](NC(=O)CCCCCN2C(=O)C=CC2=O)C(C)C)cc1] | —OH | 1316.7 |
| 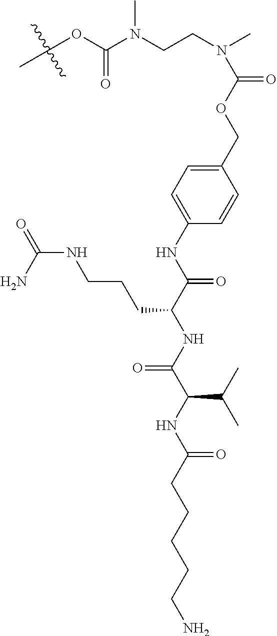 [SMILES: *OC(=O)N(C)CCN(C)C(=O)OCc1ccc(NC(=O)[C@@H](CCCNC(N)=O)NC(=O)[C@H](NC(=O)CCCCCN)C(C)C)cc1] | —OH | ND |

(XXX)

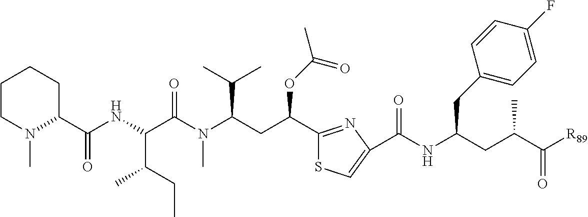

—R₈₉

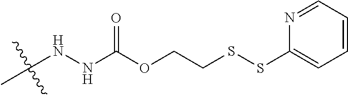 [SMILES: *NNC(=O)OCCSSc1ccccn1]

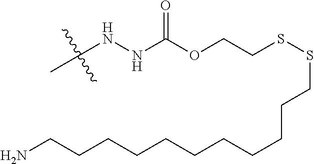 [SMILES: *NNC(=O)OCCSSCCCCCCCCCCCN]

(XXVII)

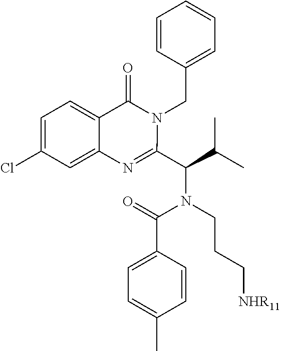

(XXVIII)

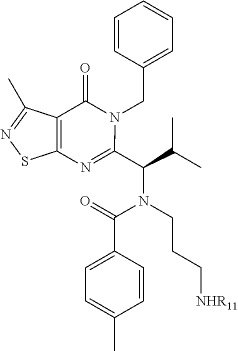

(XXIX)

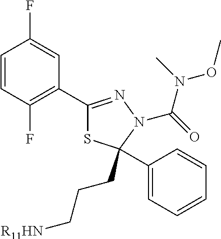

| | m/z |
|---|---|
| R₁₁ | (XXVII) |
| 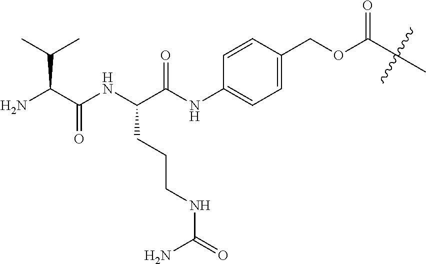 [SMILES: *C(=O)OCc1ccc(NC(=O)[C@H](CCCNC(N)=O)NC(=O)[C@@H](N)C(C)C)cc1] | 922.3 |
| 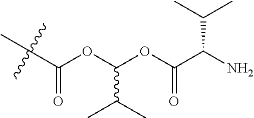 [SMILES: *C(=O)OC(OC(=O)[C@@H](N)C(C)C)C(C)C] | 732.2 |
| 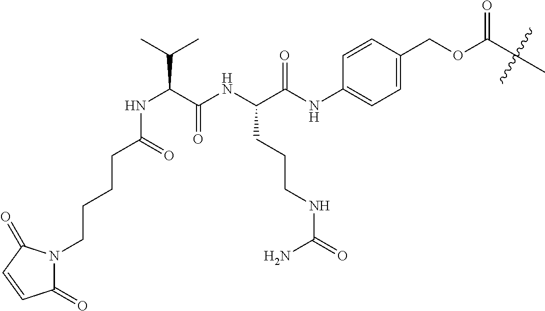 [SMILES: *C(=O)OCc1ccc(NC(=O)[C@H](CCCNC(N)=O)NC(=O)[C@@H](NC(=O)CCCCN2C(=O)C=CC2=O)C(C)C)cc1] | 1101.7 |
| 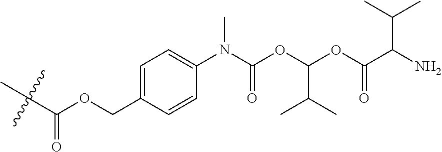 [SMILES: *C(=O)OCc1ccc(N(C)C(=O)OC(OC(=O)C(N)C(C)C)C(C)C)cc1] | ND |
| 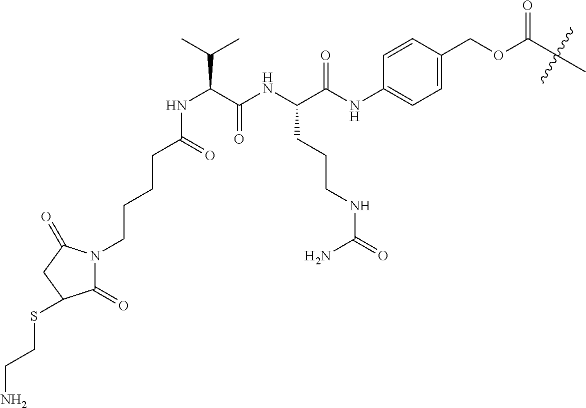 [SMILES: *C(=O)OCc1ccc(NC(=O)[C@H](CCCNC(N)=O)NC(=O)[C@@H](NC(=O)CCCCN2C(=O)CC(SCCN)C2=O)C(C)C)cc1] | ND |
| 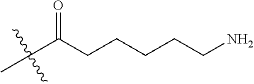 [SMILES: *C(=O)CCCCCN] | ND |
| 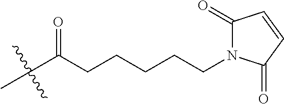 [SMILES: *C(=O)CCCCCN1C(=O)C=CC1=O] | ND |
| 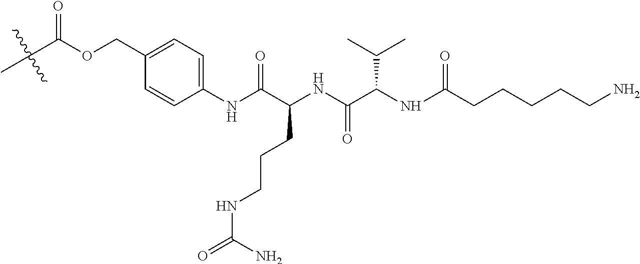 [SMILES: *C(=O)OCc1ccc(NC(=O)[C@H](CCCNC(N)=O)NC(=O)[C@@H](NC(=O)CCCCCN)C(C)C)cc1] | ND |
| 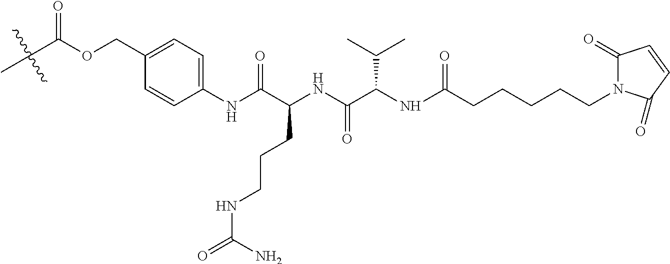 [SMILES: *C(=O)OCc1ccc(NC(=O)[C@H](CCCNC(N)=O)NC(=O)[C@@H](NC(=O)CCCCCN2C(=O)C=CC2=O)C(C)C)cc1] | ND |

### Protein-Based Recognition Molecules (PBRMs)

A protein-based recognition molecule can direct the terminally modified polymer carrier or conjugate thereof to specific tissues, cells, or locations in a cell. The terminally modified polymer carrier or conjugate may or may not carry a drug. The protein-based recognition molecule can direct the modified polymer in culture or in a whole organism, or both. In each case, the protein-based recognition molecule has a ligand that is present on the cell surface of the targeted cell(s) to which it binds with an effective specificity, affinity and avidity. In some embodiments, the protein-based recognition molecule targets the modified polymer to tissues other than the liver. In other embodiments the protein-based recognition molecule targets the modified polymer to a specific tissue such as the liver, kidney, lung or pancreas. The protein-based recognition molecule can target the modified polymer to a target cell such as a cancer cell, such as a receptor expressed on a cell such as a cancer cell, a matrix tissue, or a protein associated with cancer such as tumor antigen. Alternatively, cells comprising the tumor vasculature may be targeted. Protein-based recognition molecules can direct the polymer to specific types of cells such as specific targeting to hepatocytes in the liver as opposed to Kupffer cells. In other cases, protein-based recognition molecules can direct the polymer to cells of the reticular endothelial or lymphatic system, or to professional phagocytic cells such as macrophages or eosinophils. (In such cases the polymer itself might also be an effective delivery system, without the need for specific targeting).

In still other embodiments, the protein based recognition molecule can target the modified polymer to a location within the cell, such as the nucleus, the cytoplasm, or the endosome, for example. In specific embodiments, the protein based recognition molecule can enhance cellular binding to receptors, or cytoplasmic transport to the nucleus and nuclear entry or release from endosomes or other intracellular vesicles.

In specific embodiments the protein based recognition molecules that are suitable for conjugating with the terminally modified polymer of the invention comprise antibodies, antigens, proteins and peptides or peptide mimics. See US2013/0011419 [0131], [0134]

Exemplary antibodies or antibodies derived from Fab, Fab2, scFv or camel antibody heavy-chain fragments specific to the cell surface markers, include, but are not limited to, 5T4, AOC3, C242, CA-125, CCL11, CCR 5, CD2, CD3, CD4, CD5, CD6, CD8, CD11, CD15, CD18, CD19, CD20, CD22, CD23, CD25, CD26, CD28, CD30, CD31, CD33, CD34, CD37, CD38, CD40, CD41, CD44, CD46, CD51, CD52, CD54, CD56, CD62E, CD62P, CD62L, CD70, CD74, CD79, CD80, CD105, CD125, CD138, CD141, CD147, CD152, CD 154, CD326, CEA, clumping factor, CTLA-4, EGFR, EGFRvIII, ErbB2, ErbB3, EpCAM, folate receptor, FAP, GD2, GD3, GPNMB, HGF, HER2, HER3, HER4, ICAM, IGF-1 receptor, VEGFR1, EphA2, EphB, TRPV1, CFTR, gpNMB, CA9, Cripto, ACE, APP, adrenergic receptor-beta2, Claudine 3, Mesothelin, IL-2 receptor, IL-4 receptor, IL-13 receptor, integrins (including α₄, αᵥβ₃, αᵥβ₅, αᵥβ₆, α₁β₄, α₄β₁, α₄β₇, α₅β₁, α₆β₄, α_{IIb}β₃ intergins), IFN-α, IFN-γ, IgE, IgE, IGF-1 receptor, IL-1, IL-8, IL-12, IL-23, IL-13, IL-22, IL-4, IL-5, IL-6, interferon receptor, ITGB2 (CD18), LFA-1 (CD11a), L-selectin (CD62L), flk2/flt3, FLT3, PD-1, PD-L1, PD-L2, p150.95, Mac1, mucin, MUC1, myostatin, NCA-90, NGF, PDGFRα, phosphatidylserine, prostatic carcinoma cell, Pseudomonas aeruginosa, rabies, RANKL, respiratory syncytial virus, Rhesus factor, transferrin, SLAMF7, sphingosine-1-phosphate, TAG-72, T-cell receptor, tenascin C, TGF-1, TGF-β2, TGF-β, TNF-α, TRAIL-R₁, TRAIL-R₂, tumor antigen CTAA16.88, VEGF, VEGF-A, VEGFR2, VLA-4, VCAM, vimentin, and the like.

In one embodiment the antibodies or antibody derived from Fab, Fab2, scFv or camel antibody heavy-chain fragments specific to the cell surface markers include 5T4, CA-125, C242, CD3, CD8, CD19, CD22, CD25, CD30, CD31, CD33, CD34, CD37, CD40, CD44, CD46, CD51, CD54, CD56, CD62E, CD62P, CD62L, CD70, CD138, CD141, CD326, CEA, CTLA-4, EGFR, ErbB2, ErbB3, FAP, folate receptor, IGF-1 receptor, GD3, GPNMB, HGF, HER2, HER3, HER4, VEGF-A, VEGFR2, VEGFR1, EphA2, EpCAM, 5T4, TAG-72, tenascin C, TRPV1, CFTR, gpNMB, CA9, Cripto, ACE, APP, PDGFR α, phosphatidylserine, prostatic carcinoma cells, adrenergic receptor-beta2, Claudine 3, mesothelin, FLT3, PD-1, PD-L1, PD-L2, mucin, MUC1, Mesothelin, IL-2 receptor, IL-4 receptor, IL-13 receptor and integrins (including αᵥβ₃, αᵥβ₅, αᵥβ₆, α₁β₄, α₄β₁, α₅β₁, α₆β₄ intergins), tenascin C, TRAIL-R₂ and vimentin.

Exemplary antibodies include 3F8, abagovomab, abciximab (REOPRO), adalimumab (HUMIRA), adecatumumab, afelimomab, afutuzumab, alacizumab, ALD518, alemtuzumab (CAMPATH), altumomab, amatuximab, anatumomab, anrukinzumab, apolizumab, arcitumomab (CEA-SCAN), aselizumab, atlizumab (tocilizumab, Actemra, RoActemra), atorolimumab, bapineuzumab, basiliximab (Simulect), bavituximab, bectumomab (LYMPHOSCAN), belimumab (BENLYSTA), benralizumab, bertilimumab, besilesomab (SCINITIMUN), bevacizumab (AVASTIN), biciromab (FIBRISCINT), bivatuzumab, blinatumomab, brentuximab, briakinumab, canakinumab (ILARIS), cantuzumab, capromab, catumaxomab (REMOVAB), CC49, cedelizumab, certolizumab, cetuximab (ERBITUX), citatuzumab, cixutumumab, clenoliximab, clivatuzumab, conatumumab, CR6261, dacetuzumab, daclizumab (ZENAPAX), daratumumab, denosumab (PROLIA), detumomab, dorlimomab, dorlixizumab, ecromeximab, eculizumab (SOLIRIS), edobacomab, edrecolomab (PANOREX), efalizumab (RAPTIVA), efungumab (MYCOGRAB), elotuzumab, elsilimomab, enlimomab, epitumomab, epratuzumab, erlizumab, ertumaxomab (REXOMUN), etaracizumab (ABEGRIN), exbivirumab, fanolesomab (NEUTROSPEC), faralimomab, farletuzumab, felvizumab, fezakinumab, figitumumab, fontolizumab (HuZAF), foravirumab, fresolimumab, galiximab, gantenerumab, gavilimomab, gemtuzumab girentuximab, glembatumumab, golimumab (SIMPONI), gomiliximab, ibalizumab, ibritumomab, igovomab (INDIMACIS-125), imciromab (MYOSCINT), infliximab (REMICADE), intetumumab, inolimomab, inotuzumab, ipilimumab, iratumumab, keliximab, labetuzumab (CEA-CIDE), lebrikizumab, lemalesomab, lerdelimumab, lexatumumab, libivirumab, lintuzumab, lucatumumab, lumiliximab, mapatumumab, maslimomab, matuzumab, mepolizumab (BOSATRIA), metelimumab, milatuzumab, minretumomab, mitumomab, morolimumab, motavizumab (NUMAX), muromonab-CD3 (ORTHOCLONE OKT3), nacolomab, naptumomab, natalizumab (TYSABRI), nebacumab, necitumumab, nerelimomab, nimotuzumab (THERACIM), nofetumomab, ocrelizumab, odulimomab, ofatumumab (ARZERRA), olaratumab, omalizumab (XOLAIR), ontecizumab, oportuzumab, oregovomab (OVAREX), otelixizumab, pagibaximab, palivizumab (SYNAGIS), panitumumab (VECTIBIX), panobacumab, pascolizumab, pemtumomab (THERAGYN), pertuzumab (OMNITARG), pexelizumab, pintumomab, priliximab, pritumumab, PRO 140, rafivirumab, ramucirumab, ranibizumab (LUCENTIS), raxibacumab, regavirumab, reslizumab, rilotumumab, rituximab (RITUXAN), robatumumab, rontalizumab, rovelizumab (LEUKARREST), ruplizumab (ANTOVA), satumomab pendetide, sevirumab, sibrotuzumab, sifalimumab, siltuximab, siplizumab, solanezumab, sonepcizumab, sontuzumab, stamulumab, sulesomab (LEUKOSCAN), tacatuzumab (AFP-CIDE), tetraxetan, tadocizumab, talizumab, tanezumab, taplitumomab paptox, tefibazumab (AUREXIS), telimomab, tenatumomab, teneliximab, teplizumab, TGN1412, ticilimumab (tremelimumab), tigatuzumab, TNX-650, tocilizumab (atlizumab, ACTEMRA), toralizumab, tositumomab (BEXXAR), trastuzumab (HERCEPTIN), tremelimumab, tucotuzumab, tuvirumab, urtoxazumab, ustekinumab (STELERA), vapaliximab, vedolizumab, veltuzumab, vepalimomab, visilizumab (NUVION), volociximab (HUMASPECT), votumumab, zalutumumab (HuMEX-EGFr), zanolimumab (HuMAX-CD4), ziralimumab and zolimomab.

In some embodiments the antibodies are directed to cell surface markers for 5T4, CA-125, CEA, CD2, CD3, CD4, CD5, CD6, CD11, CD19, CD20, CD22, CD26, CD30, CD33, CD34, CD37, CD38, CD40, CD44, CD46, CD51, CD56, CD79, Cd105, CD138, CTLA-4, EphA, EphB, EpCAM, HER2, HER3, HER4, EGFR, FAP, folate receptor, HGF, integrin αᵥβ₃, integrin α₅β₁, IGF-1 receptor, GD3, GPNMB, CA9, FLT3, PD-1, PD-L1, PD-L2, mucin, MUC1, phosphatidylserine, prostatic carcinoma cells, PDGFR α, TAG-72, tenascin C, TRAIL-R₂, VEGF-A and VEGFR2. In this embodiment the antibodies are abagovomab, adecatumumab, alacizumab, altumomab, anatumomab, arcitumomab, bavituximab, bevacizumab (AVASTIN), bivatuzumab, blinatumomab, brentuximab, cantuzumab, catumaxomab, capromab, cetuximab, citatuzumab, clivatuzumab, conatumumab, dacetuzumab, edrecolomab, epratuzumab, ertumaxomab, etaracizumab, farletuzumab, figitumumab, gemtuzumab, glembatumumab, ibritumomab, igovomab, intetumumab, inotuzumab, labetuzumab, lexatumumab, lintuzumab, lucatumumab, matuzumab, mitumomab, naptumomab estafenatox, necitumumab, oportuzumab, oregovomab, panitumumab, pemtumomab, pertuzumab, pritumumab, rituximab (RITUXAN), rilotumumab, robatumumab, satumomab, sibrotuzumab, taplitumomab, tenatumomab, tenatumomab, ticilimumab (tremelimumab), tigatuzumab, trastuzumab (HERCEPTIN), tositumomab, tremelimumab, tucotuzumab celmoleukin, volociximab and zalutumumab.

In specific embodiments the antibodies directed to cell surface markers for HER2 are pertuzumab or trastuzumab and for EGFR the antibody is cetuximab and for CD20 the antibody is rituximab and for VEGF-A is bevacizumab and for CD-22 the antibody is epratuzumab or veltuzumab and for CEA the antibody is labetuzumab and for CD44 the antibody is bivatuzumab and for FAP the antibody is sibrotuzumab.

Exemplary peptides or peptide mimics include integrin targeting peptides (RGD peptides), LHRH receptor targeting peptides, ErbB2 (HER2) receptor targeting peptides, prostate specific membrane bound antigen (PSMA) targeting peptides, lipoprotein receptor LRP1 targeting, ApoE protein derived peptides, ApoA protein peptides, somatostatin receptor targeting peptides, chlorotoxin derived peptides, AOD peptides or peptide fragments, AOD-like peptides or peptide fragments, CD-NP peptides, thymosin alpha 1 peptides, ziconotide peptides, protegrin peptides, KISS1 peptides, V681-like peptides, pro insulin c-peptides, Factor IX moieties, biphalin peptides, GM-CSF moieties, 2D-VCAM-1 variant polypeptides, 2D-VCAM-1 variant polypeptide, and bombesin.

In specific embodiments the peptides or peptide mimics are AOD peptides or peptide fragments, AOD-like peptides or peptide fragments, CD-NP peptides, thymosin alpha 1 peptides, ziconotide peptides, protegrin peptides, KISS1 peptides, V681-like peptides, pro insulin c-peptides, Factor IX moieties, biphalin peptides, GM-CSF moieties, 2D-VCAM-1 variant polypeptides and 2D-VCAM-1 variant polypeptide.

Exemplary proteins and polypeptides comprise interferons such as α, β, γ; lymphokines such as IL-2, IL-3, IL-4 and IL-6; hormones such as insulin, TRH (thyrotropin releasing hormones) MSH (melanocyte-stimulating hormones), steroid hormones such as androgens and estrogens, transferrin, fibrinogen-gamma fragment, thrombospondin, claudin, apolipoprotein E, IFN-α proteins, Avibody™ proteins, peptide aptamers, Affibody molecules such as, for example, ABY-025, Ankyrin repeat proteins, ankyrin-like repeats proteins and synthetic peptides.

In some embodiments of the invention the conjugates of the invention comprise broad spectrum cytotoxins in combination with cell surface markers for HER2 such as pertuzumab or trastuzumab; for EGFR such as cetuximab; for CEA such as labetuzumab; for CD20 such as rituximab; for VEGF-A such as bevacizumab; or for CD-22 such as epratuzumab or veltuzumab.

In other embodiments of the invention the conjugates used in the invention comprise combinations of two or more protein based recognition molecules, such as, for example, combination of bispecific antibodies directed to the EGF receptor (EGFR) on tumor cells and to CD3 and CD28 on T cells; combination of bispecific antibodies directed to CD33 and FLT3; combination of antibodies or antibody derived from Fab, Fab2, scFv or camel antibody heavy-chain fragments and peptides or peptide mimetics; combination of antibodies or antibody derived from Fab, Fab2, scFv or camel antibody heavy-chain fragments and proteins; combination of two bispecific antibodies such as CD3×CD19 plus CD28×CD22 bispecific antibodies.

In embodiments of the invention, the conjugates comprise a PBRM attached to the terminus of the polymer carrier and one or more drug molecules attached to the backbone of the polymer via suitable linkers.

Table C below provides more examples of the PBRM described hereof, which are suitable for conjugation to the terminally modified polymer carrier to form the terminally modified polymer-PBRM conjugates or for conjugation to the terminally modified polymer having one or more drug molecules attached to the backbone of the polymer to form the terminally modified polymer-drug-PBRM conjugates respectively of the invention.

TABLE C
PBRM

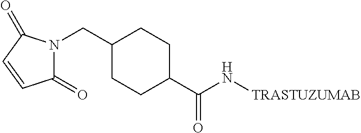

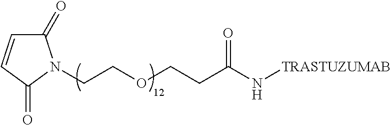

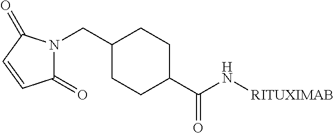

TRASTUZUMAB-Fab′-SH

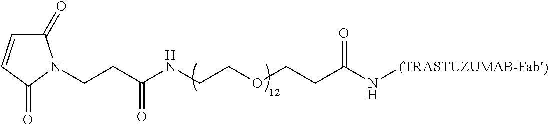

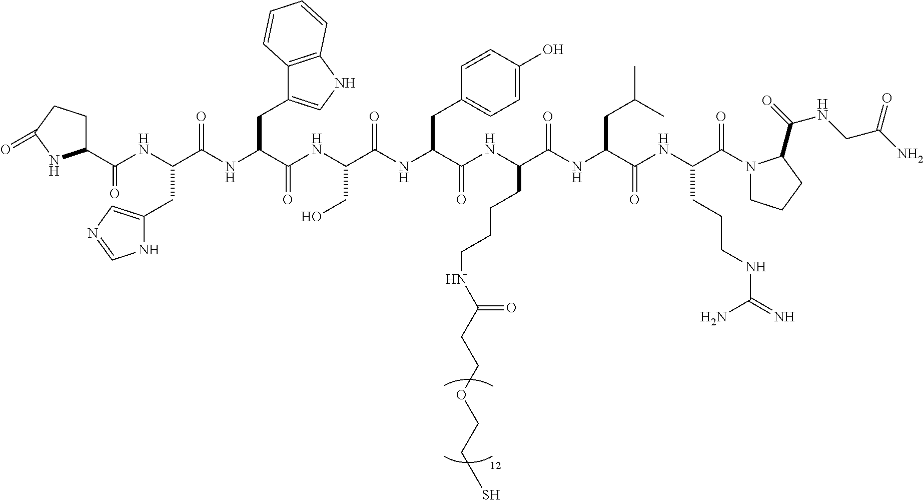

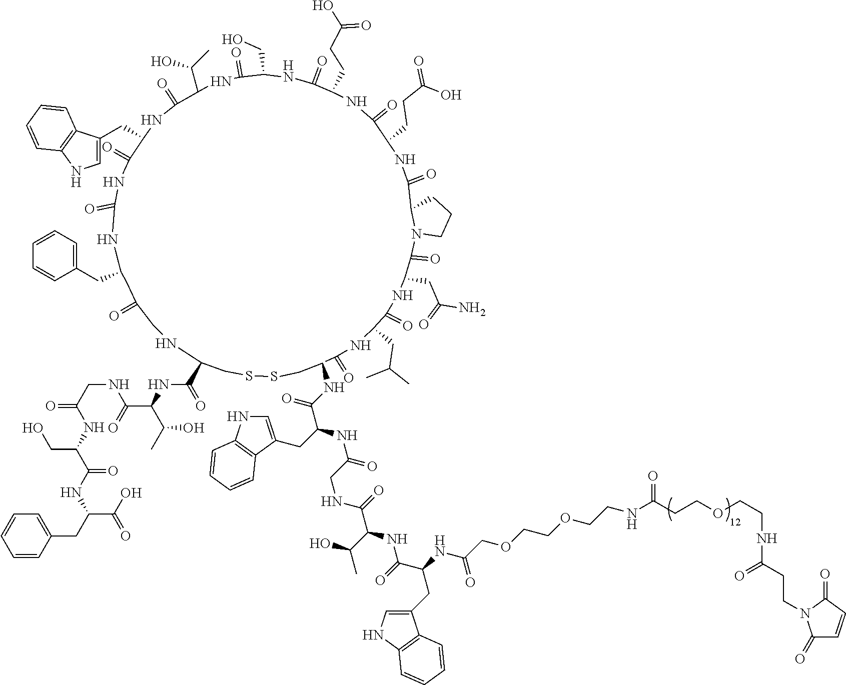

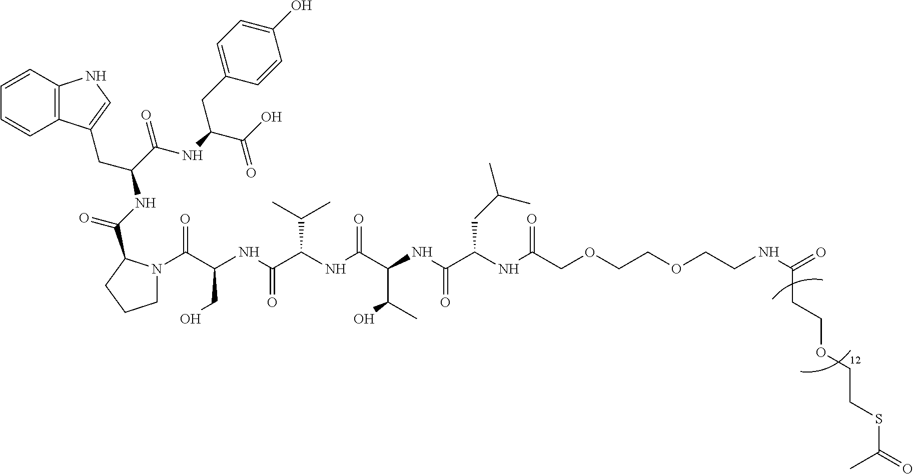

TRASTUZUMAB-Fab-SH

Linkers (L^{D} and L^{P})

As described above, the drug or PBRM can be connected to the backbone of the terminally modified polymer via a linker L^{D} or L^{P}. In some embodiments, the linker is biocleavable/biodegradable under intracellular conditions, such that the cleavage of the linker releases the drug or PBRM from the polymer unit in the intracellular environment. Examples of L^{D} or L^{P} suitable for conjugating the drug or PBRM to the terminally modified polymer of the invention are described in WO 2012/171020 and U.S. Publication No. 2013/0101546, the disclosures of which are incorporated herein by reference in their entirety.

In one embodiment, the modifier (“M”) can be covalently attached to the terminally modified polymer along the backbone of the polymer. When M is a therapeutic agent having a molecular weight ≦5 kDa, it is connected to the backbone of the terminally modified polymer via a linker L^{D}. When M is a PBRM, it is connected to the backbone of the terminally modified polymer via a linker L^{P}, wherein the linker L^{P} is distinct from the linker L^{D}. In some embodiments, the linker L^{D} or L^{P} is biocleavable/biodegradable under intracellular conditions, such that the cleavage of the linker releases the drug or PBRM from the polymer unit in the intracellular environment.

The linker L^{D} or L^{P} is any chemical moiety that is capable of linking a drug or a PBRM to a polymer backbone through chemical bonds such that the drug or PBRM and the polymer are chemically coupled (e.g., covalently bonded) to each other. In some embodiments, the linker comprises a biodegradable linker moiety (e.g., a biodegradable bond such as an ester or amide bond).

In other embodiments, the linker L^{D} or L^{P} is biodegradable under mild conditions, i.e., conditions within a cell under which the activity of the drug is not affected. Examples of suitable biodegradable linker moiety include disulfide linkers, acid labile linkers, photolabile linkers, peptidase labile linkers, and esterase labile linkers.

In some embodiments, the linker L^{D} or L^{P} is biocleavable under reducing conditions (e.g., a disulfide linker). In this embodiment the drug or PBRM moiety is linked to the polymer through a disulfide bond. The linker molecule comprises a reactive chemical group that can react with the drug. Preferred reactive chemical groups for reaction with the drug or PBRM moiety are N-succinimidyl esters and N-sulfosuccinimidyl esters. Additionally the linker molecule comprises a reactive chemical group, preferably a dithiopyridyl group that can react with the drug to form a disulfide bond. In some embodiments the linker molecules include, for example, N-succinimidyl 3-(2-pyridyldithio)propionate (SPDP), N-succinimidyl 4-(2-pyridyldithio)butanoate (SPDB), N-succinimidyl 4-(2-pyridyldithio)pentanoate (SPP), N-succinimidyl-S-acetylthioacetate (SATA) and N-succinimidyl-oxycarbonyl-alpha-methyl-alpha-(2-pyridyl-dithio)toluene or 2,5-dioxopyrrolidin-1-yl 4-(1-(pyridin-2-yldisulfanyl)ethyl)benzoate (SMPT).

In other embodiments, the biocleavable linker L^{D} or L^{P} is pH-sensitive, i.e., sensitive to hydrolysis at certain pH values. Typically, the pH-sensitive linker is hydrolysable under acidic conditions. For example, an acid-labile linker that is hydrolysable in the lysosome or endosome (e.g., a hydrazone, semicarbazone, thiosemicarbazone, cis-aconitic amide, orthoester, acetal, ketal, or the like) can be used. Such linkers are relatively stable under neutral pH conditions, such as those in the blood, but are unstable at below pH 5.5 or 5.0, the approximate pH of the lysosome. In certain embodiments, the hydrolysable linker is a thioether linker (such as, e.g., a thioether attached to the therapeutic agent via an acylhydrazone bond.

In other embodiments the linker L^{D} or L^{P} is photo-labile and is useful at the body surface and in many body cavities that are accessible to light. Furthermore, L^{D} or L^{P} is biocleavable by infrared light which can penetrate tissue. Accordingly, L^{D} or L^{P} is useful for both applications on the body surface and in the tissue.

In some embodiments, the linker L^{D} or L^{P} is biocleavable by a cleaving agent that is present in the intracellular environment (e.g., within a lysosome or endosome or caveolea). The linker can be, for example, a peptidyl linker that is cleaved by an intracellular peptidase or protease enzyme, including, but not limited to, a lysosomal or endosomal protease.

In some embodiments the linker L^{D} or L^{P} is cleaved by esterases. Only certain esters can be cleaved by esterases present inside or outside cells. Esters are formed by the condensation of a carboxylic acid and an alcohol. Simple esters are esters produced with simple alcohols, such as aliphatic alcohols, and small cyclic and small aromatic alcohols.

In yet other embodiments, the linker L^{D} or L^{P} is not biocleavable and the drug is released by antibody degradation. See, for example, U.S. Pat. No. 7,498,298, which is incorporated by reference herein in its entirety and for all purposes.

Typically, the linker L^{D} or L^{P} is not substantially sensitive to the extracellular environment. As used herein, “not substantially sensitive to the extracellular environment,” in the context of a linker, means that no more than about 20%, typically no more than about 15%, more typically no more than about 10%, and even more typically no more than about 5%, no more than about 3%, or no more than about 1% of the linkers, in a sample of Polymer Drug Conjugate, are cleaved when the Polymer Drug Conjugate presents in an extracellular environment (e.g., in plasma) for 24 hours. Whether a linker is not substantially sensitive to the extracellular environment can be determined, for example, by incubating the Polymer Drug Conjugate with plasma for a predetermined time period (e.g., 2, 4, 8, 16, or 24 hours) and then quantitating the amount of free drug present in the plasma.

In embodiments, the linker L^{D} has the structure:

—R^{L1}—C(═O)—X^{D}-M^{D1}-Y^{D}-M^{D2}-Z^{D}-M^{D3}-Q^{D}-M^{D4}-, with R^{L1} connected to an oxygen atom of the polymeric carrier and M^{D4} connected to the drug molecule to be delivered.

In embodiments, the linker L^{P} has the structure:

—R^{L2}—C(═O)—X^{P}-M^{P1}-Y^{P}-M^{P2}-Z^{P}-M^{P3}-Q-M^{P4}-, with R^{L2} connected to an oxygen atom of the polymeric carrier and M^{P4} connected to the PBRM.

For example, each of R^{L1} and R^{L2} independently is absent, alkyl, alkenyl, alkynyl, cycloalkyl, heteroalkyl, heteroalkenyl, heteroalkynyl, heterocycloalkyl, aryl, or heteroaryl.

For example, each of R^{L1} and R^{L2} independently is absent, alkyl, cycloalkyl, heteroalkyl, or heterocycloalkyl.

For example, R^{L1} is absent.

For example, R^{L2} is absent.

For example, each of X^{D} and X^{P}, independently is —O—, —S—, —N(R¹)—, or absent, in which R¹ is hydrogen, an aliphatic, heteroaliphatic, carbocyclic, or heterocycloalkyl moiety, —C(═O)R^{1B}, —C(═O)OR^{1B}, —SO₂R_{1B} or —N(R¹)— is a heterocycloalkyl moiety, wherein R^{1B} is hydrogen, an aliphatic, heteroaliphatic, carbocyclic, or heterocycloalkyl moiety.

For example, each of Y^{D}, Y^{P}, Z^{D}, Z^{P}, Q^{D}, and Q^{P}, independently, is absent or a biodegradable linker moiety selected from the group consisting of —S—S—, —C(═O)O—, —C(═O)NR²—, —OC(═O)—, —NR²C(═O)—, —OC(═O)O—, —OC(═O)NR²—, —NR²C(═O)O—, —NR²C(═O)NR, —C(OR²)O—, —C(OR²)S—, —C(OR²)NR³, —C(SR²)O—, —C(SR²)S—, —C(SR²)NR³—, —C(NR²R₃)O—, —C(NR²R₃)S—, —C(NR²R₃)NR⁴—, —C(═O)S—, —SC(═O)—, —SC(═O)S—, —OC(═O)S—, —SC(═O)O—, —C(═S)S—, —SC(═S)—, —OC(═S)—, —C(═S)O—, —SC(═S)O—, —OC(═S)S—, —OC(═S)O—, —SC(═S)S—, —C(═NR²)O—, —C(═NR²)—, —C(═NR²)NR³—, —OC(═NR²)—, —SC(═NR²)—, —NR³C(═NR²)—, —NR²SO₂—, —NR²NR³—, —C(═O)NR²NR³—, —NR²NR³C(═O)—, —OC(═O)NR²NR₃—, —NR²NR³C(═O)O—, —C(═S)NR²NR³—, —NR²NR³C(═S)—, —C(═NR⁴)NR²NR³—, —NR²NR³C(═NR⁴)—, —O(N═CR³)—, —(CR³═N)O—, —C(═O)NR²—(N═CR³)—, —(CR³═N)—NR²C(═O)—, —SO₃—, —NR²SO₂NR³—, —SO₂NR²—, and polyamide, wherein each occurrence of R², R³, and R⁴ independently is hydrogen or an aliphatic, heteroaliphatic, carbocyclic, or heterocyclic moiety, or each occurrence of —NR²— or —NR²NR³— is a heterocycloalkyl moiety.

For example, each of M^{D1}, M^{D2}, M^{D3}, M^{D4}, M^{P1}, M^{P2}, M^{P3} and M^{P4}, independently, is absent or a non-biodegradable linker moiety selected from the group consisting of alkyl, alkenyl, alkynyl, cycloalkyl, heteroalkyl, heteroalkenyl, heteroalkynyl, heterocycloalkyl, aryl, heteroaryl, and a combination thereof and each of M^{D1}, M^{D2}, M^{D3}, M^{P1}, M^{P2}, and M^{P3} optionally contains one or more —(C═O)— but does not contain any of the biodegradable linker moieties mentioned above.

For example, each of M^{D1}, M^{D2}, M^{D3}, M^{D4}, M^{P}, M^{P2}, M^{P3} and M^{P4}, independently is C₁₋₆ alkyl, C₁₋₆ alkyl-C(O)—C₀₋₆ alkyl, C₁₋₆ alkyl-NH—C₀₋₆ alkyl, C₁₋₆ alkyl-O—C₀₋₆ alkyl, C₁₋₆ alkyl-S—C₀₋₆ alkyl, C₁₋₆ alkyl-C(O)—C₁₋₆ alkyl-NH, C₁₋₆ alkyl-C(O)—C₁₋₆ alkyl-O, C₁₋₆ alkyl-C(O)—C₁₋₆ alkyl-S, C₃₋₁₀ cycloalkyl-C(O)—C₀₋₆ alkyl, 3-19 membered heterocycloalkyl-C(O)—C₀₋₆ alkyl, aryl-C(O)—C₀₋₆ alkyl, (CH₂CH₂O)₁₋₁₂, and the like.

For example, for each L^{D}, M^{D1} is not absent when X^{D} is absent.

For example, for each L^{P}, M^{P1} is not absent when X^{P} is absent.

For example, for each L^{D}, at least one of X^{D}, Y^{D}, Z^{D}, and Q^{D} is not absent.

For example, for each L^{P}, at least one of X^{P}, Y^{P}, Z^{P}, and Q^{P} is not absent.

For example, each of M^{D}I and M^{P1} independently is C₁₋₆ alkyl or C₁₋₆ heteroalkyl.

For example, each of M^{D2}, M^{D3}, M^{D4}, M^{P2}, M^{P3}, and M^{P4}, independently is absent, C₁₋₆ alkyl, cycloalkyl, heteroalkyl, heterocycloalkyl, or a combination thereof.

For example, for each L^{D}, at most two of M^{D2}, M^{D3}, and M^{D4} are absent.

For example, for each L^{P}, at most two of M^{P2}, M^{P3}, and M^{P4} are absent.

For example, for each L^{D}, one of M^{D2} and M^{D3} has one of the following structures:

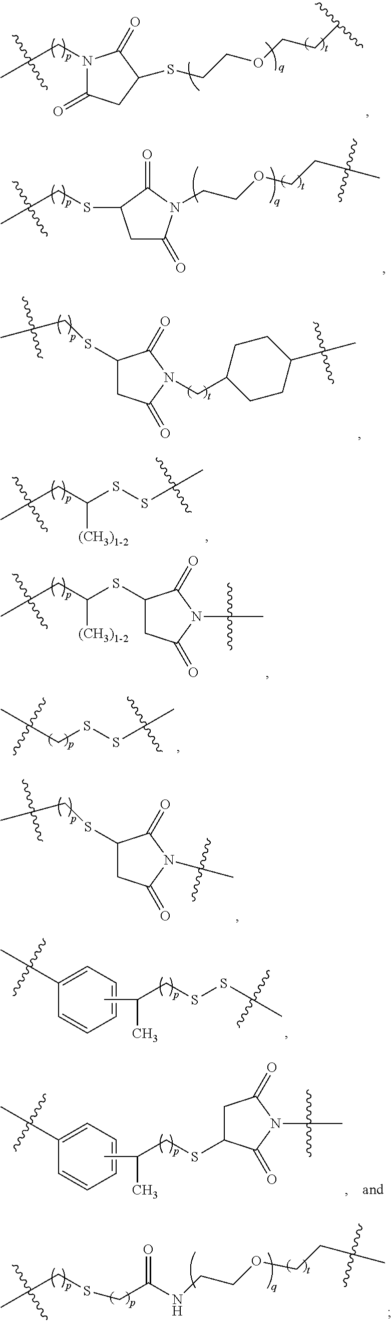

in which q is an integer from 0 to12 and each of p and t independently is an integer from 0 to 3, and the other of M^{D2} or M^{D3} is either absent or a moiety different from the above, such as C₁₋₆ alkyl.

For example, for each L^{P}, one of M^{P2} and M^{P3} has one of the following structures:

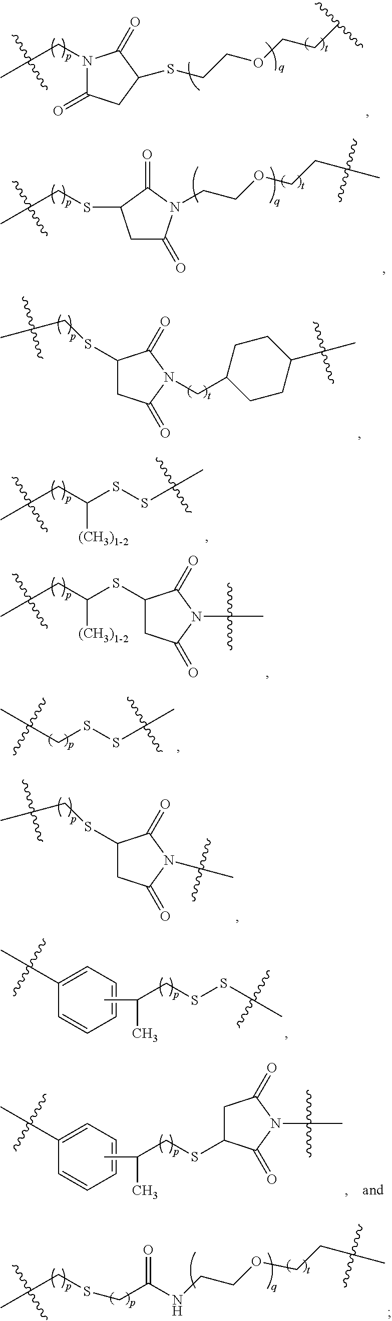

in which q is an integer from 0 to 12 and each of p and t independently is an integer from 0 to 3, and the other of M^{P2} or M^{P3} is either absent or a moiety different from the above, such as C₁₋₆ alkyl.

For example, p is 2.

For example, q is 0 or 12.

For example, t is 0 or 1.

For example, each of -M^{D2}-Z^{D}—, —Z^{D}-M^{D3}-, —Z^{D}-M^{D2}-, or -M^{D3}-Z^{D}—, independently has one of the following structures:

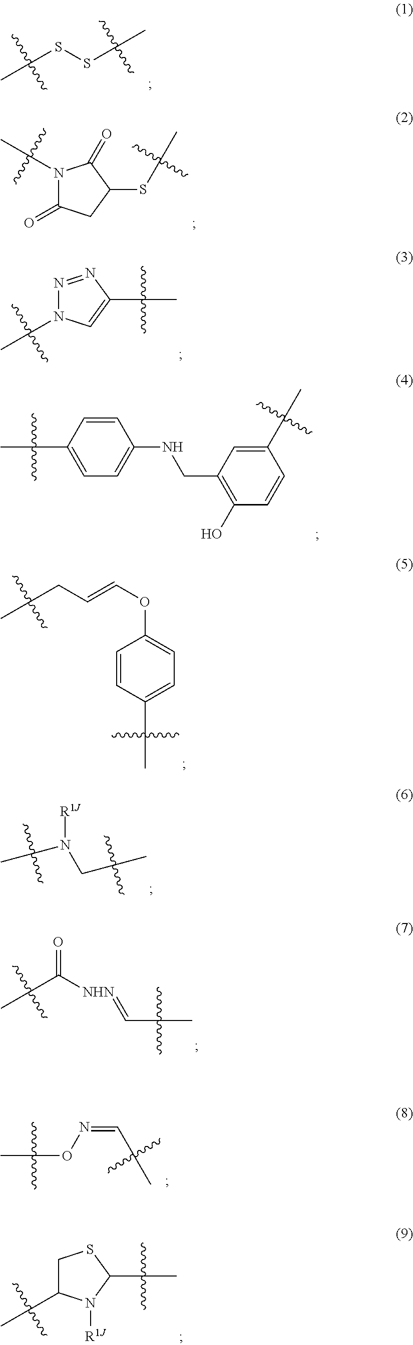

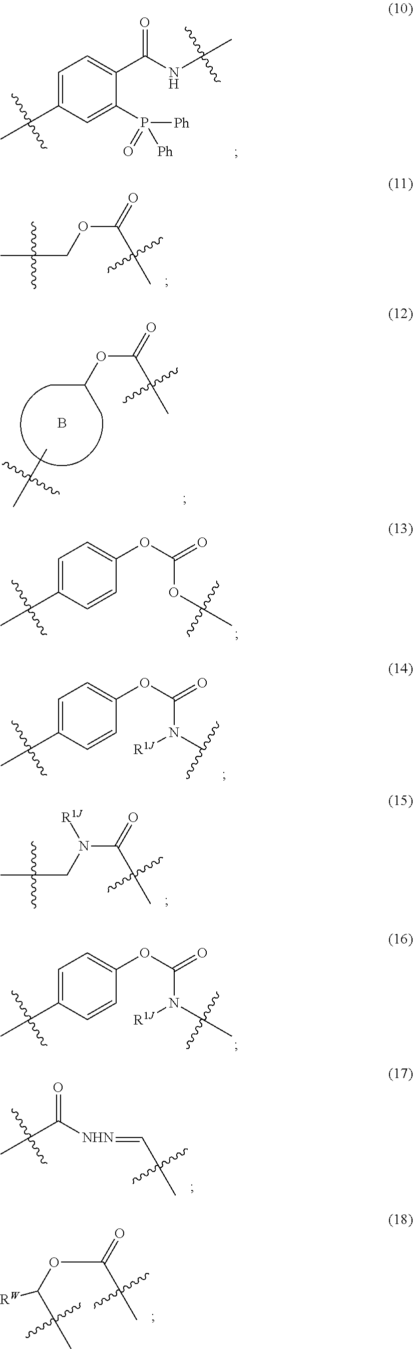

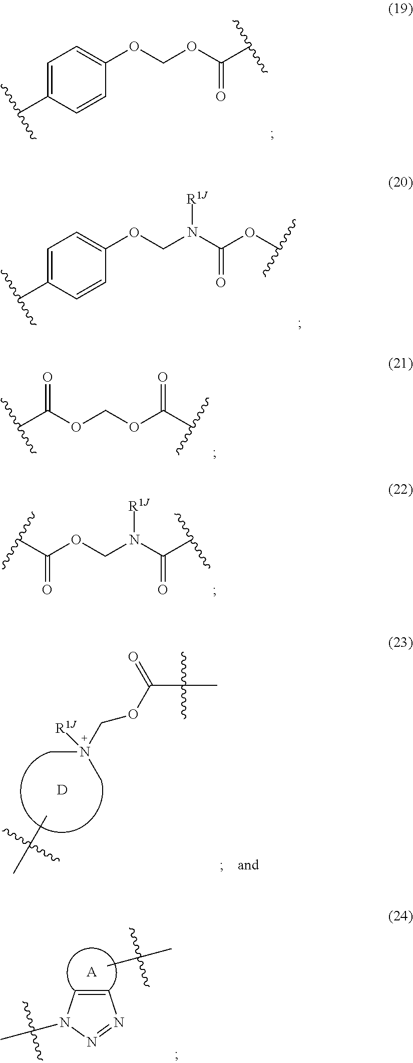

in which ring A or B independently is cycloalkyl or heterocycloalkyl; R^{W} is an aliphatic, heteroaliphatic, carbocyclic, or heterocycloalkyl moiety; R^{1J} is hydrogen, an aliphatic, heteroaliphatic, carbocyclic, or heterocycloalkyl moiety; and ring D is heterocycloalkyl.

For example, each of -M^{P2}-Z^{P}—, —Z^{P}-M^{P3}-, —Z^{P}-M^{P2}-, and -M^{P3}-Z^{P}— independently, has one of the following structures:

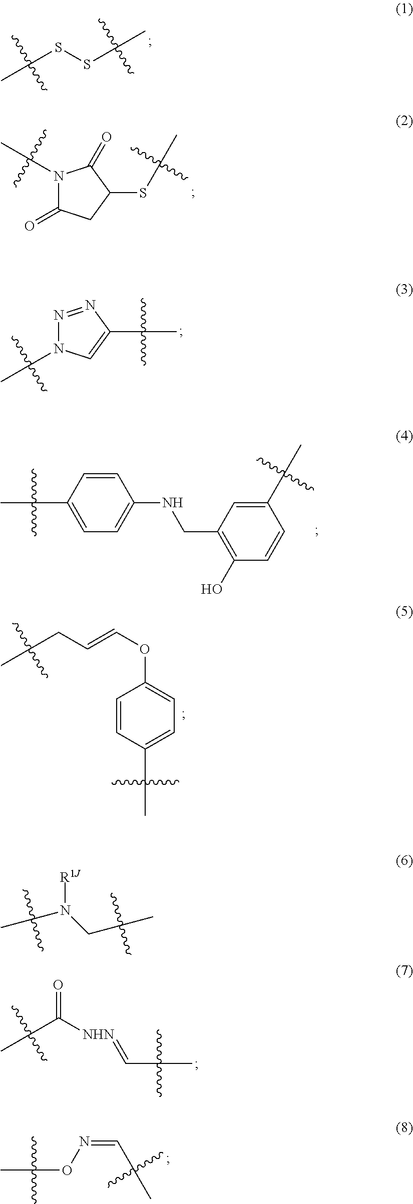

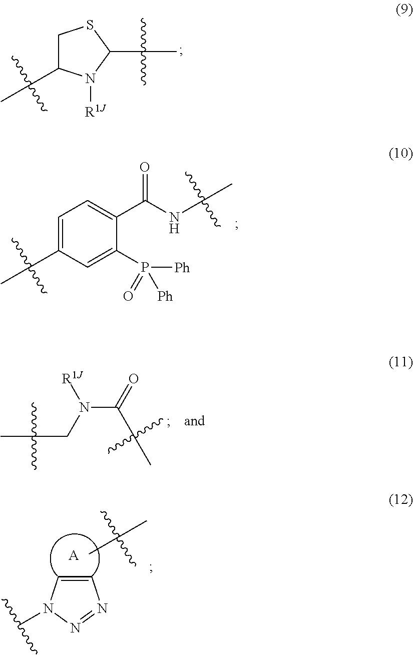

in which ring A is cycloalkyl or heterocycloalkyl and R^{1J} is hydrogen, an aliphatic, heteroaliphatic, carbocyclic, or heterocycloalkyl moiety.

For example, ring A is 5-19 membered heterocycloalkyl, e.g.,

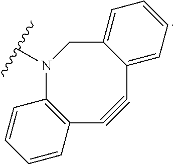 [SMILES: *N1Cc2ccccc2C#Cc2ccccc21]

For example, ring A is C₃₋₈ cycloalkyl.

For example, ring D is piperazinyl or piperidinyl.

For example, R^{W} is C₁₋₆ alkyl.

For example, R^{1J} is hydrogen or C₁₋₆ alkyl.

For example, Z^{D} is

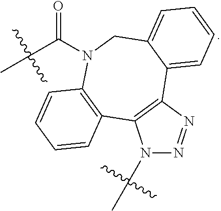 [SMILES: *C(=O)N1Cc2ccccc2-c2nnn(*)c2-c2ccccc21]

For example, Z^{P} is

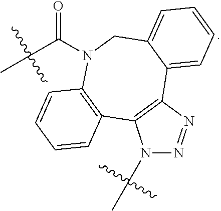 [SMILES: *C(=O)N1Cc2ccccc2-c2nnn(*)c2-c2ccccc21]

For example, X^{D} is absent, O or NH.

For example, X^{P} is absent, O or NH.

For example, each of X^{D} and X^{P}, independently is

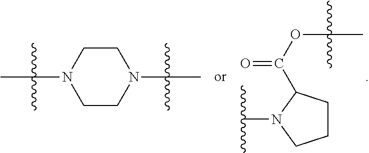 [SMILES: *N1CCN(*)CC1 ; *OC(=O)C1CCCN1*]

For example, each of Y^{D} and Y^{P} independently is —S—S—, —OCO—, —COO—, —CONH— or —NHCO—.

For example, each of Q^{D} and Q^{P} independently is absent, —S—S—, —OCO—, —COO—, —CONH—, —NHCO—, —OCONHNH—, or —NHNHCOO—.

For example, -L^{D}-D can have one of the following structures below, in which the wavy bond

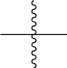

indicates that D (i.e., Drug) is either connected to the functional linker directly or via another moiety:

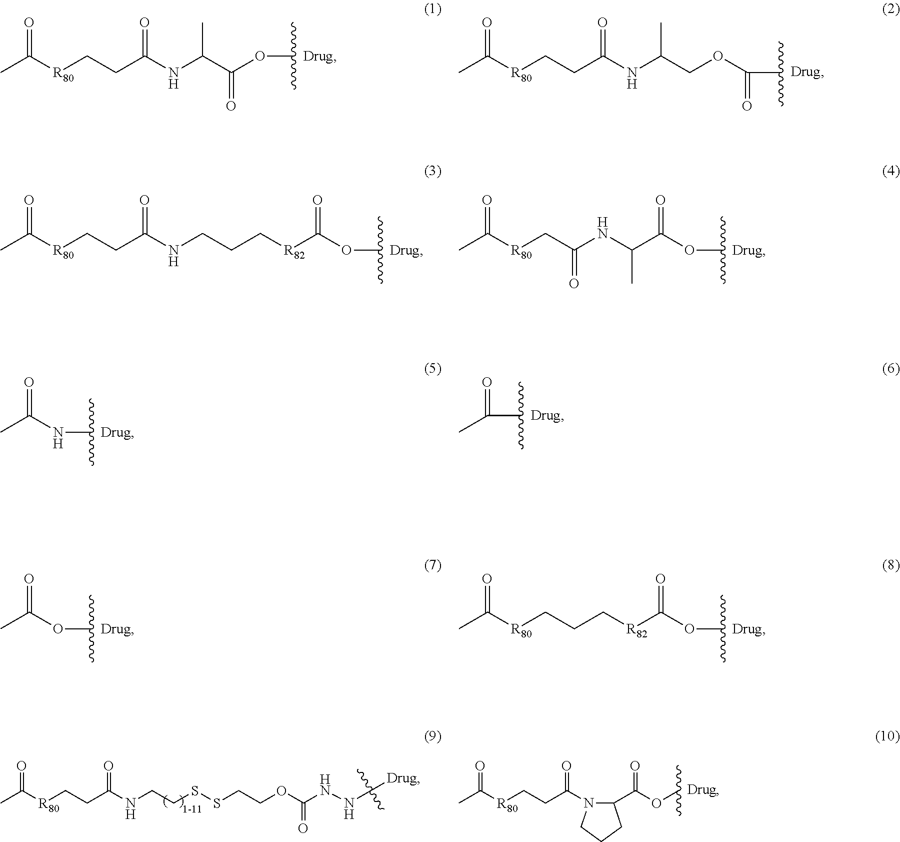

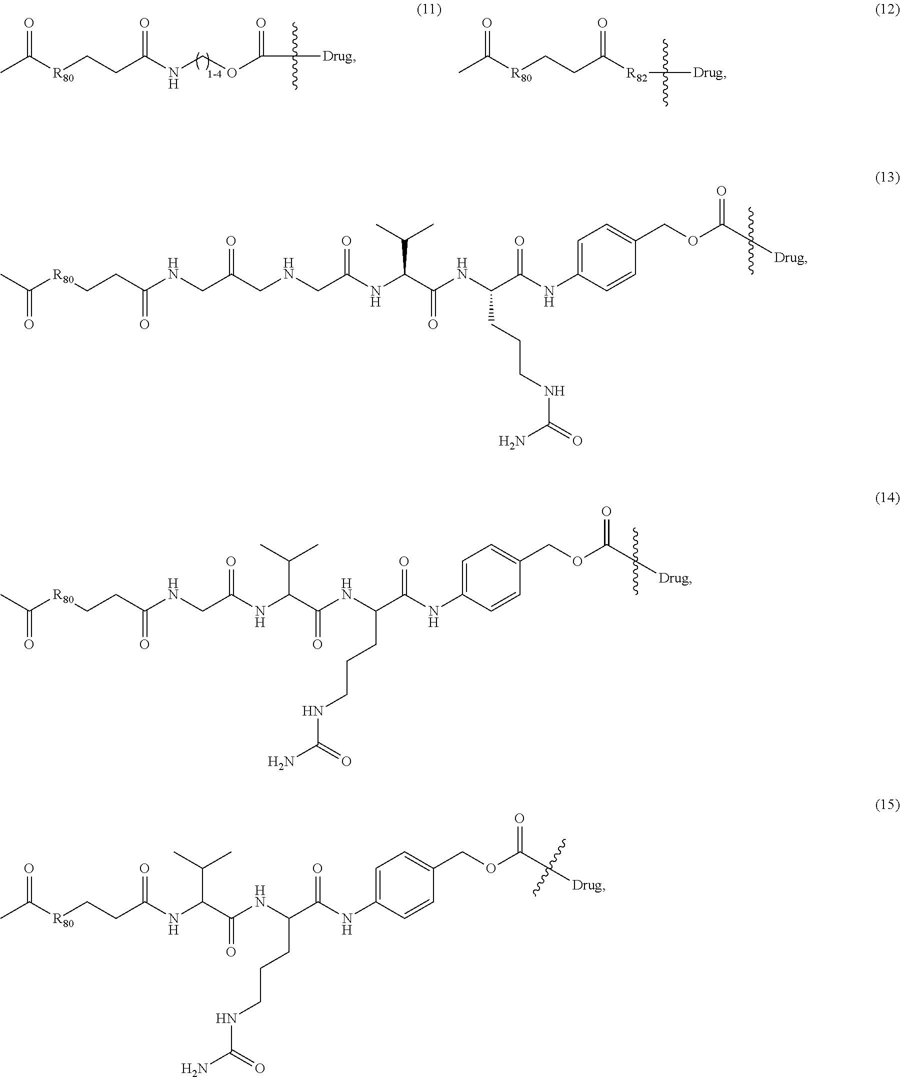

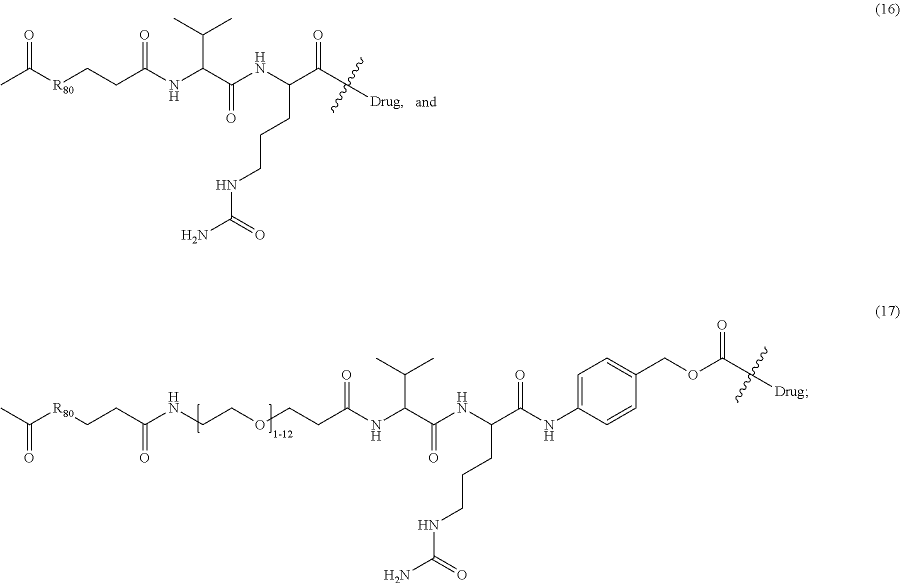

wherein R₈₀ is CH₂, —NH, or oxygen; and
R₈₂ is —NH or oxygen.

For example, polymeric carrier-L^{P}-PBRM can have one of the following structures below, in which the wavy bond

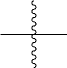

indicates that PBRM is either connected to the functional linker directly or via another moiety:

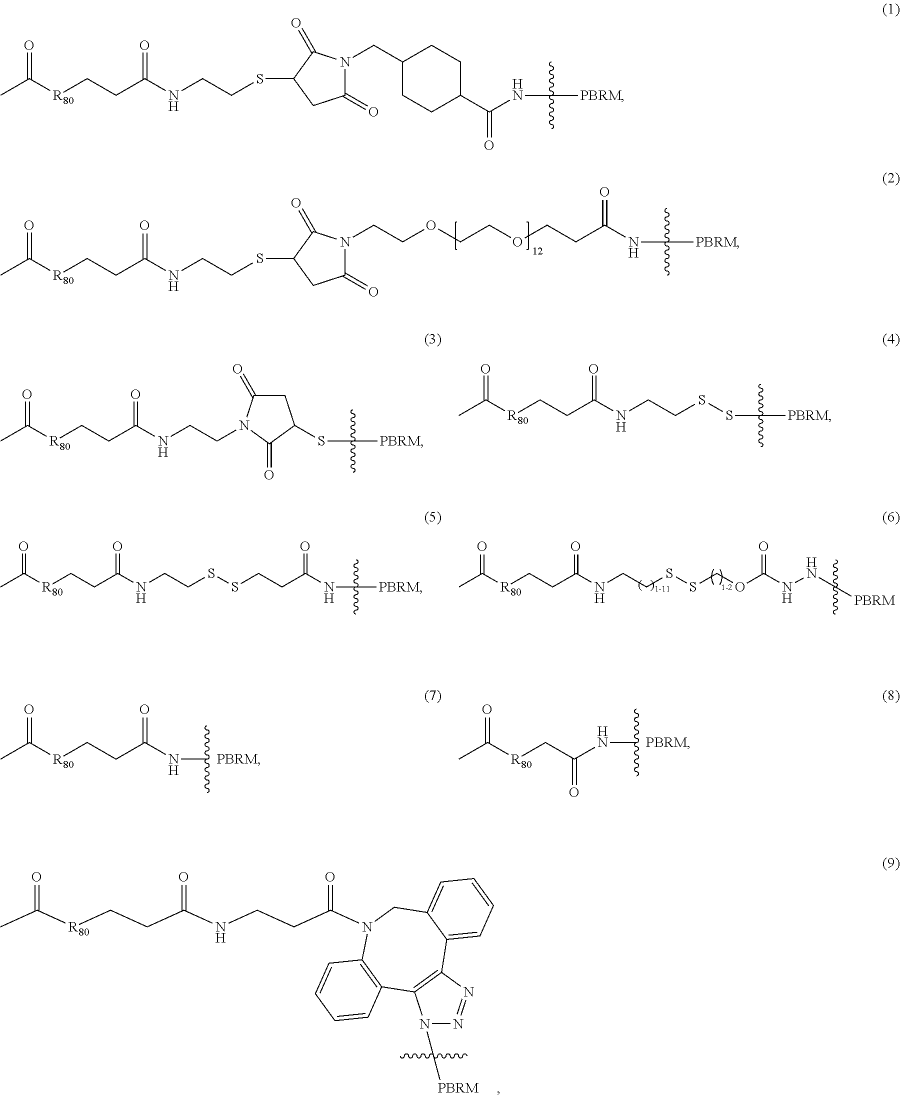

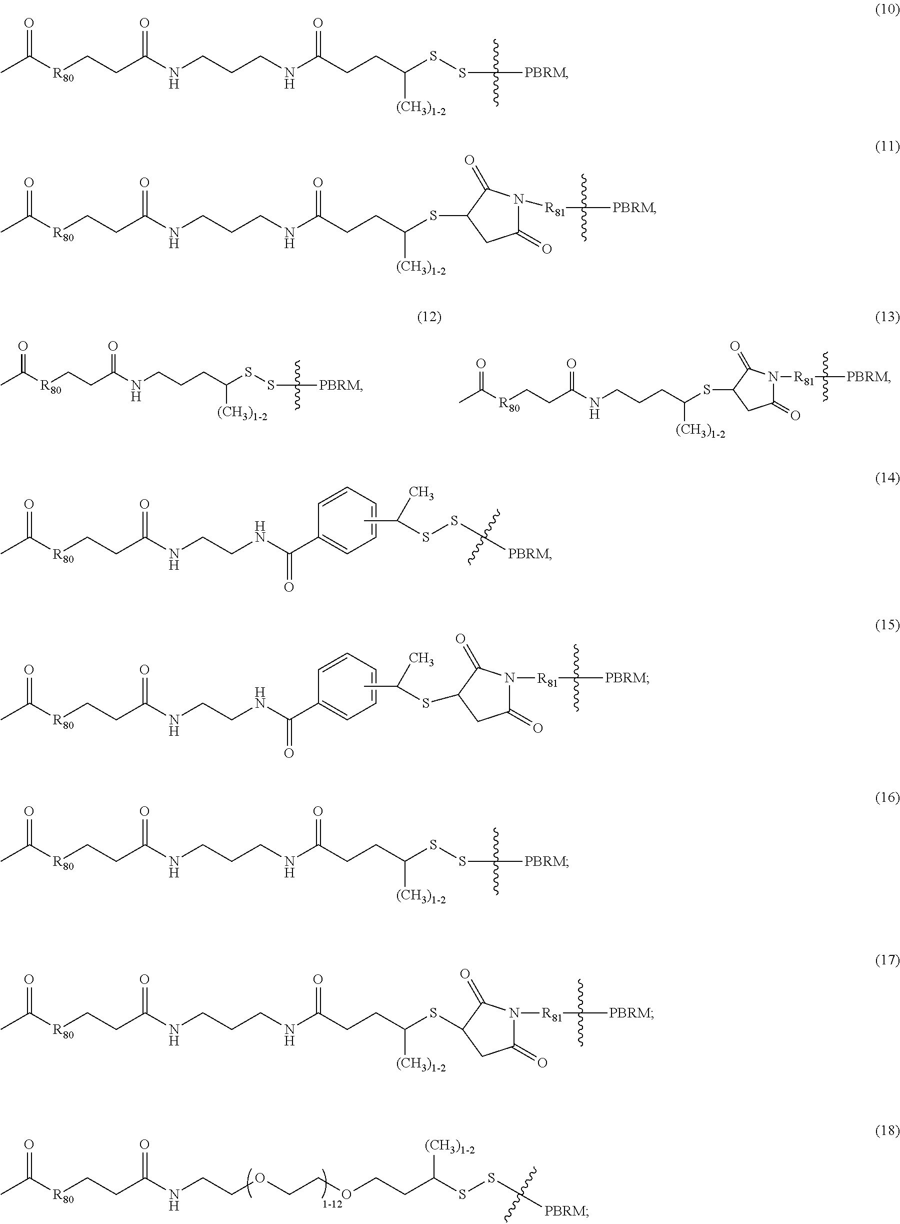

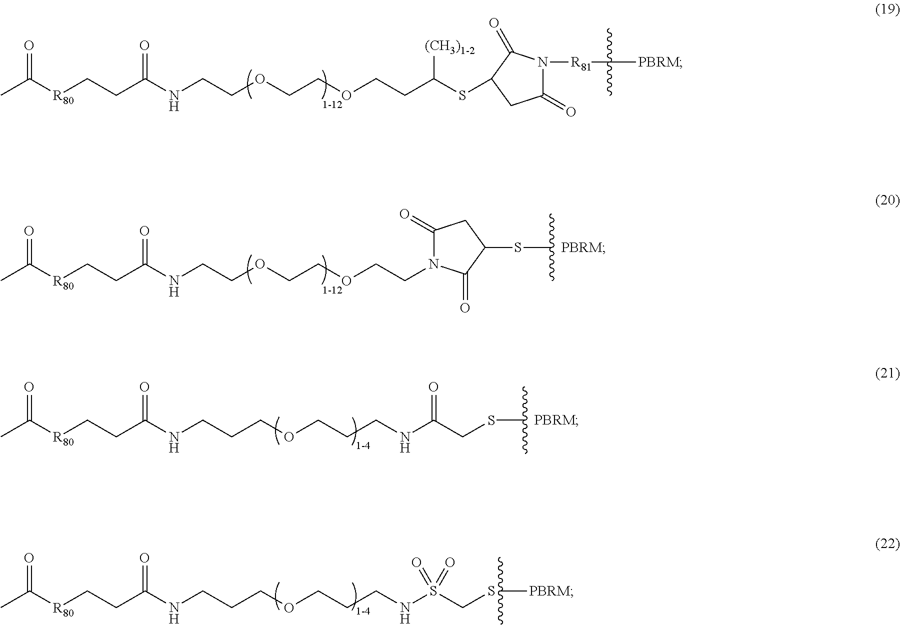

wherein:
R₈₀ is CH₂, NH or oxygen; and
R₈₁ is

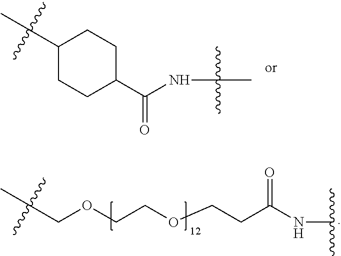

While biocleavable linkers preferably are used in the invention, a non-biocleavable linker also can be used to generate the above-described conjugate. A non-biocleavable linker is any chemical moiety that is capable of linking a drug or PBRM, to the backbone of the a terminally modified polymer in a stable, covalent manner. Thus, non-biocleavable linkers are substantially resistant to acid-induced cleavage, light-induced cleavage, peptidase-induced cleavage, esterase-induced cleavage, and/or disulfide bond cleavage, at conditions under which the drug or polymer remains active.

In one embodiment, a substantial amount of the drug moiety is not cleaved from the conjugate until the protein-polymer-drug conjugate enters a cell with a cell-surface receptor specific for the PBRM of the protein-polymer-drug conjugate, and the drug moiety is cleaved from the protein-polymer-drug conjugate when the protein-polymer-drug conjugate does enter the cell.

In another embodiment, the bioavailability of the protein-polymer-drug conjugate or an intracellular metabolite of the protein-polymer-drug conjugate in a subject is improved when compared to a drug compound or conjugate comprising the drug moiety of the protein-polymer-drug conjugate, or when compared to an analog of the compound not having the drug moiety.

In another embodiment, the drug moiety is intracellularly cleaved in a subject from the protein-polymer-drug conjugate, or an intracellular metabolite of the protein-polymer-drug conjugate.

### Conjugates

The invention also features a terminal conjugate comprising a terminally modified polymer described above and a pharmaceutically useful modifier (“M”) covalently conjugated with L^{M} or —O—CH₂—CH(OH)—CH₂—CR¹═CR²R³ of the terminally modified polymer.

In one embodiment, the terminal conjugate is of formula (I):

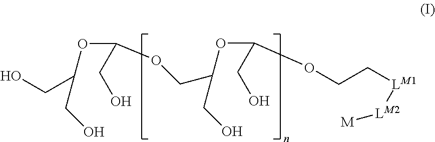

wherein
n is an integer between 1 and about 1100,
L^{M1} is —NR¹, —NR¹C(═X¹)—, —NR¹C(═X¹)Y—, —NR¹NR²—, —NR¹NR²C(═X¹)—, —NR¹NR²C(═X¹)Y—, —NR¹SO₂—, or —NR¹SO₂NR²—, with the NR¹ moiety attached to the polymer in the order as written, and
L^{M2} is —(CH₂)ₘ—W—, with (CH₂)ₘ connected to L^{M1}, in which m is an integer between 0 and 20, and W, prior to conjugating with M, is a functional group suitable for covalently conjugating with M or W is an aliphatic, heteroaliphatic, carbocyclic, or heterocyclic moiety, wherein the aliphatic, heteroaliphatic, carbocyclic, or heterocycloalkyl moiety comprises a functional group suitable for coupling with M.

The terminal conjugate may contain only one -L^{M}-M. M can either be a drug or a PBRM.

The terminally modified polymer of the invention comprises a polyacetal, e.g., a PHF having a molecular weight (i.e., MW of the unmodified PHF) ranging from about 0.5 kDa to about 300 kDa (e.g., about 1 kDa to about 150 kDa or about 2 kDa to about 75 kDa). The selection of a terminally modified polymer having a specific MW range may depend on the size of the PBRM to be conjugated with the polymer at the terminus of the polymer to form a terminal conjugate.

For example, for conjugating a PBRM having a molecular weight of 40 kDa or greater (e.g., 60 kDa or greater, 80 kDa or greater, 100 kDa or greater, 120 kDa or greater, 140 kDa or greater, 160 kDa or greater or 180 kDa or greater), the terminally modified polymer of the invention comprises a polyacetal, e.g., a PHF having a molecular weight (i.e., MW of the unmodified PHF) ranging from about 2 kDa to about 25 kDa (e.g., about 4-10 kDa). For example the PHF has a molecular weight of about 2 kDa, 4 kDa, 10 kDa, 15 kDa, 20 kDa or 25 kDa.

For example, for conjugating a PBRM having a molecular weight of 40 kDa to 200 kDa, the terminally modified polymer of the invention comprises a polyacetal, e.g., a PHF having a molecular weight (i.e., MW of the unmodified PHF) ranging from about 2 kDa to about 25 kDa (e.g., about 4-10 kDa). For example the PHF has a molecular weight of about 2 kDa, 4 kDa, 10 kDa, 15 kDa, 20 kDa or 25 kDa.

For example, for conjugating a PBRM having a molecular weight of 60 kDa to 120 kDa, the terminally modified polymer of the invention comprises a polyacetal, e.g., a PHF having a molecular weight (i.e., MW of the unmodified PHF) ranging from about 2 kDa to about 25 kDa (e.g., about 4-10 kDa). For example the PHF has a molecular weight of about 2 kDa, 4 kDa, 10 kDa, 15 kDa, 20 kDa or 25 kDa.

PBRMs in this molecular weight range, include but are not limited to, for example, camelids, Fab2, and the like.

For conjugating a PBRM having a molecular weight of 140 kDa to 180 kDa, the terminally modified polymer of the invention comprises a polyacetal, e.g., a PHF having a molecular weight (i.e., MW of the unmodified PHF) ranging from about 2 kDa to about 25 kDa (e.g., about 4-10 kDa). For example the PHF has a molecular weight of about 2 kDa, 4 kDa, 10 kDa, 15 kDa, 20 kDa or 25 kDa.

PBRMs in this molecular weight range, include but are not limited to, for example, full length antibodies, such as, IgG and IgM.

For conjugating a PBRM having a molecular weight of 200 kDa or less (e.g., 120 kDa or less, 80 kDa or less, 60 kDa or less, 40 kDa or less, 20 kDa or less or 10 kDa or less), the terminally modified polymer of the invention comprises a polyacetal, e.g., a PHF having a molecular weight (i.e., MW of the unmodified PHF) ranging from about 20 kDa to about 75 kDa (e.g., about 25-55 kDa). For example the PHF has a molecular weight of about 25 kDa, 35 kDa, 40 kDa, 50 kDa or 55 kDa.

For conjugating a PBRM having a molecular weight of 4 kDa to 80 kDa (e.g., 4-20 kDa, 20-30 kDa, or 30-70 kDa), the terminally modified polymer of the invention comprises a polyacetal, e.g., a PHF having a molecular weight (i.e., MW of the unmodified PHF) ranging from about 20 kDa to about 75 kDa (e.g., about 25-55 kDa). For example the PHF has a molecular weight of about 25 kDa, 35 kDa, 40 kDa, 50 kDa or 55 kDa). For example the PHF has a molecular weight of about 25 kDa, 35 kDa, 40 kDa, 50 kDa or 55 kDa.

For conjugating a PBRM having a molecular weight of 80 kDa or less (e.g., 70 kDa or less, 60 kDa or less, 50 kDa or less or 40 kDa or less), the terminally modified polymer of the invention comprises a polyacetal, e.g., a PHF having a molecular weight (i.e., MW of the unmodified PHF) ranging from about 20 kDa to about 75 kDa (e.g., about 25-55 kDa). For example the PHF has a molecular weight of about 25 kDa, 35 kDa, 40 kDa, 50 kDa or 55 kDa.

PBRMs in this molecular weight range, include but are not limited to, for example, antibody fragments such as, for example Fabs.

For conjugating a PBRM having a molecular weight of 30 kDa or less (e.g., about 20 kDa or less), the terminally modified polymer of the invention comprises a polyacetal, e.g., a PHF having a molecular weight (i.e., MW of the unmodified PHF) ranging from about 20 kDa to about 75 kDa (e.g., about 25-55 kDa). For example the PHF has a molecular weight of about 25 kDa, 35 kDa, 40 kDa, 50 kDa or 55 kDa.

PBRMs in this molecular weight range, include but are not limited to, for example, antibody fragments, such as, scFv.

For conjugating a PBRM having a molecular weight of 20 kDa or less (e.g., 10 kDa or less), the terminally modified polymer of the invention comprises a polyacetal, e.g., a PHF having a molecular weight (i.e., MW of the unmodified PHF) ranging from about 20 kDa to about 75 kDa (e.g., about 25-55 kDa). For example the PHF has a molecular weight of about 25 kDa, 35 kDa, 40 kDa, 50 kDa or 55 kDa.

PBRMs in this molecular weight range, include but are not limited to, for example, small proteins and peptides.

The terminal conjugates of the invention may further comprise one or more occurrences of M along the backbone of the polymeric carrier. Where M is D or a therapeutic agent, e.g., a drug, wherein the one or more occurrences of M may be the same or different. In certain other embodiment, one or more occurrences of M along the backbone is PBRM, wherein the one or more occurrences of PBRM may be the same or different. In certain other embodiments, the terminal conjugate contains one or more occurrences of D (e.g., a drug) along the backbone while the terminal M is a PBRM (e.g., an antibody, a protein or a peptide).

In certain embodiments, the conjugates are formed in several steps. These steps include (1) providing a polyacetal or polyketal that has a terminal amino (i.e., NH₂) group; (2) modifying the terminal amino group so as to obtain a terminally modified polymer containing —O—(CH₂)₂-L^{M} at one of its termini, L^{M} being a linker capable of covalently conjugating with M, and (3) coupling the terminally modified polymer with M. L^{M} as defined herein, comprises a nitrogen-containing moiety selected from the group consisting of —NR¹, —NR¹C(═X¹)—, —NR¹C(═X¹)Y—, —NR¹NR²—, —NR¹NR²C(═X¹)—, —NR¹NR²C(═X¹)Y—, —NR¹SO₂—, and —NR¹SO₂NR²—, with the NR¹ moiety attached directly or indirectly to the polymer in the order as written, in which X¹ is O, S, or NR³ and Y is O, S, or NR⁴, and each of R¹, R², R³, and R⁴ independently is H or an aliphatic, heteroaliphatic, carbocyclic, or heterocyclic moiety.

In some embodiments, the polyacetal or polyketal that has a terminal amino in step (1) above is obtained by providing a polyacetal or polyketal that has a terminal aldehyde group and reductively aminating the terminal aldehyde group to form a terminal amino group.

In another embodiment, the conjugates are formed in several steps: (1) providing a polyacetal or polyketal that has a terminal aldehyde group; (2) reacting the terminal aldehyde group with Z—CH₂—CR¹═CR²R³ to obtain a terminally modified polymer, at least one terminus of which is —O—CH₂—CH(OH)—CH₂—CR¹═CR²R³, Z being halo (e.g., Cl, Br, or I); and (3) coupling the terminally modified polymer with M.

The biodegradable biocompatible conjugates of the invention can be prepared to meet desired requirements of biodegradability and hydrophilicity. For example, under physiological conditions, a balance between biodegradability and stability can be reached. For instance, it is known that molecules with molecular weights beyond a certain threshold (generally, above 40-100 kDa, depending on the physical shape of the molecule) are not excreted through kidneys, as small molecules are, and can be cleared from the body only through uptake by cells and degradation in intracellular compartments, most notably lysosomes. This observation exemplifies how functionally stable yet biodegradable materials may be designed by modulating their stability under general physiological conditions (pH=7.5±0.5) and at lysosomal pH (pH near 5). For example, hydrolysis of acetal and ketal groups is known to be catalyzed by acids, therefore polyals will be in general less stable in acidic lysosomal environment than, for example, in blood plasma. One can design a test to compare polymer degradation profile at, for example, pH=5 and pH=7.5 at 37° C. in aqueous media, and thus to determine the expected balance of polymer stability in normal physiological environment and in the “digestive” lysosomal compartment after uptake by cells. Polymer integrity in such tests can be measured, for example, by size exclusion HPLC. One skilled on the art can select other suitable methods for studying various fragments of the degraded conjugates of this invention.

In many cases, it will be preferable that at pH=7.5 the effective size of the polymer will not detectably change over 1 to 7 days, and remain within 50% from the original for at least several weeks. At pH=5, on the other hand, the polymer should preferably detectably degrade over 1 to 5 days, and be completely transformed into low molecular weight fragments within a two-week to several-month time frame. Although faster degradation may be in some cases preferable, in general it may be more desirable that the polymer degrades in cells with the rate that does not exceed the rate of metabolization or excretion of polymer fragments by the cells. Accordingly, in certain embodiments, the conjugates of the present invention are expected to be biodegradable, in particular upon uptake by cells, and relatively “inert” in relation to biological systems. The products of carrier degradation are preferably uncharged and do not significantly shift the pH of the environment. It is proposed that the abundance of alcohol groups may provide low rate of polymer recognition by cell receptors, particularly of phagocytes. The polymer backbones of the present invention generally contain few, if any, antigenic determinants (characteristic, for example, for some polysaccharides and polypeptides) and generally do not comprise rigid structures capable of engaging in “key-and-lock” type interactions in vivo unless the latter are desirable. Thus, the soluble, crosslinked and solid conjugates of this invention are predicted to have low toxicity and bioadhesivity, which makes them suitable for several biomedical applications.

In certain embodiments of the present invention, the biodegradable biocompatible conjugates can form linear or branched structures. For example, the biodegradable biocompatible polyal conjugates of the present invention can be chiral (optically active). Optionally, the biodegradable biocompatible polyal conjugates of the present invention can be scalemic.

In certain embodiments, the conjugates of the invention are water-soluble. In certain embodiments, the conjugates of the invention are water-insoluble. In certain embodiments, the inventive conjugate is in a solid form. In certain embodiments, the conjugates of the invention are colloids. In certain embodiments, the conjugates of the invention are in particle form. In certain embodiments, the conjugates of the invention are in gel form.

### Synthetic Methods

According to the present invention, any available techniques can be used to make the inventive terminally modified polymers, their conjugates or compositions including them, and intermediates and components (e.g., carriers and modifiers) useful for making them. For example, semi-synthetic and fully synthetic methods such as those discussed in detail below may be used.

### Carriers

Methods for preparing polymer carriers (e.g., biocompatible, biodegradable polymer carriers) suitable for conjugation to modifiers are known in the art. For example, synthetic guidance can be found in U.S. Pat. Nos. 5,811,510; 5,863,990; 5,958,398; 7,838,619; and 7,790,150; and U.S. Publication No. 2006/0058512. The skilled practitioner will know how to adapt these methods to make polymer carriers for use in the practice of the invention.

### Terminally Modified Carriers

Methods for preparing terminally modified polymer carriers (e.g., PHF) of the invention are illustrated in Schemes 1-7 below.

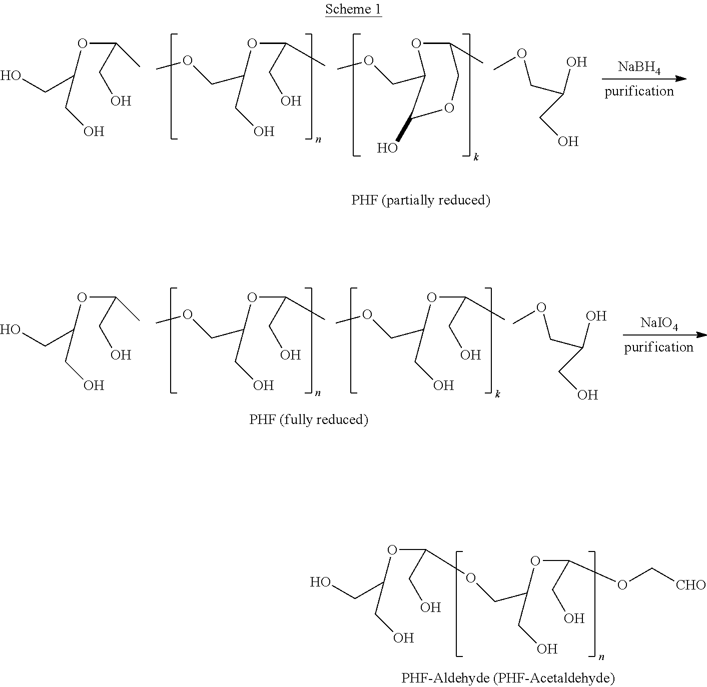

Scheme 1 above shows the synthesis of a PHF with a terminal aldehyde group (PHF—CHO). Unless otherwise specified, in the schemes or polymer formulae of the present disclosure, the disconnection or gap between the polyacetal units indicates that the units can be connected to each other in any order. For example, in Scheme 1 above, the gap between block n, block k, and the glycolic end (i.e., the end next to the k block as shown) means that either the n or k block can be next to the glycolic terminus, and that the n and k block can be randomly distributed along the polymer backbone.

PHF, consists of alternative repeating glycol and glycerol units which are threaded via an ether linkage. As show in Scheme 2 below, when PHF is treated with sodium periodate, only the ‘glycolic terminal end (A)’ that has vicinal diols undergoes oxidation resulting in cleavage of carbon-1 as formaldehyde and eventually produces a ‘glycerolic terminal end (B)’ that has another set of diols. This terminal glycerol undergoes periodate oxidation and produces a relatively stable aldehyde functionality at carbon-2.

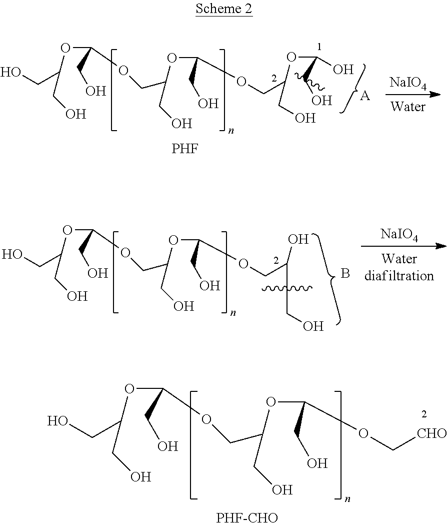

Scheme 3 below shows the synthesis of a PHF with a terminal maleimido group, i.e., Compound (6). As shown below, PHF—CHO is first converted to PHF—NH₂ via reductive amination, and the target product (6) is produced by EDC mediated coupling of 6-maeimido hexanoic acid and PHF—NH₂.

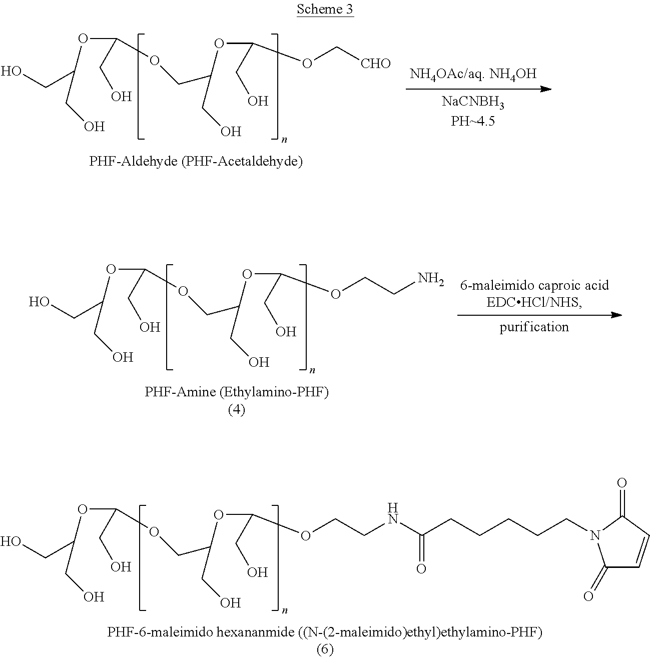

Scheme 4 below shows another route, i.e., reductive amination only, for the synthesis of a PHF with a terminal maleimido group, i.e., Compound (3).

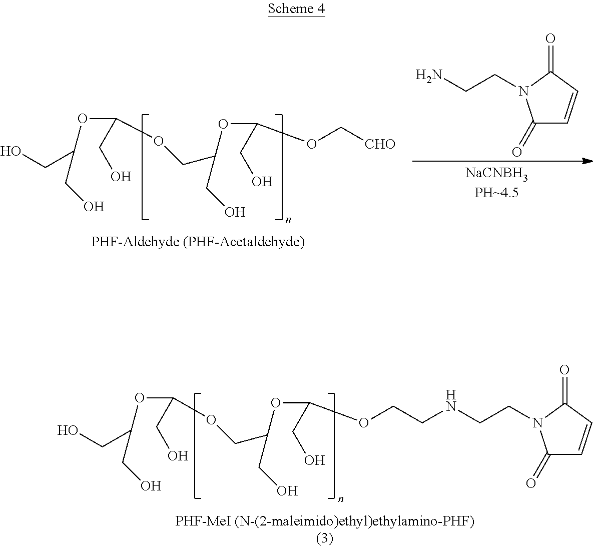

Scheme 5 below shows the synthesis of a terminally modified PHF with further modification along the backbone. In particular, 3-alanine is introduced to the PHF backbone followed by drug conjugation.

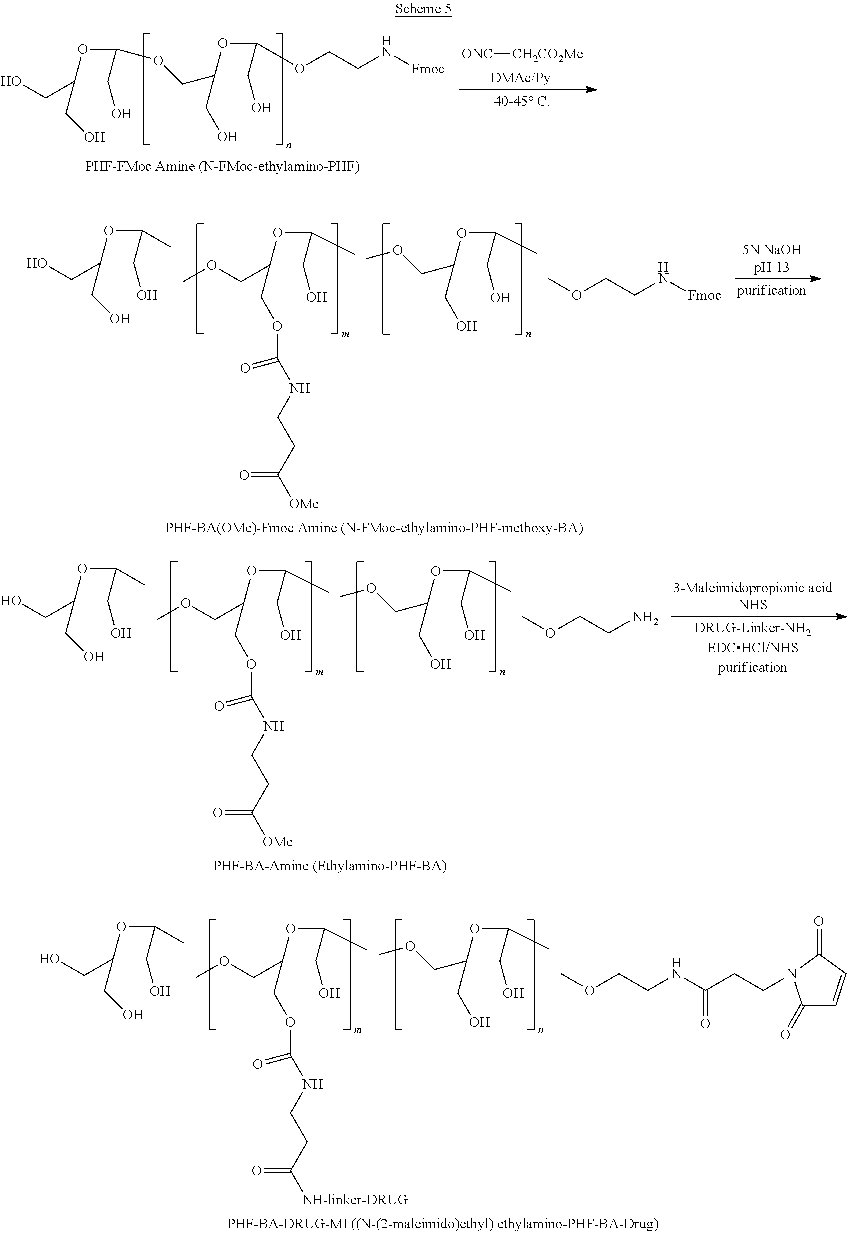

Scheme 6 below shows another route for synthesizing a terminally modified PHF with further modification along the backbone.

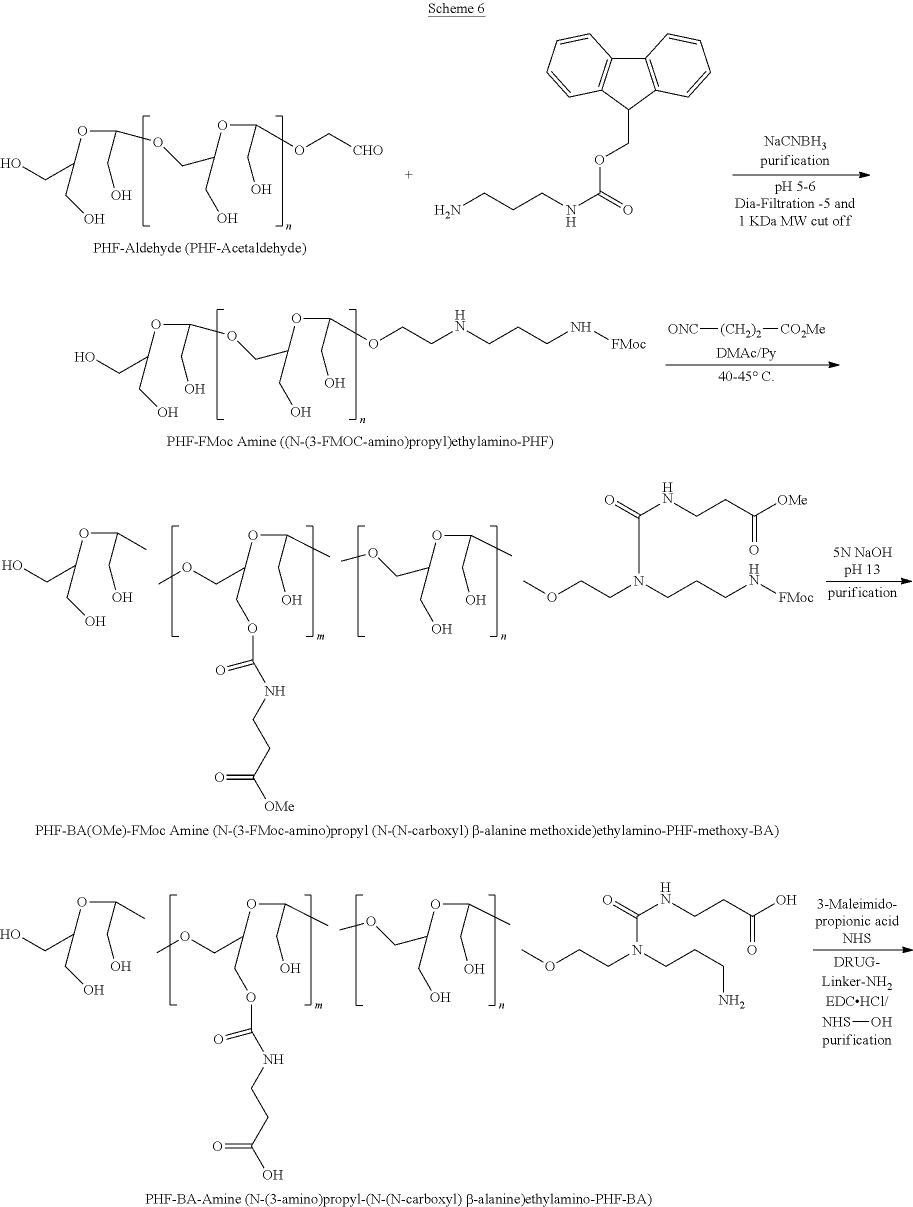

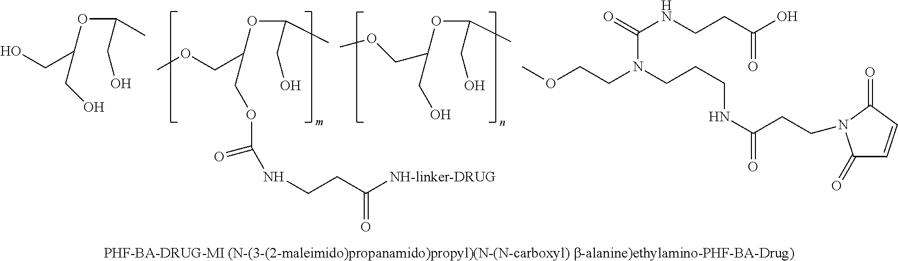

Scheme 7 below shows a route for synthesizing a terminally modified PHF containing a terminal double bond.

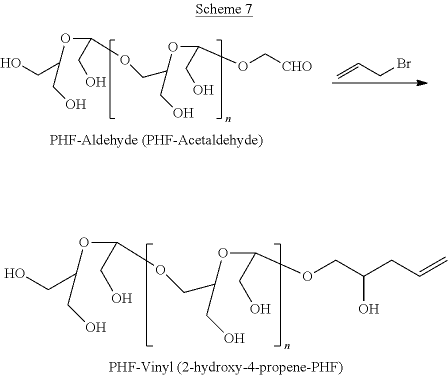

Scheme 7A below shows the synthesis of a terminally modified PHF with further modification along the backbone. In particular, glutaric acid is introduced to the PHF backbone followed by drug conjugation.

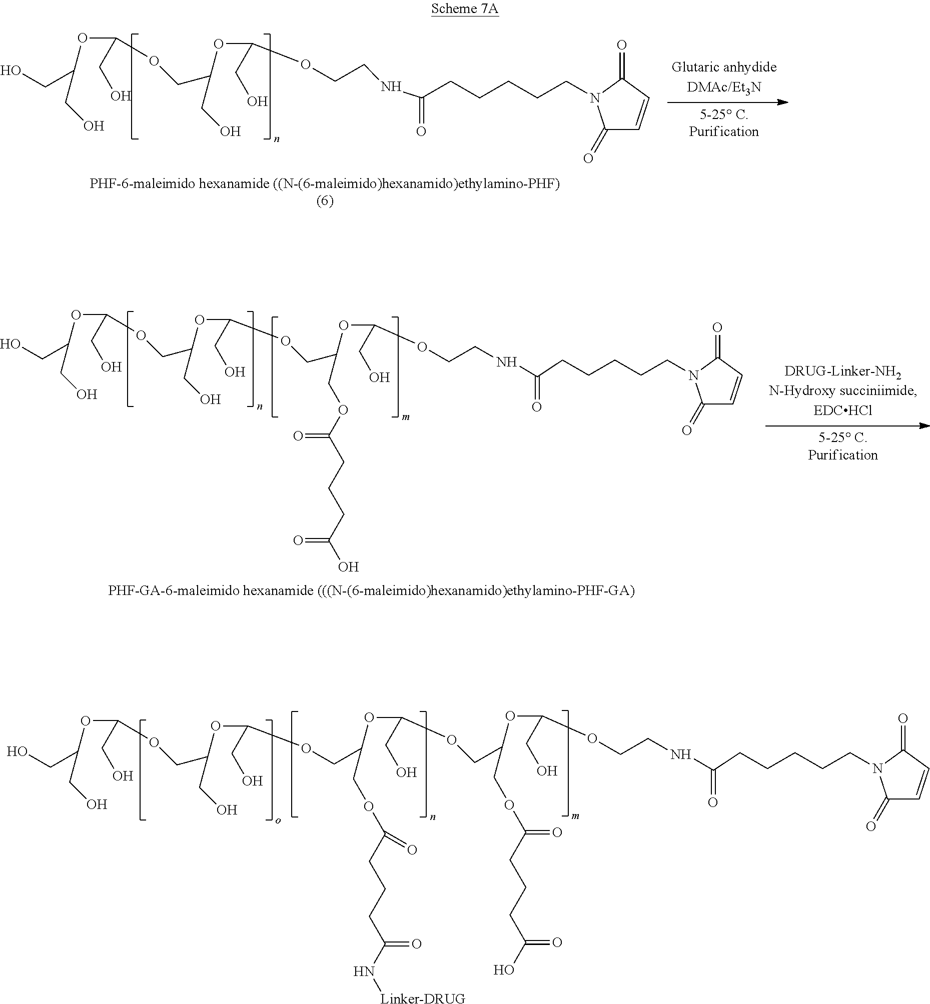

One or more PBRM can also be linked to the backbone of the terminally modified polymer using standard synthetic methods for protein conjugation, including, but not limited to, reactions based on reductive amination, Staudinger ligation, oxime formation, thiazolidine formation and the methods and reactions described herein as well as in WO 2012/171020 and U.S. Publication No. 2013/0101546, the disclosures of which are incorporated herein by reference in their entirety.

### Conjugates

The general methods of producing the terminally modified polymer have been described above. Schemes 8-11 below exemplify how the terminal conjugates are synthesized from the terminally modified polymers. All the Schemes involve maleimido-thiol chemistry. Other methods for conjugating a protein or antibody to the terminally modified polymer can also be used such as Staudinger ligation, oxime formation, thiazolidine formation and the methods and reactions described in WO 2012/171020 and U.S. Publication No. 2013/0101546, the disclosures of which are incorporated herein by reference in their entirety.

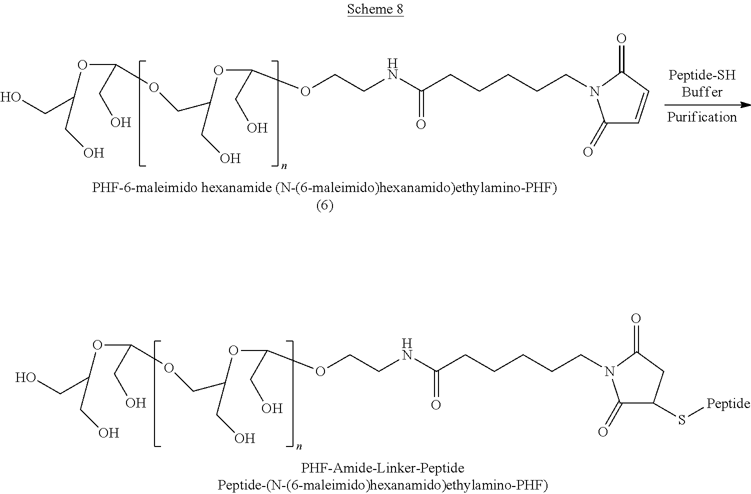

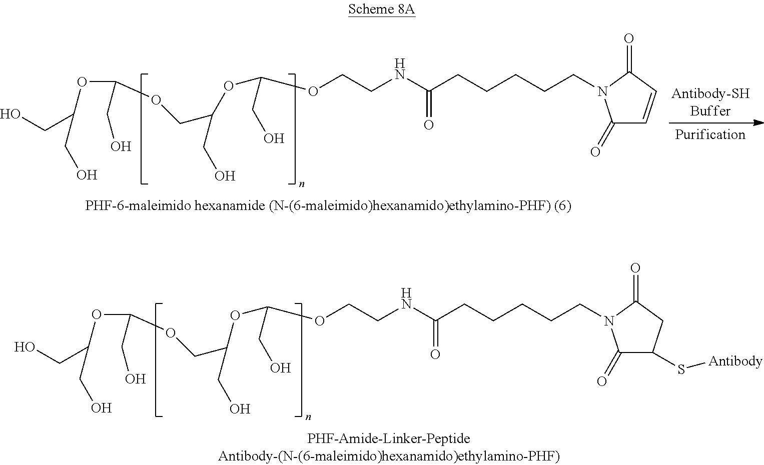

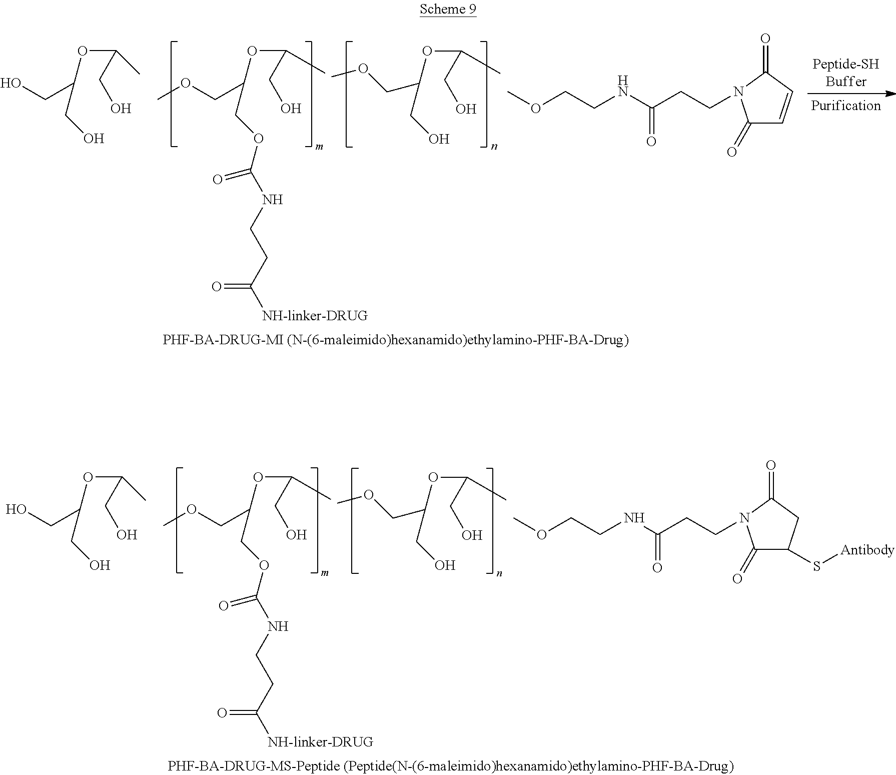

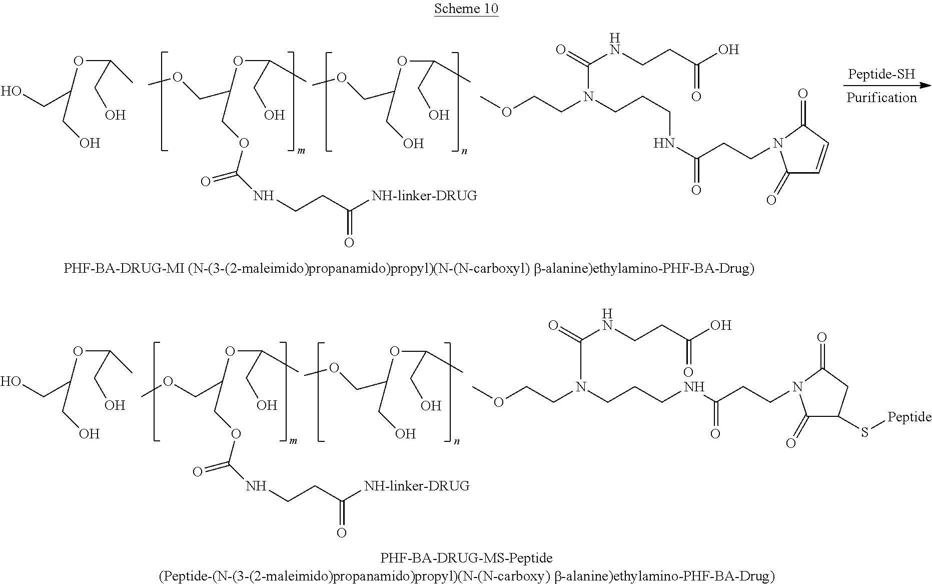

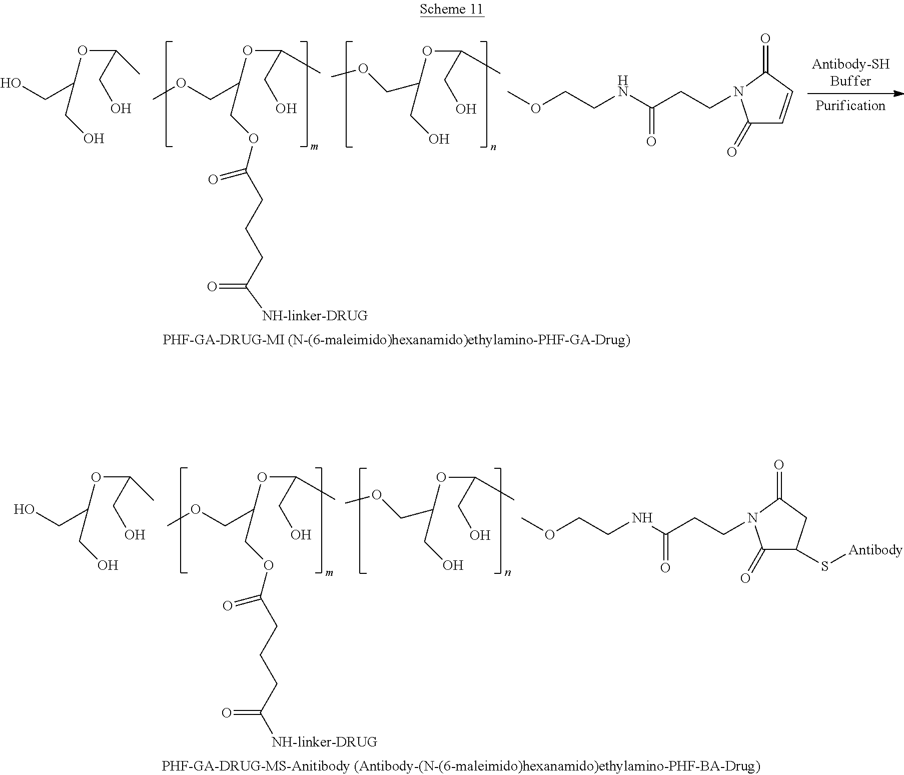

### Pharmaceutical Compositions

Also included are pharmaceutical compositions comprising one or more terminal conjugates as disclosed herein in an acceptable carrier, such as a stabilizer, buffer, and the like. The conjugates can be administered and introduced into a subject by standard means, with or without stabilizers, buffers, and the like, to form a pharmaceutical composition. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal, oral or parenteral administration including intravenous, intraarterial, subcutaneous, intraperitoneal or intramuscular injection or infusion or intracranial, e.g., intrathecal or intraventricular, administration. The conjugates can be formulated and used as sterile solutions and/or suspensions for injectable administration; lyophilized powders for reconstitution prior to injection/infusion; topical compositions; as tablets, capsules, or elixirs for oral administration; or suppositories for rectal administration, and the other compositions known in the art.

A pharmacological composition or formulation refers to a composition or formulation in a form suitable for administration, e.g., systemic administration, into a cell or subject, including for example a human. Suitable forms, in part, depend upon the use or the route of entry, for example oral, inhaled, transdermal, or by injection/infusion. Such forms should not prevent the composition or formulation from reaching a target cell (i.e., a cell to which the drug is desirable for delivery). For example, pharmacological compositions injected into the blood stream should be soluble. Other factors are known in the art, and include considerations such as toxicity and forms that prevent the composition or formulation from exerting its effect.

By “systemic administration” is meant in vivo systemic absorption or accumulation of the modified polymer in the blood stream followed by distribution throughout the entire body. Administration routes that lead to systemic absorption include, without limitation: intravenous, subcutaneous, intraperitoneal, inhalation, oral, intrapulmonary, and intramuscular. Each of these administration routes exposes the modified polymers to an accessible diseased tissue. The rate of entry of an active agent into the circulation has been shown to be a function of molecular weight or size. The use of a conjugate of this invention can localize the drug delivery in certain cells, such as cancer cells via the specificity of PBRMs.

A “pharmaceutically acceptable formulation” means a composition or formulation that allows for the effective distribution of the conjugates in the physical location most suitable for their desired activity. In one embodiment, effective delivery occurs before clearance by the reticuloendothelial system or the production of off-target binding which can result in reduced efficacy or toxicity. Non-limiting examples of agents suitable for formulation with the conjugates include: P-glycoprotein inhibitors (such as Pluronic P85), which can enhance entry of active agents into the CNS; biodegradable polymers, such as poly (DL-lactide-coglycolide) microspheres for sustained release delivery after intracerebral implantation; and loaded nanoparticles, such as those made of polybutylcyanoacrylate, which can deliver active agents across the blood brain barrier and can alter neuronal uptake mechanisms.

Also included herein are pharmaceutical compositions prepared for storage or administration, which include a pharmaceutically effective amount of the desired conjugates in a pharmaceutically acceptable carrier or diluent. Acceptable carriers, diluents, and/or excipients for therapeutic use are well known in the pharmaceutical art. For example, buffers, preservatives, bulking agents, dispersants, stabilizers, dyes, can be provided. In addition, antioxidants and suspending agents can be used Examples of suitable carriers, diluents and/or excipients include, but are not limited to: (1) Dulbecco's phosphate buffered saline, pH about 6.5, which would contain about 1 mg/ml to 25 mg/ml human serum albumin, (2) 0.9% saline (0.9% w/v NaCl), and (3) 5% (w/v) dextrose.

The term “pharmaceutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Pharmaceutically effective amounts for a given situation can be determined by routine experimentation that is within the skill and judgment of the clinician. In a preferred aspect, the disease or condition to can be treated via gene silencing.

For any conjugate, the pharmaceutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and L^{D}₅₀ (the dose lethal to 50% of the population). The dose ratio between toxic and therapeutic effects is the therapeutic index, and it can be expressed as the ratio, L^{D}₅₀/ED₅₀. Pharmaceutical compositions that exhibit large therapeutic indices are preferred. The dosage may vary within this range depending upon the dosage form employed, sensitivity of the patient, and the route of administration.

In one embodiment, the conjugates are formulated for parenteral administration by injection including using conventional catheterization techniques or infusion. Formulations for injection may be presented in unit dosage form, e.g., in ampules or in multi-dose containers, with an added preservative. The conjugates can be administered parenterally in a sterile medium. The conjugate, depending on the vehicle and concentration used, can either be suspended or dissolved in the vehicle. Advantageously, adjuvants such as local anesthetics, preservatives, and buffering agents can be dissolved in the vehicle. The term “parenteral” as used herein includes percutaneous, subcutaneous, intravascular (e.g., intravenous), intramuscular, or intrathecal injection or infusion techniques and the like. In addition, there is provided a pharmaceutical formulation comprising conjugates and a pharmaceutically acceptable carrier. One or more of the conjugates can be present in association with one or more non-toxic pharmaceutically acceptable carriers and/or diluents and/or adjuvants, and if desired other active ingredients.

The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose, a bland fixed oil can be employed including synthetic mono- or diglycerides. In addition, fatty acids such as oleic acid find use in the preparation of injectables.

The conjugates and compositions described herein may be administered in appropriate form, preferably parenterally, more preferably intravenously. For parenteral administration, the conjugates or compositions can be aqueous or nonaqueous sterile solutions, suspensions or emulsions. Propylene glycol, vegetable oils and injectable organic esters, such as ethyl oleate, can be used as the solvent or vehicle. The compositions can also contain adjuvants, emulsifiers or dispersants.

Dosage levels of the order of from between about 0.01 mg and about 140 mg per kilogram of body weight per day are useful in the treatment of the above-indicated conditions (between about 0.05 mg and about 7 g per subject per day). In some embodiments, the dosage administered to a patient is between about 0.01 mg/kg to about 100 mg/kg of the subject's body weight. In some embodiments, the dosage administered to a patient is between about 0.01 mg/kg to about 15 mg/kg of the subject's body weight. In some embodiments, the dosage administered to a patient is between about 0.1 mg/kg and about 15 mg/kg of the subject's body weight. In some embodiments, the dosage administered to a patient is between about 0.1 mg/kg and about 20 mg/kg of the subject's body weight. In some embodiments, the dosage administered is between about 0.1 mg/kg to about 5 mg/kg or about 0.1 mg/kg to about 10 mg/kg of the subject's body weight. In some embodiments, the dosage administered is between about 1 mg/kg to about 15 mg/kg of the subject's body weight. In some embodiments, the dosage administered is between about 1 mg/kg to about 10 mg/kg of the subject's body weight. The amount of conjugate that can be combined with the carrier materials to produce a single dosage form varies depending upon the host treated and the particular mode of administration. Dosage unit forms can generally contain from between about 0.01 mg and about 100 mg; between about 0.01 mg and about 75 mg; or between about 0.01 mg and about 50 mg; or between about 0.01 mg and about 25 mg; of a conjugate.

For intravenous administration, the dosage levels can comprise from about 0.01 to about 200 mg of a conjugate per kg of the animal's body weight. In one aspect, the composition can include from about 1 to about 100 mg of a conjugate per kg of the animal's body weight. In another aspect, the amount administered will be in the range from about 0.1 to about 25 mg/kg of body weight of a compound.

In some embodiments, the conjugates can be administered are as follows. The conjugates can be given daily for about 5 days either as an i.v., bolus each day for about 5 days, or as a continuous infusion for about 5 days.

Alternatively, the conjugates can be administered once a week for six weeks or longer. As another alternative, the conjugates can be administered once every two or three weeks. Bolus doses are given in about 50 to about 400 ml of normal saline to which about 5 to about 10 ml of human serum albumin can be added. Continuous infusions are given in about 250 to about 500 ml of normal saline, to which about 25 to about 50 ml of human serum albumin can be added, per 24 hour period.

In some embodiments about one to about four weeks after treatment, the patient can receive a second course of treatment. Specific clinical protocols with regard to route of administration, excipients, diluents, dosages, and times can be determined by the skilled artisan as the clinical situation warrants.

It is understood that the specific dose level for a particular subject depends upon a variety of factors including the activity of the specific conjugate, the age, body weight, general health, sex, diet, time of administration, route of administration, and rate of excretion, combination with other active agents, and the severity of the particular disease undergoing therapy.

For administration to non-human animals, the conjugates can also be added to the animal feed or drinking water. It can be convenient to formulate the animal feed and drinking water so that the animal takes in a therapeutically appropriate quantity of the conjugates along with its diet. It can also be convenient to present the conjugates as a premix for addition to the feed or drinking water.

The conjugates can also be administered to a subject in combination with other therapeutic compounds to increase the overall therapeutic effect. The use of multiple compounds to treat an indication can increase the beneficial effects while reducing the presence of side effects. In some embodiment the conjugates are used in combination with chemotherapeutic agents, such as those disclosed in U.S. Pat. No. 7,303,749. In other embodiments the chemotherapeutic agents, include, but are not limited to letrozole, oxaliplatin, docetaxel, 5-FU, lapatinib, capecitabine, leucovorin, erlotinib, pertuzumab, bevacizumab, and gemcitabine.

The present invention also provides pharmaceutical kits comprising one or more containers filled with one or more of the conjugates and/or compositions of the present invention, including, one or more chemotherapeutic agents. Such kits can also include, for example, other compounds and/or compositions, a device(s) for administering the compounds and/or compositions, and written instructions in a form prescribed by a governmental agency regulating the manufacture, use or sale of pharmaceuticals or biological products.

### Methods of Use

### Methods of Treating

In certain preferred embodiments of the invention, the terminal conjugate of the invention are used in methods of treating animals (preferably mammals, most preferably humans and includes males, females, infants, children and adults). In one embodiment, the conjugates of the present invention may be used in a method of treating animals which comprises administering to the animal a biodegradable biocompatible conjugate of the invention. For example, conjugates in accordance with the invention can be administered in the form of soluble linear polymers, copolymers, conjugates, colloids, particles, gels, solid items, fibers, films, etc. Biodegradable biocompatible conjugates of this invention can be used as drug carriers and drug carrier components, in systems of controlled drug release, preparations for low-invasive surgical procedures, etc. Pharmaceutical formulations can be injectable, implantable, etc.

In yet another aspect, the invention provides a method of treating a disease or disorder in a subject in need thereof, comprising administering to the subject an efficient amount of at least one conjugate of the invention; wherein said conjugate releases one or more therapeutic agents upon biodegradation.

In another embodiment the conjugates can be administered in vitro, in vivo and/or ex vivo to treat patients and/or to modulate the growth of selected cell populations including, for example, cancer. In some embodiments, the particular types of cancers that can be treated with the conjugates include, but are not limited to: (1) solid tumors, including but not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophogeal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma, multiforme astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma; (2) blood-borne cancers, including but not limited to acute lymphoblastic leukemia “ALL”, acute lymphoblastic B-cell leukemia, acute lymphoblastic T-cell leukemia, acute myeloblastic leukemia “AML”, acute promyelocytic leukemia “APL”, acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia “CML”, chronic lymphocytic leukemia “CLL”, hairy cell leukemia, multiple myeloma, acute and chronic leukemias, e.g., lymphoblastic myelogenous and lymphocytic myelocytic leukemias; and (3) lymphomas such as Hodgkin's disease, non-Hodgkin's Lymphoma, Multiple myeloma, Waldenstrom's macroglobulinemia, Heavy chain disease, and Polycythemia vera.

In another embodiment the conjugates can be administered in vitro, in vivo and/or ex vivo to treat autoimmune diseases, such as systemic lupus, rheumatoid arthritis, and multiple sclerosis; graft rejections, such as renal transplant rejection, liver transplant rejection, lung transplant rejection, cardiac transplant rejection, and bone marrow transplant rejection; graft versus host disease; viral infections, such as CMV infection, HIV infection, and AIDS; and parasite infections, such as giardiasis, amoebiasis, schistosomiasis, and the like.

In certain embodiments the conjugates can also be used for the manufacture of a medicament useful for treating or lessening the severity of disorders, such as, characterized by abnormal growth of cells (e.g., cancer).

In certain embodiments, the therapeutic agent is locally delivered to a specific target cell, tissue, or organ.

In certain embodiments, in practicing the method of the invention, the conjugate further comprises or is associated with a diagnostic label. In certain exemplary embodiments, the diagnostic label is selected from the group consisting of: radiopharmaceutical or radioactive isotopes for gamma scintigraphy and PET, contrast agent for Magnetic Resonance Imaging (MRI), contrast agent for computed tomography, contrast agent for X-ray imaging method, agent for ultrasound diagnostic method, agent for neutron activation, moiety which can reflect, scatter or affect X-rays, ultrasounds, radiowaves and microwaves and fluorophores. In certain exemplary embodiments, the conjugate is further monitored in vivo.

Examples of diagnostic labels include, but are not limited to, diagnostic radiopharmaceutical or radioactive isotopes for gamma scintigraphy and PET, contrast agent for Magnetic Resonance Imaging (MRI) (for example paramagnetic atoms and superparamagnetic nanocrystals), contrast agent for computed tomography, contrast agent for X-ray imaging method, agent for ultrasound diagnostic method, agent for neutron activation, and moiety which can reflect, scatter or affect X-rays, ultrasounds, radiowaves and microwaves, fluorophores in various optical procedures, etc. Diagnostic radiopharmaceuticals include γ-emitting radionuclides, e.g., indium-111, technetium-99m and iodine-131, etc. Contrast agents for MRI (Magnetic Resonance Imaging) include magnetic compounds, e.g. paramagnetic ions, iron, manganese, gadolinium, lanthanides, organic paramagnetic moieties and superparamagnetic, ferromagnetic and antiferromagnetic compounds, e.g., iron oxide colloids, ferrite colloids, etc. Contrast agents for computed tomography and other X-ray based imaging methods include compounds absorbing X-rays, e.g., iodine, barium, etc. Contrast agents for ultrasound based methods include compounds which can absorb, reflect and scatter ultrasound waves, e.g., emulsions, crystals, gas bubbles, etc. Still other examples include substances useful for neutron activation, such as boron and gadolinium. Further, labels can be employed which can reflect, refract, scatter, or otherwise affect X-rays, ultrasound, radiowaves, microwaves and other rays useful in diagnostic procedures. Fluorescent labels can be used for photoimaging. In certain embodiments a modifier comprises a paramagnetic ion or group.

In another aspect, the invention provides a method of treating a disease or disorder in a subject, comprising preparing an aqueous formulation of at least one conjugate of the invention and parenterally injecting said formulation in the subject.

In another aspect, the invention provides a method of treating a disease or disorder in a subject, comprising preparing an implant comprising at least one conjugate of the invention, and implanting said implant into the subject. In certain exemplary embodiments, the implant is a biodegradable gel matrix.

In another aspect, the invention provides a method for treating of an animal in need thereof, comprising administering a conjugate according to the methods described above.

In another aspect, the invention provides a method for eliciting an immune response in an animal, comprising administering a conjugate as in the methods described above.

In another aspect, the invention provides a method of diagnosing a disease in an animal, comprising steps of:
administering a conjugate as in the methods described above, wherein said conjugate comprises a detectable molecule; and
detecting the detectable molecule.

In certain exemplary embodiments, the step of detecting the detectable molecule is performed non-invasively. In certain exemplary embodiments, the step of detecting the detectable molecule is performed using suitable imaging equipment.

In one embodiment, a method for treating an animal comprises administering to the animal a biodegradable biocompatible conjugate of the invention as a packing for a surgical wound from which a tumor or growth has been removed. The biodegradable biocompatible conjugate packing will replace the tumor site during recovery and degrade and dissipate as the wound heals.

In certain embodiments, the conjugate is associated with a diagnostic label for in vivo monitoring.

The terminal conjugates described above can be used for therapeutic, preventative, and analytical (diagnostic) treatment of animals. The conjugates are intended, generally, for parenteral administration, but in some cases may be administered by other routes.

In one embodiment, soluble or colloidal conjugates are administered intravenously. In another embodiment, soluble or colloidal conjugates are administered via local (e.g., subcutaneous, intramuscular) injection. In another embodiment, solid conjugates (e.g., particles, implants, drug delivery systems) are administered via implantation or injection.

In another embodiment, the terminal conjugates comprising a detectable label are administered to study the patterns and dynamics of label distribution in animal body.

In another embodiment, conjugates comprising an antigen or an antigen-generating component (e.g., a plasmid) are administered to develop immunity to said antigen.

In certain embodiments, any one or more of the conjugates disclosed herein may be used in practicing any of the methods described above. In certain exemplary embodiments, the conjugate is a Trastuzumab-PHF-, Rituximab-PHF- or LHRH-PHF-drug conjugate.

Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

The synthetic processes of the invention can tolerate a wide variety of functional groups; therefore various substituted starting materials can be used. The processes generally provide the desired final compound at or near the end of the overall process, although it may be desirable in certain instances to further convert the compound to a pharmaceutically acceptable salt, ester or prodrug thereof.

Drug compounds used for the conjugates of the present invention can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; and Greene, T. W., Wuts, P. G. M., Protective Groups in Organic Synthesis, 3^{rd} edition, John Wiley & Sons: New York, 1999, incorporated by reference herein, are useful and recognized reference textbooks of organic synthesis known to those in the art. The following descriptions of synthetic methods are designed to illustrate, but not to limit, general procedures for the preparation of compounds of the present invention.

Conjugates of the present invention and the drug compounds included therein can be conveniently prepared by a variety of methods familiar to those skilled in the art. The conjugates or compounds of this invention with each of the formulae described herein may be prepared according to the following procedures from commercially available starting materials or starting materials which can be prepared using literature procedures. These procedures show the preparation of representative conjugates of this invention.

Conjugates designed, selected and/or optimized by methods described above, once produced, can be characterized using a variety of assays known to those skilled in the art to determine whether the conjugates have biological activity. For example, the conjugates can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the conjugate molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

The terminally modified polymers and conjugates described herein can be prepared by the schemes generally outlined above and by methods described in the Examples below. The term “content” as used in certain examples below, unless otherwise specified, means the molar fraction of the polymer units that are substituted with the intended moiety, such as the linker, the drug molecule, or PBRM.

### ABBREVIATIONS

The following abbreviations are used in the reaction schemes and synthetic examples, which follow. This list is not meant to be an all-inclusive list of abbreviations used in the application as additional standard abbreviations, which are readily understood by those skilled in the art of organic synthesis, can also be used in the synthetic schemes and examples.
BA β-alanine
DMAc Dimethylacetamide
EDC.HCl 1-Ethyl-3-(3-dimethyllaminopropyl)carbodiimide hydrochloride
FMoc propane diamine (9H-fluoren-9-yl)methyl 3-aminopropylcarbamate
FMoc-Ser-amide (R)-(9H-fluoren-9-yl)methyl 1-amino-3-hydroxy-1-oxopropan-2-ylcarbamate
FMoc-Ser-OH (R)-2-(((9H-fluoren-9-yl)methoxy)carbonylamino)-3-hydroxypropanoic acid
GA Glutaric acid
HATU 2-(1H-7-Azabenzotriazol-1-yl)-1,1,3,3-tetramethyluronoium hexafluorphosphate
HPLC High-performance liquid chromatography
MWCO Molecular weight cut off
NHS 1-hydroxypyrrolidone-2, 5-dione (N-hydroxysuccinimide)
NMP N-methylpyrrolidinone
NH₄OAc Ammonium acetate
PBS Phosphate buffered saline, 0.9% NaCl
PHF poly(1-hydroxymethylethylene hydroxylmethylformal), or FLEXIMER®
SEC-HPLC Size-exclusion HPLC
SEC Size exclusion chromatography
SPDP Succinimidyl 3-(2-pyridyldithio)propionate
S SPy 2-(pyridine-2-yldisulfanyl)
TEAA Triethylammonium acetate
TCEP Tris[2-carboxyethyl] phosphine
CEX Cation exchange

### General Information

PHF of varying molecular weight (6-120 kDa) have been utilized. The terminal modification of PHF was characterized and quantitative analyzed via ¹H-NMR and/or SEC-UV HPLC.

Size exclusion chromatography (SEC) was performed on a Tosoh Biosciences TSKgelG5000 column (7.8 mm×30 cm, 10 um) or Superose 12 (GE Healthcare).

Exendin-4 containing a C-terminal cysteine residue (Exendin-4-Cys) was purchased from Advanced ChemTech.

Anti-Her2 affibody, 14 K, was purchased from Affibody AB.

S-Acetylthioglycolic acid NHS ester and hydroxylamine hydrochloride were purchased from Sigma-Aldrich.

### Reduction of PHF

PHF was reduced with sodium borohydride in order to remove any partially reduced cyclic hemiacetals.

### Oxidation of PHF

PHF was oxidized with sodium periodate, NaIO₄ using different mole ratios and time to determine suitable reaction condition. The products were analyzed by ¹H-NMR and SEC-HPLC. In the proton NMR the new signals at ˜8.2 ppm were assigned to the CHO functionality in the oxidized PHF product (PHF-Aldehyde, PHF-acetaldehyde). These aldehyde protons appeared as a pool of 3-4 singlets as oppose to a single peak due to the fact that the CHO on the polymer can be derived from branching on the polymer originating from Dextran.

### Molecular Weight Analysis by SEC-HPLC

SEC-HPLC analysis was conducted on the periodate-treated PHF samples to determine the average molecular weight with the goal to develop oxidation conditions that result in PHF with minimum or no degradation of the PHF polymer backbone. But it is also important to understand that each PHF polymer backbone cleavage event will generate a site for terminal modification. Several oxidation experiments with varying amounts NalO₄ were performed and it was found that as little as 0.075 eq. of NalO₄ was sufficient to achieve the desired oxidation.

Reductive amination was conducted using aqueous conditions on a 1-3 g scale with benzyl amine, mono-FMoc propane diamine.HCl, maleimidoethylamine.HCl and NH₄OAc as amine containing compounds. The products were purified by diafiltration using 5 kDa MWCO cassette with typically >85% recovery. The ¹H-NMRs showed the disappearance of the PHF-aldehyde signal at 8.2 ppm as the result of the reductive amination. The SEC-HPLC analysis of the product showed no change in polymer molecular weight distribution.

### Example 1: PHF Oxidation

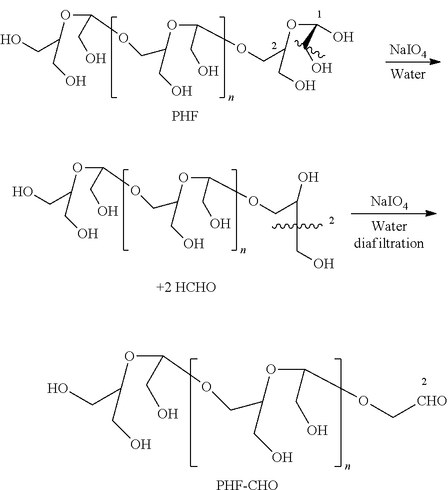

### Procedure A

To a solution of 50 kDa PHF (10.0 g, 74.6 mmol) in water (100 g) at 5-10° C. was added an aqueous solution of sodium (meta) per iodate (NaIO4, 1.28 g, 6.0 mmol) in water (10 g) over a period of 15 minutes and the resulting mixture was then stirred for 16 hours at room temperature. The reaction mixture was cooled to 5-10° C. and pH was adjusted to 6.5. The solution was purified by diafiltration using a 5 kDa MWCO Biomax membrane filter, followed by lyophilization to give the desired product; 8.9 g, 89% yield. ¹H-NMR showed the appearance of signals at ˜8.2 ppm.

### Procedure B

To a solution of 50 kDa PHF (15.0 g, 112 mmol) in water (150 g) at 5-10° C. was added an aqueous solution of sodium (meta) per iodate (NaIO4, 2.0 g, 9.4 mmol) in water (20 g) over a period of 15 minutes and the resulting mixture was then stirred for 16 hours at 5-15° C. The pH was adjusted to 6.5. The resulting solution was purified by SEC followed by diafiltration using a 5 kDa MWCO Biomax membrane filter, and lyophilized to give the title compound; 12 g, 80% yield. ¹H-NMR showed the appearance of signals at ˜8.4 ppm.

### Example 2: Reductive Amination—General Procedure

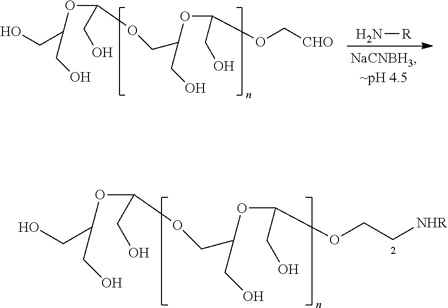

To an aqueous solution of PHF-aldehyde (2-10%, prepared as described in Example 1) was added an amine compound (6-16% w/w based on PHF-aldehyde) dissolved in aqueous ethanol (10%). The mixture was stirred for approximately 1 hour followed by the addition of sodium cyanoborohydride (1.5-3.0 mol eq, based on amine). The pH of the reaction mixture was adjusted to 4.5-5.0 using acetic acid (10% aqueous). The reaction mixture was stirred at 35-40° C. for 20-68 hours. The product was purified by diafiltration using a 5 kDa or 10 kDa MWCO membrane filter. The purified product was lyophilized to give the desired product.

### Example 3: Synthesis of PHF-benzylamine (i.e., (N-benzyl)ethylamino-PHF) (1)

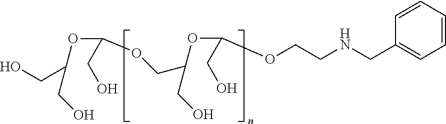

PHF-aldehyde (426 mg, 3.18 mmol, prepared as described in Example 1) in water (20 g) was reacted with benzylamine (110 mg, 1.03 mmol) and sodium cyanoborohydride (0.07 g, 1.11 mmol) dissolved in ethanol (4 g) for 16 hours using the procedure described in Example 2. After purification by diafiltration using 5 kDa MWCO membrane filter, the product was lyophilized to give the desired product as a white solid; 389 mg, 90% yield. ¹H-NMR indicated the disappearance of polymeric aldehyde signals at 8.2 ppm and appearance of new aromatic signals at 7.5 ppm.

### Example 4: Synthesis of PHF-3-FMoc propane diamine (i.e., (N-(3-FMoc-amino)propyl)ethylamino-PHF) (2)

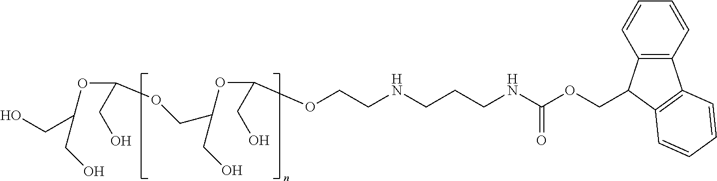

PHF-aldehyde (1.69 g, 12.6 mmol, prepared as described in Example 1) in water (65 g) was reacted for 30 minutes with 3-FMoc-1,3-propane diamine.HCl (0.1 g, 0.30 mmol) and sodium cyanoborohydride (0.1 g, 1.59 mmol) dissolved in ethanol (4 g) using the procedure described in Example 2. The pH was adjusted to 5.0 using acetic acid (10% aqueous) and the reaction mixture was stirred for an additional 67 hours at ambient temperature. After purification by diafiltration using 5 kDa MWCO membrane filter, the product was lyophilized to give the desired product as a white solid; 1.2 g, 71% yield. ¹H-NMR indicated the disappearance of polymeric aldehyde signals at 8.2 ppm and appearance of new FMoc aromatic signals at 7.3-8.0 ppm.

### Example 5: Synthesis of PHF-2-Maleimido ethylamine (i.e., (N-(2-maleimido)ethyl)ethylamino-PHF) (3)

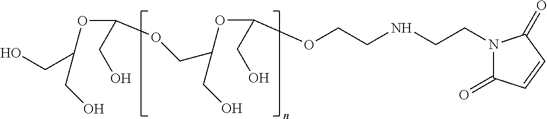

To an aqueous solution of PHF-aldehyde (30 mL at 10.4%, 3.12 g, 23.3 mmol, prepared as described in Example 1) at 5-10° C. was added 1-(2-aminoethyl)-1H-pyrrole-2,5-dione. HCl (0.25 g, 1.42 mmol) and stirred for 20 minutes, pH 4.0. Sodium cyanoborohydride (0.25 g, 3.98 mmol) was added portion wise over 5 minutes and the resulting mixture stirred at ambient temperature for ˜1 hour. The pH of the reaction mixture was adjusted to 4.5 with acetic acid (10% aqueous) and the stirring continued for 64 hours. After purification by diafiltration using 5 kDa MWCO membrane filter, the product was lyophilized to give a white solid; 2.18 g, 72% yield. ¹H-NMR indicated the disappearance of polymeric aldehyde signals at 8.2 ppm and appearance of maleimide related signals at 6.0-7.2 ppm.

The product was further purified using a 10 kDa MWCO membrane filter in order to obtain higher molecular weight product. After lyophilization, the product was obtained as a white solid; 0.8 g, 38% yield. Coupling of the final product with a peptide was unsuccessful.

### Example 6: Synthesis of PHF-Amine (i.e., Ethylamino-PHF) (4)

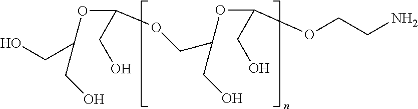

### Procedure A

PHF-aldehyde (PHF-acetaldehyde, 2.0 g, 14.9 mmol, prepared as described in Example 1) in water (46 g) was reacted for 67 hours with 35% aqueous solution of ammonium hydroxide (0.25 g, 2.50 mmol) and sodium cyanoborohydride (0.25 g, 3.98 mmol) using the procedure described in Example 2. The pH of the mixture was adjusted to 6.0 from pH 10.5 using acetic acid (10% aqueous). After purification by diafiltration using 10 kDa MWCO membrane filter, the product was lyophilized to give the desired product as a white solid; 1.57 g, 79% yield. ¹H-NMR indicated the consumption of polymeric aldehyde signals at 8.2 ppm.

### Procedure B

PHF-aldehyde (4.2 g, 31.3 mmol, prepared as described in Example 1) in water (84 g) was reacted with ammonium acetate (10 g, 130 mmol), 30% aqueous ammonium hydroxide solution (6.0 g, 111 mmol) and sodium cyanoborohydride (0.525 g, 8.36 mmol) for 24 hours at 40-45° C. The reaction solution was then cooled to ambient temperature, pH was adjusted to 6.5, then purified by SEC and diafiltration using 5 kDa MWCO membrane filter. The resulting product was lyophilized to give the title compound as a white solid; 3.0 g, 71% yield. ¹H-NMR indicated the absence of polymeric aldehyde signals at 8.2 ppm.

### Example 7: Synthesis of PHF-FMoc Ser-Amide (i.e., N-(FMoc-L-serineamido)ethylamino-PHF) (5)

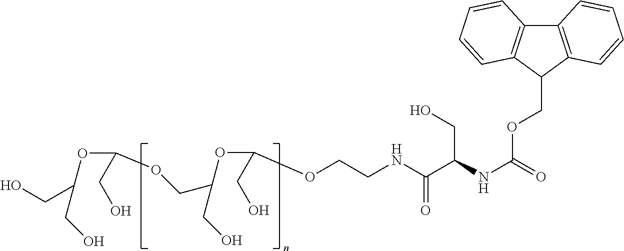

To an aqueous solution of PHF-amine (ethylamino-PHF) (36 g at 2.05%, 0.74 g, 5.52 mmol, prepared as described in Example 6) was added FMoc-Ser-OH (0.078 g, 0.238 mmol ) followed by N-hydroxy succinimide (0.07 g, 0.61 mmol). The pH of the mixture was adjusted to 4.6 using acetic acid (10% aqueous). To this mixture was added EDC.HCl (0.175 g, 0.91 mmol) in four equal portions over a period of 10 minutes. The reaction mixture was stirred at room temperature for ˜16 hours. The resulting cloudy solution was filtered through a 0.2 i filter. After purification by diafiltration using 10 kDa MWCO membrane filter, the product was lyophilized to give the desired product as a white solid; 0.7 g, 95% yield. ¹H-NMR indicated the disappearance of polymeric aldehyde signals at 8.2 ppm and appearance of new signals between 7.3-8.0 ppm assigned to FMoc.

### Example 8: PHF Reduction

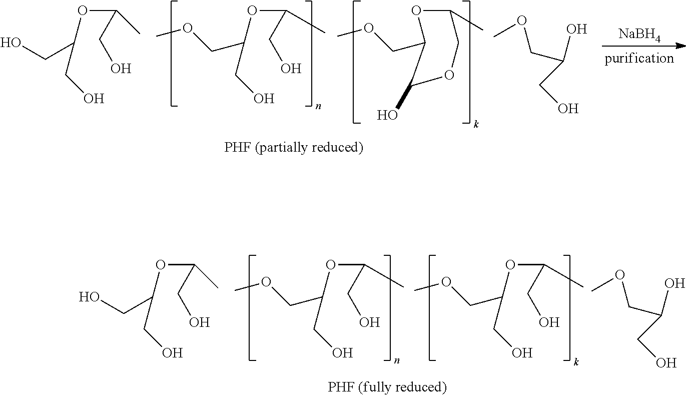

To a solution of PHF (180 kDa, 0.5 g, 3.73 mmol) in water (10 mL) at 5-10° C. was added sodium borohydride (28 mg, 0.746 mmol) in portions under an inert atmosphere. The mixture was stirred at room temperature for 4 hours, then cooled to 5-10° C. and pH was adjusted to ˜6.5 using aqueous acetic acid (20%). The product was purified by diafiltration using a 10 kDa MWCO membrane filter followed by lyophilized to obtain the title compound as white foam; 0.47 g, 93% yield. ¹H-NMR showed no signals at 8.5 ppm; MW 120 kDa.

### Example 9: PHF Oxidation

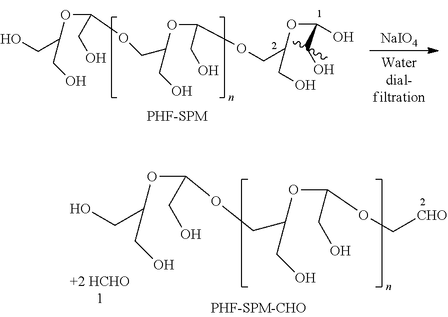

To a solution of 120 kDa PHF (prepared as described in Example 8, 0.45 g, 3.36 mmol.) in water (10 g) at 2-8° C. was added an aqueous solution of sodium (meta) periodate (NaIO4, 58 mg, 0.27 mmol) in water (1 mL) over a period of 2 minutes and then stirred for 2.5 hours at room temperature. The reaction mixture was cooled to 5-10° C. and pH was adjusted to 6. The product was purified by diafiltration using a 10 kDa MWCO membrane filter followed by lyophilization to give the title compound; 420 mg, 93% yield. ¹H-NMR showed the appearance of aldehyde related signals (singlet at ˜8.5 ppm); MW108 kDa.

### Example 10: Reductive Amination

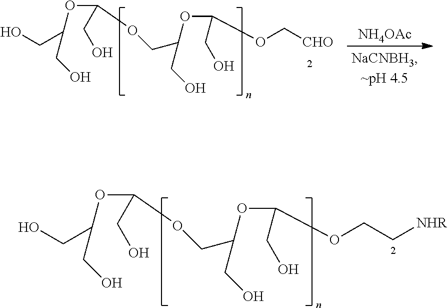

To a solution of PHF-Aldehyde (prepared as described in Example 9, 0.35 g, 2.61 mmol) dissolved in water (15 mL) was added sodium acetate (0.1 g) followed by 30% ammonium hydroxide (0.11 g) and sodium cyanoborohydride (37 mg). The mixture was stirred at 35-40° C. for 24 hours, cooled to room temperature, purified by diafiltration using a 10 kDa MWCO membrane filter, followed by lyophilization to give the title compound; 320 mg, yield 91%. ¹H-NMR showed absence of aldehyde related signals at 8.5 ppm; MW 100 kDa.

### Example 11: Coupling with 6-Maleimido Hexanoic Acid

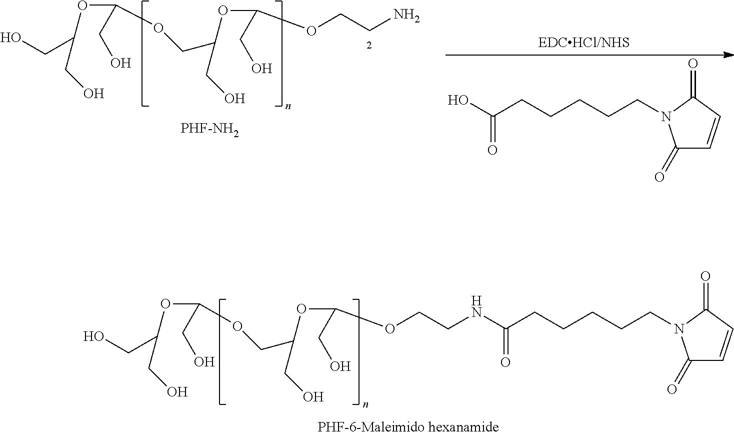

To an aqueous solution of PHF-amine (ethylamino-PHF) (prepared as described in Example 10. 0.24 g, 1.79 mmol) in water (10 mL) was added 6-maleimido hexanoic acid (23 mg, 0.109 mmol) followed by N-hydroxy succinimide (13 mg, 0.113 mmol.) and acetonitrile (0.3 mL). To this mixture was added EDC.HCl (42 mg, 0.22 mmol) in two portions over a period of 20 minutes. The reaction mixture was stirred at room temperature for ˜16 hours. The resulting hazy solution was filtered through a 0.2 micron filter, purified by diafiltration using a 10 kDa MWCO membrane filter, then lyophilized to give the an off-white solid; 225 mg, yield 8.7%. ¹H-NMR (D₂O) indicated the presence of maleimido functionality; MW 78 kDa.

### Example 12: PHF-Beta Alanine (OMe)-3-FMoc Propane Diamine (i.e., N-(3-FMoc-amino)propyl) (N—(N-carboxyl) β-alanine methoxide)ethylamino-PHF-methoxy-BA)

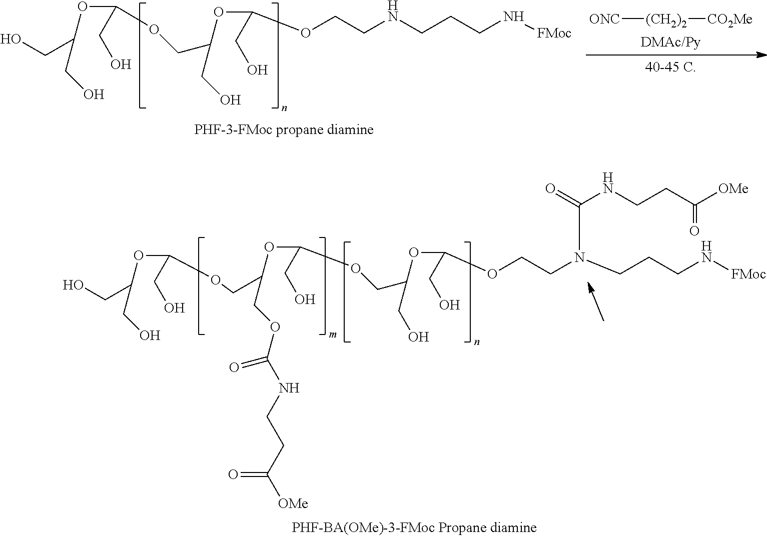

To PHF-FMoc propane amine ((N-(3-FMoc-amino)propyl)ethylamino-PHF) (prepared as described in Example 4, 385 mg, 2.9 mmol, 1.0 eq.), was added 3.85 g of dimethylacetamide (10.0 w/w volumes) and pyridine (0.4 g). The reaction mixture was stirred at 40-45° C. for 2 hours until a clear solution resulted. Methyl-3-isocyanatopropanoate (0.080 g, 0.22 mole % to PHF) was added over 5 minutes and the stirring continued for an additional 18 h. The reaction mixture was evaporated to dryness, co-evaporated with water (2×20 mL), re-dissolved in water and filtered through a 0.2 micron filter, then lyophilized to obtain the product as a white foam; 285 mg, yield 74%. ¹H-NMR indicated the presence new signals corresponding to beta-alanine methyl ester (multiplets, 2.7 and 4.0-3.5 ppm) and FMoc signals in the aromatic region. β-alanine loading was 10% by ¹H-NMR. MW 10 kDa.

### Example 13: PHF-BA-propane diamine (i.e., N-(3-amino)propyl-(N—(N-carboxyl) 3-alanine)ethylamino-PHF-BA)

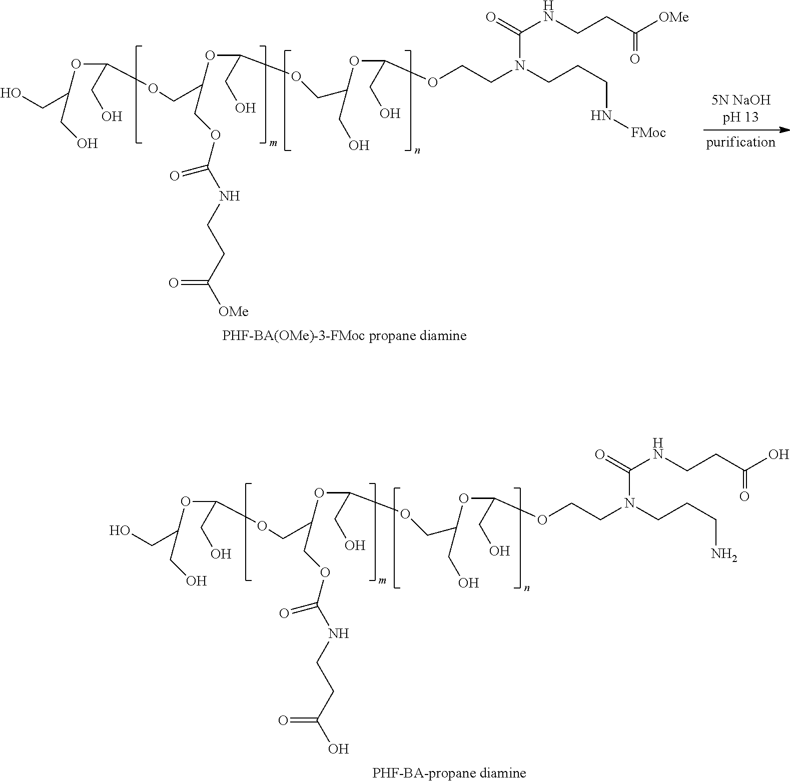

To the product of Example 12 (0.2 g, 1.5 mmol) dissolved in water (4 mL) was added 5N NaOH (0.4 g), final pH 13. The mixture was stirred at room temperature for 24 hours. The pH was adjusted to 6.5, diluted to 15 mL, filtered through a 0.2-micron filter. Purified by diafiltration using a 3K MWCO stir cell followed by lyophilization give an off-white solid; 121 mg; yield 61%. ¹H-NMR indicated the absence methyl ester signals. β-alanine loading was 10% by ¹H-NMR; MW 10 kDa.

### Example 14: PHF-BA-6-maleimido hexanamide (i.e., N-(3-(2-maleimido)propanamido)propyl)(N—(N-carboxyl) j-alanine)ethylamino-PHF-BA)

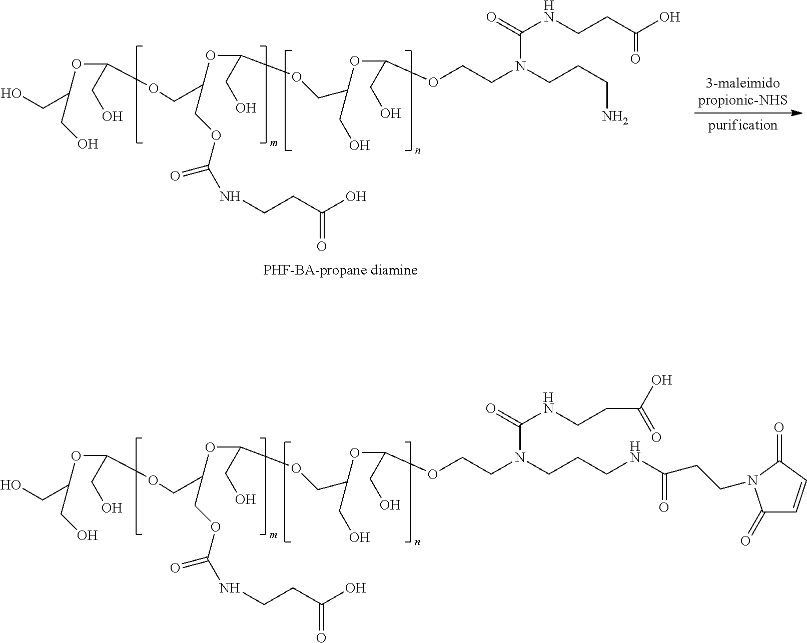

The pH of a solution of the product of Example 13 (0.10 g, 0.75 mmol) in water (4 mL) at 5-10° C. was adjust to pH 7-8, and 3-maleimidopropionic acid NHS ester (20 mg) suspended in acetonitrile (0.5 mL) was added. To the resulting mixture was added DMAc (0.1 mL) to improve solubility and then stirred for 3 hours. The clear resulting solution was filtered through 0.2μ filter and purified by diafiltration using a 3K MWCO membrane, followed by lyophilization to give an off-white solid product. 225 mg; yield 87%. ¹H-NMR (D₂O) indicated the presence of β-alanine and maleimido functionalities. MW ˜10 kDa.

### Example 15. 50 kDa PHF-6-maleimido hexanamide-Exendin-4-Cys (i.e., (Exendin-4-Cys)-(N-(2-maleimido)propanamido)propyl)-ethylamino-50 kDa PHF)

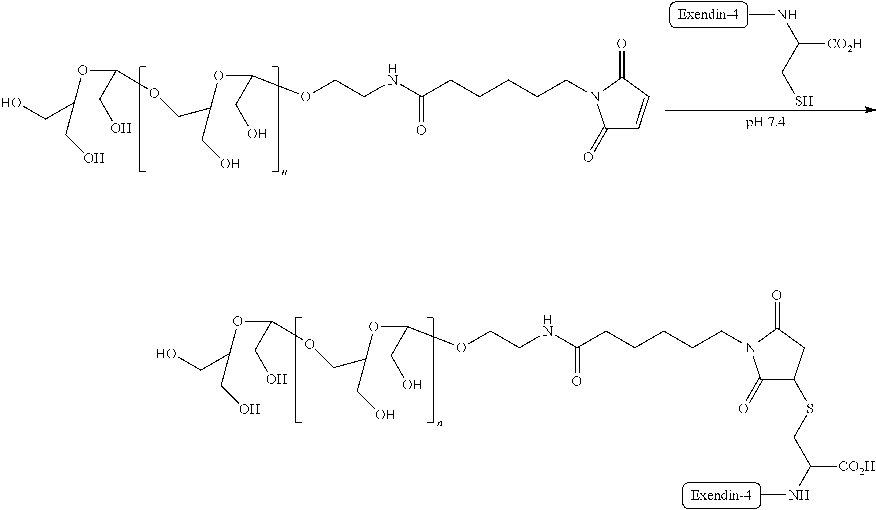

To a solution of 50 kDa PHF-6-maleimdio hexanoic acid ((N-(6-maleimido)hexanamido)ethylamino-50 kDa PHF) (0.73% maleimidio) (70 mg, 1.4 μmol, prepared as described in Example 11) in 50 mM PBS, 1 mM EDTA, pH 7.4 (2.56 mL), was added Exendin4-Cys (2.0 mg, 0.46 μmol) in 50 mM PBS (1 mL). The reaction mixture was stirred for 1 h at room temperature, followed by purification to give the title conjugate as a solution (600 μL) in NH₄OAc buffer, pH 6. The sample was free of unbound peptide as determined by SEC. Concentration of conjugated peptide: 2.36 mg/mL; 71% yield.

### Example 16: 15 kDa PHF-GA-6-maleimido hexanamide (i.e., (N-(6-maleimido)hexanamido)ethylamino-15 kDa PHF-GA)

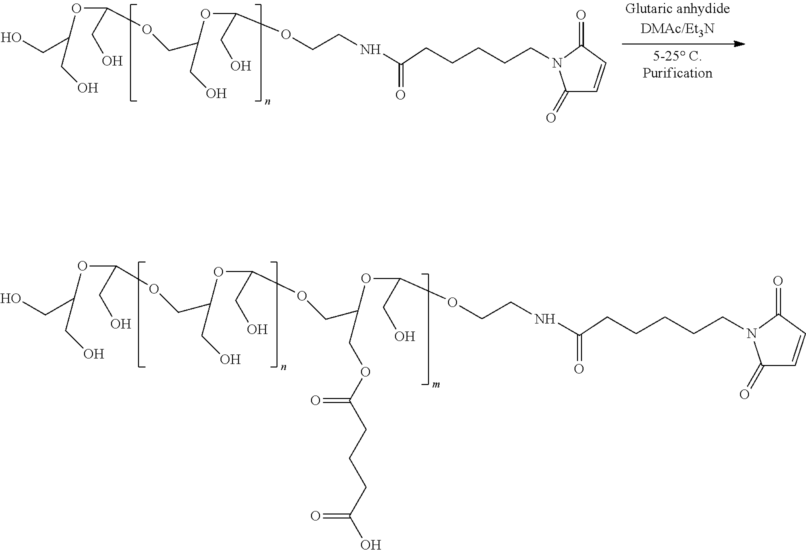

A solution of 15 kDa PHF-6-maleimido hexanamide ((N-(6-maleimido)hexanamido)ethylamino-15 kDa PHF) (150 mg, 1.071 mmol, prepared as described in Example 11 except 15 kDa PHF was used) in dimethylacetamide (1.9 g) was cooled to 5-10° C. To the resulting solution was added glutaric anhydride (40.3 mg, 0.354 mmol) followed by triethylamine (54.2 mg, 0.536 mmol) and then stirred at 10-25° C. for 18 hours. The reaction mixture was diluted with water to 10 mL and purified using a Sephadex G-25 column and 3 MWCO membrane filter and lyophilized to give the title conjugate; 175 mg, 94% yield. ¹H-NMR indicated the presence of glutaric acid ester (multiples for 2H each at 2.4 ppm, 2.3 ppm and 1.9 ppm, 30 mole %), maleimido peak (singlet at 6.8 ppm) and signals corresponding to the polymer backbone.

### Example 17: Synthesis of Auristatin F-hydroxypropylamide

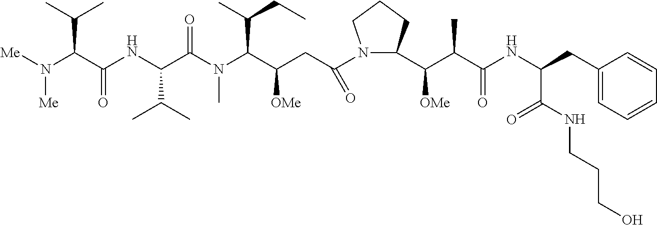 [SMILES: CC[C@H](C)[C@@H]([C@@H](CC(=O)N1CCC[C@H]1[C@H](OC)[C@@H](C)C(=O)N[C@@H](Cc1ccccc1)C(=O)NCCCO)OC)N(C)C(=O)[C@@H](NC(=O)[C@H](C(C)C)N(C)C)C(C)C]

Auristatin F (150 mg, 0.201 mmol), HATU (13.0 mg, 0.402 mmol), and diisopropylethylamine (108 μL, 0.603 mmol) were taken up in DMF (5 mL) and 3-aminopropan-1-ol (45.9 μL, 0.603 mmol) was added. The mixture was stirred at 23° C. for 45 minutes at which time LCMS analysis showed complete disappearance of the starting material. Reduction of the volume to 1.4 mL under high vacuum followed by purification via preparative HPLC (10-90 solvent B gradient over 20 minutes eluting with 0.1% TFA/Water, 0.1% TFA/CH₃CN) to give the title compound as white solid; 109 mg, 68% yield.

### Example 18: Synthesis of Auristatin F-hydroxypropylamide Boc-L-Alanine

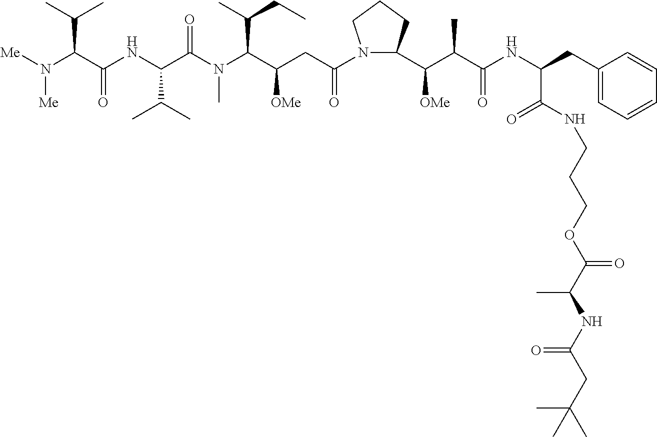 [SMILES: CC[C@H](C)[C@@H]([C@@H](CC(=O)N1CCC[C@H]1[C@H](OC)[C@@H](C)C(=O)N[C@@H](Cc1ccccc1)C(=O)NCCCOC(=O)[C@H](C)NC(=O)CC(C)(C)C)OC)N(C)C(=O)[C@@H](NC(=O)[C@H](C(C)C)N(C)C)C(C)C]

BOC-L-alanine (117.0 mg, 0.618 mmol) and DMAP (94.0 mg, 0.772 mmol) were taken up in dichloromethane and then diisopropylcarbodiimide (52.6 μL, 0.340 mmol) was added. The reaction mixture was cooled to 0° C. and stirred for 10 minutes after which auristatin F-hydroxypropylamide (124 mg, 0.154 mmol, prepared as described in Example 17) was added. The reaction mixture was warmed to 23° C. and stirred for 18 hours. Purification via preparative HPLC followed by removal of the water via lyophilization afforded the title compound as beige solid; 112 mg, 75% yield.

### Example 19: Synthesis of Auristatin F-hydroxypropylamide-L-Alanine

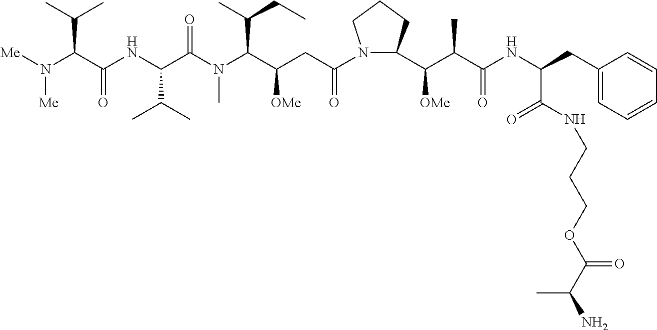 [SMILES: CC[C@H](C)[C@@H]([C@@H](CC(=O)N1CCC[C@H]1[C@H](OC)[C@@H](C)C(=O)N[C@@H](Cc1ccccc1)C(=O)NCCCOC(=O)[C@H](C)N)OC)N(C)C(=O)[C@@H](NC(=O)[C@H](C(C)C)N(C)C)C(C)C]

Auristatin F-hydroxypropylamide Boc-L-Alanine (112 mg, 0.115 mmol, prepared as described in Example 18) was taken up in dichloromethane (3 mL) and excess trifluoroacetic acid was added. The mixture was stirred at 23° C. for 1 hour and the solvent removed under high vacuum. The resulting oil was taken up in dichloromethane (1.5 mL) and precipitation from diethyl ether (30 mL to give the title compound as white solid (96.2 mg, 85%).

### Example 20: 15K PHF-GA-6-maleimido hexanamide-Auristatin F-hydroxypropylamide-L-Alanine (i.e., (N-(6-maleimido)hexanamido)ethylamino-15 kDa PHF-GA-Auristatin F-hydroxypropylamide-L-Alanine)

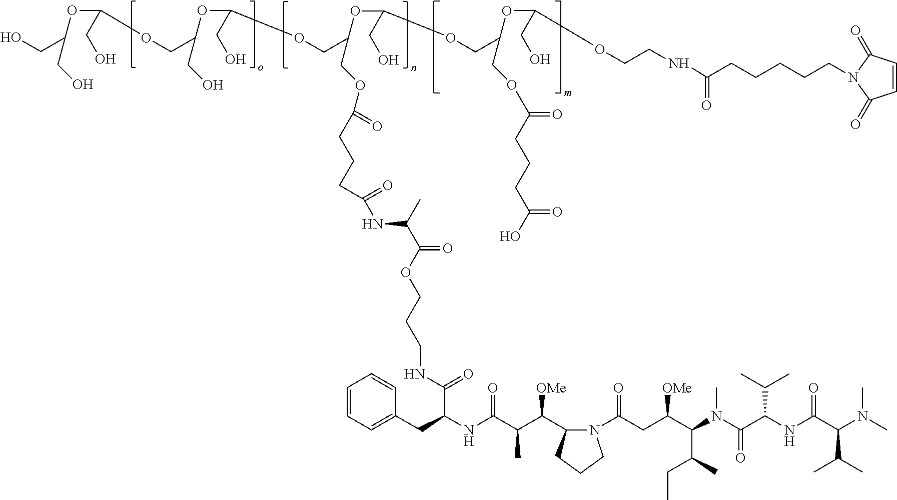

To a solution of 15K PHF-GA-6-maleimido hexanamide ((N-(6-maleimido)hexanamido)ethylamino-PHF-GA) (81 mg, 4.76 μmol, prepared as described in Example 16) and auristatin F-hydroxypropylamide-L-Alanine.TFA salt (16.90 mg, 14.29 μmol, prepared as described in Example 19) in NMP (0.7 g) and water (2.0 g) was added N-hydroxysuccinimide (NHS—OH, 4.93 mg, 42.9 μmol). The resulting solution was stirred until a clear solution was obtained and then cooled to 5-10° C. To this mixture was added EDC.HCl (14 mg) in two equal portions over a period of 30 minutes. The reaction mixture was stirred at room temperature for ˜16 hours, then diluted to ˜10 mL with water and purified by gel-filtration on Sephadex G-25 column and concentrated on a 3K MWCO membrane filter. The title compound was isolated as an aqueous solution; 8.72 mg/mL, 3.5 mL, 33% yield.

The polymer concentration was 97% mole as analyzed by SEC-HPLC; auristatin F-hydroxypropylamide content was 2.3% as analyzed by LCMS and maleimido concentration was 0.11 mole %.

### Example 21: 15 kDa PHF-GA-6-maleimido hexanamide-Auristatin F-hydroxypropylamide-L-Alanine-Trastuzumab (i.e., (Trastuzumab)(N-(6-maleimido)hexanamido)ethylamino 15 kDa PHF-GA-Auristatin F-hydroxypropylamide-L-Alanine)

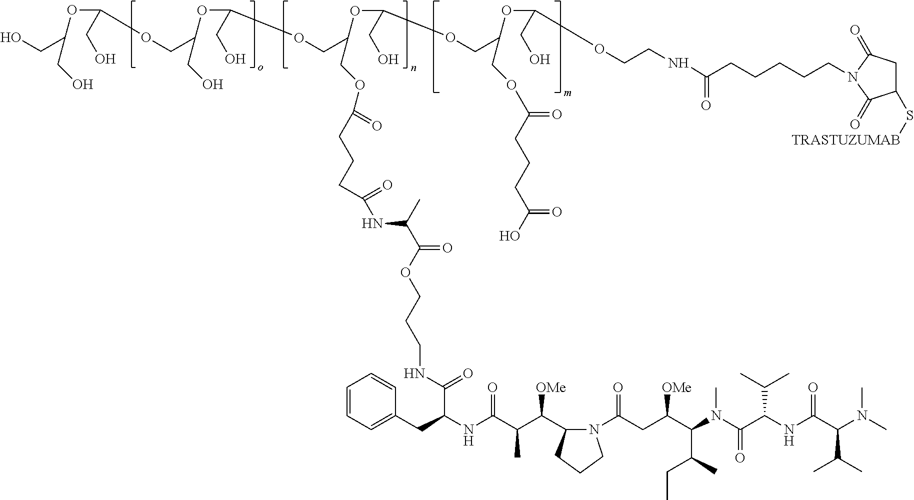

To a solution of Trastuzumab (309 μL, 5 mg, 0.034 μmol) in TEAA buffer, pH 7.4 was added a solution of TCEP (32 μL, 0.048 mg, 0.169 μmol) in TEAA buffer, pH 7.4 and the mixture was incubated for 1 h at 37° C. The reaction mixture was cooled to room temperature and a solution of 15 kDa PHF-GA (20%)-6-maleimido hexanamide (2.26%)-Auristatin F-hydroxypropylamide-L-Alanine (2.8%) (835 μL, 6.8 mg, 0.34 μmol, prepared as described in Example 20) in 50 mM PBS, pH 7.4 was added. After stirring ˜1 h at room temperature, the reaction mixture was concentrated by centrifugation on a 30 K MWCO membrane and purified by CEX chromatography to give the title compound. Yield 46% based on protein. AF-HPA to trastuzumab ratio was about 10:1 to about 14:1.

### Example 22: Synthesis of Trastuzumab-(Fab′)₂

Trastuzumab-(Fab′)₂ was prepared from immobilized pepsin (15 mL settled gel) and trastuzumab (440 mg, 2.4 μmol) according to the manufacturer's (Pierce) instructions to give the title compound; 265.2 mg, 100% yield.

### Example 23: 22 kDa PHF-GA-6-maleimido hexanamide-Auristatin F-hydroxypropylamide-L-Alanine Trastuzumab Fab′ (i.e., (Trastuzumab-Fab′)-(N-(6-maleimido)hexanamido)ethylamino 22 kDa PHF-GA-Auristatin F-hydroxypropylamide-L-Alanine)

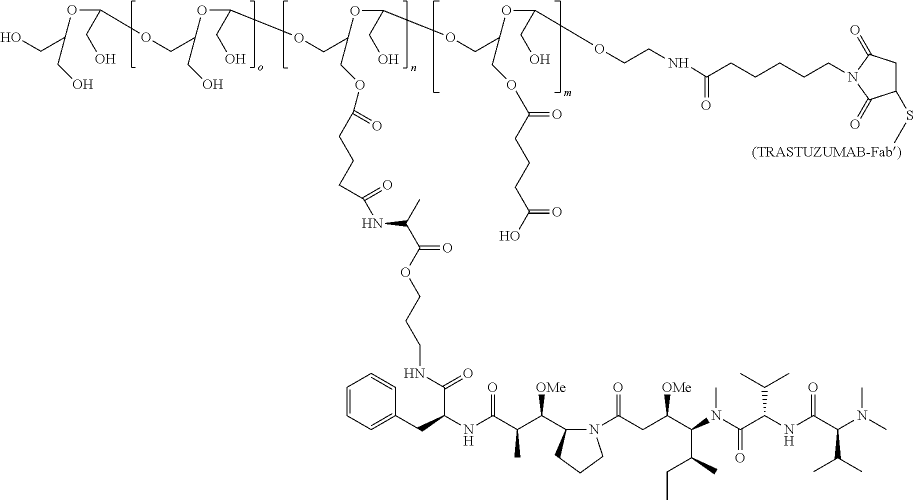

To a solution of Trastuzumab-(Fab′)₂ (158 μL, 2 mg, 0.04 μmol, prepared as described in Example 22) in TEAA buffer, pH 7.4 was added a solution of TCEP (22 μL, 0.033 mg, 0.115 μmol) in TEAA buffer, pH 7.4 and the mixture was incubated 1 h at 37° C. The reaction mixture was cooled to room temperature and a solution of 22 K PHF-GA (21%)-6-maleimido hexanamide (1.81%)-Auristatin F-hydroxypropylamide-L-Alanine (2.51%) ((N-(6-maleimido)hexanamido) (1.81%) ethylamino-22 kDa PHF-GA- (21%) Auristatin F-hydroxypropylamide-L-Alanine (2.51%)) (881 μL, 19.2 mg, 0.64 μmol, prepared as described in Example 20) in 50 mM PBS, pH 7.4 was added. After stirring ˜1 h at room temperature, the reaction mixture was concentrated by centrifugation on a 30 K MWCO membrane and purified by CEX chromatography to give the title compound. Yield 8% based on protein; AF-HPA to trastuzumab-Fab′ ratio was about 6:1 to about 8:1.

### Example 24: 22 K PHF-GA-6-maleimido hexanamide-Auristatin F-hydroxypropylamide-L-Alanine-Anti-Her2 Affibody (i.e., (Anti-Her2 affibody)-(N-(6-maleimidohexanamido)ethylamino-22 kDa PHF-GA-Auristatin F-hydroxypropylamide-L-Alanine)

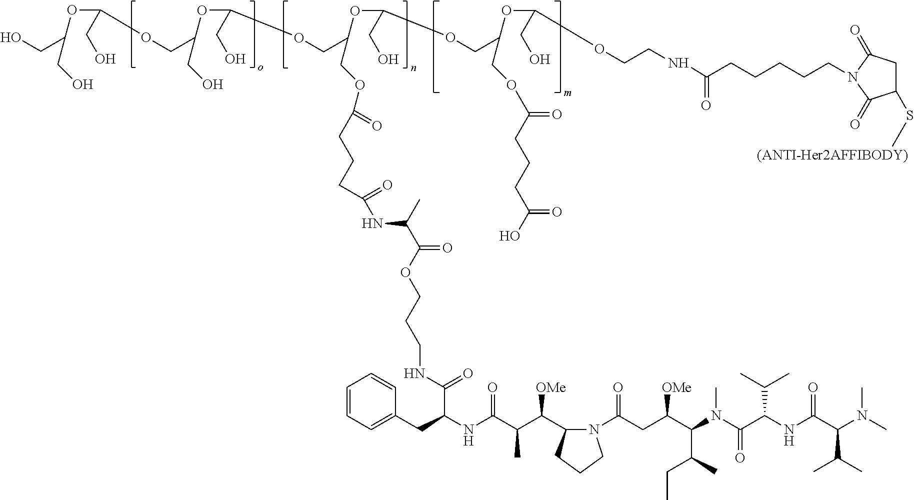

To a solution of Anti-Her2 Affibody (0.85 mL, 1.5 mg, 0.107 μmol) in TEAA buffer, pH 7.4 was added a solution of TCEP (36.8 μL, 0.0921 mg, 0.321 μmol) in TEAA buffer, pH 7.4 and the mixture was incubated 1 h at 37° C. The crude reduced protein was then purified by SEC (TSKgelG5000PW, PBS, pH 7.4, 20% CH₃CN) to remove residual DTT. A solution of 22 K PHF-GA (21%)-6-maleimido hexanamide (1.81%)-Auristatin F-hydroxypropylamide-L-Alanine (2.51%) ((N-(6-maleimido)hexanamido) (1.81%) ethylamino-22 kDa PHF-GA-Auristatin F-hydroxypropylamide-L-Alanine (2.51%) (114 μL, 2.49 mg, 0.089 μmol, prepared as described in Example 20) in NH₄OAc buffer was added. After stirring ˜2 h at room temperature and quantitative consumption of the Anti-Her2 Affibody, the reaction mixture was concentrated and purified by centrifugation on a 10 K MWCO membrane to give the title conjugate. Yield 80%; AF-HPA to anti-Her2 Affibody ratio was about 16:1 to about 20:1.

### Example 25: Assay for Activity of Terminally Modified PHF Exendin Conjugates

The activity of terminally modified PHF exendin conjugates were measured using DiscoveRx cAMP Hunter express GPCR Assay which determines the cellular formation of a second messenger such as cAMP. Exendin-4, a glucagon-like peptide-1 agonist (GLP-1 agonist) displays biological properties similar to human glucagon-like peptide-1 (GLP-1). It binds to glucagon-like peptide-1 receptor 1-G-protein coupled receptor (GPCR). Following stimulation of this receptor, intracellular signaling pathways are activated that lead to the production of intracellular second messengers, such as cAMP. Free cAMP molecules from cell lysates compete for antibody binding with a labeled enzyme donor (ED)-cAMP conjugate, which contains a small peptide fragment of β-galactosidase. In the absence of free cAMP, the ED-cAMP conjugates are captured by the cAMP-specific antibody and are unavailable for complementation with the enzyme acceptor (EA), resulting in a low signal. In the presence of free cAMP, antibody sites are occupied, allowing the ED-cAMP conjugate to complement with EA, forming an active β-galactosidase enzyme; substrate hydrolysis by this enzyme produces a chemiluminescent signal. The signal generated is in direct proportion to the amount of free cAMP bound by the antibody. GLP1R CHO-K1 Gs overexpressing cells were plated in 96-well plate and allowed to adhere overnight at 37° C. in a humidified atmosphere of 5% CO₂. The next day cells were washed with PBS, cAMP-specific antibody and Exendin-4-Cys, or Example 15 (50 kDa PHF-GA-6-maleimido hexanamide-Exendin-4-Cys) ((Exendin-4-Cys)-(N-(2-maleimido)propanamido)propyl)-ethylamino-50 kDa PHF) were added and incubated for 30 min at 37° C.; followed by the addition of the detection reagent were added and then the cells were incubated for 3 h at room temperature. Luminescent signal was measured using a SpectraMax M5 plate reader (Molecular Devices). Dose response curves were generated using SoftMAx pro software. EC₅₀ values were determined from four-parameter curve fitting.

Table I give illustrative results for activity of unconjugated Exendin-4-Cys, or Example 15 (Exendin-4-50 kDa PHF conjugate).

TABLE I

| | EC₅₀ nmol/L |
|---|---|
| Example 15 | 1.87 |
| Exendin-4-Cys | 0.21 |

### Example 26: Cell Viability Assay for PBRM-Drug Polymer Conjugates

PBRM-drug compound polymer conjugates are evaluated for their tumor viability using Cell Titer-Glo (Promega Corp). Cells are plated in black walled 96-well plate and allowed to adhere overnight at 37° C. in a humidified atmosphere of 5% CO₂. HER2 expressing cells SKBR₃, BT474, NCI-N87 and cells expressing low levels of HER2-MCF7 are plated at a density of 5,000 cells per well. The next day the medium is replaced with 50 L fresh medium and 50 L of 2x stocks of PBRM-drug polymer conjugate, drug compound polymer conjugate or drug compound is added to appropriate wells, mixed and incubated for 72 h. Cell Titer-Glo reagent is added to the wells at room temperature and the luminescent signal is measured after 10 min using a SpectraMax M5 plate reader (Molecular Devices). Dose response curves are generated using SoftMax Pro software. IC₅₀ values are determined from four-parameter curve fitting.

### Example 27: In vivo Efficacy, Pharmacokinetic and Biodistribution Studies

In order to evaluate the efficacy and pharmacokinetics of the protein drug conjugate mouse and rat subcutaneous and orthotopic xenograft models are used.

Test articles, along with appropriate controls are administered intravenously (IV) via tail-vein injection or intraperitoneally. To assess circulating levels of test article blood sample is collected at designated times via terminal cardiac-puncture. Samples are kept at room temperature for 30 min to coagulate, then centrifuged for 10 min at 1,000×g at 4° C. and immediately frozen at −80° C. Total PBRM concentrations in serum samples are measured using ELISA. Circulating drug compound concentration (conjugated and free) is determined by LC/MS methods.

To assess efficacy of the PBRM-drug compound polymer conjugates the tumor size are measured using digital calipers. Tumor volume is calculated and used to determine the delay in tumor growth.

For the determination of drug biodistribution, tumor, and major organs such as, for example, liver, kidney, spleen, lung, heart, muscles, and brain are harvested, immediately frozen in liquid nitrogen, stored at −80° C. PBRM and/or drug compound levels are determined in tissue homogenates by standard methods, such as, for example, ELISA or LC/MS/MS methods respectively.

### Example 28: Tumor Growth Response to Administration of PBRM-drug polymer conjugates

Female CB-17 SCID mice are inoculated subcutaneously with NCI-N87 cells (n=10 for each group) or BT474 tumors (n=12 or n=10 for each group). Test compounds or vehicle are dosed IV as a single dose on day 1; once every week for 3 weeks on day 1, day 8 and day 15 respectively; or once every week for 3 weeks on day 17, day 24 and day respectively. The drug compound polymer conjugate dose is determined such that it delivered the same amount of drug compound as that present in the highest dose of the corresponding PBRM-drug compound polymer conjugate is administered Tumor size is measured at several different time points using digital calipers. Tumor volume is calculated and is used to determine the delay in tumor growth. Mice are sacrificed when tumors reach a size of 1000 mm³, 800 mm³, or 700 mm³. Tumor volumes are reported as the mean±SEM for each group.

### Example 29: In Vitro Stability of PBRM-Drug Compound Polymer Conjugates

The in vitro stability of PBRM-drug compound polymer conjugates are evaluated by incubation of the PBRM-drug compound polymer conjugate in physiological saline or animal plasma at 37° C., pH 7.4. The rate of PBRM-drug compound polymer conjugate degradation is determined by monitoring the amount of drug released into the matrix by LC/MS analysis after isolation of released drug from the PBRM-drug compound polymer conjugate by liquid-liquid extraction.

### Example 30: Ligand Binding Studies by BIAcore Surface Plasmon Resonance (SPR)

The kinetic binding of the PBRM-drug compound polymer conjugate to an immobilized receptor is determined by BIAcore SPR. The binding constants for the PBRM in the PBRM-drug compound-conjugate and PBRM alone are determined using standard BIAcore procedures.

### Example 31: Mouse Plasma PK and Tissue Distribution after Administration of PBRM-Drug Compound Polymer Conjugates

The plasma PK stability and the tissue distribution of PBRM-drug compound-conjugate is determined after administration of PBRM-drug compound-conjugate in female CB-17 SCID mice with NCI-N87 tumors (n=3). The conjugated drug compound concentration is determined by LC/MS analysis. The concentration of the drug compound-PBRM-conjugate is estimated from the conjugated drug compound data. Total PBRM concentration is determined by ELISA.

### INCORPORATION BY REFERENCE

The entire disclosure of each of the patent documents and scientific articles referred to herein is incorporated by reference for all purposes.

### EQUIVALENTS

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A terminally modified polymer for covalently conjugating with a pharmaceutically useful modifier (“M”) prepared by a process comprising:
(i) providing a poly(1-hydroxymethylene hydroxymethyl-formal) (“PHF”) that has a terminal aldehyde group with the structure: 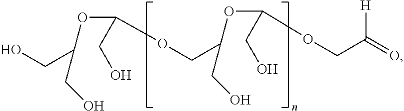
(ii) reductively aminating the terminal aldehyde group to form a terminal amino group; and
(iii) modifying the terminal amino group so as to generate the terminally modified polymer, wherein the terminally modified polymer is of the following structure: 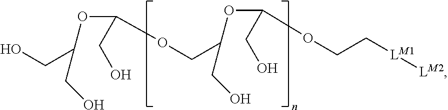
wherein n is an integer between 1 and about 1100,
L^{M1} is —NR¹, —NR¹C(═X¹)—, —NR¹C(═X¹)Y—, —NR¹NR²—, —NR¹NR²C(═X¹)—, —NR¹NR²C(═X¹)Y—, —NR¹SO₂—, or —NR¹SO₂NR²—, with the NR¹ moiety attached to the polymer in the order as written, in which X¹ is O, S, or NR³ and Y is O, S, or NR⁴, and each of R¹, R², R³, and R⁴ independently is H or an aliphatic, heteroaliphatic, carbocyclic, or heterocyclic moiety, and
L^{M2} is —(CH₂)ₘ—W, in which m is an integer between 0 and 20, and W is a functional group suitable for covalently conjugating with M or W is an aliphatic, heteroaliphatic, carbocyclic, or heterocyclic moiety, wherein the aliphatic, heteroaliphatic, carbocyclic, or heterocycloalkyl moiety comprises a functional group suitable for covalently conjugating with M.

2. The terminally modified polymer of claim 1, wherein W, when not conjugated with M, is selected from 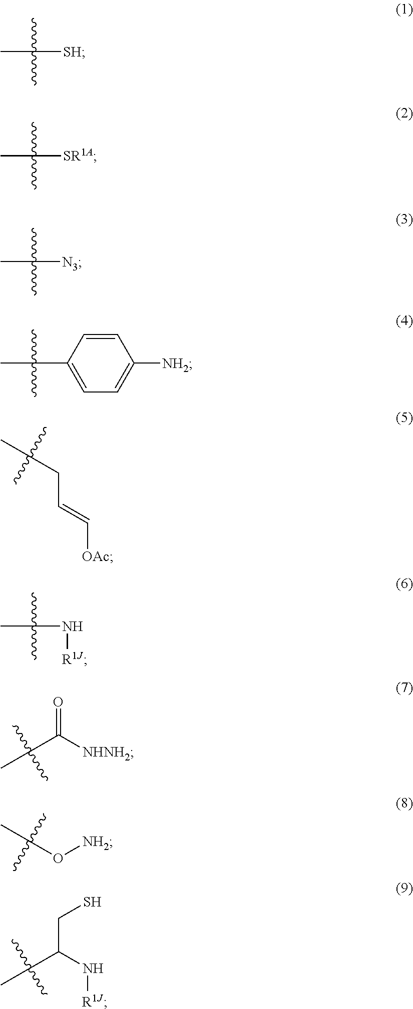 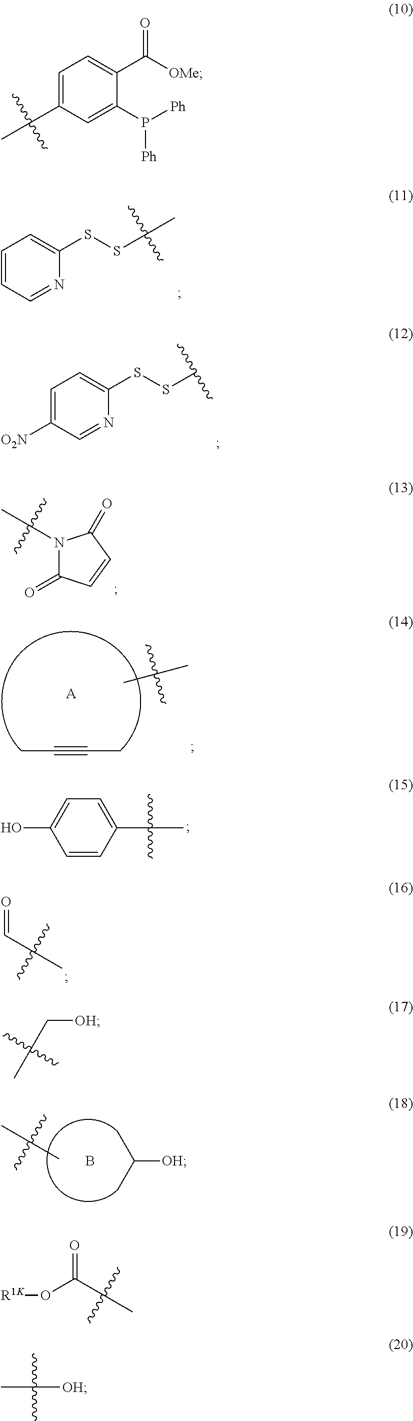 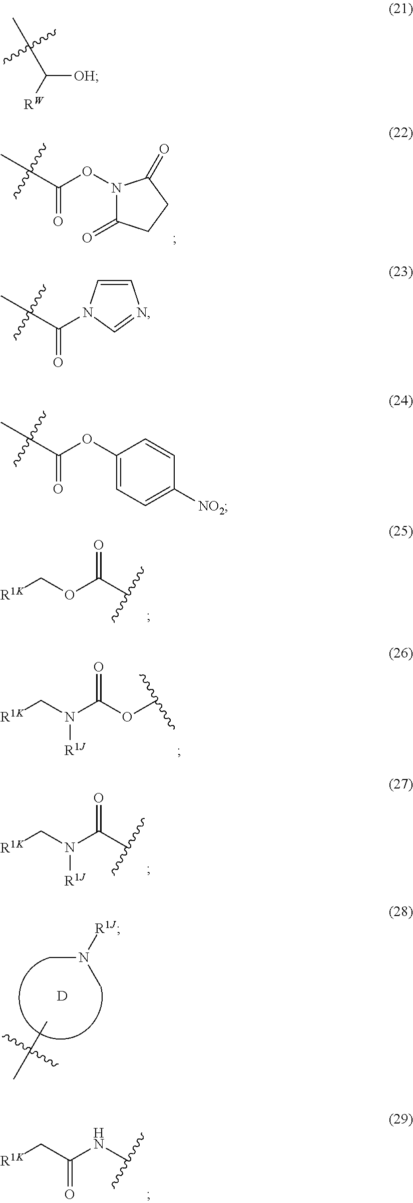 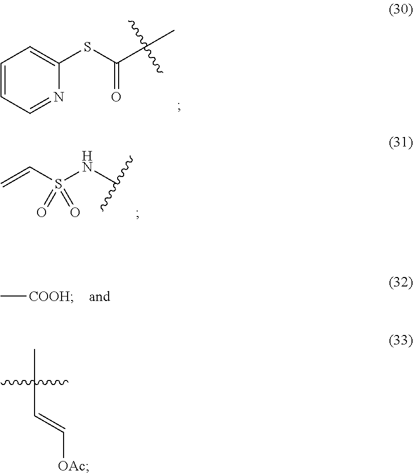
in which R^{1A} is a sulfur protecting group, each of ring A and B, independently, is cycloalkyl or heterocycloalkyl, R^{W} is an aliphatic, heteroaliphatic, carbocyclic or heterocycloalkyl moiety; ring D is heterocycloalkyl; R^{1J} is hydrogen, or an aliphatic, heteroaliphatic, carbocyclic, or heterocycloalkyl moiety; and R^{1K} is a leaving group.

3. The terminally modified polymer of claim 1, wherein each of R¹, R², R³ and R⁴ independently is H, or unsubstituted or substituted C₁₋₆ alkyl.

4. A polymer conjugate comprising a terminally modified polymer covalently conjugated to a pharmaceutically useful modifier (“M”), the polymer conjugate being of formula (I): 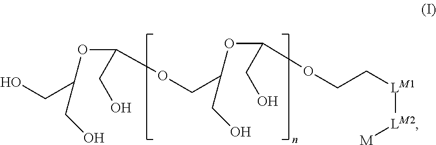
wherein
n is an integer between 1 and about 1100,
L^{M1} is —NR¹, —NR¹C(═X¹)—, —NR¹C(═X¹)Y—, —NR¹NR²—, —NR¹NR²C(═X¹)—, —NR¹NR²C(═X¹)Y—, —NR¹SO₂—, or —NR¹SO₂NR²—, with the NR¹ moiety attached to the polymer in the order as written, in which X¹ is O, S, or NR³ and Y is O, S, or NR⁴, and each of R¹, R², R³, and R⁴ independently is H or an aliphatic, heteroaliphatic, carbocyclic, or heterocyclic moiety,
L^{M2} is —(CH₂)ₘ—W, with (CH₂)ₘ connected to L^{M1}, in which m is an integer between 0 and 20, and W, prior to conjugating with M, is a functional group suitable for covalently conjugating with M or W is an aliphatic, heteroaliphatic, carbocyclic, or heterocyclic moiety, wherein the aliphatic, heteroaliphatic, carbocyclic, or heterocycloalkyl moiety comprises a functional group suitable for covalently conjugating with M;
M is selected from the group consisting of proteins, antibodies, antibody fragments, and peptides; and
wherein the polymer conjugate further comprises at least one therapeutic agent having a molecular weight ≦5 kDa (“D”) connected to the backbone of the polymer, wherein each of the at least one D is connected to the backbone via L^{D}, wherein L^{D} is a linker having the structure: 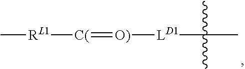
in which R^{L1} is connected to an oxygen atom of the polymer and L^{D1} is connected to D, R^{L1} is absent, alkyl, heteroalkyl, cycloalkyl, or heterocycloalkyl, 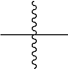
denotes direct or indirect attachment of D to L^{D1}, and L^{D1} is a carbonyl-containing moiety suitable for connecting D to the backbone of the polymer and L^{D1} contains a functional group that is capable of forming a covalent bond with a functional group of D.

5. The polymer conjugate of claim 4, wherein L^{M1}-L^{M2} further comprises 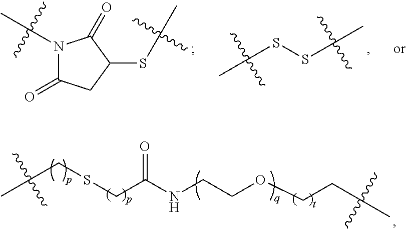
in which q is an integer from 0 to 12 and each of p and t independently is an integer from 0 to 3.

6. The polymer conjugate of claim 4, wherein L^{M1}-L^{M2} further comprises 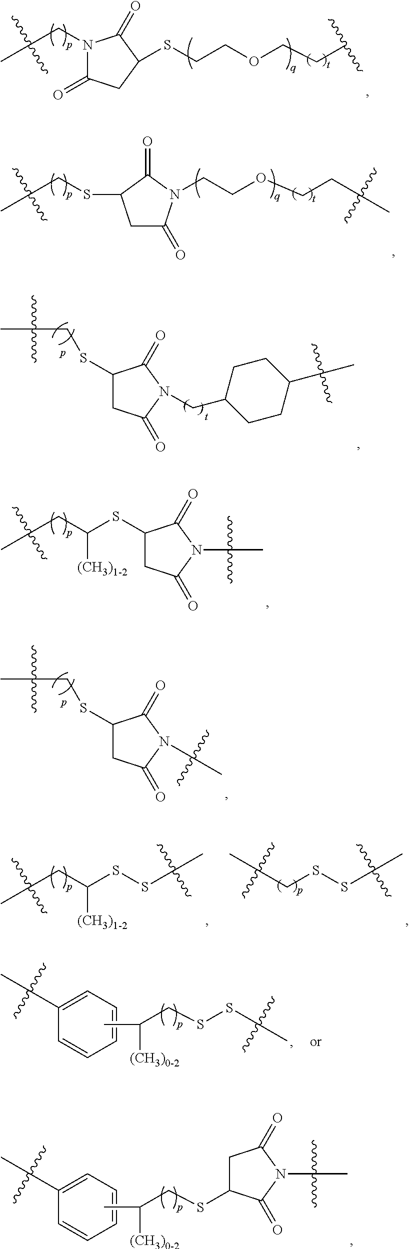
in which q is an integer from 0 to 12 and each of p and t independently is an integer from 0 to 3.

7. The polymer conjugate of claim 4, wherein M is a protein based recognition molecule having a molecular weight ≦200 kDa and PHF has a molecular weight of about 20 kDa to about 75 kDa.

8. The polymer conjugate of claim 4, wherein M is a protein based recognition molecule having a molecular weight ≧40 kDa and PHF has a molecular weight of about 2 kDa to about 25 kDa.

9. A composition comprising the polymer conjugate of claim 4 and a pharmaceutically suitable carrier.

10. A method of synthesizing the terminally modified polymer of claim 1, the method comprising:
providing a PHF that has a terminal aldehyde group with the structure: 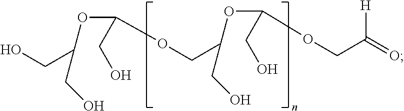
reductively aminating the terminal aldehyde group to form a terminal amino group (NH₂); and
modifying the terminal amino group so as to generate the terminally modified polymer of claim 1.

11. A method of synthesizing a terminally modified polymer of claim 1, the method comprising:
providing a PHF, having at least one terminus that is —O—(CH₂)₂—NH₂; and
reacting the —O—(CH₂)₂—NH₂ with 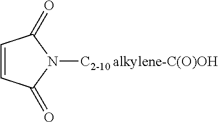
to generate the terminally modified polymer of claim 1.
